(19) United States
(12) Patent Application Publication
(10) Pub. No.: US 2025/0360148 A1
(43) Pub. Date: Nov. 27, 2025

(54) SOLID DISPERSIONS OF PSILOCYBIN

(71) Applicant: Cybin IRL Limited, Dublin (IE)

(72) Inventors: Alex Nivorozhkin, West Roxbury, MA (US); Michael Palfreyman, St. Petersburg, FL (US); Mohammed I. Shukoor, Lexington, MA (US); Kenneth L. Avery, Merrimack, NH (US); Pradip M. Pathare, Lexington, MA (US)

(73) Assignee: Cybin IRL Limited, Dublin (IE)

(21) Appl. No.: 18/867,231

(22) PCT Filed: Jun. 22, 2023
(86) PCT No.: PCT/EP2023/066900

§ 371 Date: Nov. 19, 2024

Related U.S. Application Data
(60) Provisional application No. 63/366,788, filed on Jun. 22, 2022.

(51) Int. Cl. A61K 31/675; A61K 9/16; A61K 9/20

(52) U.S. Cl. CPC A61K 31/675; A61K 9/1611; A61K 9/1623; A61K 9/1635; A61K 9/1652; A61K 9/1658; A61K 9/2009; A61K 9/2018; A61K 9/2027; A61K 9/2054; A61K 9/2063

(57) ABSTRACT

The present disclosure relates to stable pharmaceutical compositions of amorphous psilocybin and deuterated psilocybin, and to the use of such pharmaceutical compositions in the treatment of diseases associated with a serotonin 5-HT₂ receptor. The pharmaceutical compositions are formulated with solid dispersions of psilocybin or deuterated psilocybin in amorphous form dispersed in a polymer.

### CROSS-REFERENCE

This application claims priority to U.S. Provisional Application No. 63/366,788, filed on Jun. 22, 2022, incorporated by reference herein in its entirety.

### FIELD

The present disclosure relates generally to compositions of psilocybin and/or deuterated psilocybin and, in some embodiments, to serotonin 5-HT₂ receptor agonists and uses in the treatment of diseases associated with a 5-HT₂ receptor.

### BACKGROUND

Psilocybin (PY) and psilocin (PI) are tryptamine alkaloids and structural analogs of the neurotransmitter serotonin. Psilocybin is a prodrug of psilocin. That is, when consumed, psilocybin is rapidly metabolized into the active form, psilocin. Specifically, a chemical process called dephosphorylation removes the phosphate group on psilocybin, creating psilocin.

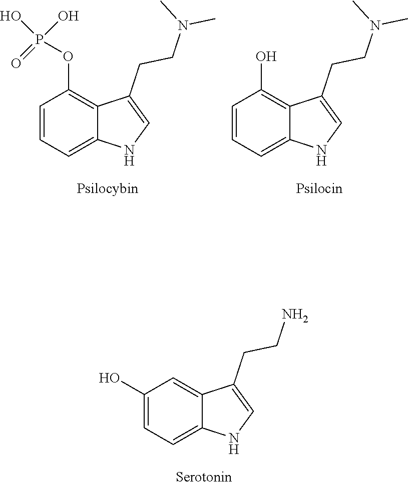 [SMILES: CN(C)CCc1c[nH]c2cccc(OP(=O)(O)O)c12 ; CN(C)CCc1c[nH]c2cccc(O)c12 ; NCCc1c[nH]c2ccc(O)cc12]

Psilocin is a short-lived and unstable molecule. Therefore, therapeutic applications involving the use of psilocin are generally accomplished by administration of the precursor, psilocybin. However, psilocybin has a slow onset of drug action (1.5-2 hours) and a long duration of drug action (2-3 hours), often requiring 7-8 hours of supervised clinical observation of a patient before discharge. Therefore, there is a need for a psilocybin formulation that has a faster/quicker therapeutic onset and a shorter duration of drug action (i.e., shorter duration of therapeutic effect) than current therapeutic applications of psilocybin.

Generally, amorphous drug forms tend to have improved solubility in water compared to their crystalline counterparts, and thus can give rise to markedly improved pharmaceutical performance such as faster onset, higher bioavailability, etc.—however, in the case of psilocybin, the amorphous form is unstable and has a tendency to crystallize. See Greenan et al., Preparation and Characterization of Novel Crystalline solvates and Polymorphs of Psilocybin and Identification of Solid Forms Suitable for Clinical Development, 2020 pre-publication; DOI:10.13140/RG.2.2.32357.14560.

Further, there are several common diseases, disorders, and conditions for which no adequate treatment, therapies, or cure exist, including:
(1) anxiety disorders, which are a class of psychiatric disorders that involve extreme fear or worry. Oftentimes, anxiety disorders interfere with daily activities, such as job performance, school work, and relationships. Anxiety disorders have a variety of causes, including changes in the brain, environmental stress, and genetics. Examples of anxiety disorders include acute stress disorder, anxiety due to a medical condition, generalized anxiety disorder, panic disorder, panic attack, a phobia, post-traumatic stress disorder, obsessive-compulsive disorder, separation anxiety disorder, social anxiety disorder, substance-induced anxiety disorder, and selective mutism;
(2) headache disorders, which are characterized by recurrent headaches and are among the most common disorders of the nervous system. Headaches are a disabling feature of headache disorders, such as migraine, tension-type headache, and cluster headaches; and
(3) eating disorders, which are characterized by irregular eating habits and severe distress or concern about body weight or shape. Eating disturbances may include inadequate or excessive food intake which can ultimately damage an individual's physical and/or psychological health. Examples of eating disorders include pica, anorexia nervosa, bulimia nervosa, rumination disorder, avoidant/restrictive food intake disorder, and binge-eating disorder.

### SUMMARY

In view of the forgoing, there is a need for new psilocybin compositions that allow psilocybin to stably exist primarily in amorphous form with an extended shelf life. The present disclosure is based at least in part on the identification of stable formulations of amorphous psilocybin and/or deuterated psilocybin which prevent/reduce amorphous to crystalline transitions, and which demonstrate improved pharmaceutical performance (e.g., faster/quicker therapeutic onset, a shorter duration of drug action) compared to crystalline dosage forms.

More specifically, the present disclosure provides stable compositions of amorphous psilocybin and/or deuterated psilocybin that modulate serotonin 5-HT₂ receptors and methods of using the same to treat diseases associated with a serotonin 5-HT₂ receptor, including anxiety disorders, headache disorders, and eating disorders. The present disclosure also provides novel compositions of amorphous psilocybin and/or deuterated psilocybin that permit, for example, once-daily dosing to selectively engage 5-HT_{2A}Rs without producing psychedelic effects, and to treat neuropsychiatric and other disorders associated with inflammation. Use of the compositions of the present disclosure may provide numerous clinical benefits, such as benefits in neural plasticity and cognitive function (as measured using e.g., Cambridge Neuropsychological Test Automated Battery (CANTAB) tests) with improvements in, for example, working memory and executive function, sustained attention, and episodic memory.

These benefits have implications for use in the treatment of various diseases, disorders, and conditions, including both psychiatric and neurological aspects thereof.

Thus, the present disclosure provides:

(1) A pharmaceutical composition, comprising:
a solid dispersion comprising a therapeutically effective amount of a compound of Formula (I) in amorphous form dispersed in a polymer,

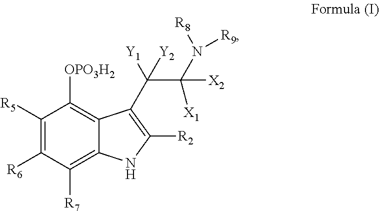

or a pharmaceutically acceptable salt, stereoisomer, a tautomer, or solvate thereof,
wherein:
R₂, R₅, R₆, and R₇ are independently selected from the group consisting of hydrogen and deuterium,
R₈ and R₉ are independently selected from the group consisting of —CH₃ and —CD₃, and
X₁, X₂, Y₁, and Y₂ are independently selected from the group consisting of hydrogen and deuterium.

(2) The pharmaceutical composition of (1), wherein R₂, R₅, R₆, and R₇ are hydrogen.

(3) The pharmaceutical composition of (1), wherein R₂, R₅, R₆, and R₇ are deuterium.

(4) The pharmaceutical composition of any one of (1) to (3), wherein R₈ and R₉ are —CH₃.

(5) The pharmaceutical composition of any one of (1) to (3), wherein R₈ and R₉ are —CD₃.

(6) The pharmaceutical composition of any one of (1) to (5), wherein X₁, X₂, Y₁, and Y₂ are deuterium.

(7) The pharmaceutical composition of any one of (1) to (6), wherein X₁ and X₂ are deuterium.

(8) The pharmaceutical composition of any one of (1) to (7), wherein Y₁ and Y₂ are deuterium.

(9) The pharmaceutical composition of any one of (1) to (5), wherein Y₁ and Y₂ are hydrogen.

(10) The pharmaceutical composition of any one of (1) to (9), wherein the compound of Formula (I) is at least one selected from the group consisting of:

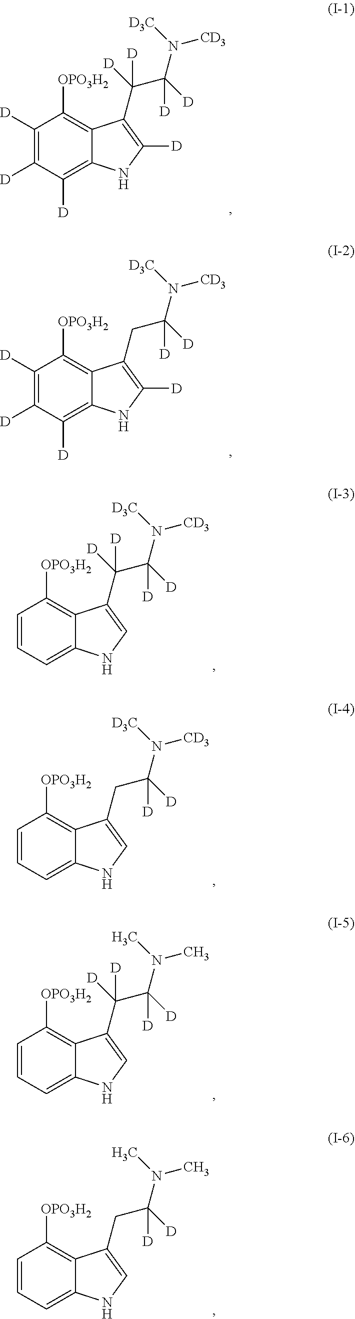

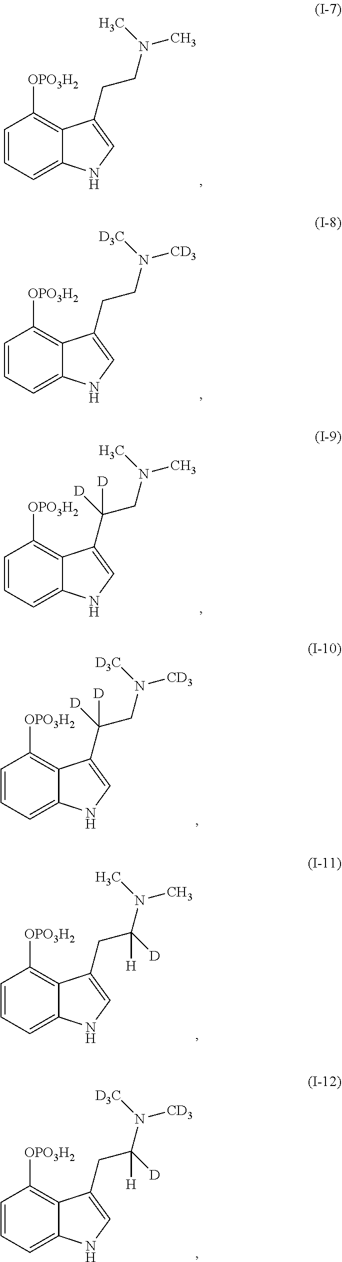

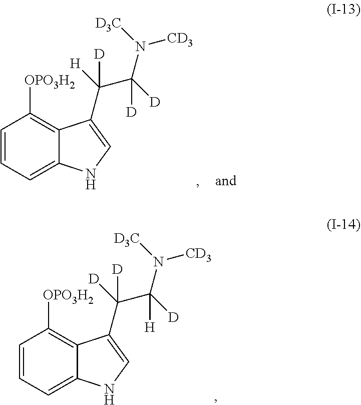

or a pharmaceutically acceptable salt, stereoisomer, a tautomer, or solvate thereof.

(11) The pharmaceutical composition of (1), wherein the compound of Formula (I) is

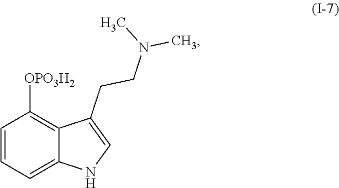

or a pharmaceutically acceptable salt, tautomer, or solvate thereof.

(12) The pharmaceutical composition of any one of (1) to (11), wherein the compound of Formula (I) is an agonist of a serotonin 5-HT₂ receptor.

(13) The pharmaceutical composition of any one of (1) to (12), wherein the compound of Formula (I) is an agonist of a serotonin 5-HT_{2A} receptor.

(14) The pharmaceutical composition of any one of (1) to (13), wherein the solid dispersion is a solid molecular complex.

(15) The pharmaceutical composition of any one of (1) to (14), wherein the compound of Formula (I) is present in the solid dispersion in an amount of 0.1 wt. % to 90 wt. %, based on a total weight of the solid dispersion.

(16) The pharmaceutical composition of any one of (1) to (15), wherein a weight ratio of the compound of Formula (I) to the polymer in the solid dispersion is from 1:9 to 9:1.

(17) The pharmaceutical composition of any one of (1) to (16), wherein the polymer is at least one selected from the group consisting of a vinyl polymer, a methacrylate, a polysaccharide, gelatin, and a cellulose polymer, or a blend or a copolymer thereof.

(18) The pharmaceutical composition of any one of (1) to (17), wherein the polymer is at least one selected from the group consisting of gelatin, polyvinyl acetate, polyvinyl alcohol, polyvinylpyrrolidone, pullulan, hydroxypropyl methyl cellulose, hydroxypropyl methyl cellulose acetate succinate, and a methacrylate copolymer, or a blend or a copolymer thereof.

(19) The pharmaceutical composition of any one of (1) to (18), wherein the polymer comprises gelatin.

(20) The pharmaceutical composition of (19), wherein the solid dispersion further comprises a pharmaceutically acceptable excipient.

(21) The pharmaceutical composition of (20), wherein the pharmaceutically acceptable excipient comprises mannitol.

(22) The pharmaceutical composition of any one of (1) to (21), wherein the polymer comprises a copolymer of vinyl pyrrolidone and vinyl acetate (PVP-VAc).

(23) The pharmaceutical composition of any one of (1) to (22), wherein the polymer comprises a methacrylate copolymer.

(24) The pharmaceutical composition of any one of (1) to (23), wherein the polymer comprises a cellulose polymer.

(25) The pharmaceutical composition of (24), wherein the cellulose polymer has a weight average molecular weight of from 150,000 g/mol to 5,000,000 g/mol.

(26) The pharmaceutical composition of (24), wherein the cellulose polymer has a weight average molecular weight of from 1,000 g/mol to 100,000 g/mol.

(27) The pharmaceutical composition of any one of (24) to (26), wherein the cellulose polymer is hydroxypropyl methyl cellulose acetate succinate.

(28) The pharmaceutical composition of any one of (24) to (26), wherein the cellulose polymer is hydroxypropyl methyl cellulose.

(29) The pharmaceutical composition of any one of (1) to (18), (24) to (26), or (28), wherein the polymer is a blend of hydroxypropyl methyl cellulose and polyvinylpyrrolidone.

(30) The pharmaceutical composition of any one of (1) to (29), further comprising a pharmaceutically acceptable excipient which is not dispersed with the solid dispersion.

(31) The pharmaceutical composition of any one of (1) to (30), further comprising sodium phosphate and/or a natural amino acid.

(32) The pharmaceutical composition of any one of (1) to (31), wherein the solid dispersion has a glass transition (Tg) onset of from 110° C. to 200° C., as determined by modulated differential scanning calorimetry (mDSC).

(33) The pharmaceutical composition of any one of (1) to (32), wherein the solid dispersion has a heat capacity change (ΔCp), in J/(g ° C.), of from 0.1 to 0.75, as determined by modulated differential scanning calorimetry (mDSC).

(34) The pharmaceutical composition of any one of (1) to (33), wherein the compound of Formula (I) is present in an amount of 0.1 to 1000 mg.

(35) The pharmaceutical composition of any one of (1) to (34), which is adapted for intraoral administration.

(36) The pharmaceutical composition of (35), wherein the pharmaceutical composition is in orodispersible dosage form.

(37) The pharmaceutical composition of (35) or (36), wherein the pharmaceutical composition is in a form of lyophilized fast dissolving tablets.

(38) The pharmaceutical composition of (35) or (36), wherein the pharmaceutical composition is in a form of lyophilized wafers.

(39) The pharmaceutical composition of any one of (1) to (34), which is adapted for oral administration.

(40) The pharmaceutical composition of any one of (1) to (34) or (39), which is adapted for extended-release.

(41) A method of treating a subject with a disease or disorder, comprising: administering to the subject the pharmaceutical composition of any one of (1) to (40).

(42) A method of treating a subject with a disease or disorder associated with a serotonin 5-HT₂ receptor, comprising: administering to the subject the pharmaceutical composition of any one of (1) to (40).

(43) The method of (42), wherein the disease or disorder is a neuropsychiatric disease or disorder or an inflammatory disease or disorder.

(44) The method of (42), wherein the disease or disorder is a central nervous system (CNS) disorder.

(45) The method of (44), wherein the central nervous system (CNS) disorder is at least one selected from the group consisting of major depressive disorder (MDD), treatment-resistant depression (TRD), post-traumatic stress disorder (PTSD), bipolar and related disorders, obsessive-compulsive disorder (OCD), generalized anxiety disorder (GAD), social anxiety disorder, a substance use disorder, an eating disorder, Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, suicidal ideation, suicidal behavior, major depressive disorder with suicidal ideation or suicidal behavior, melancholic depression, atypical depression, dysthymia, non-suicidal self-injury disorder (NSSID), chronic fatigue syndrome, Lyme's disease, gambling disorder, a paraphilic disorder, sexual dysfunction, peripheral neuropathy, and obesity.

(46) The method of (44), wherein the central nervous system (CNS) disorder is major depressive disorder (MDD).

(47) The method of (44), wherein the central nervous system (CNS) disorder is treatment-resistant depression (TRD).

(48) The method of (44), wherein the central nervous system (CNS) disorder is generalized anxiety disorder (GAD).

(49) The method of (44), wherein the central nervous system (CNS) disorder is social anxiety disorder.

(50) The method of (44), wherein the central nervous system (CNS) disorder is obsessive-compulsive disorder (OCD).

(51) The method of (44), wherein the central nervous system (CNS) disorder is cluster headaches or migraine.

(52) The method of (44), wherein the central nervous system (CNS) disorder is a substance use disorder.

(53) The method of (52), wherein the substance use disorder is alcohol use disorder.

(54) The method of (42), wherein the disease or disorder is mental distress in frontline healthcare workers.

(55) The method of (42), wherein the disease or disorder is an autonomic nervous system (ANS) condition.

(56) The method of (42), wherein the disease or disorder is a pulmonary disorder.

(57) The method of (42), wherein the disease or disorder is a cardiovascular disorder.

(58) The method of any one of (42) to (57), wherein the pharmaceutical composition is administered orally to the subject.

(59) The method of any one of (42) to (57), wherein the pharmaceutical composition is administered intraorally to the subject.

(60) The method of any one of (42) to (57), wherein the pharmaceutical composition is administered subcutaneously to the subject.

(61) The method of any one of (42) to (60), wherein the pharmaceutical composition is administered to provide the compound of Formula (I) to the subject at a psychedelic dose of about 0.083 mg/kg to about 1 mg/kg.

(62) The method of any one of (42) to (60), wherein the pharmaceutical composition is administered to provide the compound of Formula (I) to the subject at a sub-psychedelic dose of about 0.00001 mg/kg to less than about 0.083 mg/kg.

(63) The method of (42), wherein the disease or disorder is a neurological or neurodegenerative disease.

(64) The method of (63), wherein the neurological or neurodegenerative disease is Alzheimer's disease or other dementia subtype or Parkinson's disease.

(65) The method of (63) or (64), wherein the method reduces neuroinflammation in the subject compared to neuroinflammation prior to treatment commencement.

(66) The method of any one of (63) to (65), wherein the pharmaceutical composition is administered to provide the compound of Formula (I) to the subject at a sub-psychedelic dose of about 0.00001 mg/kg to less than about 0.083 mg/kg.

(67) The method of any one of (63) to (66), wherein the pharmaceutical composition is in an oral and/or extended-release dosage form.

(68) The method of any one of (63) to (67), wherein the neurological or neurodegenerative disease is Alzheimer's disease.

(69) The method of (68), wherein the method treats depression, anxiety, and/or stress associated with Alzheimer's disease.

(70) A method for decreasing time of therapeutic onset relative to a crystalline psilocybin-based drug, comprising:
administering the pharmaceutical composition of any one of (1) to (40) to a subject in need thereof.

(71) A method of reducing psychedelic side effects relative to a crystalline psilocybin-based drug, comprising:
administering the pharmaceutical composition of any one of (1) to (40) to a subject in need thereof

(72) A method of decreasing duration of therapeutic effect compared to a crystalline psilocybin-based drug, comprising:
administering the pharmaceutical composition of any one of (1) to (40) to a subject in need thereof.

(73) A method of treating a subject with one or more of an anxiety disorder, an eating disorder, and a headache disorder, comprising:
administering to the subject the pharmaceutical composition of any one of (1) to (40).

(74) A method of treating a subject with an anxiety disorder, comprising: administering to the subject the pharmaceutical composition of any one of (1) to (40).

(75) A method of treating a subject with an obsessive-compulsive and related disorder, comprising:
administering to the subject the pharmaceutical composition of any one of (1) to (40).

(76) A method of treating a subject with post-traumatic stress disorder, comprising:
administering to the subject the pharmaceutical composition of any one of (1) to (40).

(77) A method of treating a subject with a headache disorder, comprising:
administering to the subject the pharmaceutical composition of any one of (1) to (40).

(78) A method of treating a subject with a cluster headache, comprising:
administering to the subject the pharmaceutical composition of any one of (1) to (40).

(79) A method of treating a subject with a migraine, comprising:
administering to the subject the pharmaceutical composition of any one of (1) to (40).

(80) A method of treating a subject with an eating disorder, comprising:
administering to the subject the pharmaceutical composition of any one of (1) to (40).

(81) A method of treating a subject with a binge-eating disorder, comprising:
administering to the subject the pharmaceutical composition of any one of (1) to (40).

(82) A method of treating a subject with anorexia nervosa, comprising:
administering to the subject the pharmaceutical composition of any one of (1) to (40).

(83) Use of the pharmaceutical compositions of any one of (1) to (40) for treating a subject with a disease or disorder associated with a serotonin 5-HT₂ receptor.

### BRIEF DESCRIPTION OF THE DRAWINGS

The forgoing paragraphs have been provided by way of general introduction and are not intended to limit the scope of the following claims. The described embodiments, together with further advantages, will be best understood by reference to the following detailed description when considered in conjunction with the accompanying drawings, wherein:

FIG. 1 is a synthetic route to psilocybin-d₁₀ (I-3);

FIG. 2 shows the calculated X-ray power diffraction (XRPD) pattern of psilocybin Form A (Cambridge structural database (CSD) Reference Code HATCAK & TAVZID)

FIG. 3 shows the calculated XRPD pattern of psilocybin Form B (CSD Reference Code TAVZID01);

FIG. 4 shows the calculated XRPD pattern of psilocybin methanol solvate (CSD Reference Code PSILOC);

FIG. 5 shows the calculated XRPD pattern of psilocybin trihydrate (CSD Reference Code OKOKAD);

FIG. 6 shows the XRPD pattern of crystalline psilocybin methanol solvate with a small quantity of Form B (commercially available from Quality Chemical Labs);

FIG. 7 shows the XRPD pattern of Reference Example 1b;

FIG. 8 shows the XRPD pattern of Reference Example 2b;

FIG. 9 shows the XRPD pattern of Reference Example 3b;

FIG. 10 shows the XRPD pattern of Reference Example 4b;

FIG. 11 shows the XRPD pattern of Reference Example 5b;

FIG. 12 shows the XRPD pattern of Example 1;

FIG. 13 shows the XRPD pattern of Example 3;

FIG. 14 shows the XRPD pattern of Example 4;

FIG. 15 shows the XRPD pattern of Example 2;

FIG. 16 shows the XRPD pattern of Example 5;

FIG. 17 shows the high-resolution XRPD pattern of Reference Example 5b;

FIG. 18 shows the high-resolution XRPD pattern of Example 5;

FIG. 19 shows the XRPD pattern of Reference Example 1a;

FIG. 20 shows the XRPD pattern of Reference Example 2a;

FIG. 21 shows the XRPD pattern of Reference Example 3a;

FIG. 22 shows the XRPD pattern of Reference Example 4a;

FIG. 23 shows the XRPD pattern of Reference Example 5a;

FIG. 24 shows the modulated differential scanning calorimetry (mDSC) profile of crystalline psilocybin methanol solvate with a small quantity of Form B (commercially available from Quality Chemical Labs);

FIG. 25 shows the mDSC profile of Example 1;

FIG. 26 shows the mDSC profile of Example 3;

FIG. 27 shows the mDSC profile of Example 4;

FIG. 28 shows the mDSC profile of Example 5;

FIG. 29 shows a graph of the dissolution/release rate of psilocybin from Example 1 at 1, 5, and 10 minute timepoints in 0.1 N citric acid (CA) and 1× phosphate buffered saline (PBS);

FIG. 30 shows a graph of the dissolution/release rate of psilocybin from Example 3 at 1, 5, and 10 minute timepoints in 0.1 N citric acid (CA) and 1× phosphate buffered saline (PBS);

FIG. 31 shows a graph of the dissolution/release rate of psilocybin from Example 4 at 1, 5, and 10 minute timepoints in 0.1 N citric acid (CA) and 1× phosphate buffered saline (PBS);

FIG. 32 shows a graph of the dissolution/release rate of psilocybin from Example 5 at 1, 5, and 10 minute timepoints in 0.1 N citric acid (CA) and 1× phosphate buffered saline (PBS);

FIG. 33 shows a graph of the dissolution/release rate of psilocybin from Example 7 at 1, 5, and 10 minute timepoints in 0.1 N citric acid (CA) and 1× phosphate buffered saline (PBS);

FIG. 34 shows a graph of the dissolution/release rate of psilocybin from Example 8 at 1, 5, and 10 minute timepoints in 0.1 N citric acid (CA) and 1× phosphate buffered saline (PBS);

FIG. 35 shows a graph of the dissolution/release rate of psilocybin from Example 9 at 1, 5, and 10 minute timepoints in 0.1 N citric acid (CA) and 1× phosphate buffered saline (PBS);

FIG. 36 shows a graph of the dissolution/release rate of psilocybin from Example 10 at 1, 5, and 10 minute timepoints in 0.1 N citric acid (CA) and 1× phosphate buffered saline (PBS);

FIG. 37 shows a graph of the dissolution/release rate of psilocybin from Example 11 at 1, 5, and 10 minute timepoints in 0.1 N citric acid (CA) and 1× phosphate buffered saline (PBS);

FIG. 38 shows a graph of the dissolution/release rate of psilocybin from Example 12 at 1, 5, and 10 minute timepoints in 0.1 N citric acid (CA) and 1× phosphate buffered saline (PBS);

FIG. 39 shows the high-resolution XRPD pattern of Example 1 after storage at 40° C., 75% relative humidity (RH) for 27 days;

FIG. 40 shows the high-resolution XRPD pattern of Example 1 after storage at 40° C., 15% relative humidity (RH) for 27 days;

FIG. 41 shows the high-resolution XRPD pattern of Example 1 after storage at room temperature for 27 days;

FIG. 42 shows the high-resolution XRPD pattern of Example 3 after storage at 40° C., 75% relative humidity (RH) for 27 days;

FIG. 43 shows the high-resolution XRPD pattern of Example 3 after storage at 40° C., 15% relative humidity (RH) for 27 days;

FIG. 44 shows the high-resolution XRPD pattern of Example 3 after storage at room temperature for 27 days;

FIG. 45 shows the high-resolution XRPD pattern of Example 4 after storage at 40° C., 75% relative humidity (RH) for 27 days;

FIG. 46 shows the high-resolution XRPD pattern of Example 4 after storage at 40° C., 15% relative humidity (RH) for 27 days;

FIG. 47 shows the high-resolution XRPD pattern of Example 4 after storage at room temperature for 27 days;

FIG. 48 shows the high-resolution XRPD pattern of Example 5 after storage at 40° C., 75% relative humidity (RH) for 24 days;

FIG. 49 shows the high-resolution XRPD pattern of Example 5 after storage at 40° C., 15% relative humidity (RH) for 24 days;

FIG. 50 shows the high-resolution XRPD pattern of Example 5 after storage at room temperature for 24 days;

FIG. 51 shows the XRPD pattern of Example 16 after storage at 40° C. for 6 hours (t=6 hr), 24 hours (t=24 hr), 1 week (t=1 w), and 4 weeks (t=4 w);

FIG. 52 shows the mDSC profile of Example 16 after initially being prepared (t=0);

FIG. 53 shows the mDSC profile of Example 16 after storage at 40° C. for 1 week (t=1 w) and 4 weeks (t=4 w);

FIG. 54 shows the thermogravimetric analysis (TGA) profile of Example 16 after initially being prepared (t=0) and after storage at 40° C. for 24 hours (t=24 hr) and 1 week (t 5=1 w);

FIG. 55 shows the TGA profile of Example 16 after storage at 40° C. for 1 week (t=1 w) and 4 weeks (t=4 w);

FIG. 56 shows the XRPD pattern of Example 17 after storage at 40° C. for 6 hours (t=6 hr), 24 hours (t=24 hr), 1 week (t=1 w), and 4 weeks (t=4 w);

FIG. 57 shows the mDSC profile of Example 17 after initially being prepared (t=0);

FIG. 58 shows the mDSC profile of Example 17 after storage at 40° C. for 1 week (t=1 w) and 4 weeks (t=4 w);

FIG. 59 shows the TGA profile of Example 17 after initially being prepared (t=0) and after storage at 40° C. for 24 hours (t=24 hr) and 1 week (t=1 w);

FIG. 60 shows the TGA profile of Example 17 after storage at 40° C. for 1 week (t=1 w) and 4 weeks (t=4 w);

FIG. 61 shows the XRPD pattern of Example 18 after storage at 40° C. for 6 hours (t=6 hr), 24 hours (t=24 hr), 1 week (t=1 w), and 4 weeks (t=4 w);

FIG. 62 shows the mDSC profile of Example 18 after initially being prepared (t=0);

FIG. 63 shows the mDSC profile of Example 18 after storage at 40° C. for 1 week (t=1 w) and 4 weeks (t=4 w);

FIG. 64 shows the TGA profile of Example 18 after initially being prepared (t=0) and after storage at 40° C. for 24 hours (t=24 hr) and 1 week (t=1 w);

FIG. 65 shows the TGA profile of Example 18 after storage at 40° C. for 1 week (t=1 w) and 4 weeks (t=4 w);

FIG. 66 shows the XRPD pattern of Example 19 after storage at 40° C. for 6 hours (t=6 hr), 24 hours (t=24 hr), 1 week (t=1 w), and 4 weeks (t=4 w);

FIG. 67 shows the mDSC profile of Example 19 after initially being prepared (t=0) and after being stored at 40° C. for 24 hours (t=24 hr);

FIG. 68 shows the mDSC profile of Example 19 after storage at 40° C. for 1 week (t=1 w) and 4 weeks (t=4 w);

FIG. 69 shows the TGA profile of Example 19 after initially being prepared (t=0) and after storage at 40° C. for 24 hours (t=24 hr);

FIG. 70 shows the TGA profile of Example 19 after storage at 40° C. for 24 hours (t=24 hr) and 4 weeks (t=4 w);

FIG. 71 shows the plasma concentration-time profiles for psilocin after psilocybin dosing with orally disintegrating tablets (ODT) and powder in capsule (PIC) dosage forms;

FIG. 72 shows the exposure comparison between psilocin after psilocybin dosing in ODT and PIC dosage forms as assessed by Cmax; and

FIG. 73 shows the exposure comparison between psilocin after psilocybin dosing in ODT and PIC dosage forms as assessed by AUCinf.

### DETAILED DESCRIPTION

In the following detailed description of the embodiments of the instant disclosure, numerous specific details are set forth in order to provide a thorough understanding of the disclosed embodiments. However, it will be obvious to one skilled in the art that the embodiments of this disclosure may be practiced without these specific details. In other instances, well known methods, procedures, components, and circuits have not been described in detail so as not to unnecessarily obscure aspects of the embodiments of the instant disclosure.

### Definitions

Unless defined otherwise, all technical and scientific terms used herein have the same meaning as is commonly understood by one of skill in the art to which this disclosure belongs.

When it is stated that a substituent or group “comprise(s) deuterium” or is “comprising deuterium,” it is to be understood that the substituent or group may itself be deuterium, or the substituent or group may contain at least one deuterium substitution in its chemical structure. For example, when substituent “—R” is defined to comprise deuterium, it is to be understood that —R may be -D (-deuterium), or a group such as —CD₃ that is consistent with the other requirements set forth of —R.

As used herein, the term “fatty” describes a compound with a long-chain (linear) hydrophobic portion made up of hydrogen and anywhere from 4 to 26 carbon atoms, which may be fully saturated or partially unsaturated.

The phrases “pharmaceutically acceptable,” “physiologically acceptable,” and the like, are employed herein to refer to those compounds, materials, compositions, and/or dosage forms which are, within the scope of sound medical judgment, suitable for use in contact with the tissues of human beings without excessive toxicity, irritation, allergic response, or other problem or complication, commensurate with a reasonable benefit/risk ratio. When referencing salts, the phrases “pharmaceutically acceptable salt,” “physiologically acceptable salt,” and the like, means a salt which is acceptable for administration to a patient, such as a mammal (salts with counterions having acceptable mammalian safety for a given dosage regime). As is well known in the art, such salts can be derived from pharmaceutically acceptable inorganic or organic bases, by way of example, sodium, potassium, calcium, magnesium, ammonium, and tetraalkylammonium salts, and the like, and when the molecule contains a basic functionality, addition salts with inorganic acids, such as hydrochloride, hydrobromide, sulfate, sulfamate, phosphate, nitrate, perchlorate salts, and the like, and addition salts with organic acids, such as formate, tartrate, besylate, mesylate, acetate, maleate, malonate, oxalate, fumarate, benzoate, salicylate, succinate, oxalate, glycolate, hemi-oxalate, hemi-fumarate, propionate, stearate, lactate, citrate, ascorbate, pamoate, hydroxymaleate, phenylacetate, glutamate, 2-acetoxybenzoate, tosylate, ethanedisulfonate, isethionate salts, and the like. The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a physical association of a compound or salt of the present disclosure with one or more solvent molecules, whether organic, inorganic, or a mixture of both. This physical association includes hydrogen bonding. In certain instances, the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution-phase and isolable solvates. Some examples of solvents include, but are not limited to, methanol, ethanol, isopropanol, N,N-dimethylformamide, tetrahydrofuran, dimethylsulfoxide, and water. When the solvent is water, the solvate formed is a hydrate (e.g., monohydrate, dihydrate, etc.). Exemplary solvates thus include, but are not limited to, hydrates, methanolates, ethanolates, isopropanolates, etc. Methods of solvation are generally known in the art.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto, imine-enamine, and neutral/zwitterionic tautomers, or the tautomeric forms of heteroaryl groups containing a —N═C(H)—NH— ring atom arrangement, such as pyrazoles, imidazoles, benzimidazoles, triazoles, and tetrazoles. Other tautomeric ring atom arrangements are also possible. For example, compounds containing an acid and a base group within the same molecule depicted in neutral form may exist also in a zwitterionic form, as is the case for amino acid/ammonium carboxylate tautomers. Thus, compounds of the present disclosure, e.g., compounds of Formula (I), which are depicted to contain both amino and dihydrogen phosphate functionality in neutral form may also exist in zwitterionic form as the ammonium monohydrogen phosphate zwitterion.

It will be appreciated that the compounds herein can exist in different salt, solvate, stereoisomer, and tautomeric forms, and the present disclosure is intended to include all permutations of salts, solvates, stereoisomers, and tautomers, such as a solvate of a pharmaceutically acceptable salt of a stereoisomer of the subject compound.

As used herein, the term “amorphous” refers to a solid material having substantially no long range order in the position of its molecules—the molecules are arranged in a random manner so that there is effectively no well-defined arrangement, e.g., molecular packing, and no long range order. Amorphous solids are generally isotropic, i.e., exhibit similar properties in all directions and do not have definite melting points. For example, an amorphous material is a solid material having substantially no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. Thus, an “amorphous” subject compound/material is one characterized as having substantially no crystallinity—less than 10% crystallinity, less than 8% crystallinity, less than 6% crystallinity, less than 4% crystallinity, less than 2% crystallinity, less than 1% crystallinity, or 0% crystallinity—i.e., is at least 90%, at least 92%, at least 94%, at least 96%, at least 98%, or 100% amorphous, as determined for example by XRPD. For example, the % crystallinity can in some embodiments be determined by measuring the intensity of one or more peaks in the XRPD diffractogram compared to a reference peak, which may be that of an internal standard. As used herein, the phrase “primarily in amorphous form” means that greater than 50% of the subject compound/material, e.g., a compound of Formula (I) present in a composition, is in amorphous form, for example, as determined by XRPD. It is also noted that the term “primarily in amorphous form” includes the descriptor, “amorphous,” defined above, and thus includes compounds/materials which are 100% amorphous (0% crystalline). XRPD analyses can be carried out, e.g., on a Bruker AXS D2 diffractometer using Cu Kα radiation (wavelength=1.54060 Å). Other characterization techniques, such as modulated differential scanning calorimetry (mDSC) analysis, Fourier transform infrared spectroscopy (FTIR), and other quantitative methods, may also be employed to determine the percent a subject compound/material is amorphous or crystalline, including quantitative methods which provide the above percentages in terms of weight percent.

The term “stable,” “stability,” and the like, as used herein includes chemical stability and solid state (physical) stability. The term “chemical stability” means that the compound can be stored in an isolated form, or in the form of a formulation in which it is provided in admixture with for example, pharmaceutically acceptable carriers, diluents or adjuvants as described herein, under normal storage conditions, with little or no chemical degradation or decomposition. “Solid-state stability” means the compound can be stored in an isolated solid form, or the form of a solid formulation in which it is provided, for example, within a polymer(s) and any optional pharmaceutically acceptable excipient (vehicles, carriers, diluents, or adjuvants) as described herein, under normal storage conditions, with little or no solid-state transformation (e.g., hydration, dehydration, solvatization, desolvatization, crystallization, recrystallization or solid-state phase transition). For example, a compound in an amorphous form is deemed stable if at least 50% of the compound remains in the amorphous form at the end of the specified period, e.g., as determined by XRPD, including an amorphous compound which does not form any detectable crystalline peaks by XRPD analysis during the indicated period. In the context of a pharmaceutically or biologically active ingredient (for example, the compound of Formula (I)), the stability may also be measured by the ability of the compound to retain at least 50% of its activity with reference to the beginning of the specified period, or to retain certain physical or chemical properties under certain specified conditions.

A “crystalline psilocybin-based drug” is any solid dosage form formulated with a prodrug of a psilocin-type compound, the prodrug of a psilocin-type compound being primarily in crystalline form (>50% crystalline, e.g., as determined by XRPD). Prodrugs include an alkyl/aryl ester, an α-amino ester (e.g., an amino acid ester), a hemi-ester, a bis-ester, a phosphate ester, a sulfate ester, etc. of a psilocin-type compound, that when administered releases psilocin or a deuterated analog thereof (e.g., a dephosphorylated form of a compound of Formula (I)) as the active component. A crystalline psilocybin-based drug encompasses solid dosage forms containing a crystal form of psilocybin itself, including, but not limited to the following polymorphs (Cambridge structural database (CSD) Reference Codes identified in parentheses): Form A (HATCAK & TAVZID), Form B (TAVZID01), methanol solvate (PSILOC), trihydrate (OKOKAD), and ethanol solvate (KOWHOT); see Sherwood et al., “Psilocybin: crystal structure solutions enable phase analysis of prior art and recently patented examples”, Acta Cryst. (2022), C78 (1), 36-55; as well as the polymorph described as polymorph A in U.S. Pat. No. 10,519,175 or US 2022/0088041 A1.

As used herein, the term “composition” is equivalent to the term “formulation.”

The term “treating” or “treatment” as used herein means the treating or treatment of a disease or medical condition in a patient, such as a mammal (particularly a human) that includes: ameliorating the disease or medical condition, such as, eliminating or causing regression of the disease or medical condition in a patient; suppressing the disease or medical condition, for example by, slowing or arresting the development of the disease or medical condition in a patient; or alleviating one or more symptoms of the disease or medical condition in a patient. In some embodiments, prophylactic treatment can result in preventing the disease or medical condition from occurring, in a subject.

As used herein, the terms “reduce,” “decrease,” and similar terms mean a decrease of at least about 10%, about 15%, about 20%, about 25%, about 35%, about 50%, about 75%, about 80%, about 85%, about 90%, about 95%, about 97%, or more.

As used herein, the terms “improve,” “increase,” “enhance,” and similar terms indicate an increase of at least about 10%, about 15%, about 20%, about 25%, about 50%, about 75%, about 100%, about 150%, about 200%, about 300%, about 400%, about 500%, or more.

A “patient” or “subject,” used interchangeably herein, refers to human and non-human subjects, especially mammalian subjects. A patient or subject can have a condition to be treated or can be susceptible to a condition to be treated.

As used herein, and unless otherwise specified, the terms “prevent,” “preventing” and “prevention” refer to the prevention of the onset, recurrence or spread of a disease, disorder, or condition, or of one or more symptoms thereof. The terms encompass the inhibition or reduction of a symptom of the particular disease, disorder, or condition. Subjects with familial history of a disease, disorder, or condition, in particular, are candidates for preventive regimens in some embodiments. In addition, subjects who have a history of recurring symptoms are also potential candidates for the prevention. In this regard, the term “prevention” may be interchangeably used with the term “prophylactic treatment.”

“Therapeutically effective amount” refers to an amount of a compound sufficient to treat a specified disorder or disease or one or more of its symptoms and/or to prevent the occurrence of the disease or disorder (prophylactically effective amount).

As used herein, and unless otherwise specified, a “prophylactically effective amount” of an active ingredient, is an amount sufficient to prevent a disease, disorder, or condition, or prevent its recurrence. The term “prophylactically effective amount” can encompass an amount that improves overall prophylaxis or enhances the prophylactic efficacy of another prophylactic agent.

The term “administration schedule” is a plan in which the type, amount, period, procedure, etc. of the drug in the drug treatment are shown in time series, and the dosage, administration method, administration order, administration date, and the like of each drug are indicated. The date specified to be administered is determined before the start of the drug administration. The administration is continued by repeating the course with the set of administration schedules as “courses”. A “continuous” administration schedule means administration every day without interruption during the treatment course. If the administration schedule follows an “intermittent” administration schedule, then days of administration may be followed by “rest days” or days of non-administration of drug within the course. A “drug holiday” indicates that the drug is not administered in a predetermined administration schedule. For example, after undergoing several courses of treatment, a subject may be prescribed a regulated drug holiday as part of the administration schedule, e.g., prior to re-recommencing active treatment.

As used herein, and unless otherwise specified, a “neuropsychiatric disease or disorder” is a behavioral or psychological problem associated with a known neurological condition, and typically defined as a cluster of symptoms that co-exist. Examples of neuropsychiatric disorders include, but are not limited to, attention deficit disorder, attention deficit hyperactivity disorder, bipolar and manic disorders, depression, or any combinations thereof.

“Inflammatory conditions” or “inflammatory disease,” as used herein, refers broadly to chronic or acute inflammatory diseases, including, but not limited to, rheumatic diseases (e.g., rheumatoid arthritis, osteoarthritis, psoriatic arthritis) spondyloarthropathies (e.g., ankylosing spondylitis, reactive arthritis, Reiter's syndrome), crystal arthropathies (e.g., gout, pseudogout, calcium pyrophosphate deposition disease), multiple sclerosis, Lyme disease, polymyalgia rheumatica; connective tissue diseases (e.g., systemic lupus erythematosus, systemic sclerosis, polymyositis, dermatomyositis, Sjogren's syndrome); vasculitides (e.g., polyarteritis nodosa, Wegener's granulomatosis, Churg-Strauss syndrome); inflammatory conditions including consequences of trauma or ischaemia, sarcoidosis; vascular diseases including atherosclerotic vascular disease, atherosclerosis, and vascular occlusive disease (e.g., atherosclerosis, ischaemic heart disease, myocardial infarction, stroke, peripheral vascular disease), and vascular stent restenosis; ocular diseases including uveitis, corneal disease, iritis, iridocyclitis, glaucoma, and cataracts.

As used herein, the term “anxiety disorder” refers to a state of apprehension, uncertainty, and/or fear resulting from the anticipation of an event and/or situation. Non-limiting examples of anxiety disorders include acute stress disorder, anxiety due to a medical condition, generalized anxiety disorder, panic disorder, panic attack, a phobia, post-traumatic stress disorder, obsessive-compulsive disorder, separation anxiety disorder, social anxiety disorder, substance-induced anxiety disorder, or selective mutism.

As used herein, the term “post-traumatic stress disorder” refers to a condition developed after experiencing and/or witnessing a traumatic event or learning that a traumatic event has happened, e.g., to a loved one. Non-limiting examples of traumatic events include exposure to war, rape or sexual violence, a physical attack, disease, a mugging, childhood physical or sexual violence, kidnapping or being taken hostage, terrorist attacks, torture, nature disasters and/or severe accidents.

As used herein, an “obsessive-compulsive and related disorder” refers to a condition that involves obsessions or compulsions. Non-limiting examples of obsessive-compulsive and related disorder is obsessive compulsive disorder, body dysmorphic disorder, hoarding disorder, dermatillomania, trichotillomania, excoriation, substance-induced obsessive compulsive and related disorder, or an obsessive-compulsive disorder due to another medical condition, or a combination thereof.

As used herein, the term “eating disorder” refers to any of a range of psychological disorders characterized by abnormal or disturbed eating habits. Non-limiting examples of eating disorders include pica, anorexia nervosa, bulimia nervosa, rumination disorder, avoidant/restrictive food intake disorder, binge-eating disorder, other specified feeding or eating disorder, unspecified feeding or eating disorder, or combinations thereof.

As used herein, the term “headache disorder” refers to a disorder characterized by recurrent headaches.

All diseases and disorders listed herein are defined as described in the Diagnostic and Statistical Manual of Mental Disorders (DSM-5), published by the American Psychiatric Association, or in International Classification of Diseases (ICD), published by the World Health Organization.

As used herein, an “antidepressant” refers to any drug used to alleviate depression. Non-limiting examples of antidepressants include one or more of the following antidepressants: adatanserine hydrochloride; adinazolam; adinazolam mesylate; allaproclate; alletamine hydrochloride; amedalin hydrochloride; amitriptyline hydrochloride; amoxapine; aftazapine maleate; amitriptyline; azaroxane fumarate; azepindol; azipramine hydrochloride; bifenanol hydrochloride; bupropion hydrochloride; butacetin; butriftyline hydrochloride; caroxazone; cartazolate; cyclazindol; cydoxepin hydrochloride; cilobamin mesylate; citalipram; clodazone hydrochloride; clomipramine hydrochloride; cotinine fumarate; cyclindol; cyphenamine hydrochloride; cyproridol hydrochloride; cyproxymid; darledin tosylate; dapoxetine hydrochloride; dazadrol maleate; dazefinyl hydrochloride; desipramine hydrochloride; dexamidazole; deximaphene; dibenzepine hydrochloride; dioxadol hydrochloride; dothipine hydrochloride; doxepin hydrochloride; duloxetine hydrochloride; eclanamine maleate; encyclates; etoferidone hydrochloride; pantridone hydrochloride; femetosol hydrochloride; penmetamide; pezolamine fumarate; fluorothracene hydrochloride; fluoxetine; fluoxetine hydrochloride; fluparoxane hydrochloride; gamma pexin; guanoxyphen sulfate; imafen hydrochloride; imilox hydrochloride; imipramine hydrochloride; indeloxazine hydrochloride; intriptyline hydrochloride; ifrindol; isocacarazide; ketipramine fumarate; lofepramine hydrochloride; lortalamine; mapprotilin; mapprotiline hydrochloride; melitracene hydrochloride; milacemide hydrochloride; minarin hydrochloride; mirtazapine; moclobemide; modal sulphate; napaktadine hydrochloride; napamezol hydrochloride; nefazodone hydrochloride; nisoxetine; nitroputam hydrochloride; nomifensin maleate; nortriptyline hydrochloride; octriptyline phosphate; opipramol hydrochloride; oxaprotiline hydrochloride; oxypertin; paroxetine; phenelazine sulfate; pyrandamine hydrochloride; piezotiline; pridepine hydrochloride; prolinetane hydrochloride; protriptyline hydrochloride; quipazine maleate; rolycyrine; ceproxetine hydrochloride; sertraline hydrochloride; sibutramine hydrochloride; sulfides; hydrotosol; tamethalin hydrochloride; tampramine fumarate; tandamine hydrochloride; thiazime hydrochloride; tozalinone; geocetin hydrochloride; trazodone hydrochloride; trebenzomine hydrochloride; trimipramine; trimipramine maleate; venlafaxine hydrochloride; biloxazine hydrochloride; gimeldine hydrochloride; and zometapine.

As used herein, the following Medical Dictionary for Regulatory Activities (MedDRA) terms are considered to be adverse events that are psychedelic in nature: altered mood, altered state of consciousness, autoscopy, delusional perception, disinhibition, dissociation, dissociative identity disorder, dreamy state, emotional disorder, euphoric mood, feeling abnormal, hallucination, hyperacusis, hyperaesthesia, hypoaesthesia, illusion, paranoia, parosmia, photophobia, sensory disturbance, time perception altered, thinking abnormal, synaesthesia, substance-induced psychotic distress, and somatic hallucination.

As used herein, the term “dispersion” refers to a disperse system in which a first substance, the dispersed phase, is distributed, in discrete units, throughout a second substance (the continuous phase or vehicle). The size of the dispersed phase can vary considerably (e.g., single molecules, colloidal particles of nanometer dimension, to multiple microns in size). In general, the dispersed phases can be solids, liquids, or gases. In the case of a “solid dispersion,” the dispersed and continuous phases are both solids, and thus includes any solid composition having at least two components. In the present disclosure, a solid dispersion can include an amorphous solid dispersion, i.e., an amorphous drug in an amorphous polymer, an amorphous drug in crystalline polymer, or an amorphous drug in a mixture of an amorphous polymer and a crystalline polymer. In any case, the polymer may constitute the dispersed phase while the drug constitutes the continuous phase, or, the drug may constitute the dispersed phase while the polymer constitutes the continuous phase. The solid dispersion as disclosed herein may include an active ingredient (for example a compound of Formula (I)) dispersed among at least one other component, for example a polymer. In some embodiments, a solid dispersion includes the compound of Formula (I) molecularly dispersed with a polymer. The solid dispersion can exist, for example, as a one phase/homogenous system, a two phase system, etc.

The term “molecularly dispersed”, as used herein, refers to the random distribution of a compound (e.g., compound of Formula (I)) with a polymer. In some embodiments the compound is present in the polymer in a final state of subdivision, see, e.g., M. G. Vachon et al., J. Microencapsulation, 14:281-301 (1997) and Vandelli et al., J. Microencapsulation, 10: 55-65 (1993). In some embodiments, a compound (for example, a compound of Formula (I)) may be dispersed within a matrix formed by the polymer in its solid state such that the compound is immobilized in its amorphous form. Whether a compound is molecularly dispersed in a polymer may be evidenced in a variety of ways, e.g., by the resulting solid molecular complex having a single glass transition temperature.

The term “solid molecular complex” as used herein means a solid dispersion that includes compound of Formula (I) molecularly dispersed within a polymer matrix.

The term “immobilize,” as used herein with reference to the immobilization of the active ingredient in the polymer matrix, means that molecules of the active ingredient interact with molecules of the polymer in such a way that the molecules of the active ingredient are held in the aforementioned matrix and prevented from crystal nucleation due to lack of mobility. In some embodiments, the polymer may prevent intermolecular hydrogen bonding or weak dispersion forces between two or more active ingredient molecules (e.g., molecules of compound of Formula (I)). See, for example, Matsumoro and Zografi, Pharmaceutical Research, Vo. 16, No. 11, p 1722-1728, 1999.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

### Pharmaceutical Compositions

Disclosed herein is a pharmaceutical composition comprising (i) a therapeutically effective amount of a compound of Formula (I), or a pharmaceutically acceptable salt, a polymorph, stereoisomer, a tautomer, solvate, or active metabolite thereof, and (ii) a polymer. The therapeutically effective amount of a compound of Formula (I) (or a pharmaceutically acceptable salt, a polymorph, stereoisomer, a tautomer, solvate, or active metabolite thereof) and the polymer may be provided in the form of a solid dispersion (e.g., solid molecular complex), for example a solid dispersion in which the compound of Formula (I) (or a pharmaceutically acceptable salt, stereoisomer, a tautomer, or solvate thereof) is stably dispersed in amorphous form in the polymer, sometimes referred to herein as an amorphous solid dispersion (ASD). In addition to the compound of Formula (I) (or a pharmaceutically acceptable salt, a polymorph, stereoisomer, a tautomer, solvate, or active metabolite thereof) and the polymer, the solid dispersion may optionally contain one or more pharmaceutically acceptable excipients, i.e., pharmaceutically acceptable excipients dispersed with the compound of Formula (I) (or a pharmaceutically acceptable salt, a polymorph, stereoisomer, a tautomer, solvate, or active metabolite thereof) and the polymer, as part of the solid dispersion. The pharmaceutically acceptable excipient may be any one or more as set forth hereinafter. The solid dispersion may be used per se as a pharmaceutical composition. Alternatively, a pharmaceutical composition may optionally be formulated with one or more pharmaceutically acceptable excipients which are separate from/not dispersed within the solid dispersion containing the compound of Formula (I) (or a pharmaceutically acceptable salt, a polymorph, stereoisomer, a tautomer, solvate, or active metabolite thereof) and the polymer, i.e., where the pharmaceutically acceptable excipient(s) is considered to be a separate component of the dosage form, and not dispersed with the solid dispersion.

The compound of Formula (I) is provided as follows:

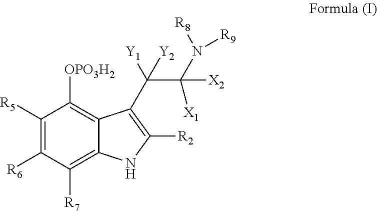

wherein:
R₂, R₅, R₆, and R₇ are independently selected from the group consisting of hydrogen and deuterium,
R₈ and R₉ are independently selected from the group consisting of —CH₃, —CH₂D, —CHD₂, and —CD₃, and
X₁, X₂, Y₁, and Y₂ are independently selected from the group consisting of hydrogen and deuterium.

In some embodiments, R₂, R₅, R₆, and R₇ are independently selected from the group consisting of hydrogen and deuterium. In some embodiments, R₂ is deuterium. In some embodiments, R₂ is hydrogen. In some embodiments, R₅ is deuterium. In some embodiments, R₅ is hydrogen. In some embodiments, R₆ is deuterium. In some embodiments, R₆ is hydrogen.

In some embodiments, R₇ is deuterium. In some embodiments, R₇ is hydrogen. R₂, R₅, R₆, and R₇ may be the same, for example, R₂, R₅, R₆, and R₇ may each be hydrogen, or alternatively, R₂, R₅, R₆, and R₇ may each be deuterium. In some embodiments, at least one of R₂, R₅, R₆, and R₇ is deuterium. In some embodiments, at least two of R₂, R₅, R₆, and R₇ are deuterium. In some embodiments, at least three of R₂, R₅, R₆, and R₇ are deuterium. In some embodiments, R₂, R₅, R₆, and R₇ are deuterium. In some embodiments, R₂, R₅, R₆, and R₇ are hydrogen.

In some embodiments, R₈ and R₉ are independently selected from the group consisting of —CH₃, —CH₂D, —CHD₂, and —CD₃. R₈ and R₉ may be the same, or different. In some embodiments, R₈ and R₉ are the same. In some embodiments, R₈ and R₉ are different. In some embodiments, R₈ and R₉ are independently selected from the group consisting of —CH₃ and —CD₃. In some embodiments, R₈ and R₉ are methyl (—CH₃). In some embodiments, R₈ and R₉ are a partially deuterated methyl group, i.e., —CDH₂ or —CD₂H. In some embodiments, R₈ and R₉ are a fully deuterated methyl group (—CD₃). In some embodiments, at least one of R₈ and R₉ is —CD₃.

In some embodiments, X₁, X₂, Y₁, and Y₂ are independently selected from the group consisting of hydrogen and deuterium. X₁ and X₂ may be the same, or different. In some embodiments, X₁ and X₂ are the same. In some embodiments, X₁ and X₂ are hydrogen. In some embodiments, X₁ and X₂ are deuterium. In some embodiments, X₁ is deuterium and X₂ is hydrogen.

Y₁ and Y₂ may be the same, or different. In some embodiments, Y₁ and Y₂ are the same. In some embodiments, Y₁ and Y₂ are hydrogen. In some embodiments, Y₁ and Y₂ are deuterium. In some embodiments, Y₁ is deuterium and Y₂ is hydrogen. In some embodiments, X₁, X₂, Y₁, and Y₂ are hydrogen. In some embodiments, X₁, X₂, Y₁, and Y₂ are deuterium.

In some embodiments, X₁, X₂, Y₁, Y₂, R₂, R₅, R₆, and R₇ are each hydrogen, and R₈ and R₉ are each —CH₃. In some embodiments, X₁, X₂, Y₁, Y₂, R₂, R₅, R₆, and R₇ are each hydrogen, and R₈ and R₉ comprise deuterium (e.g., are —CD₃ groups or a partially deuterated methyl group). In some embodiments, at least one of X₁, X₂, Y₁, Y₂, R₂, R₅, R₆, R₇, R₈, and R₉ comprises deuterium. In some embodiments, at least X₁, X₂, R₈, and R₉ comprise deuterium.

In some embodiments, at least X₁, X₂, Y₁, Y₂, R₈, and R₉ comprise deuterium. In some embodiments, X₁, X₂, Y₁, and Y₂ are deuterium, and R₈ and R₉ are a fully deuterated methyl group (—CD₃).

The compounds of Formula (I) may contain a stereogenic center. In such cases, the compounds may exist as different stereoisomeric forms, even though Formula (I) is drawn without reference to stereochemistry. Accordingly, the present disclosure includes all possible stereoisomers and includes not only racemic compounds but the individual enantiomers (enantiomerically pure compounds), individual diastereomers (diastereomerically pure compounds), and their non-racemic mixtures as well. When a compound is desired as a single enantiomer, such may be obtained by, e.g., stereospecific synthesis, as is known in the art.

In some embodiments, the compounds described herein, e.g., compounds of Formula (I), are non-stereogenic. In some embodiments, the compounds described herein, e.g., compounds of Formula (I), are racemic. In some embodiments, the compounds described herein, e.g., compounds of Formula (I), are enantiomerically enriched (one enantiomer is present in a higher percentage), including enantiomerically pure. In some embodiments, the compounds described herein, e.g., compounds of Formula (I), are provided as a single diastereomer. In some embodiments, the compounds described herein, e.g., compounds of Formula (I), are provided as a mixture of diastereomers. When provided as a mixture of diastereomers, the mixtures may include equal mixtures, or mixtures which are enriched with a particular diastereomer (one diastereomer is present in a higher percentage than another).

In some embodiments, the compound of Formula (I) is an agonist of a serotonin 5-HT₂ receptor. In some embodiments, the compound of Formula (I) is an agonist of a serotonin 5-HT_{2A} receptor.

In some embodiments, the compound of Formula (I) is selected from the group consisting of:

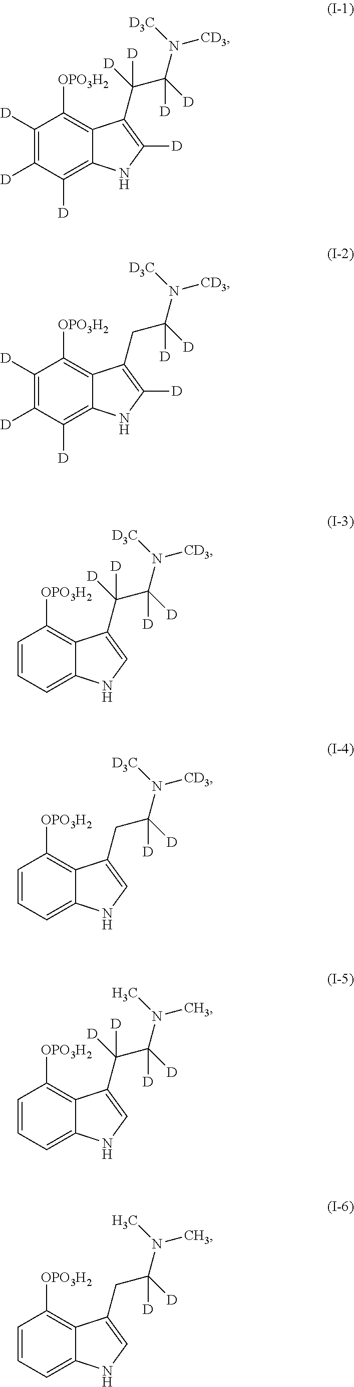

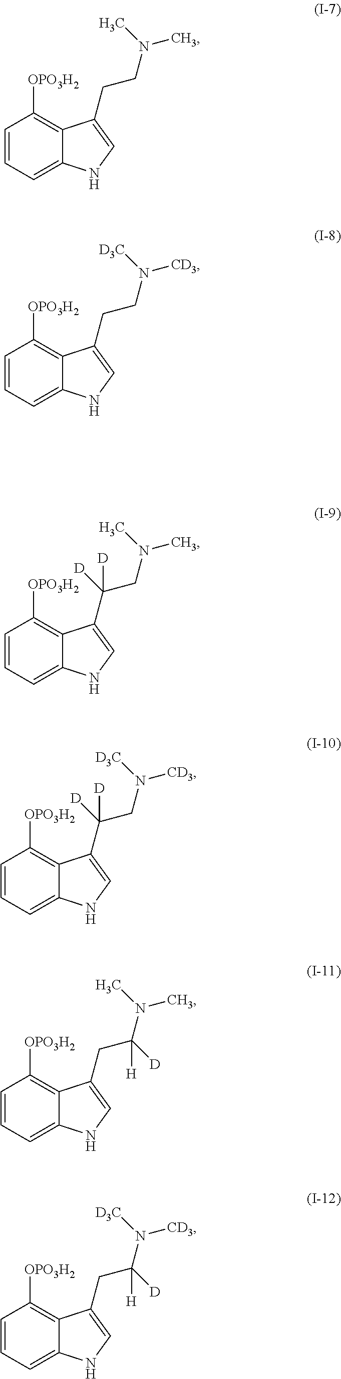

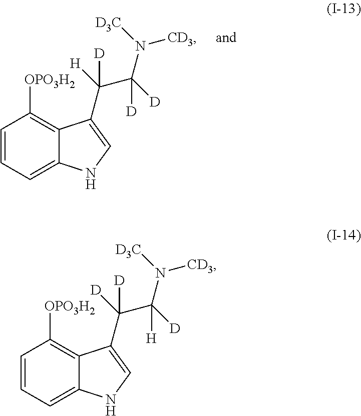

or a pharmaceutically acceptable salt, a polymorph, stereoisomer, a tautomer, solvate, or active metabolite thereof.

The compound number, IUPAC name, and substituent listing for the above-identified compounds are provided in Table 1.

TABLE 1
Exemplary compounds of Formula (I)

| | | Formula (I) | | | | | | |
|---|---|---|---|---|---|---|---|---|
| Compound identifier and name | | X₁, X₂ | Y₁, Y₂ | R₂ | R₅ | R₆ | R₇ | R₈, R₉ |
| I-1 | 3-(2-(bis(methyl-d₃)amino)ethyl-1,1,2,2-d₄)- | D, D | D, D | D | D | D | D | —CD₃, —CD₃ |
| | 1H-indol-4-yl-2,5,6,7-d₄ dihydrogen | | | | | | | |
| | phosphate | | | | | | | |
| I-2 | 3-(2-(bis(methyl-d₃)amino)ethyl-2,2-d₂)-1H- | D, D | H, H | D | D | D | D | —CD₃, —CD₃ |
| | indol-4-yl-2,5,6,7-d₄ dihydrogen phosphate | | | | | | | |
| I-3 | 3-(2-(bis(methyl-d₃)amino)ethyl-1,1,2,2-d₄)- | D, D | D, D | H | H | H | H | —CD₃, —CD₃ |
| | 1H-indol-4-yl dihydrogen phosphate | | | | | | | |
| I-4 | 3-(2-(bis(methyl-d₃)amino)ethyl-2,2-d₂)-1H- | D, D | H, H | H | H | H | H | —CD₃, —CD₃ |
| | indol-4-yl dihydrogen phosphate | | | | | | | |
| I-5 | 3-(2-(dimethylamino)ethyl-1,1,2,2-d₄)-1H- | D, D | D, D | H | H | H | H | —CH₃, —CH₃ |
| | indol-4-yl dihydrogen phosphate | | | | | | | |
| I-6 | 3-(2-(dimethylamino)ethyl-2,2-d₂)-1H-indol- | D, D | H, H | H | H | H | H | —CH₃, —CH₃ |
| | 4-yl dihydrogen phosphate | | | | | | | |
| 7 | 3-(2-(dimethylamino)ethyl)-1H-indol-4-yl | H, H | H, H | H | H | H | H | —CH₃, —CH₃ |
| | dihydrogen phosphate | | | | | | | |
| I-8 | 3-(2-(bis(methyl-d₃)amino)ethyl)-1H-indol-4- | H, H | H, H | H | H | H | H | —CD₃, —CD₃ |
| | yl dihydrogen phosphate | | | | | | | |
| I-9 | 3-(2-(dimethylamino)ethyl-1,1-d₂)-1H-indol- | H, H | D, D | H | H | H | H | —CH₃, —CH₃ |
| | 4-yl dihydrogen phosphate | | | | | | | |
| I-10 | 3-(2-(bis(methyl-d₃)amino)ethyl-1,1-d₂)-1H- | H, H | D, D | H | H | H | H | —CD₃, —CD₃ |
| | indol-4-yl dihydrogen phosphate | | | | | | | |
| I-11 | 3-(2-(dimethylamino)ethyl-2-d)-1H-indol-4-yl | H, D | H, H | H | H | H | H | —CH₃, —CH₃ |
| | dihydrogen phosphate | | | | | | | |
| I-12 | 3-(2-(bis(methyl-d₃)amino)ethyl-2-d)-1H- | H, D | H, H | H | H | H | H | —CD₃, —CD₃ |
| | indol-4-yl dihydrogen phosphate | | | | | | | |
| I-13 | 3-(2-(bis(methyl-d₃)amino)ethyl-1,2,2-d₃)-1H- | D, D | H, D | H | H | H | H | —CD₃, —CD₃ |
| | indol-4-yl dihydrogen phosphate | | | | | | | |
| I-14 | 3-(2-(bis(methyl-d₃)amino)ethyl-1,1,2-d₃)-1H- | H, D | D, D | H | H | H | H | —CD₃, —CD₃ |
| | indol-4-yl dihydrogen phosphate | | | | | | | |

In some embodiments, the compound of Formula (I) is

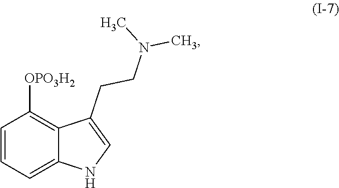

or a pharmaceutically acceptable salt, tautomer, or solvate thereof.

In some embodiments, the active metabolite of the compound of Formula (I) is psilocin or a corresponding deuterated psilocin.

In some embodiments, the compounds of the present disclosure are provided in amorphous form, e.g., as determined by XRPD and/or mDSC. Accordingly, pharmaceutical compositions may be prepared from compounds of Formula (I), in one or more amorphic forms, and may be used for treatment as set forth herein. In some embodiments, the compound of Formula (I) is an amorphous form of 3-(2-(bis(methyl-d₃)amino)ethyl-1,1,2,2-d₄)-1H-indol-4-yl-2,5,6,7-d₄ dihydrogen phosphate (I-1), as determined by X-ray powder diffraction. In some embodiments, the compound of Formula (I) is an amorphous form of 3-(2-(bis(methyl-d₃)amino)ethyl-2,2-d₂)-1H-indol-4-yl-2,5,6,7-d₄ dihydrogen phosphate (I-2), as determined by X-ray powder diffraction. In some embodiments, the compound of Formula (I) is an amorphous form of 3-(2-(bis(methyl-d₃)amino)ethyl-1,1,2,2-d₄)-1H-indol-4-yl dihydrogen phosphate (I-3), as determined by X-ray powder diffraction. In some embodiments, the compound of Formula (I) is an amorphous form of 3-(2-(bis(methyl-d₃)amino)ethyl-2,2-d₂)-1H-indol-4-yl dihydrogen phosphate (I-4), as determined by X-ray powder diffraction. In some embodiments, the compound of Formula (I) is an amorphous form of 3-(2-(dimethylamino)ethyl-1,1,2,2-d₄)-1H-indol-4-yl dihydrogen phosphate (I-5), as determined by X-ray powder diffraction. In some embodiments, the compound of Formula (I) is an amorphous form of 3-(2-(dimethylamino)ethyl-2,2-d₂)-1H-indol-4-yl dihydrogen phosphate (I-6), as determined by X-ray powder diffraction. In some embodiments, the compound of Formula (I) is an amorphous form of 3-(2-(dimethylamino)ethyl)-1H-indol-4-yl dihydrogen phosphate (I-7), as determined by X-ray powder diffraction. In some embodiments, the compound of Formula (I) is an amorphous form of 3-(2-(bis(methyl-d₃)amino)ethyl)-1H-indol-4-yl dihydrogen phosphate (I-8), as determined by X-ray powder diffraction. In some embodiments, the compound of Formula (I) is an amorphous form of 3-(2-(dimethylamino)ethyl-1,1-d₂)-1H-indol-4-yl dihydrogen phosphate (I-9), as determined by X-ray powder diffraction. In some embodiments, the compound of Formula (I) is an amorphous form of 3-(2-(bis(methyl-d₃)amino)ethyl-1,1-d₂)-1H-indol-4-yl dihydrogen phosphate (I-10), as determined by X-ray powder diffraction. In some embodiments, the compound of Formula (I) is an amorphous form of 3-(2-(dimethylamino)ethyl-2-d)-1H-indol-4-yl dihydrogen phosphate (I-11), as determined by X-ray powder diffraction. In some embodiments, the compound of Formula (I) is an amorphous form of 3-(2-(bis(methyl-d₃)amino)ethyl-2-d)-1H-indol-4-yl dihydrogen phosphate (I-12), as determined by X-ray powder diffraction. In some embodiments, the compound of Formula (I) is an amorphous form of 3-(2-(bis(methyl-d₃)amino)ethyl-1,2,2-d₃)-1H-indol-4-yl dihydrogen phosphate (I-13), as determined by X-ray powder diffraction. In some embodiments, the compound of Formula (I) is an amorphous form of 3-(2-(bis(methyl-d₃)amino)ethyl-1,1,2-d₃)-1H-indol-4-yl dihydrogen phosphate (I-14), as determined by X-ray powder diffraction.

Amorphous forms of the compounds of Formula (I) may be advantageous in terms of higher aqueous solubility and dissolution rates in water, compared to crystalline forms, thereby enabling rapid systemic absorption, higher bioavailability, and better control/more predictable therapeutic onset. Further, in some embodiments, pharmaceutical compositions may be prepared which comprise the amorphous forms of the compounds of Formula (I), e.g., as a solid dispersion (e.g., a solid molecular complex). The solid dispersion (e.g., solid molecular complex) of the present disclosure, such as those set forth herein, may act to stabilize the amorphous forms of the compounds of Formula (I), which tend to be unstable and have a tendency to crystallize. Accordingly, the solid dispersion (e.g., solid molecular complex) can be used to stabilize and deliver these amorphous forms to subjects in need of treatment, e.g., for the treatment of a condition or disease associate with a serotonin 5-HT₂ receptor.

Also disclosed herein is a pharmaceutically acceptable salt of the compound of Formula (I), or a pharmaceutically acceptable polymorph, stereoisomer, a tautomer, or solvate thereof. The acid used to form the pharmaceutically acceptable salt of the compound of Formula (I) may be a monoacid, a diacid, a triacid, a tetraacid, or may contain a higher number of acid groups. The acid groups may be, e.g., a carboxylic acid, a sulfonic acid, a phosphonic acid, or other acidic moieties containing at least one replaceable hydrogen atom. Examples of acids for use in the preparation of the pharmaceutically acceptable (acid addition) salts disclosed herein include, but are not limited to, acetic acid, 2,2-dichloroacetic acid, phenylacetic acid, acylated amino acids, alginic acid, ascorbic acid, L-aspartic acid, sulfonic acids (e.g., benzenesulfonic acid, camphorsulfonic acid, (+)-(1S)-camphor-10-sulfonic acid, ethane-1,2-disulfonic acid, ethanesulfonic acid, 2-hydroxy-ethanesulfonic acid, methanesulfonic acid, naphthalene-2-sulfonic acid, naphthalene-1,5-disulfonic acid, p-toluenesulfonic acid, ethanedisulfonic acid, etc.), benzoic acids (e.g., benzoic acid, 4-acetamidobenzoic acid, 2-acetoxybenzoic acid, salicylic acid, 4-amino-salicylic acid, gentisic acid, etc.), boric acid, (+)-camphoric acid, cinnamic acid, citric acid, cyclamic acid, cyclohexanesulfamic acid, dodecylsulfuric acid, formic acid, fumaric acid, galactaric acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (−)-D-lactic acid, (±)-DL-lactic acid, lactobionic acid, maleic acid, malic acid, (−)-L-malic acid, (+)-D-malic acid, hydroxymaleic acid, malonic acid, (±)-DL-mandelic acid, isethionic acid, 1-hydroxy-2-naphthoic acid, nicotinic acid, nitric acid, orotic acid, oxalic acid, pamoic acid, perchloric acid, phosphoric acid, L-pyroglutamic acid, saccharic acid, succinic acid, sulfuric acid, sulfamic acid, tannic acid, tartaric acids (e.g., DL-tartaric acid, (+)-L-tartaric acid, (−)-D-tartaric acid), thiocyanic acid, propionic acid, valeric acid, and fatty acids (including fatty mono- and di-acids, e.g., adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, caproic acid, etc.).

Methods for preparing pharmaceutically acceptable salt forms of pharmaceutical compounds are known by those of ordinary skill in the art. In some embodiments, the method includes:
(a) suspending the compound of Formula (I) in a solvent or mixture of solvents;
(b) contacting an acid with the compound of Formula (I) to provide a mixture;
(c) optionally heating the mixture;
(d) optionally cooling the mixture; and
(e) isolating the salt.

Various solvents may be used in the disclosed methods, including one or more protic solvents, one or more aprotic solvents, or mixtures thereof. In some embodiments, the solvent(s) used in the method of preparing the salt is/are a protic solvent(s). In some embodiments, the solvent used in the method of preparing the salt is selected from the group consisting of methanol, ethanol, propanol, isopropanol, butanol, 2-butanol, acetone, butanone, dioxanes (1,4-dioxane), water, tetrahydrofuran (THF), acetonitrile (MeCN), ether solvents (e.g., t-butylmethyl ether (TBME)), hexane, heptane, octane, and combinations thereof. In some embodiments, the solvent is ethanol. In some embodiments, the solvent is 1,4-dioxane. In some embodiments, the solvent is acetonitrile. In some embodiments, the solvent is tetrahydrofuran.

Suitable acids for use in the preparation of pharmaceutically acceptable acid addition salts may include those described heretofore. The acid may be an inorganic acid such as hydrochloric acid, or an organic acid, with organic acids being preferred. In some embodiments, the acid is an organic acid selected from the group consisting of ascorbic acid, citric acid, fumaric acid, maleic acid, malonic acid, (−)-L-malic acid, (+)-L-tartaric acid, methanesulfonic acid, benzenesulfonic acid, toluenesulfonic acid, benzoic acid, salicylic acid, succinic acid, oxalic acid, D-glucuronic acid, glutaric acid salt, and acetic acid. In some embodiments, the acid is an organic acid selected from the group consisting of benzenesulfonic acid, (+)-L-tartaric acid, fumaric acid, acetic acid, citric acid, malonic acid, succinic acid, oxalic acid, benzoic acid, and salicylic acid, with benzenesulfonic acid, succinic acid, and benzoic acid being preferred. In some embodiments, the acid is a fatty acid, such as adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, caproic acid, etc., with particular mention being made to adipic (hexandioic) acid, lauric (dodecanoic) acid, linoleic acid, myristic (tetradecanoic) acid, capric (decanoic) acid, stearic (octadecanoic) acid, oleic acid, and caprylic (octanoic) acid.

In some embodiments, a stoichiometric (or superstoichiometric) quantity of the acid is contacted with the compound of Formula (I). In some embodiments, a sub-stoichiometric (e.g., 0.5 molar equivalents) quantity of the acid is contacted with the compound of Formula (I). The use of sub-stoichiometric quantities of the acid may be desirable when, for example, the acid contains at least two acidic protons (e.g., two or more carboxylic acid groups) and the target salt is a hemi-acid salt.

In some embodiments, the mixture is heated, e.g., refluxed, prior to cooling.

In some embodiments, the mixture is cooled and the salt is precipitated out of the solution. In some embodiments, the salt is precipitated out of solution in crystalline form. In some embodiments, the salt is precipitated out of solution in amorphous form.

Isolation of the salt may be performed by various well-known isolation techniques, such as filtration, decantation, and the like. In some embodiments, the isolating step includes filtering the mixture.

After isolation, additional crystallization and/or recrystallization steps may also optionally be performed, if desired, for example to increase purity, crystallinity, etc.

In some embodiments, compounds of the present disclosure, e.g., a compound of Formula (I), or any pharmaceutically acceptable salt, polymorph, stereoisomer, or tautomer thereof, is in the form of a solvate. Examples of solvate forms include, but are not limited to, hydrates, methanolates, ethanolates, isopropanolates, etc., with hydrates and ethanolates being preferred. The solvate may be formed from stoichiometric or nonstoichiometric quantities of solvent molecules. Solvates of the compounds herein may be in the form of isolable solvates. In one non-limiting example, as a hydrate, the compound may be a monohydrate, a dihydrate, etc. Solvates of the compounds herein also include solution-phase forms. Thus, in some embodiments, the present disclosure provides solution-phase compositions of the compounds of the present disclosure, or any pharmaceutically acceptable salts, polymorphs, stereoisomers, or tautomers thereof, which are in solvated form, preferably fully solvated form.

### Solid Dispersion

Disclosed herein is a solid dispersion (e.g., solid molecular complex) which includes a compound of Formula (I), or a pharmaceutically acceptable salt, a polymorph, stereoisomer, a tautomer, solvate, or active metabolite thereof, and a polymer. In some embodiments, a solid dispersion that includes the compound of Formula (I) and a polymer is provided. In some embodiments, a solid molecular complex that includes the compound of Formula (I) and a polymer is provided. In addition to the compound of Formula (I), or a pharmaceutically acceptable salt, a polymorph, stereoisomer, a tautomer, solvate, or active metabolite thereof, and the polymer, the solid dispersion (e.g., solid molecular complex) may optionally contain one or more pharmaceutically acceptable excipients.

The solid dispersion (e.g., solid molecular complex) may comprise a single compound of Formula (I), or a pharmaceutically acceptable salt, a polymorph, stereoisomer, or solvate thereof, or a mixture of compounds of Formula (I), or their salts, polymorphs, stereoisomers, or solvates. The solid dispersion (e.g., solid molecular complex) may be formed from an isotopologue mixture of the disclosed compounds. In some embodiments, a subject compound of Formula (I) may be present in the solid dispersion (e.g., solid molecular complex) at a purity of at least 50% by weight, at least 60% by weight, at least 70% by weight, at least 80% by weight, at least 90% by weight, at least 95% by weight, at least 99% by weight, based on a total weight of isotopologues of the compound of Formula (I) present in the solid dispersion (e.g., solid molecular complex). For example, a solid dispersion (e.g., solid molecular complex) formulated with psilocybin d-10 (compound I-3; 3-(2-(bis(methyl-d₃)amino)ethyl-1,1,2,2-d₄)-1H-indol-4-yl dihydrogen phosphate), as the subject compound, may additionally contain isotopologues of the subject compound, e.g., psilocybin d-9, psilocybin d-8, etc., or salt forms, polymorphs, stereoisomers, solvates, or mixtures thereof. In some embodiments, the solid dispersion (e.g., solid molecular complex) is substantially free of other isotopologues of the compound, e.g., the solid dispersion has less than 20, 15, 10, 9, 8, 7, 6, 5, 4, 3, 2 or 1 or 0.5 mole percent of other isotopologues of the compound.

In some embodiments, any position in the compound having deuterium has a minimum deuterium incorporation of at least 10 atom %, at least 20 atom %, at least 25 atom %, at least 30 atom %, at least 40 atom %, at least 45 atom %, at least 50 atom %, at least 60 atom %, at least 70 atom %, at least 80 atom %, at least 90 atom %, at least 95 atom %, at least 99 atom % at the site of deuteration.

The solid dispersion (e.g., solid molecular complex) may be formulated with an enantiomerically pure compound of the present disclosure, e.g., a compound of Formula (I), or a racemic mixture of the compounds. As described herein, a racemic compound of Formula (I) may contain about 50% of the R- and S-stereoisomers based on a molar ratio (about 48 to about 52 mol %, or about a 1:1 ratio)) of one of the isomers. In some embodiments, the solid dispersion may be formed from combining separately produced compounds of the R- and S-stereoisomers in an approximately equal molar ratio (e.g., about 48 to 52%). In some embodiments, the solid dispersion may contain a mixture of separate compounds of the R- and S-stereoisomers in different ratios. In some embodiments, the solid dispersion contains an excess (greater than 50%) of the R-enantiomer. Suitable molar ratios of R/S may be from about 1.5:1, 2:1, 3:1, 4:1, 5:1, 10:1, or higher. In some embodiments, a solid dispersion may contain an excess of the S-enantiomer, with the ratios provided for R/S reversed. Other suitable amounts of R/S may be selected. For example, the R-enantiomer may be enriched, e.g., may be present in amounts of at least about 55% to 100%, or at least 65%, at least 75%, at least 80%, at least 85%, at least 90%, about 95%, about 98%, or 100%. In some embodiments, the S-enantiomer may be enriched, e.g., in amounts of at least about 55% to 100%, or at least 65%, at least 75%, at least 80%, at least 85%, at least 90%, about 95%, about 98%, or 100%. Ratios between all these exemplary embodiments as well as greater than and less than them while still within the disclosure, all are included. Solid dispersions may contain a mixture of the racemate and a separate compound of Formula (I), or a pharmaceutically acceptable salt, a polymorph, stereoisomer, or solvate thereof.

The solid dispersion (e.g., solid molecular complex) may be formulated with one or more polymorphs of the compounds of Formula (I), including crystalline and/or amorphous polymorphs. In some embodiments, the solid dispersion (e.g., solid molecular complex) comprises a compound of Formula (I) (or a pharmaceutically acceptable salt, stereoisomer, a tautomer, or solvate thereof) primarily in amorphous form. In some embodiments, the solid dispersion (e.g., solid molecular complex) comprises a compound of Formula (I) (or a pharmaceutically acceptable salt, stereoisomer, a tautomer, or solvate thereof) in amorphous form. In some embodiments, only the amorphous form of the compound of Formula (I) is present in the solid dispersion, e.g., solid dispersions in which no crystalline forms of the compound of Formula (I) are detectable, for example by XRPD.

In some embodiments, the solid dispersion comprises a highly pure amorphous form of the compound of Formula (I). For example, the solid dispersion may comprise a compound of Formula (I), wherein at least 90%, at least 95%, at least 99%, or at least 99.5% by weight of the compound of Formula (I) present in the solid dispersion is in amorphous form. In some embodiments, the solid dispersion comprises a highly pure amorphous form of psilocybin, wherein at least 90%, at least 95%, at least 99%, or at least 99.5% by weight of psilocybin present in the solid dispersion is amorphous psilocybin.

In some embodiments, the solid dispersion is substantially free of a crystalline form(s) of the compound of Formula (I). For example, the solid dispersion may comprise a compound of Formula (I), wherein less than 10%, less than 5%, less than 1%, less than 0.5%, or 0% by weight of the compound of Formula (I) present in the solid dispersion is in crystalline form. In some embodiments, the solid dispersion comprises a highly pure amorphous form of psilocybin, wherein less than 10%, less than 5%, less than 1%, less than 0.5%, or 0% by weight of psilocybin present in the solid dispersion is in crystalline form. In some embodiments, no single crystalline polymorph of the compound of Formula (I) is present in an amount of greater than 10%, greater than 5%, greater than 1%, greater than 0.5%, based on a total amount of the compound of Formula (I) present in the solid dispersion. In some embodiments, the solid dispersion comprises a highly pure amorphous form of psilocybin, wherein no single crystalline polymorph of psilocybin is present in an amount of greater than 10%, greater than 5%, greater than 1%, greater than 0.5%, based on a total amount of psilocybin present in the solid dispersion. Examples of crystalline polymorphs of psilocybin, which are preferably undetectable or are maintained as minimum levels herein, include, but are not limited to, Form A (HATCAK & TAVZID), Form B (TAVZID01), methanol solvate (PSILOC), trihydrate (OKOKAD), and ethanol solvate (KOWHOT); see Sherwood et al., “Psilocybin: crystal structure solutions enable phase analysis of prior art and recently patented examples”, Acta Cryst. (2022), C78 (1), 36-55; as well as the polymorph described as polymorph A in U.S. Pat. No. 10,519,175 or US 2022/0088041 A1.

In some embodiments, the amorphous form of the compound of Formula (I) (e.g., amorphous psilocybin) is chemically pure, for example has a chemical purity of greater than 97%, 98%, or 99% by HPLC. In some embodiments, the amorphous form of the compound of Formula (I) (e.g., amorphous psilocybin) has no single impurity of greater than 1%, greater than 0.5%, greater than 0.4%, greater than 0.3%, or greater than 0.2%, e.g., the impurity phosphoric acid as measured by ³¹P NMR, or the impurity psilocin measured by HPLC. In some embodiments, the amorphous form of the compound of Formula (I) (e.g., amorphous psilocybin) has a chemical purity of greater than 97 area %, greater than 98 area %, or greater than 99 area % by HPLC. In some embodiments, the amorphous form of the compound of Formula (I) (e.g., amorphous psilocybin) has no single impurity greater than 1 area %, greater than 0.5 area %, greater than 0.4 area %, greater than 0.3 area %, or greater than 0.2 area % as measured by HPLC. In some embodiments, the amorphous form of the compound of Formula (I) (e.g., amorphous psilocybin) does not contain psilocin or deuterated psilocin at a level greater than 1 area %, greater than 0.5 area %, greater than 0.4 area %, greater than 0.3 area %, or greater than 0.2 area % as measured by HPLC. In some embodiments, the amorphous form of the compound of Formula (I) (e.g., amorphous psilocybin) does not contain phosphoric acid at a level greater than 1 weight %, greater than 0.5 weight %, greater than 0.4 weight %, greater than 0.3 weight %, or greater than 0.2 weight %, as measured by ³¹P NMR. In some embodiments, the amorphous form of the compound of Formula (I) (e.g., amorphous psilocybin) has a chemical assay of at least 95 weight %, at least 96 weight %, or at least 98 weight %.

In some embodiments, the compound of Formula (I) is stable in the solid dispersion (e.g., solid molecular complex) for at least 3 weeks at 25° C., or for at least 1 month at 25° C., or for at least 2 months at 25° C., or for at least 3 months at 25° C., or for at least 4 months at 25° C., or for at least 5 months at 25° C., or for at least 6 months at 25° C., or for at least 9 months at 25° C., or for at least 12 months at 25° C., or for at least 15 months at 25° C., or for at least 18 months at 25° C., or for at least 24 months at 25° C. In some embodiments, the compound of Formula (I) is immobilized so that it is primarily in amorphous form within the solid dispersion (e.g., solid molecular complex) for at least 2 weeks of storage at 40° C. and 75% relative humidity, or for at least 3 weeks of storage at 40° C. and 75% relative humidity, or for at least 1 month of storage at 40° C. and 75% relative humidity, or for at least 2 months of storage at 40° C. and 75% relative humidity, or for at least 3 months of storage at 40° C. and 75% relative humidity, or for at least 4 months of storage at 40° C. and 75% relative humidity, or for at least 5 months of storage at 40° C. and 75% relative humidity, or for at least 6 months of storage at 40° C. and 75% relative humidity, or for at least 7 months of storage at 40° C. and 75% relative humidity, or for at least 8 months of storage at 40° C. and 75% relative humidity, or for at least 9 months of storage at 40° C. and 75% relative humidity, or for at least 10 months of storage at 40° C. and 75% relative humidity, or for at least 11 months of storage at 40° C. and 75% relative humidity, or for at least 12 months of storage at 40° C. and 75% relative humidity. Accordingly, the compound of Formula (I) is immobilized so that greater than 50%, or greater than 55%, or greater than 60%, or greater than 65%, or greater than 70%, or greater than 75%, or greater than 80%, or greater than 85%, or greater than 90%, or greater than 95%, or greater than 99% of the compound present in a composition is in amorphous form, as determined for example by XRPD, mDSC, etc.

The compound of Formula (I) may be stable within the solid dispersion (e.g., solid molecular complex), in terms of the compound retaining its biological activity and/or retaining certain physical or chemical properties under certain specified conditions. In some embodiments, the compound of Formula (I) is stable if the activity at the end of the specified period is at least 50%, or at least 60%, or at least 70%, or at least 75%, or at least 80%, or at least 85%, or at least 90%, or at least 95%, or at least 98% of the activity of the compound at the beginning of the specified period. In some embodiments, the compound of Formula (I) in an amorphous form is stable if at least 50%, or at least 60%, or at least 70%, or at least 75%, or at least 80%, or at least 85%, or at least 90%, or at least 95%, or at least 98%, or at least 99% of the compound remains in the amorphous form at the end of the specified period. In further embodiments, an amorphous compound of Formula (I) is stable if it does not form any detectable crystalline peaks in powder XRD profiles during the indicated period. In some embodiments, the specified period is 6 hours, 12 hours, 24 hours, 2 days, 3 days, 4 days, 5 days, 6 days, 1 week, 2 weeks, 3 weeks, 4 weeks, 5 weeks, 6 weeks, 7 weeks, 2 months, 3 months, 4 months, 5 months, 6 months, 7 months, 8 months, 9 months, 10 months, 11 months, 1 year, or longer, or any range therebetween.

In some embodiments, the solid dispersion has a glass transition (Tg) onset of from about 110° C., from about 115° C., from about 120° C., from about 125° C., from about 130° C., from about 135° C., from about 140° C., and up to about 200° C., up to about 195° C., up to about 190° C., up to about 185° C., up to about 180° C., up to about 175° C., up to about 170° C., up to about 165° C., up to about 160° C., up to about 155° C., up to about 150° C., up to about 145° C., as determined by mDSC.

In some embodiments, the solid dispersion has a heat capacity change (ΔCp), in J/(g ° C.), of from about 0.1, from about 0.15, from about 0.2, from about 0.25, from about 0.3, from about 0.35, and up to about 0.75, up to about 0.70, up to about 0.65, up to about 0.6, up to about 0.55, up to about 0.5, up to about 0.45, up to about 0.4, as determined by mDSC.

Methods of making solid dispersions (e.g., solid molecular complexes) are also disclosed herein. In some embodiments, the amorphous form of a compound of Formula (I) can be prepared by intermediately transforming a crystalline material into a non-crystalline form, e.g., a melt or a solution. Then, the amorphous material can be prepared by cooling (e.g., quench cooling) of the melt, rapid precipitation from solution, or evaporative techniques, e.g., spray drying or freeze-drying. In some embodiments, the amorphous form is formed by direct solid conversion from the crystalline to the amorphous form, e.g., milling. In some embodiments, the amorphous form can be formed by freeze-drying (lyophilization), spray drying, dehydration, milling, melt quenching, or hot melt extrusion.

In some embodiments, the amorphous state results in the compound of Formula (I) being molecularly dispersed in an inert carrier, e.g., a polymer. In some embodiments, achieving this amorphous state includes one or more of solvent evaporation, spray drying, and melt extrusion. Melt extrusion can use a twin screw extruder to combine an active ingredient (for example, the compound of Formula (I)) with an inert carrier (e.g., a polymer) to form a solid dispersion. Typically, the twin screw extruder is heated to facilitate mixing of the active ingredient with the inert carrier. In some embodiments, the active ingredient (a compound of Formula (I)) is an agonist of a serotonin 5-HT₂ receptor. In some embodiments, the active ingredient (a compound of Formula (I)) is an agonist of a serotonin 5-HT_{2A} receptor.

Amorphous forms of the compound of Formula (I) have improved solubility in water as compared to the crystalline form but are unstable and have a tendency to crystallize. Thus, it is desired to formulate the compound of Formula (I) so that it may stably exist primarily in amorphous form, including in amorphous form.

Solid dispersions that contain the compound of Formula (I) in crystalline form can be prepared through physical mixing processes such as admixing a crystalline compound of Formula (I) with a polymer (admixtures). However, after significant experimentation, the inventors have discovered that such admixture processing is generally not sufficient for the preparation of solid dispersions containing the compound(s) of Formula (I) in amorphous form (or ASDs). Instead, solid dispersions containing amorphous forms of a compound of Formula (I) may be accessed by intermediately transforming a crystalline material into a non-crystalline form, e.g., a melt or a solution, followed by cooling, evaporating, precipitating, or freeze-drying techniques as discussed herein.

In some embodiments, pharmaceutical compositions including the compound of Formula (I) in an amorphous form are provided. In some embodiments, pharmaceutical compositions of the compound of Formula (I), in which the compound of Formula (I) exists stably in amorphous form, may be accomplished, for example, by immobilizing the compound within a matrix formed by a polymer. Accordingly, in some embodiments, the present disclosure provides solid dispersions (e.g., solid molecular complexes) that include the compound of Formula (I). For example, the compound of Formula (I) may be dispersed within a matrix formed by a polymer in its solid state such that it is immobilized in its amorphous form. In some embodiments, the polymer may prevent intramolecular hydrogen bonding or weak dispersion forces between two or more drug molecules (e.g., the compound of Formula (I)). In some embodiments, the solid dispersion provides for a large surface area, thus further allowing for improved dissolution and bioavailability of the compound of Formula (I). In some embodiments, a solid dispersion (e.g., solid molecular complex) includes a therapeutically effective amount of the compound of Formula (I).

In some embodiments, a weight ratio of the compound of Formula (I) within the solid dispersion (e.g., solid molecular complex) to the polymer therein is from about 0.5:9.5, from about 1:9, from about 1.5:8.5, from about 2:8, from about 2.5:7.5, from about 3:7, and up to about 9:1, up to about 8:2, up to about 7.5:2.5, up to about 7:3, up to about 6.5:3.5, up to about 6:4, up to about 5.5:4.5, up to about 5:5, up to about 4.5:5.5, up to about 4:6, up to about 3.7:6.3, up to about 3.5:6.5, or any range therebetween. Other weight ratios above or below these ranges may be utilized, however, in most cases, the weight ratio of the compound of Formula (I) to the polymer in the solid dispersion is equal to or less than 5:5, for example, from about 1.5:8.5 to about 4.5:5.5, from about 2:8 to about 4:6, or about 3:7 to about 3.7:6.3.

In some embodiments, the compound of Formula (I) may be present in the solid dispersion in an amount of from about 0.1 wt. %, from about 0.5 wt. %, from about 1 wt. %, from about 5 wt. %, from about 10 wt. %, from about 15 wt. %, from about 20 wt. %, from about 25 wt. %, from about 30 wt. %, and up to about 90 wt. %, up to about 85 wt. %, up to about 80 wt. %, up to about 75 wt. %, up to about 70 wt. %, up to about 65 wt. %, up to about 60 wt. %, up to about 55 wt. %, up to about 50 wt. %, up to about 45 wt. %, up to about 40 wt. %, up to about 35 wt. %, based on a total weight of the solid dispersion, or any range therebetween. For example, the compound of Formula (I) may be present in the solid dispersion in an amount of from about 10 wt. % to about 70 wt. %, or from about 20 wt. % to about 60 wt. %, or from about 20 wt. % to about 40 wt. %, or about 26 wt. % to about 30 wt. %, based on a total weight of the solid dispersion. In some embodiments, the compound of Formula (I) is present in the solid dispersion in an amount of from about 1 wt. % to about 50 wt. %, or from about 10 wt. % to about 40 wt. %, or from about 20 wt. % to about 35 wt. %, or from about 25 wt. % to about 30 wt. %, based on a total weight of the solid dispersion.

Typically, the solid dispersion (e.g., solid molecular complex) is formulated with a polymer in an amount of not less than about 5 wt. %, based on a total weight of the solid dispersion. In some embodiments, the polymer may be present in the solid dispersion in an amount of from about 5 wt. %, from about 10 wt. %, from about 15 wt. %, from about 20 wt. %, from about 25 wt. %, from about 30 wt. %, from about 35 wt. %, from about 40 wt. %, from about 45 wt. %, from about 50 wt. %, and up to about 95 wt. %, up to about 90 wt. %, up to about 85 wt. %, up to about 80 wt. %, up to about 75 wt. %, up to about 70 wt. %, up to about 65 wt. %, up to about 60 wt. %, up to about 55 wt. %, based on a total weight of the solid dispersion, or any range therebetween. For example, the polymer may be present in the solid dispersion in an amount of from about 20 wt. % to about 95 wt. %, or in an amount of from about 20 wt. % to about 70 wt. %, based on a total weight of the solid dispersion. In some embodiments, a polymer is present in the solid dispersion in an amount of from about 0 wt. % to about 50 wt. %, or from about 5 wt. % to about 60 wt. %, or from about 10 wt. % to about 70 wt. %, based on a total weight of the solid dispersion. In some embodiments, a polymer is present in the solid dispersion in an amount greater than about 10 wt. %, or greater than about 20 wt. %, or greater than about 30 wt. %, or greater than about 40 wt. %, or greater than about 50 wt. %, based on a total weight of the solid dispersion. In some embodiments, the solid dispersion is about 30 wt. % of the compound of Formula (I) and about 70 wt. % of the polymer.

The solid dispersion (e.g., solid molecular complex) may be formulated with a single polymer, or a blend/mixture of different polymers. The polymer may be linear, branched, or crosslinked. The polymer may be a homopolymer or copolymer. In some embodiments, the polymer is a homopolymer. In some embodiments, the polymer is a copolymer. Copolymers may be made formed from two or more, three or more, or four or more monomer species, and may be linear, block, alternating, periodic, statistical/random, stereoblock, gradient, graft, star, or branched copolymers. In some embodiments, the polymer is a synthetic polymer. Examples of the synthetic polymers include, but are not limited to, (1) vinyl polymers such as polyvinyl acetate (PVAc), polyvinyl alcohol (PVA), polyvinylpyrrolidone (PVP) including crosslinked PVP, polyvinyl caprolactam (PVCap), etc.; (2) acrylates such as poly(acrylic) acid, alkyl acrylates (e.g., methyl acrylate, ethyl acrylate, butyl acrylate, etc.); (3) methacrylates, such as EUDRAGIT® type copolymers, poly(methacrylic) acid, alkyl methacrylates (e.g., methylmethacrylate, butylmethacrylate, etc.), amino methacrylate copolymers (e.g., based on N,N-dimethylaminoethyl methacrylate), poly(2-hydroxyethyl methacrylate), etc., for example EUDRAGIT® E PO (EPO; a cationic low viscosity terpolymer based on N,N-dimethylaminoethyl methacrylate-methylmethacrylate-butylmethacrylate; 2:1:1; weight average molecular weight of about 47,000 g/mol; immediate release; soluble below and permeable above pH 5.0; available from Evonik) and EUDRAGIT® L 100-55 (an anionic 1:1 copolymer of methacrylic acid-ethyl acrylate; delayed release; dissolution above pH 5.5; available from Evonik); (4) urethanes; (5) esters; and (6) oxides, such as polyethylene glycol (PEG) and polypropylene glycol (PPG).

In some embodiments, the polymer is a naturally occurring polymer or a derivative of a naturally occurring polymer. Examples of naturally occurring polymers or derivatives of naturally occurring polymers include, but are not limited to, (1) polysaccharides such as chitin, chitosan, dextran, pullulan, gum agar, gum arabic, gum karaya, locust bean gum, gum tragacanth, carrageenans, gum ghatti, guar gum, xanthan gum and scleroglucan; (2) starches such as dextrin and maltodextrin; (3) hydrophilic colloids such as pectin; (4) phosphatides such as lecithin; (5) alginates such as ammonium alginate, sodium, potassium or calcium alginate, propylene glycol alginate, etc.); (6) gelatin; (7) collagen; and (8) cellulose polymers such as ethyl cellulose (EC), methyl cellulose (MC), methylethyl cellulose (MEC), carboxymethyl cellulose (CMC) (e.g., including cross-linked CMC such as sodium croscarmellose), carboxymethyl ethyl cellulose (CMEC), hydroxyethyl cellulose (HEC), hydroxypropyl cellulose (HPC), cellulose acetate (CA), cellulose propionate (CP), cellulose butyrate (CB), cellulose acetate butyrate (CAB), cellulose acetate phthalate (CAP), cellulose acetate trimellitate (CAT), hydroxypropyl methyl cellulose (HPMC), hydroxypropyl methyl cellulose phthalate (HPMCP), hydroxypropyl methyl cellulose acetate succinate (HPMCAS), hydroxypropyl methyl cellulose acetate trimellitate (HPMCAT), and ethylhydroxy ethylcellulose (EHEC).

The polymer used in the solid dispersion may be a blend of any two or more polymers described herein (e.g., HPMC/PVP). In some embodiments, a blended polymer system containing a first polymer (e.g., HPMC) and a second polymer (e.g., PVP) is used. A weight ratio of the first polymer to the second polymer typically ranges from at least 1:99, at least 5:95, at least 10:90, at least 15:85, at least 20:80, at least 25:75, at least 30:70, at least 35:65, at least 40:60, at least 45:55, at least 50:50, and up to 99:1, up to 95:5, up to 90:10, up to 85:15, up to 80:20, up to 75:25, up to 70:30, up to 65:35, up to 60:40, up to 55:45.

The polymer used in the solid dispersion may be a copolymer of any two or more monomer species constituting the polymers described herein (e.g., copolymer of vinyl pyrrolidone and vinyl acetate, copovidone or PVP-VAc), aminomethacrylate copolymers, polyethylene glycol-polyvinyl acetate-polyvinylcaprolactame-based graft copolymer (PVAc-PVCap-PEG), acrylate and/or methacrylate copolymers, etc.). In some embodiments, a copolymer derived from a first monomer (e.g., vinyl pyrrolidone, VP) and a second monomer (e.g., vinyl acetate (VAc)) is used. A weight ratio of the first monomer to the second monomer in the copolymer typically ranges from at least 1:99, at least 5:95, at least 10:90, at least 15:85, at least 20:80, at least 25:75, at least 30:70, at least 35:65, at least 40:60, at least 45:55, at least 50:50, and up to 99:1, up to 95:5, up to 90:10, up to 85:15, up to 80:20, up to 75:25, up to 70:30, up to 65:35, up to 60:40, up to 55:45. Exemplary copolymers may include, but are not limited to, EUDRAGIT® type copolymers, KOLLIDON® VA 64 (a 60:40 copolymer of VP:VAc, 45,000-75,000 g/mol, available from BASF) and VIVAPHARM® PVP/VA 64 (a 6:4 linear random copolymer of VP:VAc, available from JRS Pharma).

The polymer may be a nonionic polymer or an ionic polymer (cationic, anionic, or contains a mixture of cationic and anionic monomer units). In some embodiments, the solid dispersion (e.g., solid molecular complex) comprises the compound of Formula (I) dispersed in a nonionic polymer. This may be accomplished by various means, including: (A) melting the polymer and dissolving the compound, and optionally any pharmaceutically acceptable excipient(s), in the polymer and then cooling the mixture; or (B) dissolving both the compound of Formula (I) and the polymer in a solvent (e.g., water, an organic solvent, or mixtures thereof), optionally with one or more pharmaceutically acceptable excipients, and removing/evaporating the solvent, for example, through lyophilization, spray drying techniques, in a rotary evaporator, etc. The resulting solid dispersion may comprise the compound of Formula (I) dispersed in the polymer primarily in amorphous form, including in amorphous form.

In some embodiments, the solid dispersion (e.g., solid molecular complex) comprises the compound of Formula (I) dispersed in an ionic polymer. Such solid dispersions may in some instances result in increased stability of the compound of Formula (I). This may be accomplished by various means, including the methods described above for use in forming a dispersion in a nonionic polymer. Because ionic polymers have pH dependent solubility in aqueous systems, the resulting solid dispersion of the compound of Formula (I) and the polymer may be formulated for stability at low pH in the stomach and to release the compound of Formula (I) in the intestine at higher pH (e.g., when an anionic polymer is used), and vice vera, may be formulated for stability at high pH but release the compound of Formula (I) in the stomach at lower pH (e.g., when a cationic polymer is used). In some embodiments, the compound of Formula (I) in such solid dispersions with an ionic polymer may thus be less capable of separating from the polymer and may be immobilized by the polymer in its amorphous form. Examples of such ionic polymers include hydroxypropyl methyl cellulose acetate succinate (HPMCAS), hydroxypropyl methyl cellulose phthalate (HPMCP), and methacrylic acid copolymers. In some embodiments, a polymer is used that is capable of immobilizing the compound of Formula (I) so that it exists primarily in an amorphous form for an extended period of time.

In some embodiments, the polymer is at least one selected from the group consisting of a vinyl polymer, a methacrylate, a polysaccharide, gelatin, and a cellulose polymer, or a blend or a copolymer thereof. In some embodiments, the polymer is at least one selected from the group consisting of gelatin, polyvinyl acetate, polyvinyl alcohol, polyvinylpyrrolidone, pullulan, a cellulose polymer described herein (e.g., HPMC, HPMCAS, HPMCP, etc.), a methacrylate copolymer, including blends and copolymers thereof. In some embodiments, the pharmaceutical composition includes a solid dispersion (e.g., solid molecular complex) comprising the compound of Formula (I) dispersed in a gelatin matrix (as the polymer component), and optionally a pharmaceutically acceptable excipient such as a non-reducing sugar (e.g., mannitol) and/or a pH modifier (e.g., sodium hydroxide). In some embodiments, the pharmaceutical composition includes a solid dispersion (e.g., solid molecular complex) comprising the compound of Formula (I) dispersed within a matrix formed by a cellulose polymer described herein. In some embodiments, the cellulose polymer is HPMC. In some embodiments, the cellulose polymer is HPMCAS. In some embodiments, the pharmaceutical composition includes a solid dispersion (e.g., solid molecular complex) comprising the compound of Formula (I) dispersed within a matrix formed by a cellulose polymer described herein and polyvinylpyrrolidone. In some embodiments, the solid dispersion comprises a blend of HPMC and PVP as the polymer component.

The disclosed solid dispersions formulated with one or more compounds of Formula (I) in amorphous form are advantageous in that the amorphous form of the compound(s) of Formula (I) provides consistent dissolution kinetics for predictable pharmacokinetic behavior and clinical outcomes, yet, the release kinetics from the solid dispersion can be tuned/controlled by selection of an appropriate polymer or polymer blend. For example, the use of hydroxypropyl methyl cellulose acetate succinate (HPMCAS) may provide solid dispersions with extended-release profiles, while the use of other polymers, such as gelatin, may be used to provide solid dispersions with immediate/rapid release profiles. In some embodiments, the solid dispersion (e.g., solid molecular complex) comprises, as polymer component, hydroxypropyl methyl cellulose acetate succinate (HPMCAS), examples of which include, but are not limited to, AQUASOLVE™ HPMCAS L, M, or H grade, such as AQUASOLVE™ HPMCAS MF (7-11% acetyl, 10-14% succinoyl, 21-25% methoxyl, 5-9% hydroxypropoxy; viscosity of 2.4-3.6 mPa s as 2% solution in water at 20° C.; less than 10 μm mean particle size), available from Ashland; and AQOAT® polymers such as AQOAT® AS-MG (9% acetyl, 11% succinoyl; 1,000 μm mean particle size; dissolution pH≥6.0), available from Shin-Etsu Chemical Co. Ltd.

Further, the release kinetics of the compound of Formula (I) from the solid dispersion can also be tuned/controlled by selection of a suitable molecular weight of polymer. In some embodiments, the solid dispersion comprises a polymer(s) having a low viscosity grade, e.g., a low molecular weight, such as a weight average molecular weight of less than or equal to 100,000 g/mol, less than or equal to 90,000 g/mol, less than or equal to 80,000 g/mol, less than or equal to 70,000 g/mol, less than or equal to 60,000 g/mol, less than or equal to 50,000 g/mol, less than or equal to 40,000 g/mol, less than or equal to 30,000 g/mol, less than or equal to 20,000 g/mol, less than or equal to 15,000 g/mol. Typically, the lower limit of weight average molecular weight for low viscosity grade polymers may be from 1,000 g/mol, from 2,000 g/mol, from 4,000 g/mol, from 6,000 g/mol, from 8,000 g/mol, from 10,000 g/mol, from 12,000 g/mol, from 14,000 g/mol. In some embodiments, the low molecular weight polymer is a low molecular weight hydroxypropyl methyl cellulose (HPMC) polymer, having a molecular weight within the above recite range, alone or as a polymer blend (e.g., blended with PVP). Examples of a low molecular weight hydroxypropyl methyl cellulose (HPMC) polymers which can be used herein include, but are not limited to, AFFINISOL™ HPMC HME 15LV (water soluble; amorphous HPMC polymer with a molecular weight of less than 100 kDa; bulk density of 0.42 g/cc; D(0.5) of 104.49 μm), METHOCEL™ E3 LV (2910 substitution type: 28-30% methoxy substitution, 7-12% hydroxypropyl substitution; viscosity of 4.0-6.0 mPa s as 2% solution in water at 20° C.), METHOCEL™ E6 premium LV (70,000-80,000 g/mol, 2910 substitution type: 28-30% methoxy substitution, 7-12% hydroxypropyl substitution; viscosity of 4.8-7.2 mPa s as 2% solution in water at 20° C.), each available from DuPont, and PHARMACOAT® 606 (2910 substitution type: 28-30% methoxy substitution, 7-12% hydroxypropyl substitution; viscosity of 6.0 mPa s as 2% solution in water at 20° C.), available from Shin-Etsu Chemical Co. Ltd. Examples of a PVP polymer which can be used herein include, but are not limited to, KOLLIDON® 12PF (weight average molecular weight of 2,500 g/mol; bulk density of 400-600 g/L; D(0.5) of 35 μm±5 μm) and KOLLIDON® 30 (also called PVP K-30, amorphous, water-soluble polyvinylpyrrolidone with a weight average molecular weight of 44,000-54,000 g/mol) each available from BASF. The selection of a low molecular weight polymer may provide solid dispersions adapted for immediate release or fast release of the compound of Formula (I). For example, immediate release may refer to dosage forms which release greater than 80 wt. % of the active ingredient within about 1 minute following administration, while the phrase fast release may refer to dosage forms in which the release of 80 wt. % of the active ingredient takes place in a range of about 1 minute to about 5 minutes following administration. While not limited to specific manufacturing techniques, solid dispersions comprising a low molecular weight polymer or polymer blend may be advantageously suited for freeze drying or spray drying preparation methods.

In some embodiments, the solid dispersion comprises a polymer having a high viscosity grade, e.g., a high molecular weight, such as a weight average molecular weight of at least 150,000 g/mol, at least 200,000 g/mol, at least 250,000 g/mol, at least 300,000 g/mol, at least 350,000 g/mol, at least 400,000 g/mol, at least 450,000 g/mol, at least 500,000 g/mol, at least 550,000 g/mol, at least 600,000 g/mol, at least 650,000 g/mol, at least 700,000 g/mol, at least 750,000 g/mol, at least 800,000 g/mol, at least 850,000 g/mol, at least 900,000 g/mol, at least 950,000 g/mol, at least 1,000,000 g/mol. The upper limit of molecular weight for high viscosity grade polymers is not particularly limited, but is typically up to 5,000,000 g/mol, 4,000,000 g/mol, 3,000,000 g/mol, or 2,000,000 g/mol. In some embodiments, the high molecular weight polymer is a high molecular weight hydroxypropyl methyl cellulose (HPMC) polymer, having a molecular weight within the above recite range, alone or as a polymer blend (e.g., blended with PVP). Examples of a high molecular weight hydroxypropyl methyl cellulose (HPMC) polymer which can be used herein include, but are not limited to, AFFINISOL™ HPMC HME 100LV or HPMC HME 4M, each available from DuPont; METHOCEL™ K100LV (164,000 g/mol), METHOCEL™ K4M (400,000 g/mol), METHOCEL™ K15M (575,000 g/mol), each available from Colorcon, Inc.; BENECEL™ K35M Pharm (2208 substitution type; 675,000 g/mol) and BENECEL™ K100LV PH PRM (2208 substitution type; 164,000 g/mol), each available from Ashland. High molecular weight polymers or polymer blends may provide solid dispersions adapted for either fast release or extended-release dosage forms, but are particularly well suited for extended-release applications where it is desirable to release the compound of Formula (I) over extended periods of time, such as for example over 10 minutes, 20 minutes, 30 minutes, 1 hour, 2 hours, 4 hours, 6 hours, 8 hours, 10 hours, 12 hours, or any range in between, or longer. For example, extended-release may refer to dosage forms in which the release of 80 wt. % of the active ingredient takes place in a range of about 5 minutes or longer, 10 minutes or longer, 15 minutes or longer, etc. following administration. While not limited to specific manufacturing techniques, solid dispersions comprising a high molecular weight polymer or polymer blend may be advantageously suited for hot melt extrusion, spray drying, or freeze drying preparation methods.

In some embodiments, the pharmaceutical composition comprises a solid dispersion (e.g., solid molecular complex) that includes the compound of Formula (I) dispersed within a matrix formed by gelatin. Various grades (e.g., various bloom numbers) and sources of gelatin, including gelatin derived from fish or mammalian sources (e.g., bovine), may be used herein. An example of a gelatin includes, but is not limited to, fish gelatin (super fine), available from Ajinomoto, USA. In some embodiments, the solid dispersion (e.g., solid molecular complex) further includes a non-reducing sugar, e.g., mannitol and/or a pH modifier (e.g., sodium hydroxide). The weight ratio of the compound of Formula (I) within the solid dispersion (e.g., solid molecular complex) to the gelatin (polymer) therein is generally within the range set forth herein, for example, from about 1:9 to about 5:5, from about 2:8 to about 4:6, from about 3:7 to about 3.7:6.3.

Various optional pharmaceutically acceptable excipients can be mixed, ground, granulated, or otherwise incorporated into the solid dispersion as described herein to form a material suitable for a particular dosage form or administration route. Potentially beneficial excipients may fall generally into the following classes: other matrix materials, fillers, or diluents; surface active agents; drug complexing agents or solubilizing agents; disintegrants, binders, lubricants, and pH modifiers (e.g., acids, bases, or buffers such as phosphate or citrate salts/buffers). To the extent of any overlap with the polymer component of the solid dispersion (e.g., solid molecular complex), the optional excipients which are considered to be part of the solid dispersion, are considered to be separate and distinct from the polymer in the solid dispersions herein.

Examples of other matrix materials, fillers, or diluents include, but are not limited to, lactose, mannitol, xylitol, microcrystalline cellulose, silicified microcrystalline cellulose, and calcium diphosphate.

Examples of surface active agents include, but are not limited to, sodium lauryl sulfate and polysorbate 80.

Examples of drug complexing agents or solubilizing agents include, but are not limited to, caffeine, xanthene, gentisic acid, cylodextrins. sodium phosphate, natural amino acids, acacia, cholesterol, diethanolamine (adjunct), glyceryl monostearate, lanolin alcohols, mono- and di-glycerides, monoethanolamine (adjunct), lecithin, oleic acid (adjunct), oleyl alcohol (stabilizer), polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, diacetate, monostearate, sodium lauryl sulfate, sodium stearate, sorbitan monolaurate, sorbitan monooleate, sorbitan monopalmitate, sorbitan monostearate, stearic acid, trolamine, and emulsifying wax.

Examples of disintegrants include, but are not limited to, sodium starch gycolate and calcium carbonate.

Examples of binders include microcrystalline cellulose, silicified microcrystalline cellulose, and sugars, such as sucrose, mannitol, glucose, dextrose, molasses, and lactose.

Examples of lubricants include, but are not limited to, magnesium stearate and calcium stearate.

Examples of pH modifiers include, but are not limited to, acids such as citric acid, acetic acid, ascorbic acid, lactic acid, aspartic acid, succinic acid, phosphoric acid, and the like; bases such as sodium acetate, potassium acetate, calcium oxide, magnesium oxide, trisodium phosphate, sodium hydroxide, calcium hydroxide, aluminum hydroxide, and the like, and buffers generally comprising mixtures of acids and the salts of said acids.

In some embodiments, there is provided a solid dispersion comprising the compound of Formula (I) primarily in amorphous form for use in medicine. In some embodiments, there is provided a solid dispersion comprising an amorphous form of the compound of Formula (I) (e.g., amorphous psilocybin) for use in medicine. In some embodiments, there is provided a solid dispersion comprising a highly pure amorphous form of the compound of Formula (I) (e.g., amorphous psilocybin) for use in medicine.

The pharmaceutical composition may, in addition to the solid dispersion (e.g., solid molecular complex), also optionally comprise therapeutically inert, inorganic or organic excipients (for example, pharmaceutically acceptable excipients) as a separate and distinct component of the dosage form from the solid dispersion. Thus, pharmaceutically acceptable excipients which are dispersed within the solid dispersion may be differentiated from pharmaceutically acceptable excipients which are not dispersed within the solid dispersion but are nonetheless present in the pharmaceutical composition, even though the same chemicals, compounds, or materials may be used for either. For purposes of illustration, a pharmaceutical composition in tablet form may contain lower and upper layers comprising one or more pharmaceutical acceptable excipients, which surround or sandwich a core layer formed from a solid dispersion comprising the compound of Formula (I) and one or more pharmaceutical acceptable excipients dispersed with a polymer. Here, the pharmaceutically acceptable excipient(s) of the lower and upper layers would be considered a distinct component of the dosage form from the pharmaceutically acceptable excipient(s) present and dispersed within the core layer (excipients within the solid dispersion). In another example, a solid dispersion comprising the compound of Formula (I) and a pharmaceutical acceptable excipient dispersed with a polymer may be coated with a pharmaceutically acceptable excipient, with the pharmaceutically acceptable excipient of the coating being a separate component from the pharmaceutically acceptable excipient present within the solid dispersion.

“Pharmaceutically acceptable excipients” may be excipients approved by a regulatory agency of the Federal or a state government or listed in the U.S. Pharmacopeia or other generally recognized pharmacopeia for use in mammals, such as humans. The term “excipient” herein refers to a vehicle, diluent, adjuvant, carrier, or any other auxiliary or supporting ingredient with which the solid dispersion containing the active ingredient (e.g., a compound of Formula (I)) is formulated for administration to a mammal. Such pharmaceutical excipients can be liquids, such as water and oils, including those of petroleum, animal, vegetable or synthetic origin, such as peanut oil, soybean oil, mineral oil, sesame oil and the like. The pharmaceutical excipients can be water, saline, gum acacia, gelatin, starch paste, talc, keratin, colloidal silica, urea, and the like. The pharmaceutical excipients can include one or more gases, e.g., to act as a carrier for administration via inhalation. In addition, auxiliary, stabilizing, thickening, lubricating, taste masking, coloring agents, and other pharmaceutical additives may be included in the disclosed compositions, such as any of those set forth herein. The pharmaceutical composition may thus be formulated with additional agents such as preserving agents, solubilizing agents, stabilizing agents, wetting agents, emulsifying agents, sweetening agents, coloring agents, flavoring agents, salts for varying the osmotic pressure, buffers, coating agents and antioxidants. The pharmaceutical composition may also contain additional therapeutically active ingredients or more than one therapeutically active ingredient/polymer complex (e.g., a solid dispersion, for example a solid molecular complex).

In some embodiments, the pharmaceutical composition includes the solid dispersion (e.g., solid molecular complex) suspended in an aqueous vehicle containing hydroxypropylcellulose (HPC). In some embodiments, the vehicle contains about 2% by weight HPC. In some embodiments, the pharmaceutical composition includes colloidal silicon dioxide (silica). In some embodiments, the addition of colloidal silicon dioxide may further improve the stability of the solid dispersion (e.g., solid molecular complex). In some embodiments, the pharmaceutical composition includes at least about 0.5% by weight colloidal silicon dioxide.

Pharmaceutical compositions are provided herein which comprise about 0.1 to about 1000 mg, about 1 to about 500 mg, about 2 to about 100 mg, about 1 mg, about 2 mg, about 3 mg, about 5 mg, about 10 mg, about 20 mg, about 30 mg, about 40 mg, about 50 mg, about 100 mg, about 250 mg, about 500 mg of one or more compounds of Formula (I) as disclosed herein. The quantity of compound of Formula (I) (on active basis) in a unit dose preparation may be varied or adjusted within the above ranges as deemed appropriate using sound medical judgment, according to the particular application, administration route, potency of the active component, etc. The pharmaceutical composition can, if desired, also contain other compatible therapeutic agents. The term “unit dosage form” or “unit dose” will be used herein to refer to compositions formulated with an amount of an active pharmaceutical ingredient (API) in a dose for administration as a single dose to a target individual. The unit dosage form may be adapted, depending on the nature of the active ingredient, the indication, the disease stage and various other factors known per se for once, twice, thrice or any other number of daily, weekly, or monthly administrations. In some embodiments, a formulated dose comprises between about 2 and about 50 mg of the compound of Formula (I) in amorphous form (e.g., amorphous psilocybin). In some embodiments, a formulated dose comprises between 2 mg and 40 mg, between 2 mg and 10 mg, between 5 mg and 30 mg, between 5 mg and 15 mg, or between 20 mg and 30 mg of the compound of Formula (I) in amorphous form (e.g., amorphous psilocybin). In some embodiments, a formulated dose comprises about 1 mg, about 5 mg, about 10 mg, or about 25 mg of the compound of Formula (I) in amorphous form (e.g., amorphous psilocybin).

In some embodiments, the pharmaceutical compositions disclosed herein are adapted for oral administration, including intraoral administration such as through the mucosal linings of the oral cavity, e.g., buccal, lingual, and sublingual administration. In some embodiments, the oral dosage form comprises 5 mg, 10 mg, 15 mg, 20 mg, 25 mg, 30 mg, 35 mg, 40 mg, 45 mg, 50 mg of the compound of Formula (I) (e.g., psilocybin), or any range therebetween.

Intraoral dosage forms allow for pre-gastric absorption of the compounds herein, e.g., when administered intraorally through the mucosal linings of the oral cavity, e.g., buccal, lingual, and sublingual administration, for increased bioavailability and faster onset compared to oral administration through the gastrointestinal tract. In some embodiments, the pharmaceutical compositions disclosed herein are in orodispersible dosage forms (ODFs). ODFs can be prepared by different techniques, such as freeze-drying (lyophilization), molding, spray drying, mass extrusion or compressing. Preferably, the ODFs are prepared by lyophilization. ODFs encompass solid dosage forms that disintegrate or dissolve in the mouth within about 90 seconds, 60 seconds, 30 seconds, 20 seconds, 10 seconds, 5 seconds, 2 seconds or less after being received in the oral cavity. In some embodiments, an orodispersible dosage form disperses in the mouth within 10, 9, 8, 7, 6, 5, 4, 3, 2, or even within 1 second. In some embodiments, the pharmaceutical compositions are in the form of orodispersible dosage forms having a disintegration time according to the United States Phamacopeia (USP) disintegration test <701> of not more than about 30 seconds, not more than about 20, not more than about 10 seconds, not more than about 5 seconds, not more than about 2 seconds. Orodispersible dosage forms having longer disintegration times according to the United States Phamacopeia (USP) disintegration test <701>, such as when adapted for extended-release, for example 2 minutes, 3 minutes, 4 minutes, 5 minutes, 10 minutes, 15 minutes, 20 minutes, 25 minutes, 30 minutes, 45 minutes, 60 minutes, or any range therebetween, or longer, are also contemplated.

In some embodiments, the pharmaceutical compositions are in the form of fast dissolving tablets (FDTs), also called oral disintegrating tablets (or orodispersible tablets) (ODTs) or fast dispersible tablets. Fast dissolving tablets can be prepared by different techniques, such as freeze-drying (lyophilization), molding, spray drying, mass extrusion or compressing. In some embodiments, fast dissolving tablets are prepared by lyophilization. In some embodiments, fast dissolving tablet refers to forms which disintegrate in less than about 90 seconds, in less than about 60 seconds, in less than about 30 seconds, in less than about 20, in less than about 10 seconds in the oral cavity, in less than about 5 seconds, or in less than about 2 seconds after being received in the oral cavity. In some embodiments, fast dissolving tablet refers to forms which dissolve in less than about 90 seconds, in less than about 60 seconds, in less than about 30 seconds, in less than about 20, in less than about 10 seconds in the oral cavity, in less than about 5 seconds, or in less than about 2 seconds after being received in the oral cavity.

In some embodiments, the pharmaceutical compositions are in the form of lyophilized FDTs. In some embodiments, the lyophilized FDTs are created by creating a porous matrix by subliming the water from pre-frozen aqueous formulation of the drug containing matrix-forming agents and other excipients such as lyoprotectants, preservatives, pH modifiers, and flavors. In some embodiments, the FDTs comprise two component frameworks of a lyophilized matrix system that work together to ensure the development of a successful formulation. In some embodiments, the first component is a water-soluble polymer such as gelatin, dextran, alginate, and maltodextrin. This component maintains the shape and provides mechanical strength to the tablets (binder). In some embodiments, the second constituent is matrix-supporting/disintegration-enhancing agents such as sucrose, lactose, mannitol, xylitol, microcrystalline cellulose, and/or calcium diphosphate, which acts by cementing the porous framework, provided by the water-soluble polymer and accelerates the disintegration of the FDT. In some embodiments, the lyophilized FDT includes gelatin and mannitol. In some embodiments, the lyophilized FDTs include gelatin, mannitol, and one or more of a lyoprotectant, a preservative, an antioxidant, a stabilizing agent, a solubilizing agent, a flavoring agent, a pH modifier, etc., with particular mention being made to sodium hydroxide. A non-limiting example of an FDT formulation is Zydis® orally dispersible tablets (available from Catalent). In some embodiments, the FDT formulation (e.g., Zydis® orally dispersible tablets) includes a solid dispersion formed from (a) one or more water-soluble polymers, such as gelatin, (b) one or more matrix materials, fillers, or diluents, such as mannitol, (c) a compound of Formula (I), or a pharmaceutically acceptable salt, polymorph, stereoisomer, or solvate thereof, and optionally (d) a lyoprotectant, a preservative, an antioxidant, a stabilizing agent, a solubilizing agent, a pH modifier, and/or a flavoring agent. In some embodiments, the FDT formulation (e.g., Zydis® orally dispersible tablets) includes gelatin, mannitol, a compound of Formula (I), or a pharmaceutically acceptable salt, polymorph, stereoisomer, tautomer, or solvate thereof, and sodium hydroxide.

In some embodiments, the lyophilized FDTs include a cellulose polymer described herein (e.g., HPMC), either alone or as a polymer blend, e.g., with polyvinylpyrrolidone.

In some embodiments, the pharmaceutical compositions are in the form of lyophilized wafers. In some embodiments, the pharmaceutical compositions are in the form of lyophilized wafers protected for the long-term storage by a specialty packaging excluding moisture, oxygen and light. In some embodiments, the lyophilized wafers are created by creating a porous matrix by subliming the water from pre-frozen aqueous formulation of the drug containing matrix-forming agents and other excipients such as lyoprotectants, preservatives, pH modifiers, and flavors. In some embodiments, the lyophilized wafer includes a thin water-soluble film matrix. In some embodiments, the wafers comprise two component frameworks of a lyophilized matrix system that work together to ensure the development of a successful formulation. In some embodiments, the first component is water-soluble polymers such as gelatin, dextran, alginate, and maltodextrin. This component maintains the shape and provides mechanical strength to the tablets (binder). In some embodiments, the second constituent is matrix-supporting/disintegration-enhancing agents such as sucrose, lactose, mannitol, xylitol, microcrystalline cellulose, and/or calcium diphosphate, which acts by cementing the porous framework, provided by the water-soluble polymer and accelerates the disintegration of the wafer. In some embodiments, the lyophilized wafers include gelatin and mannitol. In some embodiments, the lyophilized wafers include gelatin, mannitol, and one or more of a lyoprotectant, a preservative, an antioxidant, a stabilizing agent, a solubilizing agent, a flavoring agent, a pH modifier, etc., with particular mention being made to sodium hydroxide.

In some embodiments, the lyophilized wafer includes a cellulose polymer described herein (e.g., HPMC), either alone or as a polymer blend, e.g., with polyvinylpyrrolidone. In some embodiments, the wafer can comprise a monolayer, bilayer, or trilayer. In some embodiments, the monolayer wafer contains the solid dispersion of polymer, an active ingredient (e.g., a compound of Formula (I)), and optionally one or more excipients. In some embodiments, the bilayer wafer contains one or more excipients, such as a solubilizing agent, in a first layer and a solid dispersion comprising an active ingredient and polymer in the second layer. This configuration allows the active ingredient to be stored separately from the excipients and can increase the stability of the active ingredient and optionally increase the shelf life of the pharmaceutical composition compared to the case where the excipients and the active ingredient were contained in a single layer. For tri-layer wafers, each of the layers may be different or two of the layers, such as the upper and lower layers, may have substantially the same composition. In some embodiments, the lower and upper layers surround a core layer containing the solid dispersion comprising the active ingredient. In some embodiments, the lower and upper layers may contain one or more excipients, such as a solubilizing agent. In some embodiments, the lower and upper layers have the same composition. Alternatively, the lower and upper layers may contain different excipients or different amounts of the same excipient. The core layer typically contains the solid dispersion formed from polymer(s), active ingredient, optionally with one or more excipients.

Examples of pharmaceutically acceptable lyoprotectants include, but are not limited to, disaccharides such as sucrose and trehalose, anionic polymers such as sulfobutylether-β-cyclodextrin (SBECD) and hyaluronic acid, and hydroxylated cyclodextrins.

Examples of pharmaceutically acceptable preservatives include, but are not limited to, glycerin, methyl and propylparaben, benzoic acid, sodium benzoate and alcohol.

Examples of pharmaceutically acceptable antioxidants, which may act to further enhance stability of the composition, include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of pharmaceutically acceptable stabilizing agents include, but are not limited to, fatty acids, fatty alcohols, alcohols, long chain fatty acid esters, long chain ethers, hydrophilic derivatives of fatty acids, polyvinylpyrrolidones, polyvinyl ethers, polyvinyl alcohols, hydrocarbons, hydrophobic polymers, moisture-absorbing polymers, glycerol, methionine, monothioglycerol, ascorbic acid, citric acid, polysorbate, arginine, cyclodextrins, microcrystalline cellulose, silicified microcrystalline cellulose, modified celluloses (e.g., carboxymethylcellulose, sodium salt), sorbitol, and cellulose gel.

Examples of pharmaceutically acceptable solubilizing agents (or dissolution aids) include, but are not limited to, citric acid, hydroxypropylcellulose, hydroxypropylmethylcellulose, sodium stearyl fumarate, methacrylic acid copolymer LD, methylcellulose, sodium lauryl sulfate, polyoxyl 40 stearate, purified shellac, sodium dehydroacetate, fumaric acid, DL-malic acid, L-ascorbyl stearate, L-asparagine acid, adipic acid, aminoalkyl methacrylate copolymer E, propylene glycol alginate, casein, casein sodium, a carboxyvinyl polymer, carboxymethylethylcellulose, powdered agar, guar gum, succinic acid, copolyvidone, cellulose acetate phthalate, tartaric acid, dioctylsodium sulfosuccinate, zein, powdered skim milk, sorbitan trioleate, lactic acid, aluminum lactate, ascorbyl palmitate, hydroxyethylmethylcellulose, hydroxypropylmethylcelluloseacetate succinate, polyoxyethylene (105) polyoxypropylene (5) glycol, polyoxyethylene hydrogenated castor oil 60, polyoxyl 35 castor oil, poly(sodium 4-styrenesulfonate), polyvinylacetaldiethylamino acetate, polyvinyl alcohol, maleic acid, methacrylic acid copolymer S, lauromacrogol, sulfuric acid, aluminum sulfate, phosphoric acid, calcium dihydrogen phosphate, sodium dodecylbenzenesulfonate, a vinyl pyrrolidone-vinyl acetate copolymer, sodium lauroyl sarcosinate, acetyl tryptophan, sodium methyl sulfate, sodium ethyl sulfate, sodium butyl sulfate, sodium octyl sulfate, sodium decyl sulfate, sodium tetradecyl sulfate, sodium hexadecyl sulfate, and sodium octadecyl sulfate.

Flavoring agents include natural flavors extracted from plants, such as fruits, and synthetic blends of compounds which produce a pleasant taste sensation or taste masking effect. Examples of flavoring agents include, but are not limited to, aspartame, saccharin (as sodium, potassium or calcium saccharin), cyclamate (as a sodium, potassium or calcium salt), sucralose, acesulfame-K, thaumatin, neohisperidin, dihydrochalcone, ammoniated glycyrrhizin, dextrose, maltodextrin, fructose, levulose, sucrose, glucose, wild orange peel, citric acid, tartaric acid, oil of wintergreen, oil of peppermint, methyl salicylate, oil of spearmint, oil of sassafras, oil of clove, cinnamon, anethole, menthol, thymol, eugenol, eucalyptol, lemon, lime, and lemon-lime.

Cyclodextrins such as α-cyclodextrin, β-cyclodextrin, γ-cyclodextrin, hydroxyethyl β-cyclodextrin, hydroxypropyl γ-cyclodextrin, sulfated β-cyclodextrin, sulfated α-cyclodextrin, sulfobutyl ether β-cyclodextrin, or other solubilized derivatives can also be advantageously used to enhance delivery of compositions described herein.

Disclosed herein are pharmaceutical compositions in modified release dosage forms, which comprise solid dispersions as disclosed herein and one or more release controlling excipients as described herein. Suitable modified release dosage vehicles include, but are not limited to, hydrophilic or hydrophobic matrix devices, water-soluble separating layer coatings, enteric coatings, osmotic devices, multiparticulate devices, and combinations thereof. The pharmaceutical compositions may also comprise non-release controlling excipients.

Further disclosed herein are pharmaceutical compositions in enteric coated dosage forms, which comprise solid dispersions as disclosed herein and one or more release controlling excipients for use in an enteric coated dosage form. The pharmaceutical compositions may also comprise non-release controlling excipients.

Further disclosed herein are pharmaceutical compositions in effervescent dosage forms, which comprise solid dispersions as disclosed herein and one or more release controlling excipients for use in an effervescent dosage form. The pharmaceutical compositions may also comprise non-release controlling excipients.

Additionally disclosed are pharmaceutical compositions in a dosage form that has an instant releasing component and at least one delayed releasing component, and is capable of giving a discontinuous release of the active ingredient (e.g., a compound of Formula (I)) in the form of at least two consecutive pulses separated in time from about 0.1 up to about 24 hours (e.g., about 0.1, 0.5, 1, 2, 4, 6, 8, 10, 12, 14, 16, 18, 10, 22, or 24 hours). The pharmaceutical compositions comprise a solid dispersion as disclosed herein and one or more release controlling and non-release controlling excipients, such as those excipients suitable for a disruptable semipermeable membrane and as swellable substances.

Disclosed herein also are pharmaceutical compositions in a dosage form for oral administration to a subject, which comprise a solid dispersion as disclosed herein and one or more pharmaceutically acceptable excipients, enclosed in an intermediate reactive layer comprising a gastric juice-resistant polymeric layered material partially neutralized with alkali and having cation exchange capacity and a gastric juice-resistant outer layer.

In some embodiments, the pharmaceutical compositions are in the form of immediate-release capsules for oral administration, and may further comprise cellulose, iron oxides, lactose, magnesium stearate, and sodium starch glycolate.

In some embodiments, the pharmaceutical compositions are in the form of delayed-release capsules for oral administration, and may further comprise cellulose, ethylcellulose, gelatin, hypromellose, iron oxide, and titanium dioxide.

In some embodiments, the pharmaceutical compositions are in the form of enteric coated delayed-release tablets for oral administration, and may further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, and yellow ferric oxide.

In some embodiments, the pharmaceutical compositions are in the form of enteric coated delayed-release tablets for oral administration, and may further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

The pharmaceutical compositions disclosed herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In the case wherein the patient's condition does not improve, upon the doctor's discretion the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

The compounds as disclosed herein may be administered alone or in combination with one or more other active ingredients. Pharmaceutical compositions comprising a compound disclosed herein may be formulated in various dosage forms for oral, parenteral, and topical administration. The pharmaceutical compositions may also be formulated as a modified release dosage form, including delayed-, extended-, prolonged-, sustained-, pulsatile-, controlled-, accelerated-, fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126).

In some embodiments, the pharmaceutical composition is a parenteral dosage form. In some embodiments, the pharmaceutical composition is an oral dosage form. In some embodiments, the pharmaceutical composition comprises a tablet. In some embodiments, the pharmaceutical composition comprises a capsule. In some embodiments, the pharmaceutical composition comprises a dry powder. In some embodiments, the pharmaceutical composition comprises a solution. In some embodiments, more than one dosage form is administered to the subject at substantially the same time. In some embodiments, the subject may be administered the entire therapeutic dose in one tablet or capsule. In some embodiments, the therapeutic dose may be split among multiple tablets or capsules. For example, for a dose of 25 mg, the subject may be administered 5 tablets or capsules each comprising 5 mg of the compound of Formula (I) (e.g., psilocybin). Alternatively, for a dose of 10 mg, the subject may be administered 2 tablets or capsules each comprising 5 mg of the compound of Formula (I) (e.g., psilocybin).

Any of the pharmaceutical compositions described herein can comprise a solid dispersion (e.g., a solid molecular complex) comprising a compound of Formula (I), or a pharmaceutically acceptable salt, a polymorph, stereoisomer, a tautomer, solvate, or active metabolite thereof, and a polymer.

### A. Oral Administration

The pharmaceutical compositions disclosed herein may be provided in solid, semisolid, or liquid dosage forms for oral administration. As used herein, oral administration includes gastric (enteral) delivery, for example whereby the medication is taken by mouth and swallowed, as well as intraoral administration such as through the mucosal linings of the oral cavity, e.g., also includes buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the solid dispersions (e.g., solid molecular complex) containing the active ingredient(s) and a polymer(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remains intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); silicified microcrystalline cellulose, and mixtures thereof. Suitable fillers include, but are not limited to, talc, calcium carbonate, microcrystalline cellulose, silicified microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions disclosed herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions disclosed herein varies upon the type of formulation, and is readily discernible to those of ordinary skill in the art. The pharmaceutical compositions disclosed herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions disclosed herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many excipients may serve several functions, even within the same formulation.

The pharmaceutical compositions disclosed herein may be disclosed as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the solid dispersions comprising the active ingredient, e.g., in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The dosage form may be an immediate release (IR) or fast release dosage form, examples of which include, but are not limited to, an immediate release (IR) tablet or an immediate release (IR) capsule. In addition to the solid dispersion of the present disclosure, which contains active ingredient (e.g., a compound of Formula (I)) dispersed in a polymer, dosage forms adapted for immediate or fast release may also include one or more pharmaceutically acceptable excipients which readily disperse, dissolve, or otherwise breakdown in the gastric environment so as not to delay or prolong dissolution/absorption of the active. Examples of pharmaceutically acceptable excipients for immediate or fast release dosage forms include, but are not limited to, one or more binders/granulators, matrix materials, fillers, diluents, disintegrants, dispersing agents, solubilizing agents, lubricants, and/or performance modifiers. In some embodiments, the immediate or fast release (IR) dosage form is an immediate release (IR) or fast release tablet comprising one or more of the following pharmaceutically acceptable excipients: microcrystalline cellulose, silicified microcrystalline cellulose, sodium carboxymethylcellulose, magnesium stearate, mannitol, crospovidone, and sodium stearyl fumarate. In some embodiments, the immediate release (IR) or fast release dosage form comprises microcrystalline cellulose, silicified microcrystalline cellulose, sodium carboxymethylcellulose, and magnesium stearate as pharmaceutically acceptable excipients. In some embodiments, the immediate release (IR) or fast release dosage form comprises mannitol, crospovidone, and sodium stearyl fumarate as pharmaceutically acceptable excipients.

The pharmaceutical compositions disclosed herein may be disclosed as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the solid dispersion containing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms disclosed herein may be encapsulated in a capsule. Suitable liquid and semisolid dosage forms include solutions and suspensions in propylene carbonate, vegetable oils, or triglycerides. The capsules may also be coated as known by those of skill in the art in order to modify or sustain dissolution of the active ingredient.

The pharmaceutical compositions disclosed herein may be disclosed in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) disclosed herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates. In some embodiments, examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Cyclodextrins such as α-cyclodextrin, β-cyclodextrin, γ-cyclodextrin, hydroxyethyl β-cyclodextrin, hydroxypropyl γ-cyclodextrin, sulfated β-cyclodextrin, sulfated α-cyclodextrin, sulfobutyl ether β-cyclodextrin, or other solubilized derivatives can also be advantageously used to enhance delivery of compositions described herein.

The pharmaceutical compositions disclosed herein for oral administration may be also disclosed in the forms of liposomes, micelles, microspheres, or nanosystems.

The pharmaceutical compositions disclosed herein may be disclosed as non-effervescent or effervescent, granules and powders, to be reconstituted into a liquid dosage form. Pharmaceutically acceptable excipients used in the non-effervescent granules or powders may include diluents, sweeteners, and wetting agents. Pharmaceutically acceptable excipients used in the effervescent granules or powders may include organic acids and a source of carbon dioxide.

Coloring and flavoring agents can be used in all of the above dosage forms.

The pharmaceutical compositions disclosed herein may be co-formulated with other active ingredients which do not impair the desired therapeutic action, or with substances that supplement the desired action, such as drotrecogin-α, and hydrocortisone.

### B. Parenteral Administration

The pharmaceutical compositions disclosed herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions disclosed herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH modifiers, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH modifiers include, but are not limited to, sodium hydroxide, hydrochloric acid, citric acid, and lactic acid. Suitable complexing agents include, but are not limited to, cyclodextrins, including ca-cyclodextrin, β-cyclodextrin, hydroxypropyl-3-cyclodextrin, sulfobutylether-β-cyclodextrin, and sulfobutylether 7-O-cyclodextrin (CAPTISOL®, CyDex, Lenexa, Kans.).

The pharmaceutical compositions disclosed herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

In some embodiments, the pharmaceutical compositions are disclosed as ready-to-use sterile solutions. In some embodiments, the pharmaceutical compositions are disclosed as sterile dry soluble products, including lyophilized powders and hypodermic tablets, to be reconstituted with a vehicle prior to use. In some embodiments, the pharmaceutical compositions are disclosed as ready-to-use sterile suspensions. In some embodiments, the pharmaceutical compositions are disclosed as sterile dry insoluble products to be reconstituted with a vehicle prior to use. In some embodiments, the pharmaceutical compositions are disclosed as ready-to-use sterile emulsions.

The pharmaceutical compositions may be formulated as a suspension, solid, semi-solid, or thixotropic liquid, for administration as an implanted depot. In some embodiments, the pharmaceutical compositions disclosed herein are dispersed in a solid inner matrix, which is surrounded by an outer polymeric membrane that is insoluble in body fluids but allows the active ingredient in the pharmaceutical compositions to diffuse through.

Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinyl alcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical compositions disclosed herein may be administered topically to the skin, orifices, or mucosa. Topical administration, as described herein, includes (intra)dermal, conjuctival, intracorneal, intraocular, ophthalmic, auricular, transdermal, nasal, vaginal, uretheral, respiratory, and rectal administration.

The pharmaceutical compositions disclosed herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions disclosed herein may also comprise liposomes, micelles, microspheres, nanosystems, and mixtures thereof.

Pharmaceutically acceptable excipients suitable for use in the topical formulations disclosed herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The ointments, pastes, creams and gels may contain, in addition to an active ingredient(s), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active ingredient(s), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal delivery devices (e.g., patches) have the added advantage of providing controlled delivery of active ingredient(s) to the body. That is, solid dispersions comprising the active ingredient(s) of the present disclosure (e.g., a compound of Formula (I)) can be administered via a transdermal patch at a steady state concentration, whereby the active ingredient(s) is gradually administered over time, thus avoiding drug spiking and adverse events/toxicity associated therewith.

Transdermal patch dosage forms herein may be formulated with various amounts of the active ingredient(s), depending on the disease/condition being treated, the active ingredient(s) employed, the permeation and size of the transdermal delivery device, the release time period, etc. For example, a unit dose preparation may be varied or adjusted e.g., from 5 mg, 10 mg, 15 mg, 20 mg, 25 mg, 30 mg, 35 mg, 40 mg, 45 mg, 50 mg, to 100 mg, 95 mg, 90 mg, 85 mg, 80 mg, 75 mg, 70 mg, 65 mg, 60 mg, 55 mg of the compound of Formula (I), or otherwise as deemed appropriate using sound medical judgment, according to the particular application and the potency of the active ingredient.

Transdermal patches formulated with the solid dispersion may be suitable for microdosing to achieve durable therapeutic benefits, with decreased toxicity. In some embodiments, the compound of Formula (I) may be administered via a transdermal patch at serotonergic, but sub-psychoactive concentrations, for example, over an extended period such as over a 8, 24, 48, 72, 84, 96, or 168 hour time period.

In addition to the solid dispersion of the present disclosure, and any optional pharmaceutically acceptable excipient(s), the transdermal patch may also include one or more of a pressure sensitive adhesive layer, a backing, and a release liner, as is known to those of ordinary skill in the art.

In some embodiments, the solid dispersion is dissolved/dispersed directly into a polymer matrix forming the pressure sensitive adhesive layer. In some embodiments, the compound of Formula (I) (or a pharmaceutically acceptable salt, a polymorph, stereoisomer, a tautomer, solvate, or active metabolite thereof) of the present disclosure may be dissolved/dispersed directly into a polymer matrix forming the pressure sensitive adhesive layer, that is, where the pressure sensitive adhesive layer acts as the polymer component of the solid dispersion. Such transdermal patches are called drug-in-adhesive (DIA) patches. Preferred DIA patch forms are those in which the active ingredient(s) is distributed uniformly throughout a pressure sensitive adhesive polymer matrix. In some embodiments, the active ingredient(s) may be provided in a solid dispersion in a layer which is separate from the pressure sensitive adhesive layer. In any case, the transdermal patch dosage forms may optionally be formulated with suitable excipient(s) such as carriers, permeation agents/absorption enhancers, humectants, etc. to increase the flux across the skin.

Examples of carrier agents may include, but are not limited to, C₈-C₂₂ fatty acids, such as oleic acid, undecanoic acid, valeric acid, heptanoic acid, pelargonic acid, capric acid, lauric acid, and eicosapentaenoic acid; C₈-C₂₂ fatty alcohols such as octanol, nonanol, oleyl alcohol, decyl alcohol and lauryl alcohol; lower alkyl esters of C₈-C₂₂ fatty acids such as ethyl oleate, isopropyl myristate, butyl stearate, and methyl laurate; di(lower)alkyl esters of C₆-C₂₂ diacids such as diisopropyl adipate; monoglycerides of C₈-C₂₂ fatty acids such as glyceryl monolaurate; tetrahydrofurfuryl alcohol polyethylene glycol ether; polyethylene glycol, propylene glycol; 2-(2-ethoxyethoxy)ethanol; diethylene glycol monomethyl ether; alkylaryl ethers of polyethylene oxide; polyethylene oxide monomethyl ethers; polyethylene oxide dimethyl ethers; glycerol; ethyl acetate; acetoacetic ester; N-alkylpyrrolidone; cyclodextrins, such as α-cyclodextrin, β-cyclodextrin, γ-cyclodextrin, or derivatives such as 2-hydroxypropyl-β-cyclodextrin; and terpenes/terpenoids, such as limonene, linalool, myrcene, pinene such as α-pinene, caryophyllene, citral, eucolyptol, and the like; including mixtures thereof.

Examples of permeation agents/absorption enhancers include, but are not limited to, sulfoxides, such as dodecylmethylsulfoxide, octyl methyl sulfoxide, nonyl methyl sulfoxide, decyl methyl sulfoxide, undecyl methyl sulfoxide, 2-hydroxydecyl methyl sulfoxide, 2-hydroxy-undecyl methyl sulfoxide, 2-hydroxydodecyl methyl sulfoxide, and the like; surfactant-lecithin organogel (PLO), such as those formed from an aqueous phase with one or more of poloxamers, CARBOPOL and PEMULEN, a lipid phase formed from one or more of isopropyl palmitate and PPG-2 myristyl ether propionate, and lecithin; fatty acids, esters, and alcohols, such as oleyloleate and oleyl alcohol; keto acids such as levulinic acid; glycols and glycol ethers, such as diethylene glycol monoethyl ether; including mixtures thereof.

Examples of humectants/crystallization inhibitors include, but are not limited to, polyvinylpyrrolidone-co-vinyl acetate, polymethacrylate, and mixtures thereof.

The pressure sensitive adhesive layer may be formed from polymers including, but not limited to, acrylics (polyacrylates including alkyl acrylics), polyvinyl acetates, natural and synthetic rubbers (e.g., polyisobutylene), ethylenevinylacetate copolymers, polysiloxanes, polyurethanes, plasticized polyether block amide copolymers, plasticized styrene-butadiene rubber block copolymers, and mixtures thereof. The pressure-sensitive adhesive layer used in the transdermal patch of the present disclosure may be formed from an acrylic polymer pressure-sensitive adhesive, preferably an acrylic copolymer pressure sensitive adhesive. The acrylic copolymer pressure sensitive adhesive may be obtained by copolymerization of one or more alkyl (meth)acrylates (e.g., 2-ethylhexyl acrylate); aryl (meth)acrylates; arylalkyl (meth)acrylate; and (meth)acrylates with functional groups such as hydroxyalkyl (meth)acrylates (e.g., hydroxyethyl acrylate, 2-hydroxypropyl acrylate, 3-hydroxypropyl acrylate, 4-hydroxybutyl acrylate, 2-hydroxyethyl methacrylate, 2-hydroxypropyl methacrylate, 3-hydroxypropyl methacrylate, and 4-hydroxybutyl methacrylate), carboxylic acid containing (meth)acrylates (e.g., acrylic acid), and alkoxy (meth)acrylates (e.g., methoxyethyl acrylate); optionally with one or more copolymerizable monomers (e.g., vinyl pyrrolidone, vinyl acetate, etc.). Specific examples of acrylic pressure-sensitive adhesives may include, but are not limited to, DURO-TAK products (Henkel) such as DURO-TAK 87-900A, DURO-TAK 87-9301, DURO-TAK 87-4098, DURO-TAK 87-2074, DURO-TAK 87-235A, DURO-TAK 87-2510, DURO-TAK 87-2287, DURO-TAK 87-4287, DURO-TAK 87-2516, DURO-TAK 387-2052, and DURO-TAK 87-2677.

The backing used in the transdermal patch of the present disclosure may include flexible backings such as films, nonwoven fabrics, Japanese papers, cotton fabrics, knitted fabrics, woven fabrics, and laminated composite bodies of a nonwoven fabric and a film. Such a backing is preferably composed of a soft material that can be in close contact with a skin and can follow skin movement and of a material that can suppress skin rash and other discomforts following prolonged use of the patch. Examples of the backing materials include, but are not limited to, polyethylene, polypropylene, polyethylene terephthalate, polybutylene terephthalate, polyethylene naphthalate, polystyrene, nylon, cotton, acetate rayon, rayon, a rayon/polyethylene terephthalate composite body, polyacrylonitrile, polyvinyl alcohol, acrylic polyurethane, ester polyurethane, ether polyurethane, a styrene-isoprene-styrene copolymer, a styrene-butadiene-styrene copolymer, a styrene-ethylene-propylene-styrene copolymer, styrene-butadiene rubber, an ethylene-vinyl acetate copolymer, or cellophane, for example. Preferred backings do not adsorb or release the active ingredient(s). In order to suppress the adsorption and release of the active ingredient(s), to improve transdermal absorbability of the active ingredient(s), and to suppress skin rash and other discomforts, the backing preferably includes one or more layers composed of the material above and has a water vapor permeability. Specific examples of backings may include, but are not limited to, 3M COTRAN products such as 3M COTRAN ethylene vinyl acetate membrane film 9702, 3M COTRAN ethylene vinyl acetate membrane film 9716, 3M COTRAN polyethylene membrane film 9720, 3M COTRAN ethylene vinyl acetate membrane film 9728, and the like.

The release liner used in the transdermal patch of the present disclosure may include, but is not limited to, a polyester film having one side or both sides treated with a release coating, a polyethylene laminated high-quality paper treated with a release coating, and a glassine paper treated with a release coating. The release coating may be a fluoropolymer, a silicone, a fluorosilicone, or any other release coating known to those of ordinary skill in the art. The release liner may have an uneven surface in order to easily take out the transdermal patch from a package. Examples of release liners may include, but are not limited to SCOTCHPAK products from 3M such as 3M SCOTCHPAK 9744, 3M SCOTCHPAK 9755, 3M SCOTCHPAK 9709, and 3M SCOTCHPAK 1022.

Other layers such as abuse deterrent layers formulated with one or more irritants (e.g., sodium lauryl sulfate, poloxamer, sorbitan monoesters, glyceryl monooleates, spices, etc.), may also be employed.

Methods disclosed herein using a transdermal patch dosage form provide for systemic delivery of small doses of active ingredient(s), preferably over extended periods of time such as up to 168 hour time periods, for example from 2 to 96 hours, or 4 to 72 hours, or 8 to 24 hours, or 10 to 18 hours, or 12 to 14 hours. In particular, the compound(s) of Formula (I) of the present disclosure can be delivered in small, steady, and consistent doses such that deleterious or undesirable side-effects can be avoided. In some embodiments, the compounds of Formula (I) of the present disclosure are administered transdermally at serotonergic, but sub-psychoactive concentrations.

Therefore, provided herein are methods of treating a disease or disorder associated with a serotonin 5-HT₂ receptor, such as a central nervous system (CNS) disorder, a psychological disorder, or an autonomic nervous system (ANS), comprising administering the solid dispersion od the present disclosure via a transdermal patch. Here, the compound of Formula (I) is capable of diffusing from the polymer matrix of the transdermal patch (e.g., from the pressure sensitive adhesive layer or from a separate polymer layer) across the skin of the subject and into the bloodstream of the subject.

An exemplary drug-in-adhesive (DIA) patch formulation may comprise 5 to 30 wt. % of a compound of Formula (I) (psilocybin, psilocybin-d₁₀ etc.), 5 to 35 wt. % polymer/crystallization inhibitor (e.g., HPMC, HPMCAS, polyvinylpyrrolidone-co-vinyl acetate, polymethacrylate, etc.), 30 to 70 wt. % pressure sensitive adhesive (e.g., DURO-TAK 387-2052, DURO-TAK 87-2677, and DURO-TAK 87-4098), 1 to 10 wt. % permeation agents/absorption enhancers (e.g., oleyloleate, oleyl alcohol, levulinic acid, diethylene glycol monoethyl ether, etc.), each based on a total weight of the DIA patch formulation, though it should be understood that many variations are possible in light of the teachings herein.

Automatic injection devices offer a method for delivery of the pharmaceutical compositions disclosed herein to patients. The compositions disclosed herein may be administered to a patient using automatic injection devices through a number of known devices, a non-limiting list of which includes transdermal, subcutaneous, and intramuscular delivery.

In some transdermal, subcutaneous, or intramuscular applications, a composition disclosed herein is absorbed through the skin. Passive transdermal patch devices often include an absorbent layer or membrane that is placed on the outer layer of the skin. The membrane typically contains a dose of a substance that is allowed to be absorbed through the skin to deliver the composition to the patient. Typically, only substances that are readily absorbed through the outer layer of the skin may be delivered with such transdermal patch devices.

Other automatic injection devices disclosed herein are configured to provide for increased skin permeability to improve delivery of the disclosed compositions. Non-limiting examples of structures used to increase permeability to improve transfer of a composition into the skin, across the skin, or intramuscularly include the use of one or more microneedles, which in some embodiments may be coated with a composition disclosed herein. Alternatively, hollow microneedles may be used to provide a fluid channel for delivery of the disclosed compositions below the outer layer of the skin. Other devices disclosed herein include transdermal delivery by iontophoresis, sonophoresis, reverse iontophoresis, or combinations thereof, and other technologies known in the art to increase skin permeability to facilitate drug delivery.

The pharmaceutical compositions may also be administered topically by electroporation, iontophoresis, phonophoresis, sonophoresis and microneedle or needle-free injection, such as POWDERJECT™ (Chiron Corp., Emeryville, Calif), and BIOJECT™ (Bioject Medical Technologies Inc., Tualatin, Oreg.).

The pharmaceutical compositions disclosed herein may be disclosed in the forms of ointments, creams, and gels. Suitable ointment vehicles include oleaginous or hydrocarbon vehicles, including such as lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinyl alcohol; cellulosic polymers, such as hydroxypropyl cellulose, hydroxyethyl cellulose, hydroxypropyl methylcellulose, hydroxypropyl methyl cellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions disclosed herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable excipients utilized in rectal and vaginal suppositories include bases or vehicles, such as stiffening agents, which produce a melting point in the proximity of body temperature, when formulated with the pharmaceutical compositions disclosed herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to about 3 g.

The pharmaceutical compositions disclosed herein may be administered ophthalmically in the forms of solutions, suspensions, ointments, emulsions, gel-forming solutions, powders for solutions, gels, ocular inserts, and implants.

### D. Intranasal and Inhalation Administration

The pharmaceutical compositions disclosed herein may be administered intranasally or by inhalation to the respiratory tract. The pharmaceutical compositions may be disclosed in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be disclosed as a dry powder for insufflation, alone or in combination with an inert carrier such as lactose or phospholipids; and nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient disclosed herein, a propellant as solvent; and/or a surfactant, such as sorbitan trioleate, oleic acid, or an oligolactic acid.

Administration may also be carried out with a dry powder inhaler (DPI). In such DPI devices, the solid dispersion itself can form the powder or the powder can be formed from pharmaceutical compositions of the solid dispersion and additional pharmaceutically acceptable excipients. Pharmaceutically acceptable excipients used to formulate such carrier powders are known in the art (see, e.g., H. Hamishehkar, et al., “The Role of Carrier in Dry Powder Inhaler”, Recent Advances in Novel Drug Carrier Systems, pp. 39-66, (2012)). In either case, the active ingredient (e.g., a compound of Formula (I)) is releasably bound within the solid dispersion such that upon inhalation, the moisture in the lungs releases the active ingredient from the solid dispersion to make the active ingredient available for systemic absorption. In some embodiments, the active ingredient is delivered by use of a dry powder inhaler (DPI).

DPI is generally formulated with powders or powder mixtures with coarse carrier particles and micronized drug particles with aerodynamic particle diameters of 1-5 μm (see Iida et al., “Preparation of dry powder inhalation by surface treatment of lactose carrier particles.” Chem Pharm Bull, 2003, 51(1): 1-5). Carrier particles are often used to improve drug particle flowability, thus improving dosing accuracy and minimizing the dose variability observed with drug formulations alone while making them easier to handle during manufacturing operations. Carrier particles should have several characteristics such as physico-chemical stability, biocompatibility and biodegradability, compatible with the drug substance and must be inert, available and economical. The choice of carrier particle (both content and size) is well within the purview of one of ordinary skill in the art. The most common carrier particles are made of lactose or other sugars, with α-lactose monohydrate being the most common lactose grade used in the inhalation field for such particulate carriers.

The pharmaceutical compositions disclosed herein may be micronized to a size suitable for delivery by inhalation, such as about 50 micrometers or less, or about 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying. In some embodiments, dry powders for use in DPI administration are produced using spray drying techniques.

Capsules, blisters, and cartridges for use in an inhaler or insufflator may be formulated to contain a powder mix of the pharmaceutical compositions disclosed herein; a suitable powder base, such as lactose or starch; and a performance modifier, such as l-leucine, mannitol, or magnesium stearate. The lactose may be anhydrous or in the form of the monohydrate. Other suitable excipients include, but are not limited to, dextran, glucose, maltose, sorbitol, xylitol, fructose, sucrose, and trehalose. The pharmaceutical compositions disclosed herein for inhaled/intranasal administration may further comprise a suitable flavor, such as menthol and levomenthol, or sweeteners, such as saccharin or saccharin sodium.

The pharmaceutical compositions disclosed herein for topical administration may be formulated to be immediate release or modified release, including delayed-, sustained-, pulsed-controlled-, targeted, and programmed release.

### E. Modified Release

The pharmaceutical compositions disclosed herein may be formulated as a modified release dosage form. As used herein, the term “modified release” refers to a dosage form in which the rate or place of release of the active ingredient(s) is different from that of an immediate dosage form when administered by the same route. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

### 1. Matrix Controlled Release Devices

The pharmaceutical compositions disclosed herein in a modified release dosage form may be fabricated using a matrix controlled release device known to those skilled in the art (see, Takada et al in “Encyclopedia of Controlled Drug Delivery,” Vol. 2, Mathiowitz ed., Wiley, 1999).

In some embodiments, the pharmaceutical compositions disclosed herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinylpyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Evonik); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In further embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate, and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients in the pharmaceutical compositions.

The pharmaceutical compositions disclosed herein in a modified release dosage form may be prepared by methods known to those skilled in the art, including direct compression, dry or wet granulation followed by compression, melt-granulation followed by compression.

### 2. Osmotic Controlled Release Devices

The pharmaceutical compositions disclosed herein in a modified release dosage form may be fabricated using an osmotic controlled release device, including a one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

Another class of osmotic agents are osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol, organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

A semipermeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membranes are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can be substantially modulated via the thickness and porosity of the semipermeable membrane, the composition of the core, and the number, size, and position of the delivery ports.

The pharmaceutical compositions in an osmotic controlled-release dosage form may further comprise additional conventional excipients as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In some embodiments, the pharmaceutical compositions disclosed herein are formulated as AMT controlled-release dosage form, which comprises an asymmetric osmotic membrane that coats a core comprising the active ingredient(s) and other pharmaceutically acceptable excipients. The AMT controlled-release dosage forms can be prepared according to conventional methods and techniques known to those skilled in the art, including direct compression, dry granulation, wet granulation, and a dip-coating method.

In some embodiments, the pharmaceutical compositions disclosed herein are formulated as ECS controlled-release dosage form, which comprises an osmotic membrane that coats a core comprising the active ingredient(s), a polymer (e.g., hydroxylethyl cellulose), and optionally other pharmaceutically acceptable excipients.

### 3. Multiparticulate Controlled Release Devices

The pharmaceutical compositions disclosed herein in a modified release dosage form may be fabricated a multiparticulate controlled release device, which comprises a multiplicity of particles, granules, or pellets, ranging from about 10 μm to about 3 mm, about 50 μm to about 2.5 mm, or from about 100 m to about 1 mm in diameter. Such multiparticulates may be made by the processes know to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

### 4. Targeted Delivery

The pharmaceutical compositions disclosed herein may also be formulated to be targeted to a particular tissue, receptor, or other area of the body of the subject to be treated, including liposome-, resealed erythrocyte-, and antibody-based delivery systems.

### Method of Making Solid Dispersions (e.g., Solid Molecular Complexes)

Also provided are methods of making solid dispersions (e.g., solid molecular complexes) as disclosed herein and pharmaceutical compositions comprising the solid dispersions (e.g., solid molecular complexes). In some embodiments, the compound of Formula (I) may be microprecipitated with a polymer as disclosed herein. Methods of making the solid dispersion, or pharmaceutical composition, may be accomplished by any means known in the art, for example: spray drying; freeze-drying (lyophilization); solvent-controlled precipitation; pH-controlled precipitation; hot melt extrusion; and supercritical fluid technology. Each of these methods is described in more detail below.

After forming the solid dispersion using the various methods, it can be recovered by procedures known to those skilled in the art, for example by filtration, conveying to a collector, centrifugation, washing, etc. The recovered solid dispersion can be subjected to drying or additional drying steps (e.g., in air, an oven, or a vacuum) and the resulting solid can be optionally milled, pulverized or micronized to a fine powder by means known in the art. The powder form of the solid dispersion can then be used as is (can be used per se as the pharmaceutical composition) or combined with a pharmaceutically acceptable excipient to form a pharmaceutical composition.

### 1. Spray Drying

Solid dispersions can be obtained by spray drying a liquid mixture comprising an active ingredient (e.g., a compound of Formula (I) or a pharmaceutically acceptable salt, a polymorph, stereoisomer, a tautomer, solvate, or active metabolite thereof), a suitable polymer(s), an optional pharmaceutically acceptable excipient, and an appropriate solvent system. The solvent system may be a single solvent or mixture of solvents, including organic solvents having a low boiling point (e.g., ethanol, methanol, acetone, dichloromethane (DCM), methyl acetate, ethyl acetate, isopropyl acetate, 2-butanone, methanol, 1-propanol, propan-2-ol, acetonitrile, chloroform, etc.), solvents including organic solvents with a medium/high boiling point (e.g., water, acetic acid, 3-pentanone, 4-methyl-2-pentanone, dimethylsulfoxide, dimethylformamide, etc.), or mixtures thereof. In some embodiments, the solvent system is a mixture of organic solvents, e.g., dichloromethane and methanol. While not limited thereto, a % solid loading of the liquid mixture typically ranges from about 0.1 wt. %, from about 0.5 wt. %, from about 1 wt. %, from about 1.5 wt. %, from about 2 wt. %, and up to about 5 wt. %, up to about 4.5 wt. %, up to about 4 wt. %, up to about 3.5 wt. %, up to about 3 wt. %, up to about 2.5 wt. %.

Spray drying is a process that converts the liquid mixture to a dried particulate form, through atomization of the liquid mixture and removal of the solvent. Atomization may be done, for example, through a nozzle or on a rotating disk. By means of spray drying, the solvent is evaporated by flash evaporation, for example at a temperature close to the boiling point thereof, leaving the compound of Formula (I) precipitated in a matrix formed by the polymer. Optionally, a secondary drying process such as fluidized bed drying or vacuum drying, may be used to reduce residual solvents to pharmaceutically acceptable levels.

Typically, spray drying involves contacting a highly dispersed liquid mixture and a sufficient volume of hot air or gas to produce evaporation and drying of the liquid droplets. The liquid mixture to be spray dried can be any solution, suspension, coarse suspension, slurry, colloidal dispersion, or paste that may be atomized using the selected spray drying apparatus. In a standard procedure, the liquid mixture is sprayed into a current of warm filtered air or gas that evaporates the solvent and conveys the dried product to a collector (e.g., a cyclone or directly to a membrane filter bag). The spent air may then be exhausted with the solvent, or alternatively the spent air may be sent to a condenser to capture and optionally recycle the solvent. A commercially available spray dry apparatus may be used to conduct the spray drying. For example, commercial spray dryers are manufactured by Buchi Ltd., and NIRO® and PHARMASD™ spray dryers from GEA (see, US 2004/0105820; US 2003/0144257). Spray dyers-spray chillers/congealers such as PROCEPT 4M8-Trix available from Procept, may also be used. For example, a pressure nozzle, a two-fluid electrosonic nozzle, a two-fluid nozzle, a three-fluid nozzle, a cooled nozzle, a heated nozzle, an ultrasonic nozzle, or a rotary atomizer can be used.

Techniques and methods for spray drying may be found in Perry's Chemical Engineering Handbook, 6th Ed., R. H. Perry, D. W. Green & J. O. Maloney, eds.), McGraw-Hill book co. (1984); and Marshall “Atomization and Spray Drying” 50, Chem. Eng. Prog. Monogr. Series 2 (1954). In general, the spray drying may be conducted with an inlet temperature of from about 40° C., from about 45° C., from about 50° C., from about 60° C., from about 70° C., to about 200° C., to about 150° C., to about 100° C., to about 75° C., e.g., about 50° C. The spray drying may generally be conducted with an outlet temperature of from about 15° C., from about 20° C., from about 25° C., to about 100° C., to about 75° C., to about 50° C., to about 40° C., to about 30° C., e.g., about 27° C.

Removal of the solvent may optionally involve a subsequent drying step, such as tray drying, fluid bed drying (e.g., from about room temperature to about 100° C., e.g., about 60° C.), vacuum drying, microwave drying, rotary drum drying, or biconical vacuum drying (e.g., from about room temperature to about 100° C., e.g., about 60° C. or lower).

Fluidized spray drying techniques may also be employed herein. The process of fluidized spray drying combines spray drying and fluid bed drying technologies. Agglomerated powders are obtained based on the integrated fluid bed or belt and a multi-stage process where moist powder, produced during the first drying stage, forms agglomerates, which are post-dried and cooled in the following stages. Briefly, a pressure nozzle, a two-fluid electrosonic nozzle, a two-fluid nozzle, or a rotary atomizer sprays the liquid mixture down into the spray dryer towards the fluid bed. Agglomeration incorporating finer, recycled material takes place in the spray dryer, and agglomerated particles fall to the bed. Agglomerated particles may be further dried in the bed. Exhaust air outlets through the roof causing further agglomeration in the zone of spraying.

As an example, in the spray dryer, the liquid mixture is sprayed from the atomization nozzle mounted on top of the drying chamber into the drying air and down the spray chamber. The vigorous fluidization of moist powder in the fluid bed located at the chamber base, plus recycle of fines from the cyclone attachment, result in spray drying taking place in a powder-laden atmosphere. Particles of higher moisture content can be handled in the drying chamber due to the resulting powdering effect. Drying can be completed at lower powder and exhaust air temperatures. The degree of agglomeration and thus the particle size distribution can be influenced by changing the operation conditions and the location where fines are re-introduced into the drying chamber. By adjusting the operation conditions, a solid dispersion with properties favorable for downstream processing, can be obtained.

### 2. Lyophilization

Solid dispersions (e.g., solid molecular complexes) may be prepared through lyophilization of aqueous formulations comprising water (and optionally one or more co-solvents), an active ingredient (e.g., a compound of Formula (I) or a pharmaceutically acceptable salt, a polymorph, stereoisomer, a tautomer, solvate, or active metabolite thereof), a suitable polymer(s) (e.g., gelatin), and any desired optional pharmaceutically acceptable excipient (e.g., mannitol).

Typically, a water-soluble polymer and any desired pharmaceutically acceptable excipient may be dissolved in water or aqueous solvent system comprising water and organic solvent(s), examples of which are set forth herein. Optional heating may be employed if desired, for example from about 40° C., from about 45° C., from about 50° C., from about 55° C., to about 100° C., to about 90° C., to about 80° C., to about 70° C., to about 60° C., to ensure complete dissolution of components. Optional cooling may also be employed prior to addition of the active ingredient. The active ingredient may then be mixed with the aqueous mixture (e.g., via stirring, vortexing, etc.), followed by any desired pH adjustment using a pH modifier (e.g., sodium hydroxide solution). The aqueous formulation may be charged into blister pockets, if desired, for producing unit dosage forms. Flash freezing may then be performed, e.g., using liquid nitrogen, dry ice, or cryogenic equipment, and the frozen mixture may then be subjected to low pressure (vacuum) conditions, preferably while being held at reduced temperature (e.g., 0° C. or below, −5° C. or below, −10° C. or below, −15° C. or below).

By means of lyophilization, the solvent (water and optionally one or more co-solvents) is evaporated under vacuum (low vapor pressure), leaving the compound of Formula (I) precipitated in a matrix formed by the polymer(s) and any optional excipients present.

### 3. Solvent Controlled Precipitation

In solvent controlled precipitation processes, an active ingredient (e.g., a compound of Formula (I) or a pharmaceutically acceptable salt, a polymorph, stereoisomer, a tautomer, solvate, or active metabolite thereof), a suitable polymer(s), and any desired optional pharmaceutically acceptable excipient may be dissolved in a solvent, e.g., dimethylacetamide, dimethylformamide, dimethyl sulfoxide (DMSO), N-methyl pyrrolidone (NMP), etc. The resulting solution is added to an aqueous phase comprising water adjusted to an appropriate pH (for example, in some embodiments, a pH of 3 or less). The aqueous phase may be set to any desired temperature, such as from about 0° C. to about 7° C., or about 2° C. to about 5° C. This causes the compound of Formula (I) to microprecipitate in a matrix formed by the polymer. The microprecipitate may be washed several times with aqueous medium until the residual solvent falls below an acceptable limit for that solvent. An “acceptable limit” for each solvent is determined pursuant to the International Conference on Harmonization (ICH) guidelines.

In some embodiments, a solution comprising the compound of Formula (I), an organic solvent (e.g., dimethylformamide, dimethylacetamide (DMA), dimethyl sulfoxide (DMSO), N-methyl pyrrolidone (NMP), and the like) and the polymer is formed. For example, the organic solvent can be DMA at 20 to 25° C. The solution may be formed by first dissolving the compound of Formula (I) into the organic solvent. Then, while stirring, the polymer is added. The mixture may be optionally heated to, for example to between about 50° C. to about 110° C., e.g., about 70° C.

The aqueous phase may be an acidic aqueous solution such as dilute HCl (e.g., 0.01 N HCl) The aqueous phase may be set to any desired temperature, typically between 0° C. and about 60° C., or between 5° C. and 15° C.

The aqueous phase may be circulated through the mixing chamber of a high shear mixer while the solution comprising the active ingredient, polymer, and any excipients is dosed into the chamber while the chamber is operating. Dosing may be accomplished with, for example, a gear pump, a hose pump, or a syringe pump. In some embodiments, dosing is accomplished using a gear pump with an injector nozzle pointed into the mixing chamber. The mixing chamber can comprise a rotor and a stator. The rotor and the stator may, for example, each have either one or two rows of teeth. In some embodiments, the rotor and the stator each have one row of teeth. The tip speed of the rotor can be set at between about 15 and about 25 m/sec.

During the mixing process, the active ingredient (e.g., the compound of Formula (I)) and the polymer precipitate, producing a suspension of particles of the solid dispersion in aqueous-organic media. The suspension may then be subjected to a number of passes through a dispersing unit in order to adjust the particle size of the particles of the solid dispersion. The suspension may then be centrifuged and washed with the aqueous phase several times in order to remove the organic solvent and then washed once with pure water. The obtained product may then be delumped and dried to isolate the solid dispersion of the present disclosure. During the drying process, the temperature can be kept below 40° C., if needed, to prevent recrystallization of the compound of Formula (I).

Potentially beneficial excipients may fall generally into the following classes: other matrix materials or diluents, surface active agents, drug complexing agents or solubilizing agents, fillers, disintegrants, binders, lubricants, and pH modifiers (e.g., acids, bases, or buffers). Examples of other matrix materials, fillers, or diluents include lactose, mannitol, xylitol, microcrystalline cellulose, silicified microcrystalline cellulose, calcium diphosphate, and starch. Examples of surface active agents include sodium lauryl sulfate and polysorbate 80. Examples of drug complexing agents or solubilizing agents include the polyethylene glycols, caffeine, xanthene, gentisic acid and cylodextrins. Examples of disintegrants include sodium starch gycolate, sodium alginate, carboxymethyl cellulose sodium, methyl cellulose, and croscarmellose sodium. Examples of binders include methyl cellulose, microcrystalline cellulose, starch, and gums such as guar gum, and tragacanth. Examples of lubricants include magnesium stearate and calcium stearate. Examples of pH modifiers include acids such as citric acid, acetic acid, ascorbic acid, lactic acid, aspartic acid, succinic acid, phosphoric acid, and the like; bases such as sodium acetate, potassium acetate, calcium oxide, magnesium oxide, trisodium phosphate, sodium hydroxide, calcium hydroxide, aluminum hydroxide, and the like, and buffers generally comprising mixtures of acids and the salts of said acids. At least one function of inclusion of such pH modifiers is to control the dissolution rate of the drug, matrix polymer, or both, thereby controlling the local drug concentration during dissolution.

Excipients may be incorporated into the amorphous solid dispersion during or after its formation. In addition to the above excipients, use of any conventional materials and procedures for formulation and preparation of suitable dosage forms (e.g., oral dosage forms) using the pharmaceutical compositions disclosed herein known by those skilled in the art may be used.

4. pH-Controlled Precipitation

A pH-controlled precipitation process involves the microprecipitation of an active ingredient (e.g., a compound of Formula (I) or a pharmaceutically acceptable salt, a polymorph, stereoisomer, a tautomer, solvate, or active metabolite thereof), a suitable polymer(s), and any desired optional pharmaceutically acceptable excipient. In this process, the active ingredient (e.g., the compound of Formula (I)), the polymer, and any desired excipients are dissolved at a high pH and precipitated by lowering the pH of the solution, or vice versa.

In some embodiments, the polymer is insoluble at low pH. The compound of Formula (I) and the polymer are dissolved in an organic solvent such as dimethylformamide, dimethylacetamide (DMA), dimethyl sulfoxide (DMSO), N-methyl pyrrolidone (NMP), and the like. The pH of the solution is then lowered through use of an acid. The acid may be added to the solution of the compound of Formula (I) and polymer, the solution of the compound of Formula (I) and polymer may be added to the acid, or the solution and the acid may be combined and mixed simultaneously. At the lowered pH, both the compound of Formula (I) and the polymer simultaneously precipitate out, resulting in the solid dispersion containing the compound of Formula (I) embedded in a matrix formed by the polymer. The resulting solid dispersion may then be washed with water to remove the organic solvent, and dried to pharmaceutically acceptable levels.

### 5. Hot Melt Extrusion Process

Microprecipitation of the active ingredient (e.g., compound of Formula (I) or a pharmaceutically acceptable salt, a polymorph, stereoisomer, a tautomer, solvate, or active metabolite thereof) and optionally a pharmaceutically acceptable excipient in a polymer can be achieved in some embodiments by a hot melt extrusion process. Here, the components are mixed and fed continuously to a temperature-controlled extruder causing the compound of Formula (I) to be molecularly dispersed, together with any excipients present, in the molten polymer.

Hot melt extruders typically contain four primary parts: a motor that controls the rotation of the screws, the screws (primary source of shear and moving the material), the barrels that house the screws and provide temperature control, and the die (the exit port) that controls the shape and size of the extrudates. The desired materials constituting the solid dispersions (usually granular or in powder form) are generally fed into the extruder feeding port at a controlled rate while the extruder screws are rotating. The material is then conveyed forward using the rotation of screw and the friction of the material against the barrel surface. Depending on the type of extruder, a single screw or a twin screw may be used to operate either in counter or co-rotating mode. The screws can be appropriately designed to achieve a desired degree of mixing. In general, the barrels are segmented to enable temperature adjustment in each zone throughout the screw length. The exit port (the die system) controls the shape and size of the extrudates.

The resulting extrudate may then be cooled, e.g., to room temperature to produce the solid dispersion in extrudate form, which may be milled, e.g., into a fine powder.

### 6. Supercritical Fluid Process

In this process, the active ingredient (e.g., compound of Formula (I) or a pharmaceutically acceptable salt, a polymorph, stereoisomer, a tautomer, solvate, or active metabolite thereof), optional pharmaceutically acceptable excipient, and a polymer are dissolved in a supercritical fluid such as liquid nitrogen or liquid carbon dioxide. The supercritical fluid is then removed by evaporation leaving the compound of Formula (I) microprecipitated in the matrix formed by the polymer. In a different method, the compound of Formula (I) and a polymer are dissolved in a suitable solvent. A microprecipitated powder can then be formed by spraying the solution in a supercritical fluid which acts as an antisolvent.

Methods of making solid dispersions (e.g., solid molecular complexes) of the present disclosure are not limited to the above-described methods, and other methods known to those of ordinary skill in the art may also be used, for example, solution casting to make solid dispersions in film form.

In any of the above methods, determination of whether the compound of Formula (I) has been successfully immobilized in amorphous form in the solid dispersion (i.e., whether an amorphous solid dispersion has been formed) can be made by various means, including X-ray powder diffraction. In addition, the glass transition temperature of the solid dispersion can be measured using modulated DSC and this can also provide information whether the dispersion is a multiphase or uniphase. A uniphase is indicative of such immobilization.

### Therapeutic Applications and Methods

Provided herein are methods of treating a subject with a disease or disorder comprising administering to the subject one or more (e.g., 1, 2, 3, 4, 5, or more) pharmaceutical compositions comprising a solid dispersion of a compound of Formula (I) (or a pharmaceutically acceptable salt, a polymorph, stereoisomer, a tautomer, solvate, or active metabolite thereof) as disclosed herein.

Also disclosed is a method of treating a subject with a disease or disorder associated with a serotonin 5-HT₂ receptor comprising administering to the subject one or more pharmaceutical compositions comprising a solid dispersion of a compound of Formula (I) (or a pharmaceutically acceptable salt, a polymorph, stereoisomer, a tautomer, solvate, or active metabolite thereof) as disclosed herein.

The dosage and frequency (single or multiple doses) of administration can vary depending upon a variety of factors, including, but not limited to, the active ingredient(s) to be administered; the disease/condition being treated; route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

Therapeutically effective amounts for use in humans may be determined from animal models. For example, a dose for humans can be formulated to achieve a concentration that has been found to be effective in animals. The dosage in humans can be adjusted by monitoring response to the treatment and adjusting the dosage upwards or downwards.

Dosages may be varied depending upon the requirements of the subject and the active ingredient (e.g., a compound of Formula (I)) being employed. The dose administered to a subject, in the context of the pharmaceutical compositions presented herein, should be sufficient to affect a beneficial therapeutic response in the subject over time. The size of the dose also will be determined by the existence, nature, and extent of any adverse side effects. Generally, treatment is initiated with smaller dosages, which are less than the optimum dose of the active ingredient. Thereafter, the dosage is increased by small increments until the optimum effect under circumstances is reached.

Dosage amounts and intervals can be adjusted individually to provide levels of the administered compounds (e.g., a compound of Formula (I)) effective for the particular clinical indication being treated. This will provide a therapeutic regimen that is commensurate with the severity of the individual's disease state.

Routes of administration may include oral routes (e.g., enteral/gastric delivery, intraoral administration such buccal, lingual, and sublingual routes), parenteral routes (e.g., intravenous, intraarterial, intraperitoneal, intrathecal, intraventricular, intraurethral, intrasternal, intracranial, intramuscular, intrasynovial, and subcutaneous administration), and topical routes (e.g., (intra)dermal, conjuctival, intracorneal, intraocular, ophthalmic, auricular, transdermal, nasal, vaginal, uretheral, respiratory, and rectal administration), or others sufficient to affect a beneficial therapeutic response.

Administration may follow a continuous administration schedule, or an intermittent administration schedule. The administration schedule may be varied depending on the active ingredient employed, the condition being treated, the administration route, etc. For example, administration may be performed once a day (QD), or in divided dosages throughout the day, such as 2-times a day (BID), 3-times a day (TID), 4-times a day (QID), or more. In some embodiments administration may be performed nightly (QHS). In some embodiments, the compounds/pharmaceutical compositions may be administered as needed (PRN). Administration may also be performed on a weekly basis, e.g., once a week, twice a week, three times a week, four times a week, every other week, or other administration schedules deemed appropriate using sound medical judgement, such as one treatment per treatment course.

The dosing can be continuous (7 days of administration in a week) or intermittent, for example, depending on the pharmacokinetics and a particular subject's clearance/accumulation of the active ingredient. If intermittently, the schedule may be, for example, 4 days of administration and 3 days off (rest days) in a week or any other intermittent dosing schedule deemed appropriate using sound medical judgement. For example, intermittent dosing may involve administration of a single dose within a treatment course. The dosing whether continuous or intermittent is continued for a particular treatment course, typically at least a 28-day cycle (1 month), which can be repeated with or without a drug holiday. Longer or shorter courses can also be used such as 14 days, 18 days, 21 days, 24 days, 35 days, 42 days, 48 days, or longer, or any range therebetween. The course may be repeated without a drug holiday or with a drug holiday depending upon the subject. Other schedules are possible depending upon the presence or absence of adverse events, response to the treatment, patient convenience, and the like.

Dosing/administration of the compound of Formula (I), or a pharmaceutically acceptable salt, a polymorph, stereoisomer, a tautomer, solvate, or active metabolite thereof, can be accomplished in any of the embodiments described herein, through administration of the disclosed solid dispersions/pharmaceutical compositions. In some embodiments, the compound of Formula (I) (e.g., psilocybin) is administered at therapeutically effective intervals. In some embodiments, a therapeutically effective interval may be about 2 weeks, about 3 weeks, about 4 weeks, about 5 weeks, about 6 weeks, about 7 weeks, about 8 weeks, about 9 weeks, about 10 weeks, about 11 weeks, or about 12 weeks. In some embodiments, a therapeutically effective interval may be about 1 month, about 3 months, about 6 months, or about 12 months. In some embodiments, the compound of Formula (I) (e.g., psilocybin) is administered once per day. In some embodiments, the compound of Formula (I) (e.g., psilocybin) is administered at least once per week or at least twice per week. In some embodiments, the compound of Formula (I) (e.g., psilocybin) is administered at least once per month or at least twice per month. In some embodiments, the compound of Formula (I) (e.g., psilocybin) is administered at least once every three months, at least once every six months, or at least once every 12 months.

In some embodiments, a first dose and a second dose of the compound of Formula (I) (e.g., psilocybin) are administered to the subject. In some embodiments, the first dose is about 1 mg and the second dose is about 1 mg. In some embodiments, the first dose is about 10 mg and the second dose is about 10 mg. In some embodiments, the first dose is about 25 mg and the second dose is about 25 mg. In some embodiments, the first dose is about 10 mg and the second dose is about 25 mg. In some embodiments, the first dose is about 25 mg and the second dose is about 10 mg. In some embodiments, the first dose is about 1 mg and the second dose is about 10 mg. In some embodiments, the first dose is about 1 mg and the second dose is about 25 mg. In some embodiments, the first dose is about 10 mg and the second dose is about 1 mg. In some embodiments, the first dose is about 25 mg and the second dose is about 1 mg.

In some embodiments a second dose of the compound of Formula (I) (e.g., psilocybin) is administered from about one week to about 12 weeks after a first dose. In some embodiments, a second dose of the compound of Formula (I) (e.g., psilocybin) is administered about one week after a first dose. In some embodiments, a second dose of the compound of Formula (I) (e.g., psilocybin) is administered about two weeks after a first dose. In some embodiments, a second dose of the compound of Formula (I) (e.g., psilocybin) is administered about three weeks after a first dose. In some embodiments, a second dose of the compound of Formula (I) (e.g., psilocybin) is administered about four weeks after a first dose. In some embodiments, a second dose of the compound of Formula (I) (e.g., psilocybin) is administered about five weeks after a first dose. In some embodiments, a second dose of the compound of Formula (I) (e.g., psilocybin) is administered about six weeks after a first dose.

In some embodiments, the pharmaceutical compositions of the disclosure may be used as a standalone therapy. In some embodiments, the pharmaceutical compositions of the disclosure may be used as an adjuvant/combination therapy.

Utilizing the teachings provided herein, an effective prophylactic or therapeutic treatment regimen can be planned that does not cause substantial toxicity or adverse side effects (e.g., caused by sedative or psychotomimetic toxic spikes in plasma concentration of any of the compounds Formula (I)), and yet is entirely effective to treat the clinical symptoms demonstrated by the particular patient. This planning should involve the careful choice of active ingredient (e.g., a compound of Formula (I) or a pharmaceutically acceptable salt, a polymorph, stereoisomer, a tautomer, solvate, or active metabolite thereof) and dosage form by considering factors such as compound potency, release kinetics, relative bioavailability, patient body weight, presence and severity of adverse side effects, preferred mode of administration, and the toxicity profile of the selected agent.

A therapeutically effective dose of the pharmaceutical composition disclosed herein may vary depending on the variety of factors described above, but is typically that which provides the compound of Formula (I) in an amount of about 0.00001 mg to about 10 mg per kilogram body weight of the recipient, or any range in between, e.g., about 0.00001 mg/kg, about 0.00005 mg/kg, about 0.0001 mg/kg, about 0.0005 mg/kg, about 0.001 mg/kg, about 0.005 mg/kg, about 0.01 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg of the compound of Formula (I) (active).

In some embodiments, a dose of the pharmaceutical composition disclosed herein is that which provides about 1 mg to about 100 mg of the compound of Formula (I) (e.g., psilocybin). For example, the dose may be about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg of the compound of Formula (I). In some embodiments, the dose of the compound of Formula (I) (e.g., psilocybin) is between about 0.1 mg to about 100 mg, about 1 mg to about 50 mg, or about 5 mg to about 30 mg. In some embodiments, the dose of the compound of Formula (I) (e.g., psilocybin) is about 1 mg, about 10 mg, or about 25 mg. In some embodiments, the dose of the compound of Formula (I) (e.g., psilocybin) is in the range of about 0.001 mg to about 1 mg. In some embodiments, the dose of the compound of Formula (I) (e.g., psilocybin) is in the rage of about 100 mg to about 250 mg. In some embodiments, the dose of the compound of Formula (I) (e.g., psilocybin) is about 25 mg. In some embodiments, the compound of Formula (I) (e.g., psilocybin) is primarily in amorphous form, preferably in highly pure amorphous form.

In some embodiments, an adult oral dose comprises about 1 mg to about 40 mg, about 2 to about 30 mg, or about 15 to about 30 mg of the compound of Formula (I) (e.g., psilocybin), for example about 1 mg, about 5 mg, about 10 mg, or about 25 mg of the compound of Formula (I) (e.g., psilocybin). In some embodiments, an adult oral dose comprises about 25 mg of the compound of Formula (I) (e.g., psilocybin). In some embodiments, the compound of Formula (I) (e.g., psilocybin) is primarily in amorphous form, preferably in highly pure amorphous form.

The pharmaceutical compositions may be administered to provide the compound of Formula (I), or a pharmaceutically acceptable salt, a polymorph, stereoisomer, a tautomer, solvate, or active metabolite thereof, at a psychedelic dose. Psychedelic dosing, by mouth or otherwise, may range from about 0.083 mg/kg, about 0.09 mg/kg, about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, and up to about 1 mg/kg, about 0.95 mg/kg, about 0.9 mg/kg, about 0.85 mg/kg, about 0.8 mg/kg, about 0.75 mg/kg, about 0.7 mg/kg, about 0.65 mg/kg, about 0.6 mg/kg, about 0.55 mg/kg of the compound of Formula (I) (active). Higher dosing may also be used in some embodiments, as described above. In some embodiments, psychedelic doses are administered once by mouth, with the possibility of repeat doses at least one week apart. In some instances, no more than 5 doses are given in any one course of treatment. Courses can be repeated as necessary, with or without a drug holiday. Such acute treatment regimens may be accompanied by psychotherapy, before, during, and/or after the psychedelic dose. These treatments are appropriate for a variety of mental health disorders disclosed herein, examples of which include, but are not limited to, major depressive disorder (MDD), therapy resistant depression (TRD), anxiety disorders, and substance use disorders (e.g., alcohol use disorder, opioid use disorder, amphetamine use disorder, nicotine use disorder, smoking, and cocaine use disorder).

The pharmaceutical compositions may be administered to provide the compound of Formula (I), or a pharmaceutically acceptable salt, a polymorph, stereoisomer, a tautomer, solvate, or active metabolite thereof, at serotonergic, but sub-psychoactive concentrations to achieve durable therapeutic benefits, with decreased toxicity, and may thus be suitable for low dosing or microdosing. For example, when administered by mouth, the dose range for sub-psychedelic dosing may range from about 0.00001 mg/kg, about 0.00005 mg/kg, about 0.0001 mg/kg, about 0.0005 mg/kg, about 0.001 mg/kg, about 0.005 mg/kg, about 0.006 mg/kg, about 0.008 mg/kg, about 0.009 mg/kg, about 0.01 mg/kg, and less than about 0.083 mg/kg, about 0.08 mg/kg, about 0.075 mg/kg, about 0.07 mg/kg, about 0.06 mg/kg, about 0.05 mg/kg, about 0.04 mg/kg, about 0.03 mg/kg, about 0.02 mg/kg of the compound of Formula (I) (active). In some embodiments, sub-psychedelic doses are administered orally up to every day, for a treatment course (e.g., 1 month). However, there is no limitation on the number of doses at sub-psychedelic doses—dosing can be less frequent or more frequent as deemed appropriate. Courses can be repeated as necessary, with or without a drug holiday. In some embodiments, a “micro-dose” of the compound of Formula (I) is administered to a subject. A micro-dose may comprise, for example, about 0.05 mg to about 2.5 mg of the compound of Formula (I) (e.g., psilocybin), such as about 1.0 mg. In the case of micro-dosing the regime may comprise a regular, continuous regime of, for example, daily administration, every other day administration, or weekly, administration. Such dosing may be absent of psychological support

Sub-psychedelic dosing can also be carried out, for example, by transdermal delivery, subcutaneous administration, modified, controlled, slow, or extended-release dosage forms, including, but not limited to, depot dosage forms, implants, patches, and pumps, which can be optionally remotely controlled, matrix controlled release devices, osmotic controlled release devices, multiparticulate controlled release devices, ion-exchange resins, enteric coatings, multilayered coatings, microspheres, liposomes, and combinations thereof. Here, doses would achieve similar blood levels as low oral dosing, but would nevertheless be sub-psychedelic.

Sub-psychedelic doses can be used, e.g., for the chronic treatment a variety of diseases or disorders disclosed herein, examples of which include, but are not limited to, inflammation, pain and neuroinflammation. In such settings where chronic administration is performed over extended periods of time, the stabilized forms of the compounds provided in the present disclosure become increasingly valuable.

In some embodiments, one dose of the compound of Formula (I) (e.g., psilocybin) is administered to the subject. In some embodiments, multiple doses of the compound of Formula (I) (e.g., psilocybin) are administered to the subject. For example, at least 2, at least 3, at least 4, at least 5, at least 6, at least 7, at least 8, at least 9, at least 10, at least 15, at least 20, at least 25, at least 30, or at least 50 doses of the compound of Formula (I) (e.g., psilocybin) may be administered to the subject. In some embodiments, the same dose is administered to a subject during each administration. In some embodiments, a different dose is administered to a subject during each administration. In some embodiments, the dose of the compound of Formula (I) (e.g., psilocybin) administered to the subject is increased over time. In some embodiments, the dose of the compound of Formula (I) (e.g., psilocybin) administered to the subject is decreased over time.

The administering physician can provide a method of treatment that is prophylactic or therapeutic by adjusting the amount and timing of any of the compounds described herein on the basis of observations of one or more symptoms of the disorder or condition being treated. In some embodiments, the subject is a mammal. In some embodiments, the mammal is a human.

It is to be understood by one of skill in the art that the methods of treatment comprising administering to the subject the pharmaceutical composition comprising the disclosed solid dispersions for the treatment of one or more indications as described herein also include: the use of the solid dispersions comprising the compound of Formula (I) in the manufacture of a medicament for the treatment of one or more indications as described herein; and the use of the solid dispersions comprising the compound of Formula (I) for the treatment of one or more indications as described herein.

In some embodiments, a method of treating a subject in need thereof comprises administering to the subject a solid dispersion formulated with a therapeutically effective dose of the compound of Formula (I) (e.g., psilocybin). In some embodiments, a method of treating a subject in need thereof comprises administering to the subject a solid dispersion formulated with a therapeutically effective dose of the compound of Formula (I) (e.g., psilocybin) in a controlled environment, wherein the subject is provided with psychological support.

In some embodiments, a method of treating a subject in need thereof comprises at least one of the following:
(i) administering to the subject a therapeutically effective dose of the compound of Formula (I) in a controlled environment, wherein the subject is provided with psychological support;
(ii) having the subject participate in one or more pre-administration psychological support session(s); and/or
(iii) having the subject participate in one or more post-administration psychological support session(s).

After administration, the subject may not feel the effects of the drug for about 15 minutes, 30 minutes, 45 minutes, 60 minutes, or about 90 minutes, or any range therebetween. This period after administration and before the onset of effects will be referred to herein as the initial stage of the treatment session. The time marked by the onset of the drug's effects will be referred to herein as the early stage of the treatment session.

In some embodiments, the subject will experience peak effects at about 0.5 hours, 1.0 hours, 1.5 hours to about 3.5 hours after administration. The time period marked by the peak drug experience will be referred to herein as the peak stage of the treatment session.

In some embodiments, the effects of the compound of Formula (I) may substantially wear off from about 2 hours, 3 hours, 4 hours, to about 6 hours after administration. This time period will be referred to as the late stage of the treatment session.

In some embodiments, the subject's ability to reach a non-dual state (e.g., a mystical experience), or a sense of unity, boundlessness, ego-dissolution or transcendence correlates with positive clinical outcome. Each of these terms may be commonly defined as the breakdown of the usual relationship between self and other, whereby the subject might feel a oneness and increased sense of connectedness to the surrounding environment and/or the world at large. In some embodiments, low levels of emotional arousal-which could indicate avoidance, lack of involvement or intellectualization-might, in some embodiments, be correlated with little or no improvement in treatment outcomes.

Factors that may influence the subjective experience may include, for example, (i) dose, (ii) the mindset of the participant prior to the session, (iii) the setting of the session, (iv) the subject's ability to focus and stay with the experience, and/or (v) the subject's prior experience with psychedelics. These, and other factors, will be described in more detail below, along with ways to maximize therapeutic benefit of the treatment session.

### Pre-Administration Psychological Support Sessions

In some embodiments, the subject participates in at least one psychological support session before administration begins (“pre-administration psychological support session”). In some embodiments, a pre-administration psychological support session may be held about 1 month prior to the administration. In some embodiments, a pre-administration psychological support session may be held about 2 weeks prior to the administration. In some embodiments, a pre-administration psychological support session may be held about 1 week prior to the administration. In some embodiments, a pre-administration psychological support session may be held about 3 days prior to the administration. In some embodiments, a pre-administration psycho-logical support session may be held about 1 day prior to the administration. In some embodiments, a pre-administration psychological support session may be held on the same day as and prior to administration.

In some embodiments, the subject may participate in one, two, three, four, five, six, seven, or eight pre-administration psychological support sessions. In some embodiments, the subject may participate in at least two pre-administration psychological support sessions. In some embodiments, the subject may participate in at least three pre-administration psychological support sessions. In some embodiments, the subject may participate in pre-administration psychological support sessions at least once per week, for at least two or three weeks prior to the administration session. In some embodiments, the subject may additionally participate in a pre-administration psychological support session the day before the administration session.

The pre-administration psychological support sessions may be individual sessions, wherein a subject meets one-on-one with a therapist. In some embodiments, the psychological support sessions may be group sessions, wherein more than one subject meets with a single therapist, or more than one therapist. In some embodiments, one or more of the subject's family members or friends may be present at the pre-administration psychological support session(s).

In some embodiments, the goals of the pre-administration session may include (i) establishing therapeutic alliance between subject and therapist; (ii) answering the subject's questions and addressing any concerns; and/or (iii) demonstrating and practicing the skills of self-directed inquiry and experiential processing. In some embodiments, the pre-administration psychological support sessions focus on discussion of possible psychedelic effects, and/or preparing subjects for the dosing session by practicing relevant therapeutic techniques to reduce avoidance and anxiety, eliciting relevant therapeutic goals, building rapport, and/or establishing therapeutic alliance. During the psychological support session, skills of self-directed inquiry and experiential processing may be demonstrated and/or practiced. In some embodiments, breathing exercises meant to promote calm and/or ease anxiety may be demonstrated and/or practiced. In some embodiments, the breathing exercise comprise instructing the subject to focus on their breath and/or sensations associated with the breath throughout the body. For example, the subject may be instructed to breathe in for a count of four, to hold their breath for a moment, and then to breathe out for a count of eight. In some embodiments, the therapist and subject may discuss the most helpful ways to support in case of emotional distress during the treatment session. In some embodiments, the subject is given access (e.g., online access) to materials concerning the safety and mechanism of action of psilocybin.

In some embodiments, the pre-administration psychological support sessions will serve to establish a therapeutic goal for the treatment session. In some embodiments, the subject suggests the therapeutic goal for herself or himself. In some embodiments, the therapist suggests the therapeutic goal to the subject. In some embodiments, the subject is reminded of the therapeutic goal during the pre-administration psychological support session.

In some embodiments, the therapists are trained to counsel the subject before, during, and/or after the treatment sessions. In some embodiments, the therapist will have mental health training. In some embodiments, the therapist will be a clinical psychologist, a psychiatrist, a social worker, a doctor or a nurse. In some embodiments, the therapist will meet the following criteria: (i) demonstrate independent clinical experience with direct subject care in areas that require counselling and psychotherapeutic skills; (ii) current unrestricted professional license and/or good professional standing with no history of suspension, professional misconduct or disciplinary actions; and/or (iii) high level of openness to learning new approaches and receiving feedback.

### Psychological Support During Treatment Sessions

During the treatment session, the subject may be supervised by one or more trained therapists. The therapist supervising the subject during the treatment session may be the same therapist from the subject's pre-administration psychological support session(s), or may be a different therapist. The therapist(s) may provide psychological support to the subject as necessary. As used herein, the term “psychological support” refers to any measure(s) taken by the therapist during the subject's treatment session to ensure the safety of the subject and maximize the clinical effectiveness of the treatment session. For example, the psychological support may be anything done by the therapist to (1) to ensure psychological safety of the subject; (2) to allow the subject's subjective experience to unfold naturally within the boundaries of the therapeutic intention set at the preparation; (3) to maintain participant's attention and awareness on the experience of the present moment thus allowing exposure and processing of the challenging emotional states and personal memories; and/or (4) to generate insights and solutions for the resolution of challenging personal situations, conflicts and traumatic experiences. In some embodiments, support can be in the form of therapeutic touch, verbal reassurance, guided imagery and/or relaxation or breathing exercises. In some embodiments, the support may comprise reminders, encouragement, or active guiding. Typically, only one technique is applied at a time to allow for minimal intervention and interference with the subject's unique process.

In some embodiments, the main therapeutic goals of the therapist during the treatment session are to (i) minimize extreme anxiety, and (ii) provide appropriate support that enables the skills and processes of self-directed inquiry and experiential processing. In some embodiments, the therapist demonstrates genuine presence, patience, curiosity, and/or openness during the treatment session. “Presence” refers to being totally available and present with the subject during all stages of the treatment session, and exuding calmness at all times. “Curiosity” refers to interest and willingness to understand the subject's experience, without making assumptions. “Patience” means that the therapist facilitates the participant taking as much time as needed to explore their experiences without controlling the natural urge to help or direct the experience. “Openness” is the ability of the therapist to remain cognitively and experientially open, including a capacity to be curious about how the subject's mind may uniquely choreograph the unfolding content of a session. This includes welcoming all emotions and expressions that might occur.

In some embodiments, the psychological support may comprise curious questioning. In this technique, brief, but detailed, questioning of subjects is used to help the subjects shift and sustain their attention towards different levels of cognition and emotions (“How does that make you feel?”) Due to the applicability across a range of mental states and within various settings, the technique of curious questioning can typically be used safely and consistently during the treatment session, regardless of the quality or intensity of the experience of each subject.

In some embodiments, the level of psychological support will vary during the various stages of the subject's treatment experience (e.g., the initial stage, the early stage, the peak stage, and the late stage). In some embodiments, the type of psychological support will vary during the various stages of the subject's treatment experience (e.g., the initial stage, the early stage, the peak stage, and the late stage). Because non-dual, ego-dissolution or “unitive” experiences have been shown to positively correlate with the magnitude and durability of the clinical response, the therapist will, in some embodiments, attend to such states with particular care.

In some embodiments, a subject may experience of a compromised sense of self during the subject's treatment experience. In some embodiments, this is interpreted from a psychoanalytic perspective as a disruption of ego-boundaries, which results in a blurring of the distinction between self-representation and object-representation, and precludes the synthesis of self-representations into a coherent whole. In some embodiments, non-dual, ego-dissolution or “unitive” experiences refer to an altered state of consciousness in which there is a reduction in the self-referential awareness that defines normal waking consciousness, resulting in a compromised sense of “self” and instead only a undivided background awareness, often characterized by a sense of unity or “oneness” that exceeds sensory or cognitive apprehension. In some embodiments, a non-dual experience is state of consciousness in which the subject-object dichotomy in normal waking consciousness is substituted for a unified background awareness that is centerless and undivided. In some embodiments, an ego dissolution experience is a spontaneously occurring state of consciousness where there is a reduction in the self-referential awareness that defines normal waking consciousness, resulting in a compromised sense of “self”. In some embodiments, a unitive experience is an experience characterized by a sense of unity or “oneness” that exceeds sensory or cognitive apprehension.

At the initial and early stage of the treatment session, psychological support may be used to reduce severe and/or prolonged anxiety. Anxiety prior to or during the onset of effects is not uncommon, and the therapists may be specially trained to recognize and actively manage subjects through such periods of anxiety until the subject is comfortable enough to continue on their own. In some embodiments, therapists validate the subject's feelings of anxiety without providing interpretations of perceptual disturbances or guiding subjects towards a particular image or memory, other than encouraging them to stay relaxed and open to the emergent experiences. For example, in some embodiments, the therapist may help alleviate anxiety using a grounding exercise. In such an exercise, the subject may be encouraged to pay attention to the sounds around them or to sensations on their skin when touching the bed/couch, ground, or other objects.

At the initial and early stage of the treatment session, the therapist may encourage the subject to lie down, practice relaxation and breathing exercises, and/or listen to calming music. In some embodiments, the therapist may remind the subject of the intention for the treatment session. For example, the therapist may ask the subject “What does feeling better or recovery feel like?” or any number of similar questions. Such reminders prior to the onset of or at the onset of effects provide an implicit direction for the subjective experience during the treatment session. In some embodiments, the therapist may remind the subject that their primary task during this session is to simply collect new and interesting experiences which can then be discussed with the therapist after the session. The therapist may remind the participant of the purpose of the therapy and the role of experiential processing, namely allowing the participant to be open and curious to whatever arises and encountering thoughts and feelings previously unknown to them. In some embodiments, the therapist emphasizes that this process inherently requires letting go and a willing passivity to the psychedelic experience.

During the acute onset of action, the subject might experience perceptual changes in visual, auditory or olfactory modes, and a range of unusual physical sensations. These experiences could be anxiety-provoking. In some embodiments, the therapist may practice reassuring “arm holding”. This is where, upon the subject's request, a therapist will place his or her hand on the subject's wrist, arm, hand, or shoulder, as a way of helping the subject feel secure during this phase. This exercise may have been previously practiced during the pre-administration psychological support session.

In some embodiments, the therapist may encourage the subject to put on an eye mask, such as a Mindfold eyeshade. In some embodiments, the therapist encourages the subject to put on the eye mask before, during, or after the onset of the effects.

In some embodiments, the therapist may encourage the subject to put on headphones and listen to music. In some embodiments, the headphones reduce outside noise (e.g., “noise-cancelling” headphones). In some embodiments, the music is calming music such as instrumental (e.g., classical) music. In some embodiments, the music comprises nature sounds and/or the sound of moving water (e.g., ocean sounds). In some embodiments, the music comprises isochronic tones. In some embodiments, the music comprises moments of silence. In some embodiments, the music is emotionally evocative. In some embodiments, the music comprises a playlist which mirrors the pharmacodynamics of a typical high-dose treatment session: the initial stage, the early stage, the peak stage, and the late stage. In some embodiments, listening to music helps the subject to focus on their internal experience.

In case of prolonged anxiety or distress, therapists may, in some embodiments, actively guide participants through such experiences without interpreting or judging the experiences or giving advice. Once participants are comfortable, the therapist may encourage them to again engage in introspection.

During the peak and late stages of the treatment session, the therapist may encourage subjects to face and explore their experience, including the challenging ones. Therapists may direct subjects to participate self-directed inquiry and experiential processing to develop a different perspective on their personal challenges and conflicts, and to generate their own solutions. Such self-generated insights are not only therapeutic because of the emotional resolution, but also empowering to subjects.

As used herein, the term “self-directed inquiry” refers to directing attention to internal states. Subjects are encouraged to be curious about experiences in the present moment, including foreground and background thoughts, emotions, and physical sensations. During the preparation and integration stages, this inquiry might mean asking specific and detailed questions to help direct attention to internal states. However, during the period of drug action, inquiry might simply mean an attitude of openness to inner experiences.

As used herein, “experiential processing” refers to a participant's ability to maintain full attention on the experiences that come into awareness through self-directed enquiry. This includes a willingness and ability to be with and/or move ‘in and through’ even uncomfortable or challenging thoughts, feelings, sensations or emotions, until discomfort is diminished or resolved.

In some embodiments, the therapist will employ a transdiagnostic therapy. In some embodiments, the transdiagnostic therapy is a Method of Levels (MOL) therapy. In still further embodiments, the MOL therapy comprises Self-Directed Enquiry and Experiential Processing. Typically, MOL uses brief, but detailed, curious questioning to help subjects shift and sustain their attention towards different levels of cognition and emotions (Carey, 2006; Carey, Man-sell & Tai, 2015). The emphasis within MOL is on identifying and working with a subject's underlying distress as opposed to just their symptoms. Such MOL related methods and techniques can include: (1) Self-directed enquiry-directing attention to internal states. Participants are encouraged to be curious about experiences in the present moment, including foreground and background thoughts, emotions, and physical sensations; during the preparation and integration stages, such enquiry can mean asking specific and detailed questions to help direct attention to internal states, although for some embodiments, during the period of drug action, enquiry can refer to an attitude of openness to inner experiences; and (2) Experiential processing-sustained focus on the experience; which refers to a participant's ability to maintain full attention on the experiences that come into awareness through self-directed enquiry. This includes a willingness and ability to be with and/or move ‘in and through’ even uncomfortable or challenging thoughts, feelings, sensations or emotions, until discomfort is diminished or resolved.

In some embodiments, the psychological support comprises mindfulness-based therapy or CBT cognitive behavioral therapy (CBT). In some embodiments, the psychological support is informed by a functional theory of human behavior called Perceptual Control Theory.

Occasionally, the subject will try to avoid emerging experiences or distract him/herself while trying to regain cognitive control over the unusual state of their mind. Such distractions may take different forms. For example, the subject might want to engage in a conversation or prematurely describe in detail their experience, visions or insights. When this occurs, the therapist may aim to remain as silent as possible, thereby enabling the subject and his/her inner experience to direct the course of the treatment session. In some embodiments, the therapist may use active listening skills paired with prompts to encourage the subject to continue focusing attention on present experiences, particularly if the participant engages the therapist in conversation. In another example, a subject might ask to go to the bathroom or have a drink of water. The sudden and urgent character of such requests might suggest that they are really trying to avoid emerging material. In such cases, the therapist may encourage the subject to stay with the experience by simply redirecting their attention. For example, the therapist may say something like, “We will take a bathroom break at the end of this piece of music” or “I will get you water in a little while. Why don't you put the eye shades back on and relax for a few minutes?” If the subject is trying to avoid a difficult experience, they might listen to the suggestion and relax.

In some embodiments, spontaneous movement such as shaking, stretching or dancing while engaging with the experience is accepted and often encouraged, unless the movement seems to be a way to distract oneself from the experience. In some embodiments, if the subject continues to move around a lot, reminders to periodically return to a lying down position and to actively focus inwards may be provided.

The therapist is not required to understand, support or even have an opinion about the nature or content of the subject's experiences, but the therapist may validate them and convey openness toward the subject's own view of them without dismissing or pathologizing any experience based on its unusual content. These experiences may provide the subject with a perspective that goes beyond identification with their personal narrative. In some embodiments, the therapist will validate one or more of the subject's experiences. In some embodiments, validation of the experiences simply means acknowledging the courage of opening up to the experience and the possibility that any experience will serve the intention of the session.

In some embodiments, a therapist provides psychological support for approximately 4-8 hours immediately after administration. In some embodiments, the therapist uses guided imagery and/or breathing exercises to calm the subject and/or focus the subject's attention. In some embodiments, the therapist holds the hand, arm, or shoulder of the subject. In some embodiments, the therapist counsels the subject to do one or more of the following: (1) to accept feelings of anxiety, (2) to allow the experience to unfold naturally, (3) to avoid psychologically resisting the experience, (4) to relax, and/or (5) to explore the subject's own mental space.

In some embodiments, the therapist avoids initiating conversation with the subject, but responds if the subject initiates conversation. Typically, active intervention is kept to a minimum during the treatment experience. In some embodiments, the subject is encouraged to explore their own mental space, and simple guided imagery may be used to assist relaxation. “Guided imagery” refers to an exercise wherein the subject is asked to imagine a scene (e.g., “Invite a scene, perhaps a landscape, and tell me where you find yourself”; “Imagine a place that feels safe to you.”).

### Post-Administration Psychological Support Session

In some embodiments, subjects may be encouraged to engage in post-administration integration sessions with their therapist. Integration is a process that involves processing, or embodying, a psychedelic experience within a therapeutic context. The process initially begins by the subject verbalizing and reflecting upon any experience from the treatment session, and discussing it openly with their therapist. Successful integration of a therapy experience accommodates for emotional changes and comprises of translating experiences into new insights, perspectives, and subsequently new behaviors that can be used to benefit the subject's quality of life. New perspectives might in turn influence the participant's current knowledge or values and lead to new ways of relating to cognitions, emotions, behaviors and physical experiences.

In some embodiments, the goals and supportive methods used by the therapist throughout integration sessions should remain consistent, regardless of the intensity or content of the subjective experience explored by the subject. That said, the methods of support used by the therapist should accommodate for the full range of experiences a subject might have faced.

The integration process is not one that should be limited to the sessions with the therapist, and is a process that will likely continue to unfold beyond the visits in clinic. The therapist might encourage the participant to use methods such as spending time in nature, exercise, or creative expression to help facilitate the process further. The subject might also be encouraged to discuss experiences with their friends, family, and/or support network. The role of the integration sessions is not to cover and work on every experience, but to empower the participant by building their capacity to experientially process information safely. This enables the participant to continue self-directed integration, even outside of study visits.

In some embodiments, the subject participates in at least one psychological support session after administration (“post-administration psychological support session”). In some embodiments, a post-administration psychological support session may be held on the same day as the treatment session, after the effects of the drug (e.g., a compound of Formula (I)) have substantially worn off. In some embodiments, a post-administration psychological support session may be held the day after the treatment session. In some embodiments, a post-administration psychological support session may be held two days after the treatment session. In some embodiments, a post-administration psychological support session may be held three days after the treatment session. In some embodiments, a post-administration psychological support session may be held about one week after the treatment session. In some embodiments, a post-administration psychological support session may be held about two weeks after the treatment session. In some embodiments, a post-administration psychological support session may be held about one month after the treatment session. In some embodiments, a post-administration psychological support session may be held about three months after the treatment session. In some embodiments, a post-administration psychological support session may be held about six months after the treatment session. In some embodiments, a post-administration psychological support session may be held about twelve months after the treatment session.

In some embodiments, the subject may participate in one, two, three, four, five, six, seven, or eight post-administration psychological support sessions. In some embodiments, the subject may participate in at least two, or at least three post-administration psychological support sessions.

The post-administration psychological support sessions may be individual sessions, wherein a subject meets one-on-one with a therapist. In some embodiments, the psychological support sessions may be group sessions, wherein more than one subject meets with a single therapist, or more than one therapist. In some embodiments, one or more of the subject's family members or friends may be present at the post-administration psychological support session(s).

In some embodiments, the post-administration psychological support session may focus on integration of the treatment experience. Integration may involve processing a psychedelic experience in a therapeutic context. Integration may comprise psychological and somatic processing of the experience and a successful assimilation of insights into the subject's life for the purpose of growth, healing and/or well-being. During an integration session, a subject may be encouraged to talk about and reflect upon their experiences during the treatment session. In some embodiments, integration may comprise an external expression of the treatment experience, such as choice of words, tone of voice, gestures, and/or particular physical activities (yoga, exercise, bodywork, etc.) In some embodiments, integration comprises creatively expressing any insights or experiences gained during a treatment experience, for example through poetry, art, music/singing, dance, writing or drawing. In some embodiments, the subject may be encouraged to reflect on both the thoughts and the feelings that he or she underwent during the treatment session, as well as to express those ideas and emotions into a concrete form that can serve as a tool for continuing to remember and integrate those lessons into the future. In some embodiments, the subject may be encouraged to acknowledge and connect with the range of the emotional cognitive and physical experiences of the treatment session, and relate them to current experiences in their life situation. This may be accomplished, for example, by discussing them initially with their therapist, and perhaps later with their family, friends, and support circle. Integration helps accommodate changes in emotional states as new insights are generated and integrated. When further explored through oscillating attention between foreground and background thoughts and emotions, such insights may lead to natural and effortless changes in perspectives or behaviors. In some embodiments, the integration process is not limited to initial integration meetings with the therapist, but continues to unfold spontaneously through a participant's own processing and actions in every day life.

In the case of a low-intensity experience, the integration process might focus on the mental content that emerged during the hours of relaxation and introspection. This might also include reactions to what might have been an unremarkable experience, such as feeling of disappointment, anger, relief etc.

### Psychological Support Provided Remotely

In some embodiments, psychological support may be provided remotely to a subject. For example, a therapist providing psychological support may not be in the same room, the same building, or in the same facility as a subject. Remote psychological support may be provided, for example by telephone (i.e., by voice call), by video call or video conference, by text, or by email.

In some embodiments, a pre-administration therapy session is conducted remotely. In some embodiments, a post-administration therapy session (e.g., an integration session) is conducted remotely.

In some embodiments, psychological support is provided remotely during the subject's treatment session. For example, in some embodiments, the subject takes the compound of Formula (I), via a solid dispersion dosage form, in his or her own home, and a therapist provides psychological support by voice call, video call, text, email, etc., for the hours (e.g., at least 2-8 hours or 4-8 hours) after the subject has taken the drug. In some embodiments, the subject takes the compound of Formula (I) in an administration facility as described herein, and the therapist provides psychological support to the subject a therapist provides psychological support by voice call, video call, text, email, etc., for the hours (e.g., at least 2-8 hours or 4-8 hours) after the subject has taken the drug

In some embodiments, remote psychological support is provided to the subject using a digital or electronic system. In some embodiments, the digital or electronic system may comprise one or more of the following features: 1) The digital or electronic system securely connects patients with one or more therapists or physicians for “virtual visits.” These virtual visits may be introductory or routine; 2) The digital or electronic system allows a subject to qualify, prequalify, or register for a clinical trial, or a psychological support session; 3) The digital or electronic system is configured to help therapists and/or physicians manage and interact with patients, for example, the electronic system may allow the therapist to share documents with subjects, keep notes about sessions, or schedule future sessions; 4) The digital or electronic system is configured to provide alerts for crisis intervention, for example, the digital or electronic system may allow the subject to contact the therapist if they are feeling anxiety or otherwise urgently need to talk to the therapist; 5) The digital or electronic system is configured to help prepare the subject for a visit with their therapist and/or physician, for example, the digital or electronic system may contain information regarding drug (e.g., psilocybin or a deuterated form thereof), the therapeutic protocol, etc.; 6) The digital or electronic system is configured to allow the therapist to provide psychological support during the subject's treatment session, for example, the system may comprise a video calling or chat feature; 7) The digital or electronic system is configured to allow the therapist to provide psychological support during a post-administration session (e.g., an integration session); 8) The digital or electronic system is configured to track the subject's adherence to the treatment regimen or goals; 9) The digital or electronic system is configured to assess one or more clinical endpoints in the subject, for example the system may comprise one or more questionnaires or exercises for the subject to complete, and the results may be made available to the subject's physician and/or therapist.

In some embodiments, the digital or electronic system is an “app” for use on a mobile phone or a computer. In some embodiments, the digital or electronic system is a website. In some embodiments, the digital or electronic system comprises a “chat” feature which allows communication between the subject and the therapist in real time. In some embodiments, the website comprises a video calling feature, which allows for the therapist to communicate with the subject using video communication. In some embodiments, the digital or electronic system is configured to allow a single therapist to provide psychological support to one or more subjects at or around the same time.

In some embodiments, psychological support sessions may be pre-recorded (e.g., audio or video recording) and provided to the subject for use at the subject's convenience via the digital or electronic system.

### Administration Facility, “Set and Setting”

As used herein, the term “set and setting” refers to the subject's mindset (“set”) and the physical and social environment (“setting”) in which the user has the treatment session. In some embodiments, the compound of Formula (I) may be administered in a particular set and setting. In some embodiments, the set and setting is controlled, to the extent possible, to maximize therapeutic benefit of the treatment session.

In some embodiments, administration is performed in a facility specifically designed for treatment. Administration to the subject in a facility where the subject feels safe and comfortable may help ease anxiety in the subject, and may facilitate maximum clinical benefit. Administration may be performed, for example, in the subject's home or at a clinical facility.

In some embodiments, administration is performed in a facility (e.g., a room) with a substantially non-clinical appearance, for example, in a room that comprises soft furniture (e.g., plush couches, chairs, or pillows) and/or plants. In some embodiments, the room may be decorated using muted colors (e.g., greyed, dulled, or desaturated colors). In some embodiments, the light in the room is dimmed and/or light levels are kept or adjust to be relatively low. In some embodiments, the room lighting is adjusted for intensity and/or color. In some embodiments, a virtual reality or augmented reality system (e.g., computer with visual/graphical and auditory outputs) is used. In some embodiments, the room comprises a sound system, for example a high-resolution sound system. In some embodiments, the sound system can allow for simultaneous ambient and earphone listening. In some embodiments, the subject may bring meaningful photographs or objects into the administration room.

In some embodiments, the room comprises a couch. In some embodiments, the room comprises a bed. In some embodiments the room comprises more than one couch or bed, such as 2, 3, 4, 5, 6, 7, 8, 9, or 10 couches or beds. In some embodiments, the subject sits on or lies in the couch or bed for approximately 2-8 hours or 4-8 hours, or a substantial fraction thereof, immediately after administration. In some embodiments, the subject listens to music for approximately 2-8 hours or 4-8 hours, or a substantial fraction thereof, immediately after administration. In some embodiments, the subject wears an eye mask for approximately 2-8 hours or 4-8 hours, or a substantial fraction thereof, immediately after administration. In some embodiments, the subject is provided with a weighted blanket.

In some embodiments, each subject is supervised by one therapist during the treatment session. In some embodiments, each subject is supervised by more than one therapist during the treatment session, such as two therapists, three therapists, four therapists, or five therapists. In some embodiments, one therapist may supervise multiple subjects, wherein each subject is participating in a treatment session. For example, one therapist may supervise two, three, four, five, six, seven, eight, nine, or ten subjects.

Embodiments of the disclosure include use of additional tools and/or technique(s) with dosage/administration, including various transcranial magnetic stimulation (TMS) methods and protocols, for example, prior or subsequent to one or more dosing(s), biofeedback devices, etc.

Some embodiments can be used with a digital health product or digital solution. Teachings of the disclosure include utilization of such digital health products and/or related digital biomarkers as diagnostic and/or prognostic tools for patient monitoring and management pre-treatment, during treatment, and/or post treatment. Digital biomarkers can include, but are not limited to number of and/or time of phone calls/e-mails/texts; word length in text communication; gestures used (taps, swipes, or other); gyroscope derived information e.g. orientation of the phone; acceleration of the phone; keystroke patterns; location derived information from GPS; facial expressions and/or microexpressions; voice or vocal markers; natural language processing; social media use; sleep patterns; specific words or emojis used or not used; and/or the like. For example, in one embodiment, a digital health product can be utilized to determine dosing amount and/or dosing frequency, indicator of a need for re-dosing, re-dosing amount, a warning or alert, as tracking of compliance, etc.

In some embodiments, methods of treatment can include providing a clearance time for a subject or patient, such one or more medications is not present or substantially cleared from the system of the subject/patient. For example, methods of treatment can be configured such that, upon administration, the subject is not taking other serotonergic medications such as: selective-serotonin reuptake inhibitors, selective norepinephrine reuptake inhibitors, tricyclic anti-depressants, monoamine oxidase inhibitors and/or antipsychotics. In some embodiment, the method of treatment includes treatment concurrently with one or more medications, including but not limited to selective-serotonin reuptake inhibitors, selective norepinephrine reuptake inhibitors, tricyclic antidepressants, and/or monoamine oxidase inhibitors. In some embodiments, the method includes treatment such that subjects or patients take concomitant compounds or medications, including but not limited to benzodiazepines, cannabidiol (CBD) and/or other cannabinoids (e.g., THC (tetrahydrocannabinol); THCA (tetrahydrocannabinolic acid); CBD (cannabidiol); CBDA (cannabidiolic acid); CBN (cannabinol); CBG (cannabigerol); CBC (cannabichromene); CBL (cannabicyclol); CBV (cannabivarin); THCV (tetrahydrocannabivarin); CBDV (cannabidivarin); CBCV (cannabichromevarin); CBGV (cannabigerovarin); CBGM (cannabigerol monomethyl ether); CBE (cannabielsoin); CBT (cannabicitran); and/or the like), magnesium, levomefolic acid, e.g., for a period of time prior to, just prior to, and/or at the same time as receiving the compound of Formula (I) in the form of the solid dispersion.

In some embodiments, the method includes treatment such that a subject has not taken one or more medications, particularly has not taken one or more serotonergic medications for at least 2 days, at least, 3 days, at least 4 days, at least 5 days, at least six days, at least 1 week, at least 2, 3, or 4 weeks before administration of the compound of Formula (I).

In some embodiments, the method and/or treatment can comprise subperceptual-dosing (e.g., a dose of less than 3 mg, 2.5 mg, 2 mg, 1.5 mg, 1 mg, 0.9 mg, 0.8 mg, 0.7 mg, 0.6 mg, 0.5 mg, 0.4 mg, 0.3 mg, 0.2 mg, or 0.1 mg) prior to and/or following the administration of a relatively larger single dose or multiple doses (given a few days to a few weeks apart), where the relatively larger single dose or multiple doses is one or more of 5 mg or more, 10 mg or more, 15 mg or more, 20 mg or more, 25 mg or more, 30 mg or more, 35 mg or more, 40 mg or more, 45 mg or more, 50 mg or more.

Embodiments of the disclosure include methods utilizing a digital biomarker, for example, as a diagnostic and/or prognostic tool for patient management pre-, during and/or post treatment, wherein the digital biomarker is one or more biomarkers associated with executive function, cognitive control, working memory, processing speed, and/or emotional valence.

In some embodiments the digital biomarker is identified from patterns in smartphone use such as swipes, taps, and other touchscreen activities, and can be scientifically validated to provide measurements of subject status, such as cognition and mood, including, by way of non-limiting example, as disclosed in one or more of the following, each of which is herein expressly incorporated by reference for all purposes: US20170086727, US20170258382, US20170258383, US20170287348, U.S. Ser. No. 10/148,534, U.S. Pat. No. 9,737,759, and/or U.S. Ser. No. 10/231,651.

Biomarkers which may serve as a diagnostic and/or prognostic tool for patient management pre, during and/or post treatment may be identified using one or more of: number of and/or time of phone calls/e-mails/texts; word length in text communication; gestures used (taps, swipes, or other); gyroscope derived information e.g. orientation of the phone; acceleration of the phone; keystroke patterns; location derived information from GPS; facial expressions and/or microexpressions; voice or vocal markers; natural language processing; social media use; sleep patterns; specific words or emojis used or not used; and/or the like. In some embodiments, health components and/or connected biomonitors and/or smart devices/wearables can be utilized to collect information to be used in diagnostic and/or prognostic outputs. For example, in some embodiments, a heart rate monitor or similar device can collect a subject's data and heart rate variability (for example only, as disclosed in U.S. Ser. No. 10/058,253, the entirety of which is herein incorporated by reference) can be used to assess/determine a metric relating to the subject's current emotional state, relative change in emotional state, etc., which can be used in determining a new or follow-on treatment plan, adjusting a treatment plan, etc.

In accordance with a further aspect of the disclosure there is provided a method of assessing a subject pre, during and/or post treatment of a central nervous system disorder to determine whether to provide treatment or a further treatment comprising monitoring one or more biomarkers associated with executive function, cognitive control, working memory, processing speed, and emotional valence, and determining the treatment based on an outcome. The method can further comprise the step of administering the compound of Formula (I) for a first or a subsequent time.

In some embodiments, the biomarker is identified from patterns in smartphone use such as swipes, taps, and other touchscreen activities, and are scientifically validated to provide measurements of cognition and mood. For example, in some instances, the pattern is identified using one or more of: number of and/or time of phone calls/e-mails/texts; word length in text communication; gestures used (taps, swipes, or other); gyroscope derived information e.g. orientation of the phone; acceleration of the phone; keystroke patterns; location derived information from GPS; facial expressions and/or microexpressions; voice or vocal markers; natural language processing; social media use; sleep patterns; specific words or emojis used or not used; and/or the like.

Embodiments include a method of assessing a subject pre, during and/or post treatment of a central nervous system disorder to determine whether to provide treatment or a further treatment with the compound of Formula (I) comprising monitoring one or more biomarkers associated with executive function, cognitive control, working memory, processing speed, and emotional valence, and determining the treatment based on an outcome; the method can further comprise administering the compound of Formula (I) for a first or a subsequent time.

In some embodiments, the disclosure provides for treating 2 or more subjects, the method comprising administering to each subject a therapeutically effective dose of the compound of Formula (I) via a solid dispersion dosage form at the same time or substantially the same time (e.g., dosed within several minutes of each other, within 5, 10, 15, 20, 25, or 30 min of each other), wherein each subject is aware of the other subject also receiving treatment. In some embodiments, the subjects are in the same room. In some embodiments, the subjects are in different rooms.

In some embodiments, the disclosure provides a method of treating a subject, the method comprising administering to the subject a therapeutically effective dose of the compound of Formula (I) via a solid dispersion dosage form, and providing a virtual reality/immersive reality digital tool. In some embodiments, the light in the room is dimmed and/or light levels are kept or adjusted to be relatively low. In some embodiments, darkened glasses or eye shades are provided. In some embodiments, the room lighting is adjusted for intensity and/or color. In some embodiments, a virtual reality or augmented reality system (e.g., computer with visual/graphical and auditory outputs) is used.

### Subjects

In some embodiments, the subject is a male. In some embodiments, the subject is a female. In some embodiments, the female subject is pregnant or post-partum. In some embodiments, the subject is attempting to reduce or eliminate their use of a pharmaceutical agent, such as an antidepressant or an anti-epileptic drug. In some embodiments, the subject is attempting to reduce or eliminate their use of the pharmaceutical agent before becoming pregnant, having surgery or other medical procedure, or starting to use a different pharmaceutical agent.

The subject may be a geriatric subject, a pediatric subject, a teenage subject, a young adult subject, or a middle aged subject. In some embodiments, the subject is less than about 18 years of age. In some embodiments, the subject is at least about 18 years of age. In some embodiments, the subject is about 5-10, about 10-15, about 15-20, about 20-25, about 25-30, about 30-35, about 35-40, about 40-45, about 45-50, about 50-55, about 55-60, about 60-65, about 65-70, about 70-75, about 75-80, about 85-90, about 90-95, or about 95-100 years of age.

The subject may have a chronic disease or a terminal disease. In some embodiments, the subject may have a life-altering disease or condition (such as the loss of a limb or onset of blindness).

The subject may have recently been diagnosed with a disease, disorder, or condition. For example, the subject may have been diagnosed within 1 month, within 3 months, within 6 months, or within 1 year. In some embodiments, the subject may have been living with a disease, disorder, or condition for an extended period time, such as at least 6 months, at least 1 year, at least 3 years, at least 5 years, or at least 10 years.

In some embodiments, the subject may be a cancer patient, such as a Stage 4 or terminal cancer patient. In some embodiments, the subject may have been determined to have a limited time to live, such as less than 1 year, less than 6 months, or less than 3 months.

The subject may have previously taken a psychedelic drug, or may have never previously taken a psychedelic drug. For example, the subject may or may not have previously taken psilocybin, a psilocybin mushroom (“magic mushroom”), LSD (lysergic acid diethylamide or acid), mescaline, or DMT (N,N-Dimethyltryptamine).

In some embodiments, the subject may have previously taken one or more serotonergic antidepressants (e.g., selective serotonin reuptake inhibitors (SSRis)). In some embodiments, the subject has never previously taken a serotonergic antidepressant. In some embodiments, the subject has not taken any serotonergic antidepressants for at least 2 weeks, at least 4 weeks, or at least 6 weeks prior to receiving treatment with the compound of Formula (I).

In some embodiments, the subject may have previously received electroconvulsive therapy (ECT). In some embodiments, the subject has not received any ECT for at least 2 weeks, at least 4 weeks, or at least 6 weeks prior to receiving treatment with the compound of Formula (I).

The subject may have a medical condition that prevents the subject from receiving a particular medical therapy (such as an SSRI or ECT). In some embodiments, the subject may have previously had an adverse reaction to a particular medical therapy (such as an SSRI or ECT). In some embodiments, a prior medical therapy (such as an SSRI or ECT) was not effective in treating a disease, disorder, or condition in the subject.

Diseases, Disorders, and/or Conditions to be Treated

Provided herein are methods of treating a subject in need thereof, the method comprising administering to the subject a pharmaceutical composition comprising a solid dispersion of the present disclosure formulated with a compound of Formula (I), or a pharmaceutically acceptable salt, a polymorph, stereoisomer, a tautomer, solvate, or active metabolite thereof.

The subjects treated herein may have a disease or disorder associated with a serotonin 5-HT₂ receptor.

In some embodiments, the disease or disorder is a neuropsychiatric disease or disorder or an inflammatory disease or disorder. In some embodiments, the neuropsychiatric disease or disorder is not schizophrenia or cognitive deficits in schizophrenia.

In some embodiments, the disease or disorder is a central nervous system (CNS) disorder, including, but not limited to, major depressive disorder (MDD), treatment-resistant depression (TRD), post-traumatic stress disorder (PTSD), bipolar and related disorders (including, but not limited to, bipolar I disorder, bipolar II disorder, cyclothymic disorder), obsessive-compulsive disorder (OCD), generalized anxiety disorder (GAD), social anxiety disorder, substance use disorders (including, but not limited to, alcohol use disorder, opioid use disorder, amphetamine use disorder, nicotine use disorder, smoking, and cocaine use disorder), eating disorders (including, but not limited to anorexia nervosa, bulimia nervosa, binge-eating disorder, etc.), Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, suicidal ideation, suicidal behavior, major depressive disorder with suicidal ideation or suicidal behavior, melancholic depression, atypical depression, dysthymia, non-suicidal self-injury disorder (NSSID), chronic fatigue syndrome, Lyme's disease, gambling disorder, paraphilic disorders (including, but not limited to, pedophilic disorder, exhibitionistic disorder, voyeuristic disorder, fetishistic disorder, sexual masochism or sadism disorder, and transvestic disorder, etc.), sexual dysfunction (e.g., low libido, hypoactive sexual desire disorder (HSDD), etc.), peripheral neuropathy, and obesity.

In some embodiments, the disease or disorder is major depressive disorder (VIDD).

In some embodiments, the disease or disorder is treatment-resistant depression (TRD).

In some embodiments, the disease or disorder is an anxiety-related disorder, such as generalized anxiety disorder (GAD), social anxiety disorder, panic disorder, a phobia-related disorder (e.g., phobias related to flying, heights, specific animals such as spiders/dogs/snakes, receiving injections, blood, etc., agoraphobia), separation anxiety disorder, selective mutism, etc. In some embodiments, the disease or disorder is generalized anxiety disorder (GAD). In some embodiments, the disease or disorder is social anxiety disorder.

In some embodiments, the disease or disorder is a compulsive disorder, such as obsessive-compulsive disorder (OCD), body-focused repetitive behavior, hoarding disorder, gambling disorder, compulsive buying, compulsive internet use, compulsive video gaming, compulsive sexual behavior, compulsive eating, compulsive exercise, etc. In some embodiments, the disease or disorder is obsessive-compulsive disorder (OCD).

In some embodiments, the disease or disorder is headaches (e.g., cluster headache, migraine, etc.).

In some embodiments, the disease or disorder is a substance use disorder. In some embodiments, the disease or disorder is alcohol use disorder. In some embodiments, the disease or disorder is smoking disorder and the therapy is used for smoking cessation.

Pharmaceutical compositions of the present disclosure may provide cognitive benefits to subject's suffering from neurological and neurodegenerative diseases such as Alzheimer's disease and other dementia subtypes, Parkinson's disease, etc. For example, emerging psychedelic research/clinical evidence indicates that psychedelics, such as psilocybin, may be useful as disease-modifying treatments in subjects suffering from neurodegenerative diseases such as Alzheimer's disease and other forms of dementia. See Vann Jones, S. A. and O'Kelly, A. “Psychedelics as a Treatment for Alzheimer's Disease Dementia” Front. Synaptic Neurosci., 21, August 2020; Kozlowska, U., Nichols, C., Wiatr, K., and Figiel, M. (2021), “From psychiatry to neurology: Psychedelics as prospective therapeutics for neurodegenerative disorders” Journal of Neurochemistry, 00, 1-20; Garcia-Romeu, A., Darcy, S., Jackson, H., White, T., Rosenberg, P. (2021), “Psychedelics as Novel Therapeutics in Alzheimer's Disease: Rationale and Potential Mechanisms” In: Current Topics in Behavioral Neurosciences. Springer, Berlin, Heidelberg. For example, psychedelics such as psilocybin are thought to stimulate neurogenesis, provoke neuroplastic changes, and to reduce neuroinflammation. Thus, in some embodiments, the solid dispersions of the present disclosure are used for the treatment of neurological and neurodegenerative disorders. In some embodiments, the solid dispersions of the present disclosure are used for the treatment of Alzheimer's disease. In some embodiments, the solid dispersions of the present disclosure are used for the treatment of dementia. In some embodiments, the solid dispersions of the present disclosure are used for the treatment of Parkinson's disease. As described above, such treatment may stimulate neurogenesis, provoke neuroplastic changes, and/or provide neuroinflammatory benefits (e.g., reduced neuroinflammation compared to prior to the beginning of treatment), and as a result, may slow or prevent disease progression, slow or reverse brain atrophy, and reduce symptoms associated therewith (e.g., memory loss in the case of Alzheimer's and related dementia disorders). While not limited thereto, pharmaceutical compositions adapted for oral and/or extended-release dosing are appropriate for such treatment methods, with sub-psychedelic dosing being preferred.

Further, many of the behavioral issues associated with chronic and/or life-threatening illnesses, including neurodegenerative disorders such as Alzheimer's disease, may benefit from treatment with the solid dispersions disclosed herein. Indeed, depression, anxiety, or stress can be common among patients who have chronic and/or life-threatening illnesses such as Alzheimer's disease, autoimmune diseases (e.g., systemic lupus erythematosus, rheumatoid arthritis, and psoriasis), cancer, coronary heart disease, diabetes, epilepsy, HIV/AIDS, hypothyroidism, multiple sclerosis, Parkinson's disease, and stroke. For example, depression is common in Alzheimer's disease as a consequence of the disease, as well as being a risk factor for the disease itself. Symptoms of depression, anxiety, or stress can occur after diagnosis with the disease or illness. Patients that have depression, anxiety, or stress concurrent with another medical disease or illness can have more severe symptoms of both illnesses and symptoms of depression, anxiety, or stress can continue even as a patient's physical health improves. Pharmaceutical compositions described herein can be used to treat depression, anxiety, and/or stress associated with a chronic or life-threatening disease or illness.

Accordingly, in some embodiments, the methods herein are used to treat symptoms, e.g., depression, anxiety, and/or stress, associated with a chronic and/or life-threatening disease or disorder. In some embodiments, the disease or disorder is Alzheimer's disease. In some embodiments, the methods herein are used for the treatment of depression, anxiety, and/or stress associated with Alzheimer's disease. In some embodiments, the disease or disorder is Parkinson's disease. In some embodiments, the methods herein are used for the treatment of depression, anxiety, and/or stress associated with Parkinson's disease. In some embodiments, the disease or disorder is cancer related depression and anxiety. As discussed above, oral and/or extended-release dosing is appropriate for such applications, particularly when blood concentrations of active ingredient (e.g., a compound of Formula (I)) are kept below the psychedelic threshold.

In some embodiments, the disease or disorder is a neurological and developmental disorder such as autism spectrum disorder, including Asperger's syndrome. For example, Asperger's syndrome is a subtype of autism spectrum disorder that is treatable with anxiety drugs.

In some embodiments, the disease or disorder is a genetic condition that causes learning disabilities and cognitive impairment. An example of such a genetic condition is fragile X syndrome, which can cause mild to moderate intellectual disabilities in most males and about one-third of affected females. Subjects with fragile X syndrome may display anxiety, hyperactive behavior (e.g., fidgeting and impulsive actions), attention deficit disorder, and/or features of autism spectrum disorder, and these signs and symptoms may be treated with the methods herein.

In some embodiments, the disease or disorder is mental distress, e.g., mental distress in frontline healthcare workers.

In some embodiments, the disease or disorder includes conditions of the autonomic nervous system (ANS).

In some embodiments, the disease or disorder includes pulmonary disorders including asthma and chronic obstructive pulmonary disorder (COPD).

In some embodiments, the disease or disorder includes cardiovascular disorders including atherosclerosis.

### Anxiety Disorders

In some embodiments, a method of treating one or more anxiety disorders in a subject in need thereof comprising administering to the subject an effective amount a compound of Formula (I), via a solid dispersion, is disclosed herein.

As used herein, the term “anxiety disorder” refers to a state of apprehension, uncertainty, and/or fear resulting from the anticipation of an event and/or situation. Anxiety disorders cause physiological and psychological signs or symptoms. Non-limiting examples of physiological symptoms include muscle tension, heart palpitations, sweating, dizziness, shortness of breath, tachycardia, tremor, fatigue, worry, irritability, and disturbed sleep. Non-limiting examples of psychological symptoms include fear of dying, fear of embarrassment or humiliation, fear of an event occurring, etc. Anxiety disorders also impair a subject's cognition, information processing, stress levels, and immune response. In some embodiments, the methods disclosed herein treat chronic anxiety disorders. As used herein, a “chronic” anxiety disorder is recurring.

Anxiety disorders are thought to be the most prevalent class of psychiatric disorders, with over 60 million people in Europe affected each year. While, according to epidemiological surveys, it is estimated that one third of the population is affected by an anxiety disorder during their lifetime.

Non-limiting examples of anxiety disorders include acute stress disorder, anxiety due to a medical condition, generalized anxiety disorder, panic disorder, panic attack, a phobia, post-traumatic stress disorder, obsessive-compulsive disorder, separation anxiety disorder, social anxiety disorder, substance-induced anxiety disorder, or selective mutism.

In some embodiments, the subject in need thereof develops an anxiety disorder after experiencing the effects of a disease. The effects of a disease include diagnosis of an individual with said disease, diagnosis of an individual's loved ones with said disease, social isolation due to said disease, quarantine from said disease, or social distancing as a result of said disease. In some embodiments, an individual is quarantined to prevent the spread of the disease. In some embodiments, the disease is COVID-19, SARS, or MERS. In some embodiments, a subject develops an anxiety disorder after job loss, loss of housing, or fear of not finding employment.

In some embodiments, an anxiety disorder comprises a medical diagnosis based on the criteria and classification from Diagnostic and Statistical Manual of Medical Disorders, 5th Ed. In one embodiment, an anxiety disorder comprises a medical diagnosis based on an independent medical evaluation.

In some embodiments, a method of treating a generalized anxiety disorder in a subject in need thereof is disclosed herein. Generalized anxiety disorder is characterized by excessive anxiety and worry, fatigue, restlessness, increased muscle aches or soreness, impaired concentration, irritability, and/or difficulty sleeping. In some embodiments, a subject with generalized anxiety disorder does not have associated panic attacks.

In some embodiments, a method of treating a social anxiety disorder in a subject in need thereof is disclosed herein. As used herein, “social anxiety disorder” is a marked fear or anxiety about one or more social situations in which the individual is exposed to possible scrutiny by others. Non-limiting examples of situations which induce social anxiety include social interactions (e.g., having a conversation, meeting unfamiliar people), being observed (e.g., eating or drinking), and performing in front of others (e.g., giving a speech). In some embodiments, the social anxiety disorder is restricted to speaking or performing in public. In some embodiments, treating according to the methods of the disclosure reduces or ameliorates a symptom of social anxiety disorder. In some embodiments, the reduction/amelioration in a symptom of a social anxiety disorder can be measured using any one of the assessments described herein. In some embodiments, after treating the symptom is reduced compared to prior to treating by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, a method of treating panic disorder in a subject in need thereof is disclosed herein. A subject with panic disorder experiences panic attacks, which are sudden feelings of terror when there is no real danger. A subject experiencing a panic attack may feel as if he or she is losing control. In some embodiments, a subject with a panic disorder has physical symptoms, including but not limited to, a fast heartbeat, chest or stomach pain, breathing difficulty, weakness or dizziness, sweating, feeling hot or cold, tingly or numb hands. In some embodiments, treating according to the methods of the disclosure reduces the incidence of panic attacks compared to prior to said treatment. In some embodiments, treating according to the methods of the disclosure reduces the number of panic attacks per month by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, a method of treating a phobia in a subject in need thereof is disclosed herein. A phobia is an irrational fear of something that is unlikely to cause harm. Phobias cause anxiety, fear, and panic. In some embodiments, the phobia is agoraphobia, acrophobia, aerophobia, arachnophobia, astraphobia, autophobia, claustrophobia, homophobia, hydrophobia, ophidiophobia, zoophobia, alektorophobia, onomatophobia, pogonophobia, nephophobia, and cryophobia. In some embodiments, the subject has agoraphobia. In some embodiments, the phobia is a specific phobia. In some embodiments, the specific phobia is an animal phobia, natural environment phobia, blood-injection-injury phobia, or situational phobia. In some embodiments, after treating according to the disclosure, one or more symptoms of a phobia are reduced.

In some embodiments, a method of treating a post-traumatic stress disorder (PTSD) in a subject in need thereof is disclosed herein. As used herein, the term “post-traumatic stress disorder” refers to a condition developed after experiencing and/or witnessing a traumatic event or learning that a traumatic event has happened to a loved one. Non-limiting examples of traumatic events include exposure to war, rape or sexual violence, a physical attack, disease, a mugging, childhood physical or sexual violence, kidnapping or being taken hostage, terrorist attacks, torture, nature disasters and/or severe accidents. In some embodiments, the post-traumatic stress disorder is caused by trauma associated with a pandemic due to an infectious disease, such as, but not limited to COVID-19. For example, the trauma may be caused by a pandemic, such as COVID-19, a mandated quarantine, social isolation, damage to one's health, or damage to a family member's health as a result of the infectious disease. In some embodiments, trauma results from job loss. In some embodiments, trauma results from loss of housing. In some embodiments, trauma results from fear of not finding employment.

In some instances, a person shows symptoms of post-traumatic stress disorder within a week of experiencing of the traumatic event. In some instances, a person shows symptoms of post-traumatic stress disorder within a month of experiencing of the traumatic event. In some instances, a person shows symptoms of post-traumatic stress disorder within a year of experiencing of the traumatic event. In some instances, a person shows symptoms of post-traumatic stress disorder after a year or more of experiencing of the traumatic event. In some instances, post-traumatic stress disorder comprises a person re-experiencing the trauma event through intrusive distressing recollections of the event, flashbacks, and/or nightmares. In some instances, a symptom of post-traumatic stress disorder comprises emotional numbness and avoidance of places, people, and activities that are reminders of the trauma. In some instances, a symptom of post-traumatic stress disorder comprises increased arousal such as difficulty sleeping and concentrating, feeling anxious, and being easily irritated and angered.

In some embodiments, a method of treating moral injury in a subject in need thereof is disclosed herein. Moral injury may occur in individuals/populations with PTSD or at risk of developing PTSD (e.g. veterans or physicians). Moral injury may result from when an individual transgresses their own moral boundaries, often in high-stakes situations and where pressure is applied by higher authorities.

In some embodiments, a method of treating acute stress disorder in a subject in need thereof is disclosed herein. A subject may be determined to have acute stress disorder if the subject exhibits PTSD symptoms that have been present for about three days to about 1 month.

In some embodiments, a method of treating adjustment disorder in a subject in need thereof is disclosed herein. A subject may be determined to have adjustment disorder when the traumatic event experienced by a subject and/or the subject's symptoms do not meet the criteria required for a diagnosis of PTSD.

In some embodiments, a method of treating an obsessive-compulsive and related disorder in a subject in need thereof is disclosed herein. In some embodiments, the obsessive-compulsive and related disorder is obsessive compulsive disorder, body dysmorphic disorder, hoarding disorder, dermatillomania, trichotillomania, excoriation, substance-induced obsessive compulsive and related disorder, or an obsessive-compulsive disorder due to another medical condition, or a combination thereof.

In some embodiments, a method of treating obsessive-compulsive disorder (OCD) in a subject in need thereof is disclosed herein. An estimated 24% of adults meet the DSM criteria for OCD. There are mixed findings regarding gender differences, but OCD appears to be more common in males in childhood and in females in adolescence and adulthood. Up to 15% of those diagnosed with OCD have a history of suicide attempts. Pregnant and postpartum women have been found to exhibit higher prevalence of OCD than general populations. The prevalence of OCD in those over the age of 15 in Canada is estimated to be 1 in 100 people, this diagnosis was associated with adverse childhood experiences and likelihood of having lower income. Mortality rate of those with OCD is significantly higher even after adjusting for somatic comorbidities. Quality of life is significantly lower in those with OCD. OCD involves the presence of impulses or urges associated with anxiety (obsessions) and repetitive behaviors or mental acts that exist in response to obsessions (compulsions).

A subject is diagnosed with obsessive compulsive disorder if the obsessions and compulsions significantly impact daily life. In some embodiments, a subject with OCD does not realize that the obsessions and compulsions are excessive or unreasonable. As used herein, an “obsession” is an intrusive, repetitive, and/or persistent thought, urge, and/or image that causes distress. In some embodiments, obsessions cannot be suppressed or ignored. In some embodiments, a subject with OCD is unaware that these thoughts are not a real threat but are generated by their mind.

As used herein, a “compulsion” is excessive and repetitive ritualistic behavior that must be performed to prevent something bad from happening. In some embodiments, a compulsion is performed by a subject to reduce anxiety caused by obsessive thoughts. In some embodiments, a subject with OCD treated according to the methods described herein, performing one or more compulsions takes up at least about 15 minutes to about 20 hours per day, for example, about 15 minutes per day, about 30 minutes per day, about 45 minutes per day, about 1 hour per day, about 1.5 hours per day, about 2 hours per day, about 2.5 hours per day, about 3 hours per day, about 3.5 hours per day, about 4 hours per day, about 4.5 hours per day, about 5 hours per day, about 5.5 hours per day, about 6.0 hours per day, about 6.5 hours per day, about 7 hours per day, about 7.5 hours per day, about 8 hours per day, about 8.5 hours per day, about 9 hours per day, about 9.5 hours per day, about 10 hours per day, about 10.5 hours per day, about 11 hours per day, about 11.5 hours per day, about 12 hours per day, about 12.5 hours per day, about 13 hours, about 13.5 hours, about 14 hours, about 14.5 hours, about 15 hours, about 15.5 hours per day, about 16 hours per day, about 16.5 hours per day, about 17 hours per day, about 17.5 hours per day, about 18 hours per day, about 18.5 hours per day, about 19 hours per day, about 19.5 hours per day, or about 20 hours per day. In some embodiments, after treating according to the methods of the disclosure, the length of time a subject spends performing compulsions compared to prior to said treating decreases by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%.

In some embodiments, the obsessive compulsive and related disorder is trichotillomania. Trichotillomania is a disorder that involves recurrent, irresistible urges to pull out hair from your scalp, eyebrows, or other areas of your body. In some embodiments, a subject with trichotillomania repeatedly attempts to decrease or stop the recurrent hair pulling. In some embodiments, a subject with trichotillomania has hair loss that may cause bald spots. In some embodiments, a subject with trichotillomania ingests the pulled hair or parts thereof. In some embodiments, a subject with trichotillomania that ingests the pulled hair or parts thereof develops a trichobezoar or a hairball in the intestines or stomach. In some embodiments, after treating according to the methods of the disclosure, a subject's trichotillomania improves compared to prior to said treatment.

In some embodiments, the subject with an anxiety disorder has one or more comorbidities. Non-limiting examples of comorbidities include endocrine and/or metabolic diseases, gastrointestinal diseases, cardiovascular diseases, psychiatric disorders, or a neuroses. In some embodiments, the comorbidity is anxiety disorder, depressive disorder, psychotic disorder, developmental disorder, eating disorder, attention-deficit hyperactivity disorder (ADID), COVID-19, disorders associated with COVID-19, attention deficit disorder (ADD), autism spectrum disorder (ASD), tic disorder, bipolar disorder, conduct disorder, mental retardation, or a combination thereof. In some embodiments, the comorbidity is a mood disorder, major depressive disorder, bipolar disorder, schizophrenia, an eating disorder, attention deficit/hyperactivity disorder, epilepsy, cardiovascular disease, migraine, irritable bowel syndrome, dementia, Alzheimer's disease, Parkinson's disease, or combinations thereof. In some embodiments, the comorbidity is depression, generalized anxiety, agoraphobia, or panic disorder. In some embodiments, the comorbidity is major depression. In some embodiments, the comorbidity is an eating disorder. In some embodiments, the comorbidity is ADHD. In some embodiments, the comorbidity is ASD. In some embodiments, the comorbidity is COVID-19, disorders associated with COVID-19, or both. In some embodiments, the comorbidity is COVID-19. In some embodiments, the comorbidity is a disorder associated with COVID-19. Non-limiting examples of disorders associated with COVID-19 include post-traumatic stress disorder, anxiety disorder, acute stress disorder, adjustment disorder, or panic disorder. In some embodiments, the comorbidity is major depressive disorder, bipolar disorder, irritable bowel disease, or irritable bowel syndrome.

In some embodiments, no other treatment is administered to the subject to treat anxiety disorder after administration of the pharmaceutical composition/solid dispersion of the present disclosure.

In some embodiments, the subject with anxiety disorder is administered at least one additional therapy and/or therapeutic. In some embodiments, administration of an additional therapy and/or therapeutic is prior to administration of the pharmaceutical composition/solid dispersion of the present disclosure. In some embodiments, administration of an additional therapy and/or therapeutic is after administration of the pharmaceutical composition/solid dispersion of the present disclosure. In some embodiments, administration of an additional therapy and/or therapeutic is concurrent with administration of the pharmaceutical composition/solid dispersion of the present disclosure.

In some embodiments, the additional therapy is an antidepressant, an anticonvulsant, lisdexamfetamine dimesylate, an antipsychotic, an anxiolytic, an anti-inflammatory drug, a benzodiazepine, an analgesic drug, a cardiovascular drug, or combinations thereof.

In some embodiments, the additional therapy is a benzodiazepine. In some embodiments, the benzodiazepine is diazepam or alprazolam.

In some embodiments, the additional therapy is a N-methyl-D-aspartate (NMDA) receptor antagonist. In some embodiments, the NMDA receptor antagonist is ketamine.

In some embodiments, the additional therapy is an antidepressant. In some embodiments, an antidepressant indirectly affects a neurotransmitter receptor, e.g., via interactions affecting the reactivity of other molecules at a neurotransmitter receptor. In some embodiments, an antidepressant is an agonist. In one embodiment, an antidepressant is an antagonist. In some embodiments, an antidepressant acts (either directly or indirectly) at more than one type of neurotransmitter receptor. In some embodiments, an antidepressant is chosen from buproprion, citalopram, duloxetine, escitalopram, fluoxetine, fluvoxamine, milnacipran, mirtazapine, paroxetine, reboxetine, sertraline, and venlafaxine.

In some embodiments, the antidepressant is a tricyclic antidepressant (“TCA”), selective serotonin reuptake inhibitor (“SSRI”), serotonin and noradrenaline reuptake inhibitor (“SNRI”), dopamine reuptake inhibitor (“DRI”), noradrenaline reuptake Monoamine oxidase inhibitor (“MAOI”), including inhibitor (“NRU”), dopamine, serotonin and noradrenaline reuptake inhibitor (“DSNRI”), a reversible inhibitor of monoamine oxidase type A (RIMA), or combination thereof. In some embodiments, the antidepressant is a TCA. In some embodiments, the TCA is imipramine or clomipramine. In some embodiments, the antidepressant is an SRI. In some embodiments, the SSRI is escitalopram, paroxetine, sertraline, fluvoxamine, fluoxetine, or combinations thereof. In some embodiments, the SNRI is venlafaxine. In some embodiments, the additional therapy is pregabalin.

In some embodiments, the additional therapeutic is an anticonvulsant. In some embodiments, the anticonvulsant is gabapentin, carbamazepine, ethosuximide, lamotrigin, felbamate, topiramate, zonisamide, tiagabine, oxcarbazepine, levetiracetam, divalproex sodium, phenytoin, fosphenytoin. In some embodiments, the anticonvulsant is topiramate.

In some embodiments, the additional therapeutic is an antipsychotic. In some embodiments, the antipsychotic is a phenothiazine, butryophenone, thioxanthene, clozapine, risperidone, olanzapine, or sertindole, quetiapine, aripiprazole, zotepine, perospirone, a neurokinin-3 antagonist, such as osanetant and talnetant, rimonabant, or a combination thereof.

In some embodiments, the additional therapeutic is an anti-inflammatory drug. In some embodiments, the anti-inflammatory drug is a nonsteroidal anti-inflammatory drugs (NSAIDS), steroid, acetaminophen (COX-3 inhibitors), 5-lipoxygenase inhibitor, leukotriene receptor antagonist, leukotriene A4 hydrolase inhibitor, angiotensin converting enzyme antagonist, beta blocker, antihistaminic, histamine 2 receptor antagonist, phosphodiesterase-4 antagonist, cytokine antagonist, CD44 antagonist, antineoplastic agent, 3-hydroxy-3-methylglutaryl coenzyme A inhibitor (statins), estrogen, androgen, antiplatelet agent, antidepressant, Helicobacter pylori inhibitors, proton pump inhibitor, thiazolidinedione, dual-action compounds, or combination thereof.

In some embodiments, the additional therapeutic is an anti-anxiolytic. In one embodiment, an anxiolytic is chosen from alprazolam, an alpha blocker, an antihistamine, a barbiturate, a beta blocker, bromazepam, a carbamate, chlordiazepoxide, clonazepam, clorazepate, diazepam, flurazepam, lorazepam, an opioid, oxazepam, temazepam, or triazolam.

In some embodiments, the subject with anxiety disorder is administered at least one therapy. Non-limiting examples of therapies include transcranial magnetic stimulation, cognitive behavioral therapy, interpersonal psychotherapy, dialectical behavior therapy, mindfulness techniques, or acceptance, commitment therapy, or combinations thereof.

In some embodiments, at least one sign or symptom of an anxiety disorder is improved following the administration of the compound of Formula (I) in the form of a solid dispersion as disclosed herein. In some embodiments, a sign or symptom of an anxiety disorder is measured according to a diary assessment, an assessment by a clinician or caregiver, or a clinical scale. In some embodiments, treatment causes a demonstrated improvement in one or more of the following: State-Trait Anxiety Inventory (STAI), Beck Anxiety Inventory (BAI), Hospital Anxiety and Depression Scale (HADS), Generalized Anxiety Disorder questionnaire-IV (GADQ-IV), Hamilton Anxiety Rating Scale (HARS), Leibowitz Social Anxiety Scale (LSAS), Overall Anxiety Severity and Impairment Scale (OASIS), Hospital Anxiety and Depression Scale (HADS), Patient Health Questionnaire 4 (PHQ-4), Social Phobia Inventory (SPIN), Brief Trauma Questionnaire (BTQ), Combat Exposure Scale (CES), Mississippi Scale for Combat-Related PTSD (M-PTSD), Posttraumatic Maladaptive Beliefs Scale (PMBS), Perceived Threat Scale (DRRI-2 Section: G), PTSD Symptom Scale-Interview for DSM-5 (PSS-I-5), Structured Interview for PTSD (SI-PTSD), Davidson Trauma Scale (DTS), Impact of Event Scale-Revised (IES-R), Posttraumatic Diagnostic Scale (PDS-5), Potential Stressful Events Interview (PSEI), Stressful Life Events Screening Questionnaire (SLESQ), Spielberger's Trait and Anxiety, Generalized Anxiety Disorder 7-Item Scale, The Psychiatric Institute Trichotillomania Scale (PITS), The MGH Hairpulling Scale (MGH-HPS), The NIMH Trichotillomania Severity Scale (NIMH-TSS), The NIMH Trichotillomania Impairment Scale (NIMH-TIS), The Clinical Global Impression (CGI), the Brief Social Phobia Scale (BSPS), The Panic Attack Questionnaire (PAQ), Panic Disorder Severity Scale, Florida Obsessive-Compulsive Inventory (FOCI), The Leyton Obsessional Inventory Survey Form, The Vancouver Obsessional Compulsive Inventory (VOCI), The Schedule of Compulsions, Obsessions, and Pathological Impulses (SCOPI), Padua Inventory-Revised (PI—R), Quality of Life (QoL), The Clinical Global Improvement (CGI) scale, The Yale-Brown Obsessive-Compulsive Scale (Y—BOCS), The Yale-Brown Obsessive-Compulsive Scale Second Edition (Y-BOCS-II), The Dimensional Yale-Brown Obsessive-Compulsive Scale (DY-BOCS), The National Institute of Mental Health-Global Obsessive-Compulsive Scale (NIMH-GOCS), The Yale-Brown Obsessive-Compulsive Scale Self-Report (Y—BOCS—SR), The Obsessive-Compulsive Inventory-Revised (OCI-R), and the Dimensional Obsessive-Compulsive Scale (DOCS), or a combination thereof.

In some embodiments, after treating with the compound of Formula (I) in the form of a solid dispersion as disclosed herein, a sign or symptom of OCD is improved as demonstrated on one of the following scales: Florida Obsessive-Compulsive Inventory (FOCI), The Leyton Obsessional Inventory Survey Form, The Vancouver Obsessional Compulsive Inventory (VOCI), Schedule of Compulsions, Obsessions, and Pathological Impulses (SCOPI), Padua Inventory-Revised (PI—R), Quality of Life (QoL), The Clinical Global Improvement (CGI) scale, The Yale-Brown Obsessive-Compulsive Scale (Y—BOCS), The Yale-Brown Obsessive-Compulsive Scale Second Edition (Y-BOCS-II), The Dimensional Yale-Brown Obsessive-Compulsive Scale (DY-BOCS), The National Institute of Mental Health-Global Obsessive-Compulsive Scale (NIMH-GOCS), The Yale-Brown Obsessive-Compulsive Scale Self-Report (Y—BOCS—SR), The Obsessive-Compulsive Inventory-Revised (OCI-R), and The Dimensional Obsessive-Compulsive Scale (DOCS).

In some embodiments, after treating with the compound of Formula (I) in the form of a solid dispersion as disclosed herein, a sign or symptom of trichotillomania is improved as demonstrated on one of the following scales: the Psychiatric Institute Trichotillomania Scale (PITS), the Massachusetts General Hospital (MGH) Hairpulling Scale (MGH-HPS), the National Institute for Mental Health (NIMH) Trichotillomania Severity Scale (NIMH-TSS), the NIMH Trichotillomania Impairment Scale (NIMH-TIS).

In some embodiments, treating according to the methods of the disclosure results in an improvement in an anxiety disorder compared to pre-treatment of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein.

In some embodiments, treating according to the methods of the disclosure results in an improvement in an obsessive compulsive and related disorder compared to pre-treatment of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein.

In some embodiments, treating according to the methods of the disclosure results in an improvement in post-traumatic stress disorder compared to pre-treatment of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein.

In some embodiments, after administration of the compound of Formula (I) in the form of a solid dispersion as disclosed herein, a subject has improved cognition, information processing, stress levels, immune system, or combinations thereof.

In some embodiments, after administration of the compound of Formula (I) in the form of a solid dispersion as disclosed herein, at least one symptom of anxiety disorder is alleviated for a period of at least about 5 minutes to at least about 1 year, for example, at least one symptom of anxiety is alleviated for a period of at least about 5 minutes, at least about 15 minutes, at least about 30 minutes, at least about 45 minutes, at least about 1 hour, at least about 2 hours, at least 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, at least about 2 months, at least about 3 months, at least about 4 months, at least about 5 months, at least about 6 months, at least about 7 months, at least about 8 months, at least about 9 months, at least about 10 months, at least about 11 months, at least about 12 months, or more. In some embodiments, at least one symptom of anxiety is alleviated for a period of at least 1 month after administration of the compound of Formula (I) in the form of a solid dispersion as disclosed herein. In some embodiments, at least one symptom of anxiety is alleviated for a period of at least 3 months after administration of the compound of Formula (I) in the form of a solid dispersion as disclosed herein. In some embodiments, at least one symptom of anxiety is alleviated for a period of at least 1 year after administration of the compound of Formula (I) in the form of a solid dispersion as disclosed herein.

In some embodiments, after administration of the compound of Formula (I) in the form of a solid dispersion as disclosed herein, at least one symptom of anxiety disorder is alleviated within about 5 minutes to about 1 week, for example, about 5 minutes, about 15 minutes, about 30 minutes, about 45 minutes, about 1 hour, about 2 hours, at least 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 1 day, about 2 days, about 3 days, about 4 days, about 5 days, about 6 days, or about 7 days. In some embodiments, at least one symptom of anxiety is alleviated within 24 hours of administration of the compound of Formula (I) in the form of a solid dispersion as disclosed herein. In some embodiments, at least one symptom of anxiety is alleviated within 1 week of administration of the compound of Formula (I) in the form of a solid dispersion as disclosed herein.

### Eating Disorders

In some embodiments, a method of treating one or more eating disorders in a subject in need thereof is disclosed herein.

As used herein, the term “eating disorder” refers to any of a range of psychological disorders characterized by abnormal or disturbed eating habits. In some embodiments, the eating disorder is pica, anorexia nervosa, bulimia nervosa, rumination disorder, avoidant/restrictive food intake disorder, binge-eating disorder, or combinations thereof. In some embodiments, the methods disclosed herein treat chronic eating disorders. As used herein, a “chronic” eating disorder is recurring.

In some embodiments, an eating disorder comprises a medical diagnosis based on the criteria and classification from the Diagnostic and Statistical Manual of Medical Disorders, 5th Ed. In one embodiment, an eating disorder comprises a medical diagnosis based on an independent medical evaluation.

In some embodiments, a method of treating pica in a subject in need thereof is disclosed herein. Pica is a disorder characterized by an appetite for substances that are largely non-nutritive. Individuals with the disorder pica compulsively eat non-nutritive items. Substances may include ice, hair, cigarette ashes, glue, paper, drywall, paint, sharp objects, metal, stone, soil, glass, feces, chalk, dirt, or dust. In some embodiments, an individual with pica also has an intellectual disability, schizophrenia, or OCD. In some embodiments, an individual with pica has a nutritional deficiency. In some embodiments, the nutritional deficiency, is low levels of zinc or iron.

In some embodiment, a subject with pica has experienced the disorder for at least 1 month, for example 1 month, 2 months, 3 months, 4 months, 5 months, 6 months, 7 months, 8 months, 9 months, 10 months, 11 months, 12 months, 1 years, 2 years, 3 years, 4 years, 5 years, 6 years, 7 years, 8 years, 9 years, 10 years, or more.

In some embodiments, after treating according to the methods of the disclosure, an individual with pica stops eating non-nutritive items for at least 1 day, at least 2 days, at least 3 days, at least 4 days, at least 5 days, at least 6 days, at least 7 days, at least 2 weeks, at least 3 weeks, at least 4 weeks, at least 5 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years, or more. In some embodiments, after treating according to the methods of the disclosure, an individual with pica stops eating non-nutritive items within about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 1 day, about 2 days, about 3 days, about 4 days, about 5 days, about 6 days, or about 7 days.

In some embodiments, a method of treating anorexia nervosa (AN) in a subject in need thereof is disclosed herein. AN is a disorder characterized by intense fear of gaining weight, abnormally low body weight, and a distorted perception of weight. Individuals with AN often severely restrict the amount of food they eat. They may control calorie intake by vomiting after eating or by misusing laxatives, diet aids, diuretics, or enemas. They may also try to lose weight by exercising excessively. In some embodiments, a subject with AN has signs and symptoms, including but not limited to, extreme weight loss or not making expected developmental weight gains, thin appearance, abnormal blood counts, fatigue, insomnia, dizziness or fainting, bluish discoloration of the fingers, thin hair that breaks or falls out, soft downy hair covering the body, absence of menstruation, constipation and abdominal pain, dry or yellowish skin, intolerance of cold, irregular heart rhythms, low blood pressure, dehydration, swelling of arms or legs, or eroded teeth and calluses on the knuckles from induced vomiting.

In some embodiments, after treating according to the methods of the disclosure, one or more symptoms of AN improve for at least 1 day, at least 2 days, at least 3 days, at least 4 days, at least 5 days, at least 6 days, at least 7 days, at least 2 weeks, at least 3 weeks, at least 4 weeks, at least 5 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years, or more.

In some embodiments, after treating according to the methods of the disclosure, one or more symptoms of AN improve within about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 1 day, about 2 days, about 3 days, about 4 days, about 5 days, about 6 days, or about 7 days.

In some embodiments, a method of treating bulimia nervosa (BN) in a subject in need thereof is disclosed herein. BN is a disorder characterized by distortion of body image and an obsessive desire to lose weight in which bouts of extreme overeating are followed by depression and self-induced vomiting, purging, use of laxatives, diuretics, or fasting. In some embodiments, the binge eating and inappropriate compensatory behaviors both occur on average at least once a week for three months. Signs and symptoms of BN include, but are not limited to, behaviors and attitudes that indicate that weight loss, dieting, and control of food are becoming primary concerns; evidence of binge eating, including disappearance of large amounts of food in short periods of time or lots of empty wrappers and containers indicating consumption of large amounts of food; evidence of purging behaviors, including frequent trips to the bathroom after meals, signs and/or smells of vomiting, presence of wrappers or packages of laxatives or diuretics; appears uncomfortable eating around others; develops food rituals (e.g. eats only a particular food or food group (e.g. condiments), excessive chewing, doesn't allow foods to touch); skips meals or takes small portions of food at regular meals; disappears after eating, often to the bathroom; any new practice with food or fad diets, including cutting out entire food groups (no sugar, no carbs, no dairy, vegetarianism/veganism); fear of eating in public or with others; steals or hoards food in strange places; drinks excessive amounts of water or non-caloric beverages; uses excessive amounts of mouthwash, mints, and gum; hides body with baggy clothes; maintains excessive, rigid exercise regimen despite weather, fatigue, illness, or injury due to the need to “burn off” calories; shows unusual swelling of the cheeks or jaw area; has calluses on the back of the hands and knuckles from self-induced vomiting; teeth are discolored, stained; creates lifestyle schedules or rituals to make time for binge-and-purge sessions; withdraws from usual friends and activities; looks bloated from fluid retention; frequently diets; shows extreme concern with body weight and shape; frequent checking in the mirror for perceived flaws in appearance; has secret recurring episodes of binge eating (eating in a discrete period of time an amount of food that is much larger than most individuals would eat under similar circumstances); feels lack of control over ability to stop eating; purges after a binge (e.g. self-induced vomiting, abuse of laxatives, diet pills and/or diuretics, excessive exercise, fasting); extreme mood swings; self-injury (cutting and other forms of self-harm without suicidal intention), substance abuse, impulsivity, diabulimia, noticeable fluctuations in weight, both up and down; body weight is typically within the normal weight range; may be overweight; stomach cramps, other non-specific gastrointestinal complaints (constipation, acid reflux, etc.); difficulties concentrating; abnormal laboratory findings (anemia, low thyroid and hormone levels, low potassium, low blood cell counts, slow heart rate); dizziness; fainting/syncope; feeling cold all the time; sleep problems; cuts and calluses across the top of finger joints (a result of inducing vomiting); dental problems, such as enamel erosion, cavities, and tooth sensitivity; dry skin; dry and brittle nails; swelling around area of salivary glands; fine hair on body; thinning of hair on head, dry and brittle hair (lanugo); cavities, or discoloration of teeth, from vomiting; muscle weakness; yellow skin (in context of eating large amounts of carrots); cold, mottled hands and feet or swelling of feet; menstrual irregularities—missing periods or only having a period while on hormonal contraceptives (this is not considered a “true” period); poor wound healing; or impaired immune functioning.

In some embodiments, after treating according to the methods of the disclosure, one or more symptoms of BN improve for at least 1 day, at least 2 days, at least 3 days, at least 4 days, at least 5 days, at least 6 days, at least 7 days, at least 2 weeks, at least 3 weeks, at least 4 weeks, at least 5 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years, or more.

In some embodiments, after treating according to the methods of the disclosure, one or more symptoms of BN improve within about 1 hour, about 2 hours, about 3 hours, about 4 hours, about 5 hours, about 6 hours about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 1 day, about 2 days, about 3 days, about 4 days, about 5 days, about 6 days, or about 7 days.

In some embodiments, a method of treating binge-eating disorder (BED) in a subject in need thereof is disclosed herein. Binge-eating disorder (BED) is the most common eating disorder and is characterized by the uncontrolled consumption of large volumes of food over a short time-period, causing psychological distress. To meet diagnosis, these episodes must be recurrent, occur on average at least once a week for three months, and involve three of the following: consuming food faster than normal; consuming food until uncomfortably full; consuming large amounts of food when not hungry; consuming food alone due to embarrassment; feeling disgusted, depressed, or guilty after eating a large amount of food. Compensatory behavior associated with bulimia nervosa should not be involved, and binging should not occur during an episode of bulimia nervosa or anorexia nervosa.

Many factors have been associated with the cause of BED. High levels of depressive symptoms, body dissatisfaction, poor family cohesion, and low self-esteem are significant predictors of binge eating behaviors. The global pooled prevalence of BED is estimated as 0.9%. The prevalence is higher in women (1.4%) than in men (0.4%). An online survey of 22,397 adults with BED in the US found that only 3.2% reported receiving support from a healthcare provider. The WHO mental health survey found lifetime prevalence estimates of BED to be 1.4% with median age of onset in the late teens to early 20's. No ethnic differences have been found in the prevalence of eating disorders or differences in the relation of risk factors for future ED onset. BED is more common in obese populations, as 20-50% of those seeking weight control treatment report binge-eating episodes. However, only 1-2% of this population meet the full DSM criteria for BED.

In some embodiments, treating according to the methods of the disclosure improves one or more symptom of BED. Symptoms of BED include eating unusually large amounts of food in a specific amount of time, feeling that eating behavior is out of control, eating when full or not hungry, eating rapidly during binge episodes, eating until uncomfortably full, eating alone or in secret, feeling depressed, disgusted, ashamed, guilty, or upset about your eating, or frequently dieting, possibly without weight loss.

In some embodiments, after treating according to the methods of the disclosure, one or more symptoms of BED improve for at least 1 day, at least 2 days, at least 3 days, at least 4 days, at least 5 days, at least 6 days, at least 7 days, at least 2 weeks, at least 3 weeks, at least 4 weeks, at least 5 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years, or more.

In some embodiments, after treating according to the methods of the disclosure, one or more symptoms of BED improve within about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 1 day, about 2 days, about 3 days, about 4 days, about 5 days, about 6 days, or about 7 days.

In some embodiments, a method of treating rumination disorder in a subject in need thereof is disclosed herein. Rumination disorder is an eating disorder in which an individual regurgitates food that has been swallowed. In some embodiments, a subject with rumination disorder swallows again the regurgitated food. In some embodiments, a subject with rumination disorder spits out the regurgitated food. In some embodiments, a subject with rumination disorder chews the regurgitated food. In some embodiments, a subject with rumination disorder chews and swallows the regurgitated food. In some embodiments, rumination disorder may cause unhealthy weight loss, malnutrition, dental erosion, bad breath, embarrassment, and/or social isolation.

In some embodiments, after treating according to the methods of the disclosure, the frequency of rumination decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In some embodiments, signs and symptoms of rumination disorder improve for at least 1 day, at least 2 days, at least 3 days, at least 4 days, at least 5 days, at least 6 days, at least 7 days, at least 2 weeks, at least 3 weeks, at least 4 weeks, at least 5 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years, or more.

In some embodiments, after treating according to the methods of the disclosure, signs and symptoms of rumination disorder improve within about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 1 day, about 2 days, about 3 days, about 4 days, about 5 days, about 6 days, or about 7 days.

In some embodiments, a method of treating avoidant/restrictive food intake disorder (ARFID) in a subject in need thereof is disclosed herein. ARFID is an eating or feeding disturbance characterized by one or more of apparent lack of interest in eating or food; avoidance based on the sensory characteristics of the food; and concern about aversive consequences of eating). ARFID manifests as persistent failure to meet appropriate nutritional and/or energy needs, nutritional deficiency, dependence on enteral feeding or oral nutritional supplements, or marked interference with psychosocial functioning. In some embodiments, subjects with ARFID have ASD, ADH, or intellectual disabilities.

In some embodiments, after treating according to the methods of the disclosure, one or more signs, symptoms, or consequences of ARFID improve. In some embodiments, the improvement is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the subject with an eating disorder has one or more comorbidities. Non-limiting examples of comorbidities include endocrine and/or metabolic diseases, gastrointestinal diseases, cardiovascular diseases, psychiatric disorders, or a neuroses. In some embodiments, the comorbidity is anxiety disorder, depressive disorder, psychotic disorder, developmental disorder, eating disorder, attention-deficit hyperactivity disorder (ADHD), attention deficit disorder (ADD), autism spectrum disorder (ASD), tic disorder, bipolar disorder, conduct disorder, mental retardation, or a combination thereof. In some embodiments, the comorbidity is a mood disorder, major depressive disorder, bipolar disorder, schizophrenia, an eating disorder, attention deficit/hyperactivity disorder, epilepsy, cardiovascular disease, migraine, irritable bowel syndrome, dementia, Alzheimer's disease, Parkinson's disease, or combinations thereof. In some embodiments, the comorbidity is depression, generalized anxiety, agoraphobia, or panic disorder. In some embodiments, the comorbidity is an anxiety disorder, a mood disorder, an impulse control disorder, a substance use disorder, or any disorder. In some embodiments, the comorbidity is hypertension, dyslipidemias, sleep problems/disorders, pain, functional gastrointestinal symptoms/disorders, menstrual dysfunction, pregnancy complications, intracranial hypertension, polycystic ovary syndrome, or combinations thereof. The following data from the National Comorbidity Survey—Replication (NCS—R) present the lifetime comorbidity of eating disorders with other core disorders:
anorexia nervosa (%): any anxiety disorder (47.9), any mood disorder (42.1), any impulse control disorder (30.8), any substance use disorder (27.0), any disorder (56.2);
bulimia nervosa (%): any anxiety disorder (80.6), any mood disorder (70.7), any impulse control disorder (63.8), any substance use disorder (36.8), any disorder (94.5);
binge-eating disorder (%): any anxiety disorder (65.1), any mood disorder (46.4), any impulse control disorder (43.3), any substance use disorder (23.3), any disorder (78.9).

In some embodiments, the comorbidity is another eating disorder. In some embodiments, the subject in need has at least two eating disorders. In some embodiments, the subject in need has at least three eating disorders. In some embodiments, the subject in need has at least four eating disorders.

In some embodiments, the comorbidity is a psychiatric disorder selected from schizophrenia, schizoaffective disorder, bipolar disorder, major depressive disorder, anxiety disorder, obsessive compulsive disorder, post-traumatic stress disorder, attention deficit hyperactivity disorder, autism, alcohol use disorder, drug use disorder, or suicide attempt.

In some embodiments, the comorbidity is major depression. In some embodiments, the comorbidity is an eating disorder. In some embodiments, the comorbidity is obesity, one or more conditions related to obesity, or both. In some embodiments, the one or more conditions related to obesity are one or more of metabolic syndrome, diabetes, hypertension, dyslipidaemias, sleep problems or disorders, or pain. In some embodiments, the comorbidity is ADHD. In some embodiments, the comorbidity is ASD. In some embodiments, the comorbidity is major depressive disorder, bipolar disorder, irritable bowel disease, or irritable bowel syndrome. In some embodiments, the one or more comorbidities is reproductive dysfunction, polycystic ovary syndrome, or menstrual dysfunction. In some embodiments, the comorbidity is a psychiatric disorder selected from schizophrenia, schizoaffective disorder, bipolar disorder, major depressive disorder, anxiety disorder, obsessive compulsive disorder, post-traumatic stress disorder, attention deficit hyperactivity disorder, autism, alcohol use disorder, drug use disorder, or suicide attempt.

In some embodiments, no other treatment is administered to the subject to treat an eating disorder after administration of the pharmaceutical composition/solid dispersion of the present disclosure.

In some embodiments, the subject with an eating disorder is administered at least one additional therapy and/or therapeutic. In some embodiments, administration of an additional therapy and/or therapeutic is prior to administration of the pharmaceutical composition/solid dispersion of the present disclosure. In some embodiments, administration of an additional therapy and/or therapeutic is after administration of the pharmaceutical composition/solid dispersion of the present disclosure. In some embodiments, administration of an additional therapy and/or therapeutic is concurrent with administration of the pharmaceutical composition/solid dispersion of the present disclosure.

In some embodiments, the additional therapy is an antidepressant, an anticonvulsant, lisdexamfetamine dimesylate, an antipsychotic, an anxiolytic, an anti-inflammatory drug, a benzodiazepine, an analgesic drug, a cardiovascular drug, an opioid antagonist, or combinations thereof.

In some embodiments, the additional therapy is a benzodiazepine. In some embodiments, the benzodiazepine is diazepam or alprazolam.

In some embodiments, the additional therapy is a N-methyl-D-aspartate (NMDA) receptor antagonist. In some embodiments, the NMDA receptor antagonist is ketamine.

In some embodiments, the additional therapy is an antidepressant. In some embodiments, an antidepressant indirectly affects a neurotransmitter receptor, e.g., via interactions affecting the reactivity of other molecules at a neurotransmitter receptor. In some embodiments, an antidepressant is an agonist. In one embodiment, an antidepressant is an antagonist. In some embodiments, an antidepressant acts (either directly or indirectly) at more than one type of neurotransmitter receptor. In some embodiments, an antidepressant is chosen from buproprion, citalopram, duloxetine, escitalopram, fluoxetine, fluvoxamine, milnacipran, mirtazapine, paroxetine, reboxetine, sertraline, and venlafaxine.

In some embodiments, the antidepressant is a tricyclic antidepressant (“TCA”), selective serotonin reuptake inhibitor (“SSRI”), serotonin and noradrenaline reuptake inhibitor (“SNRI”), dopamine reuptake inhibitor (“DRI”), noradrenaline reuptake Monoamine oxidase inhibitor (“MAOI”), including inhibitor (“NRU”), dopamine, serotonin and noradrenaline reuptake inhibitor (“DSNRI”), a reversible inhibitor of monoamine oxidase type A (RIMA), or combination thereof. In some embodiments, the antidepressant is a TCA. In some embodiments, the TCA is imipramine or clomipramine. In some embodiments, the antidepressant is an SRI. In some embodiments, the SSRI is escitalopram, paroxetine, sertraline, fluvoxamine, fluoxetine, or combinations thereof. In some embodiments, the SNRI is venlafaxine. In some embodiments, the additional therapy is pregabalin.

In some embodiments, the additional therapy is an anticonvulsant. In some embodiments, the anticonvulsant is gabapentin, carbamazepine, ethosuximide, lamotrigin, felbamate, topiramate, zonisamide, tiagabine, oxcarbazepine, levetiracetam, divalproex sodium, phenytoin, fosphenytoin. In some embodiments, the anticonvulsant is topiramate.

In some embodiments, the additional therapy is an antipsychotic. In some embodiments, the antipsychotic is a phenothiazine, butryophenone, thioxanthene, clozapine, risperidone, olanzapine, or sertindole, quetiapine, aripiprazole, zotepine, perospirone, a neurokinin-3 antagonist, such as osanetant and talnetant, rimonabant, or a combination thereof.

In some embodiments, the additional therapy is an anti-inflammatory drug. In some embodiments, the anti-inflammatory drug is a nonsteroidal anti-inflammatory drugs (NSAIDS), steroid, acetaminophen (cyclooxygenase-3 (COX-3) inhibitors), 5-lipoxygenase inhibitor, leukotriene receptor antagonist, leukotriene A4 hydrolase inhibitor, angiotensin converting enzyme antagonist, beta blocker, antihistaminic, histamine 2 receptor antagonist, phosphodiesterase-4 antagonist, cytokine antagonist, CD44 antagonist, antineoplastic agent, 3-hydroxy-3-methylglutaryl coenzyme A inhibitor (statins), estrogen, androgen, antiplatelet agent, antidepressant, Helicobacter pylori inhibitors, proton pump inhibitor, thiazolidinedione, dual action compounds, or combination thereof.

In some embodiments, the additional therapy is an anti-anxiolytic. In one embodiment, an anxiolytic is chosen from alprazolam, an alpha blocker, an antihistamine, a barbiturate, a beta blocker, bromazepam, a carbamate, chlordiazepoxide, clonazepam, clorazepate, diazepam, flurazepam, lorazepam, an opioid, oxazepam, temazepam, or triazolam.

In some embodiments, the additional therapy is an opioid antagonist. Non-limiting examples of opioid antagonists include naloxone, naltrexone, nalmefene, nalorphine, nalrphine dinicotinate, levallrphan, samidorphan, nalodeine, alvimopan, methylnaltrexone, naloxegol, 6-naltrexol, axelopran, bevenopran, methylsamidorphan, naldemedine, buprenorphine, decozine, butorphanol, levorphanol, nalbuphine, pentazocine, and phenazocine.

In some embodiments, the additional therapy is a cardiovascular drug. Non-limiting examples of cardiovascular drugs include digoxin or (3β,5β,12β)-3-[(O-2,6-dideoxy-β-D-ribo-hexopyranosyl-(1→4)-O-2,6-dideoxy-β-D-ribo-hexopyranosyl-(1→4)-2,6-dideoxy-β-D-ribohexopyranosyl)oxy]-12,14-dihydroxy-card-20(22)-enolide, lisinopril, captopril, ramipril, trandolapril, benazepril, cilazapril, enalapril, moexipril, perindopril, quinapril, fludrocortisone, enalaprilate, quinapril, perindopril, apixaban, dabigatran, edoxaban, heparin, rivaroxaban, warfarin, aspirin, clopidogrel, dipyridamole, prasugrel, ticagrelor, azilsartan, candesartan, eprosartan, irbesartan, losartan, olmesartan, telmisartan, valsartanscaubitril, acebutolol, atenolol, betaxolol, bisoprolol, metoprolol, nadolol, propranolol, sotalol, amlodipine, diltiazem, felodipine, nifedipine, nimodipine, nisolidipine, verapamil, statins, nicotinic acids, diuretics, vasodilators, and combinations thereof.

In some embodiments, the subject with an eating disorder is administered at least one therapy. Non-limiting examples of therapies include transcranial magnetic stimulation, cognitive behavioral therapy, interpersonal psychotherapy, dialectical behavior therapy, weight loss therapy, mindfulness techniques, acceptance and commitment therapy, and combinations thereof.

In some embodiments, at least one sign or symptom of an eating disorder is improved following administration of the compound of Formula (I) in the form of a solid dispersion as disclosed herein. In some embodiments, a sign or symptom of an eating disorder is measured according to a diary assessment, an assessment by a clinician or caregiver, or a clinical scale. Non-limiting examples of clinical scales, diary assessments, and assessments by a clinician or caregiver include: the Mini International Neuropsychiatric Interview (MINI), the McLean Screening Instrument for Borderline Personality Disorder (MSI-BPD), the Eating Disorder Examination (EDE), the Eating Disorder Questionnaire (EDE-Q), the Eating Disorder Examination Questionnaire Short Form (EDE-QS), the Physical Appearance State and Trait Anxiety Scale-State and Trait version (PASTAS), Spielberger State-Trait Anxiety Inventory (STAI), Eating Disorder Readiness Ruler (ED-RR), Visual Analogue Rating Scales (VAS), the Montgomery-Asberg Depression Rating Scale (MADRS), Yale-Brown Cornell Eating Disorder Scale (YBC-EDS), Yale-Brown Cornell Eating Disorder Scale Self Report (YBC-EDS-SRQ), the Body Image State Scale (BISS), Clinical impairment assessment (CIA) questionnaire, the Eating Disorder Inventory (EDI) (e.g. version 3: EDI-3), the Five Dimension Altered States of Consciousness Questionnaire (5D-ASC), the Columbia-Suicide Severity Rating Scale (C—SSRS), the Life Changes Inventory (LCI), and combinations thereof.

In some embodiments, the Mini International Neuropsychiatric Interview (MINI) (for example, version 7.0.2) is a diagnostic interview instrument for psychiatric disorders in the Diagnostic and Statistical Manual of Mental Disorders (DSM-5) and the International Classification of Diseases-10. In some embodiments, the McLean Screening Instrument for Borderline Personality Disorder (MSI-BPD) is utilized to diagnose borderline personality disorder (BPD). The MSI-BPD contains 10 items based on the DSM-5 BPD criteria; the first 8 items represent the first eight criteria in the DSM-5 for BPD diagnosis, while the last two questions assess the paranoia and dissociation criteria for BPD. Scores for the MSI-BPD range from 0 to 10, with each item rated as “1” if present and “0” if absent. A score of 7 or higher indicates a likelihood for a subject to meet criteria for BPD. In some embodiments, after treating according to the methods of the disclosure, a subject's MSI-BPD score decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, one or more of the Eating Disorder Examination (EDE), the Eating Disorder Questionnaire (EDE-Q), or the Eating Disorder Examination Questionnaire Short Form (EDE-QS) are utilized to assess the range and severity of eating disorders. The EDE is a structured clinical interview (investigator rated), whereas the EDE-Q and EDE-QS are self-report questionnaires. The EDE is a standardized interview used to measure the severity of the characteristic psychopathology of eating disorders. In some embodiments, after treatment according to the methods of the disclosure, a subject's EDE score decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

The EDE-Q is a self-reported interview, which assesses a subject's eating disorder severity over 28 days. In some embodiments, after treatment according to the methods of the disclosure, a subject's EDE-Q score decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

The EDE-QS is a self-reported interview of eating disorder severity, which assesses the severity of a subject's eating disorder symptoms over seven days. The EDE-QS assesses the frequency of different forms of problematic overeating behaviors, including binge eating (labeled objective bulimic episodes (OBEs) and different forms of inappropriate weight compensatory behaviors (e.g., purging methods). In some embodiments, after treatment according to the methods of the disclosure, a subject's EDE-QS score decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the Physical Appearance State and Trait Anxiety Scale-State and Trait version (PASTAS) is utilized to evaluate the safety and efficacy of the compound of Formula (I) administered as a solid dispersion for the treatment of eating disorders. The PASTAS is a self-reported 16-item measure that assesses anxiety about physical appearance. Subjects rate how often they experience anxiety about sites of their body on a Likert scale from 0 to 4. A score of 0 is “never,” and a score of 4 is “almost always.” In some embodiments, PASTAS is used to evaluate anxiety that is associated with eating disorders. In some embodiments, after treatment according to the methods of the disclosure, a subject's PASTAS score decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the Spielberger State-Trait Anxiety Inventory (STAI) is utilized to evaluate the safety and efficacy of the compound of Formula (I) administered as a solid dispersion for the treatment of eating disorders. The STAI is a widely used instrument that contains separate self-report scales for measuring “state” and “trait” anxiety. “State” anxiety is transient anxiety due to a stressful stimulus, whereas “trait” anxiety is the predisposition of a subject to react with anxiety to stressful situations. is the STAI is self-reported and contains 40 items scored by a 4 point Likert scale with semantic guides taking approximately 10 minutes. The STAI contains 20 questions related to state anxiety and 20 questions related to trait anxiety. Each section is scored between 20 and 80.

In some embodiments, the Eating Disorder Readiness Ruler (ED-RR) is utilized to evaluate the safety and efficacy of the compound of Formula (I) administered as a solid dispersion for the treatment of eating disorders. The ED-RR is a self-reported, two-part 18-item questionnaire that examines readiness to change in nine dimensions of eating disorder behavior (restriction, weight-shape over evaluation, binge eating, vomiting, laxative use, fasting, diuretic use, weight-gain phobia, exercise). The first part of the ED-RR the subjective readiness of a subject to change on a Likert scale from 1-10. The second part of the ED-RR measures whether the motivation to change is for others or the self. The second part of the ED-RR is scored on a scale from 0% to 100%, where a score closer to 0% indicates that the motivation to change is for others, and a score closer to 100% indicates that the motivation to change is for self. Higher readiness to change has shown a correlation with a decrease in eating disorder symptoms over time. In some embodiments, after treating according to the methods of the disclosure, a subject's readiness to change improves by about 5%, about 10%, about 15%, about 20%, about 25%, about 30%, about 35%, about 40%, about 45%, about 50%, about 55%, about 60%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, or more, compared to prior to said treatment. In some embodiments, a subject's ED-RR score increases by about 1 point, about 2 points, about 3 points, about 4 points, about 5 points, about 6 points, about 7 points, about 8 points, about 9 points, or about 10 points after treating according to the methods of the disclosure.

In some embodiments, the Visual Analogue Rating Scales (VAS) are utilized to evaluate the safety and efficacy of the compound of Formula (I) administered as a solid dispersion for the treatment of eating disorders. The VAS is self-reported ratings of hunger, fullness and desire to eat. Subjects mark the degree of their experience in relation to the anchors along a 100 mm continuum. In some embodiments, after treating according to the methods of the disclosure a subject's VAS score improves by about 5%, about 10%, about 15%, about 20%, about 25%, about 30%, about 35%, about 40%, about 45%, about 50%, about 55%, about 60%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, or more compared to prior to said treating.

In some embodiments, the Montgomery-Asberg Depression Rating Scale (MADRS) is utilized to evaluate the safety and efficacy of the compound of Formula (I) administered as a solid dispersion for the treatment of eating disorders. The MADRS is a clinician rated scale that measures depression severity, which contains 10 items, each scored from 0 (normal) to 6 (severe), for a total possible score of 60. A higher score denotes greater severity of depression. In some embodiments, after treating according to the methods of the disclosure, the subject's MADRS score decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the Yale-Brown Cornell Eating Disorder Scale (YBC-EDS) and/or Yale-Brown Cornell Eating Disorder Scale Self Report (YBC-EDS-SRQ) are utilized to assess the severity and type of symptoms observed to be present in subject with eating disorders. The YBC-EDS is an interview administered by a physician, whereas the YBC-EDS-SRQ is a self-reported questionnaire. The YBC-EDS-SRQ assesses the current severity of a subject's eating disorder and the worst severity of a subject's eating disorder.

In some embodiments, the Body Image State Scale (BISS) is utilized to evaluate the safety and efficacy of the compound of Formula (I) administered as a solid dispersion for the treatment of eating disorders. The BISS is a 6 item self-reported scale that measures a subject's affective/evaluative body image states. The BISS is used to investigate the momentary affective experience of body image in response to a variety of hypothetical situations. The six items are: 1) dissatisfaction with physical appearance, 2) dissatisfaction with body size and shape, 3) dissatisfaction with weight, 4) feelings of physical unattractiveness, 5) current feeling about one's look relative to how one usually feels, and 6) evaluation of one's appearance relative to how the average person looks. Each item is evaluated on a 9-point bipolar Likert like scale. The scale can be administered in a natural context as well as in a range of positive or negative hypothetical situations. A higher BISS score indicates better body image. In some embodiments, after a subject is treated according to the methods of the disclosure, the subject's BISS score increases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 145%, about 150%, about 160%, about 170%, about 180%, about 190%, about 200%, about 210%, about 220%, about 230%, about 240%, about 250%, about 260%, about 270%, about 280%, about 290%, or about 300%.

In some embodiments, a subject's body mass index is evaluated to determine the safety and efficacy of the compound of Formula (I) administered as a solid dispersion for the treatment of eating disorders.

In some embodiments, the Clinical impairment assessment (CIA) questionnaire is utilized to evaluate the safety and efficacy of the compound of Formula (I) administered as a solid dispersion for the treatment of eating disorders. The CIA is a self-reported 16-item scale of the level of social and psychological impairment caused by an eating disorder in the past 28 days. The subscales are in mood, self-perception, cognitive functioning, interpersonal functioning and work preference. The items are rated on a four point Likert scale. In some embodiments, a score of 16 or greater suggests that a subject has an eating disorder. In some embodiments, after treating according to the methods of the disclosure, a subject's CIA score decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the Eating Disorder Inventory (EDI), for example version 3 (EDI-3), is utilized to evaluate the safety and efficacy of the compound of Formula (I) administered as a solid dispersion for the treatment of eating disorders. The EDI-3 is a self-reported questionnaire ranked on a Likert scale from 0-4. The EDI-3 contains 12 subscales, three of which are specific to eating disorders (drive for thinness, bulimia, body dissatisfaction), and nine of which measure general psychological traits (low self-esteem, personal alienation, interpersonal security, interpersonal alienation, interceptive deficits, emotional dysregulation, perfectionism, asceticism, and maturity fear). In some embodiments, after treating according to the methods of the disclosure, a subject's EDI-3 composite decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the Five Dimension Altered States of Consciousness Questionnaire (5D-ASC) is utilized to evaluate the safety and efficacy of the compound of Formula (I) administered as a solid dispersion for the treatment of eating disorders. The 5D-ASC measures the acute drug effects using five primary dimensions and respective subdimensions to assess alterations in mood, perception, and experience of self in relation to environment and thought disorder. The 5 dimensions include: oceanic bound-lessness, anxious ego dissolution, visionary restructuralization, auditory alterations, and reduction of vigilance.

In some embodiments, the Columbia-Suicide Severity Rating Scale (C—SSRS) is utilized to evaluate the safety and efficacy of the compound of Formula (I) administered as a solid dispersion for the treatment of eating disorders. The C—SSRS is used to assess suicide potential or tendency as a study entry criteria and monitored throughout the study. In some embodiments, after treatment according to the methods of the disclosure, a subject's C—SSRS score decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, a qualitative interview is utilized to evaluate the safety and efficacy of the compound of Formula (I) administered as a solid dispersion for the treatment of eating disorders.

In some embodiments, electrocardiogram (ECG) is utilized to evaluate the safety and efficacy of the compound of Formula (I) administered as a solid dispersion for the treatment of eating disorders. In some embodiments, the ECG is a standard 12-lead ECG.

In some embodiments, vital signs are utilized to evaluate the safety and efficacy of the compound of Formula (I) administered as a solid dispersion for the treatment of eating disorders. Non-limiting examples of vital signs include weight, blood pressure, respiratory rate, body temperature, and pulse rate.

In some embodiments, clinical laboratory tests are utilized to evaluate the safety and efficacy of the compound of Formula (I) administered as a solid dispersion for the treatment of eating disorders. In some embodiments, the clinical laboratory tests include liver function tests. In some embodiments, the clinical laboratory tests employ blood samples and/or urine samples. In some embodiments, blood is tested for levels of hemoglobin, hematocrit, red blood cell count, mean corpuscular haemoglobin, mean corpuscular volume, mean corpuscular haemoglobin concentration, white blood cell count (with differential), and platelets. In some embodiments, blood is tested for levels of albumin, alkaline phosphatase, alanine aminotransferase (ALT), amylase, aspartate aminotransferase (AST), bicarbonate, bilirubin (direct, indirect, and total), calcium, chloride, creatine kinase, creatinine, gamma-glutamyltransferase, glucose, lactate dehydrogenase, lipase, magnesium, phosphate, potassium, protein-total, sodium, urea (blood urea nitrogen), and uric acid. In some embodiments, urine samples are tested for pregnancy, illicit drugs, blood, glucose, ketones, protein, pH, specific gravity, nitrite, leukocytes, bilirubin, and urobilinogen.

In some embodiments, adverse events (AEs) are measured to determine the safety and efficacy of the compound of Formula (I) administered as a solid dispersion for the treatment of an eating disorder. In some embodiments, the adverse events are classified as mild, moderate, or serious. A mild AE does not interfere in a significant manner with the subject's normal level of functioning. A moderate AE produces some impairment of functioning but is not hazardous to the subject's health. A severe AE produces significant impairment of functioning or incapacitation and is a definite hazard to the subject's health. Non-limiting examples of adverse events include euphoric mood, Euphoric mood, Dissociative disorder, Hallucination, Psychotic disorder, Cognitive disorder, Disturbance in attention, Mood alterations, impaired psychomotor skills, inappropriate affect, overdose, intentional product misuse. Serious adverse events include death, life-threatening events, insubject hospitalization or prolongation of existing hospitalization, Persistent or significant disability/incapacity, congenital anomaly/birth defect in the offspring of a subject who received the compound of Formula (I). An event is life threatening if the subject was at immediate risk of death from the event as it occurred; i.e., it does not include a reaction that if it had occurred in a more serious form might have caused death. For example, drug-induced hepatitis that resolved without evidence of hepatic failure would not be considered life threatening even though drug-induced hepatitis can be fatal. Important medical events that may not result in death, be life-threatening, or require hospitalization, may be considered a serious adverse event when, based upon appropriate medical judgment, they may jeopardize the subject and may require medical or surgical intervention to prevent one of the outcomes listed in this definition. Such events include, but are not limited to: intensive treatment in an emergency room or at home for allergic bronchospasm, blood dyscrasias or convulsions that do not result in in subject hospitalization, and development of drug dependency or drug abuse.

In some embodiments, treating according to the methods of the disclosure results in an improvement in an eating disorder compared to pre-treatment of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein.

In some embodiments, after treating according to the methods of the disclosure, the subject in need thereof has a decreased eating disorder examination short form (EDE-QS) score compared to prior to said treating. In some embodiments, after treating according to the methods of the disclosure, the subject in need thereof has a decreased Physical Appearance State and Trait Anxiety Scale (PASTAS) score compared to prior to said treating. In some embodiments, after treating according to the methods of the disclosure, the subject in need thereof has a higher willingness to change compared to prior to said treating as evaluated by the Eating Disorder Readiness Ruler (ED-RR). In some embodiments, after treating according to the methods of the disclosure, the subject in need thereof has a decreased Montgomery-Asberg Depression Rating Scale (MADRS) score compared to prior to said treating. In some embodiments, after treating according to the methods of the disclosure, the subject in need thereof has an increased Body Image State Scale (BISS) compared to prior to said treating.

In some embodiments, after administration of the compound of Formula (I) administered as a solid dispersion, a subject with an eating disorder has improved cognition, information processing, stress levels, immune system, or combinations thereof.

In some embodiments, after administration of the compound of Formula (I) administered as a solid dispersion, the eating disorder or at least one symptom thereof is alleviated for a period of at least about 5 minutes to at least about 1 year, for example, at least one symptom of anxiety is alleviated for a period of at least about 5 minutes, at least about 15 minutes, at least about 30 minutes, at least about 45 minutes, at least about 1 hour, at least about 2 hours, at least 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, at least about 2 months, at least about 3 months, at least about 4 months, at least about 5 months, at least about 6 months, at least about 7 months, at least about 8 months, at least about 9 months, at least about 10 months, at least about 11 months, at least about 12 months, or more. In some embodiments, the eating disorder or at least one symptom thereof is alleviated for a period of at least 1 month after administration of the compound of Formula (I). In some embodiments the eating disorder or at least one symptom thereof is alleviated for a period of at least 3 months after administration of the compound of Formula (I). In some embodiments, the eating disorder or at least one symptom thereof is alleviated for a period of at least 1 year after administration of the compound of Formula (I).

In some embodiments, after administration of the compound of Formula (I) administered as a solid dispersion, the eating disorder or at least one symptom thereof is alleviated within about 5 minutes to about 1 week, for example, about 5 minutes, about 15 minutes, about 30 minutes, about 45 minutes, about 1 hour, about 2 hours, at least 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 1 day, about 2 days, about 3 days, about 4 days, about 5 days, about 6 days, or about 7 days. In some embodiments, the eating disorder or at least one symptom thereof is alleviated within 24 hours of administration of the compound of Formula (I) administered as a solid dispersion. In some embodiments, the eating disorder or at least one symptom thereof is alleviated within 1 week of administration of the compound of Formula (I) administered as a solid dispersion.

### Headache Disorders

In some embodiments, a method of treating one or more headache disorders in a subject in need thereof is disclosed herein.

As used herein, the term “headache disorder” refers to a disorder characterized by recurrent headaches. Headache disorders are among the most common disorders of the nervous system. Headache disorders include migraine, tension-type headache, cluster headache, and chronic daily headache syndrome.

In some embodiments, a method of treating cluster headaches in a subject in need thereof is disclosed herein. In some embodiments, at least one sign or symptom of cluster headache is improved following treatment. In some embodiments, the sign or symptom of cluster headache is measured according to a diary assessment, a physical or psychological assessment by clinician, an imaging test, or a neurological examination.

Cluster headache is a primary headache disorder and belongs to the trigeminal autonomic cephalalgias. The definition of cluster headaches is a unilateral headache with at least one autonomic symptom ipsilateral to the headache. Attacks are characterized by severe unilateral pain predominantly in the first division of the trigeminal nerve-the fifth cranial nerve whose primary function is to provide sensory and motor innervation to the face. Attacks are also associated with prominent unilateral cranial autonomic symptoms and subjects often experience agitation and restlessness during attacks.

In some embodiments, a subject with cluster headaches has cluster headache attacks that last from 15 to 180 minutes and occur up to eight times a day. They usually occur at approximately the same time each day and often occur at night. In some embodiments, the cluster headaches are episodic. Episodic cluster headaches (ECH) are experienced by 85-90% of subjects and are when subjects experience daily attacks for weeks to months and then have remission for months to years. Subjects often have one to two episodes a year and these are often in spring or autumn. In some embodiments, the cluster headaches are chronic cluster headaches (CCH). An individual that has CCH experiences attacks lasting more than a year without remission, or remission that lasts less than a month. CCH is experienced by 15-20% of subjects that experience cluster headaches.

Approximately 0.1% of the population suffer from cluster headaches. The disorder has historically been considered to have a male preponderance with a high male-to-female ratio, which differs between ECH and CCH. This ratio is highest in subjects where the age of onset is between 20 to 49 years old; 7.2:1 in ECH and 11.0:1 in CCH. The ratio is lowest when the age of onset is after 50; 2.3:1 in ECH and 0.6:1 in CCH. These ratios were determined by observations of 554 cluster headache patients examined between 1963 and 1997. The authors suggested that these ratios could be related to sex hormone regulation and environmental factors. Recent studies have reported a decreasing male predominance with a ratio of 2:1.

In some embodiments, a subject with cluster headaches also experiences nausea and/or vomiting. In some embodiments, a subject with cluster headaches experiences unilateral pain, excessive tearing, facial flushing, a droopy eyelid, a constricted pupil, eye redness, swelling under or around one or both eyes, sensitivity to light, nausea, agitation, and restlessness.

In some embodiments, after treating according to the methods of the disclosure, the frequency of cluster headaches decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to said treating.

In some embodiments, after treating according to the methods of the disclosure, the length of a cluster headache attack decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to said treating.

In some embodiments, after treating according to the methods of the disclosure a sign or symptom of cluster headache decreases compared to prior to said treatment. In some embodiments, the sign or symptom of cluster headache is measured according to a diary assessment, a physical or psychological assessment by clinician, an imaging test, an electroencephalogram, a blood test, a neurological examination, or combination thereof. In some embodiments, the blood test evaluates blood chemistry and/or vitamins.

In some embodiments, a method of treating migraines in a subject in need thereof is disclosed herein. A migraine is a moderate to severe headache that affects one half or both sides of the head, is pulsating in nature, and last from 2 to 72 hours. Symptoms of migraine include headache, nausea, sensitivity to light, sensitivity to sound, sensitivity to smell, dizziness, difficulty speaking, vertigo, vomiting, seizure, distorted vision, fatigue, or loss of appetite. Some subjects also experience a prodromal phase, occurring hours or days before the headache, and/or a postdromal phase following headache resolution. Prodromal and postdromal symptoms include hyperactivity, hypoactivity, depression, cravings for particular foods, repetitive yawning, fatigue and neck stiffness and/or pain.

In some embodiments, the migraine is a migraine without aura, a migraine with aura, a chronic migraine, an abdominal migraine, a basilar migraine, a menstrual migraine, an ophthalmoplegic migraine, an ocular migraine, an ophthalmic migraine, or a hemiplegic migraine. In some embodiments, the migraine is a migraine without aura. A migraine without aura involves a migraine headache that is not accompanied by a headache. In some embodiments, the migraine is a migraine with aura. A migraine with aura is primarily characterized by the transient focal neurological symptoms that usually precede or sometimes accompany the headache. Less commonly, an aura can occur without a headache, or with a non-migraine headache. In some embodiments, the migraine is a hemiplegic migraine. A hemiplegic migraine is a migraine with aura and accompanying motor weakness. In some embodiments, the hemiplegic migraine is a familial hemiplegic migraine or a sporadic hemiplegic migraine. In some embodiments, the migraine is a basilar migraine. A subject with a basilar migraine has a migraine headache and an aura accompanied by difficulty speaking, world spinning, ringing in ears, or a number of other brainstem-related symptoms, not including motor weakness. In some embodiments, the migraine is a menstrual migraine. A menstrual migraine occurs just before and during menstruation. In some embodiments, the subject has an abdominal migraine. Abdominal migraines are often experienced by children. Abdominal migraines are not headaches, but instead stomach aches. In some embodiments, a subject with abdominal migraines develops migraine headaches. In some embodiments, the subject has an ophthalmic migraine also called an “ocular migraine”. Subjects with ocular migraines experience vision or blindness in one eye for a short time with or after a migraine headache. In some embodiments, a subject has an ophthalmoplegic migraine. Ophthalmoplegic migraines are recurrent attacks of migraine headaches associated with paresis of one or more ocular cranial nerves.

In some embodiments, the subject in need of treatment experiences chronic migraines. As defined herein, a subject with chronic migraines has more than fifteen headache days per month. In some embodiments, the subject in need of treatment experiences episodic migraines. As defined herein, a subject with episodic migraines has less than fifteen headache days per month.

In some embodiments, after treating according to the methods of the disclosure, the frequency of migraine decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to said treating.

In some embodiments, after treating according to the methods of the disclosure, the length of a migraine headache decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to said treating.

In some embodiments, after treating according to the methods of the disclosure a sign or symptom of migraine decreases compared to prior to said treatment. In some embodiments, the sign or symptom of migraine is measured according to a diary assessment, a physical or psychological assessment by clinician, an imaging test, or a neurological examination.

In some embodiments, a method of treating chronic daily headache syndrome (CDHS) in a subject in need thereof is disclosed herein. A subject with CDHS has a headache for more than four hours on more than 15 days per month. Some subjects experience these headaches for a period of six months or longer. CHDS affects 4% of the general population. Chronic migraine, chronic tension-type headaches, new daily persistent headache, and medication overuse headaches account for the vast majority of chronic daily headaches.

In some embodiments, after treating according to the methods of the disclosure, a chronic daily headache experienced by the subject in need is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, after treating according to the methods of the disclosure, the number of chronic daily headaches experienced by the subject in need per month is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, after treating according to the methods of the disclosure, the number of chronic daily headaches experienced by the subject in need per month is reduced by about 1 headache per month, about 2 headaches per month, about 3 headaches per month, about 4 headaches per month, about 5 headaches per month, about 6 headaches per month, about 7 headaches per month, about 8 headaches per month, about 9 headaches per month, about 10 headaches per month, about 11 headaches per month, about 12 headaches per month, about 13 headaches per month, about 14 headaches per month, about 15 headaches per month, or more.

In some embodiments, the subject with a headache disorder has one or more comorbidities. Non-limiting examples of comorbidities include endocrine and/or metabolic diseases, gastrointestinal diseases, cardiovascular diseases, psychiatric disorders, or a neuroses. In some embodiments, the comorbidity is anxiety disorder, depressive disorder, psychotic disorder, developmental disorder, eating disorder, attention-deficit hyperactivity disorder (ADHD), attention deficit disorder (ADD), autism spectrum disorder (ASD), tic disorder, bipolar disorder, conduct disorder, mental retardation, or a combination thereof. In some embodiments, the comorbidity is a mood disorder, major depressive disorder, bipolar disorder, schizophrenia, an eating disorder, attention deficit/hyperactivity disorder, epilepsy, cardiovascular disease, migraine, irritable bowel syndrome, dementia, Alzheimer's disease, Parkinson's disease, or combinations thereof. In some embodiments, the comorbidity is depression, generalized anxiety, agoraphobia, or panic disorder. In some embodiments, the comorbidity is major depression. In some embodiments, the comorbidity is an eating disorder. In some embodiments, the comorbidity is ADHD. In some embodiments, the comorbidity is ASD. In some embodiments, the comorbidity is major depressive disorder, bipolar disorder, irritable bowel disease, or irritable bowel syndrome.

In some embodiments, the comorbidity is sleep apnea, depression, anxiety, aggressive behavior, suicidal ideation, or bipolar disorder. In some embodiments, the comorbidity is tobacco use, sleep apnea, depression, anxiety, aggressive behavior, suicidal ideation, or bipolar disorder. In some embodiments, the comorbidity is stroke, vascular brain lesions, coronary heart disease, patent foramen ovale, hypertension, depression, anxiety, bipolar disorder, panic disorder, suicide, restless leg syndrome, epilepsy, inflammatory bowel disease, or asthma.

In some embodiments, the subject in need thereof is a male. In some embodiments, the subject in need thereof is a female. In some embodiments, the subject in need thereof is a pregnant woman. In some embodiments, the subject in need thereof is a post-partum woman. In some embodiments, the subject in need thereof is psilocybin-naive. In some embodiments, the subject in need thereof has prior psilocybin experience.

In some embodiments, no other treatment is administered to the subject to treat headache disorder after administration of the compound of Formula (I) administered as a solid dispersion.

In some embodiments, the subject with headache disorder is administered at least one additional therapy and/or therapeutic. In some embodiments, administration of an additional therapy and/or therapeutic is prior to administration of the compound of Formula (I) administered as a solid dispersion. In some embodiments, administration of an additional therapy and/or therapeutic is after administration of the compound of Formula (I) administered as a solid dispersion. In some embodiments, administration of an additional therapy and/or therapeutic is concurrent with administration of the compound of Formula (I) administered as a solid dispersion.

In some embodiments, the additional therapy is oxygen therapy, triptans, ergotamine, lidocaine, somatostatin and somatostatin analogues, corticosteroids, methysergide, verapamil, lithium, topiramate, valproic acid, botulinum toxin, clomiphene, lysergic acid diethylamide, prednisolone, dihydroergotamine, a beta blocker, a vitamin, an antidepressant, an anticonvulsant, lisdexamfetamine dimesylate, an antipsychotic, an anxiolytic, an anti-inflammatory drug, a benzodiazepine, an analgesic drug, a cardiovascular drug, or combinations thereof.

In some embodiments, the additional therapy is an antidepressant. In some embodiments, the antidepressant is amitriptyline. In some embodiments, an antidepressant indirectly affects a neurotransmitter receptor, e.g., via interactions affecting the reactivity of other molecules at a neurotransmitter receptor. In some embodiments, an antidepressant is an agonist. In one embodiment, an antidepressant is an antagonist. In some embodiments, an antidepressant acts (either directly or indirectly) at more than one type of neurotransmitter receptor. In some embodiments, an antidepressant is chosen from buproprion, citalopram, duloxetine, escitalopram, fluoxetine, fluvoxamine, milnacipran, mirtazapine, paroxetine, reboxetine, sertraline, and venlafaxine.

In some embodiments, the antidepressant is a tricyclic antidepressant (“TCA”), selective serotonin reuptake inhibitor (“SSRI”), serotonin and noradrenaline reuptake inhibitor (“SNRI”), dopamine reuptake inhibitor (“DRI”), noradrenaline reuptake monoamine oxidase inhibitor (“MAOI”), including inhibitor (“NRU”), dopamine, serotonin and noradrenaline reuptake inhibitor (“DSNRI”), a reversible inhibitor of monoamine oxidase type A (RIMA), or combination thereof. In some embodiments, the antidepressant is a TCA. In some embodiments, the TCA is imipramine or clomipramine. In some embodiments, the anti-depressant is an SRI. In some embodiments, the SSRI is escitalopram, paroxetine, sertraline, fluvoxamine, fluoxetine, or combinations thereof. In some embodiments, the SNRI is venlafaxine. In some embodiments, the additional therapy is pregabalin. In some embodiments, the antidepressant is amitriptyline.

In some embodiments, the additional therapeutic is an anticonvulsant. In some embodiments, the anticonvulsant is gabapentin, carbamazepine, ethosuximide, lamotrigine, felbamate, topiramate, zonisamide, tiagabine, oxcarbazepine, levetiracetam, divalproex sodium, phenytoin, fosphenytoin. In some embodiments, the anticonvulsant is topiramate.

In some embodiments, the additional therapeutic is a beta blocker. Non-limiting examples of beta blockers include acebutolol, atenolol, betaxolol, bisoprolol, carteolol, carvedilol, labetalol, metoprolol, nadolol, nebivolol, penbutolol, pindolol, propranolol, sotalol, or timolol. In some embodiments, the beta blocker is propranolol.

In some embodiments, the additional therapeutic is a non-steroidal anti-inflammatory drug, acetaminophen, caffeine, a triptan, a dihydroergotamine, an opioid, an anti-nausea drug, a blood pressure-lowering medication, an anti-depressant, an anti-seizure drug, a botox injection, a monoclonal antibody directed to calcitonin gene-related peptide, acupuncture, black room, change of diet, hot/cold therapy, magnesium intake, massages, riboflavin/B2 intake, transcranial magnetic stimulation, or any combination thereof.

In some embodiments, at least one sign or symptom of headache disorder is improved following administration of the compound of Formula (I) administered as a solid dispersion. In some embodiments, a sign or symptom of a headache disorder is measured according to a diary assessment, an assessment by a clinician or caregiver, or a clinical scale. In some embodiments, treatment of the present disclosure causes a demonstrated improvement in one or more of the following: the Visual Analog Scale, Numeric Rating Scale, the Short Form Health Survey, Profile of Mood States, the Pittsburgh Sleep Quality Index, the Major Depression Inventory, the Perceived Stress Scale, the 5-Level EuroQoL-5D, the Headache Impact Test; the ID-migraine; the 3-item screener; the Minnesota Multiphasic Personality Inventory; the Hospital Anxiety and Depression Scale (HADS), the 50 Beck Depression Inventory (BDI; both the original BD151 and the second edition, BDI-1152), the 9-item Patient Health Questionnaire (PHQ-9), the Migraine Disability Assessment Questionnaire (MI-DAS), the Migraine-Specific Quality of Life Questionnaire version 2.1 (MSQ v2.1), the European Quality of Life-5 Dimensions (EQ-5D), the Short-form 36 (SF-36), or a combination thereof.

In some embodiments, treating according to the methods of the disclosure results in an improvement in a headache disorder compared to pre-treatment of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein.

In some embodiments, treating according to the methods of the disclosure results in an improvement in migraine compared to pre-treatment of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein. In some embodiments, the assessment is the Visual Aura Rating Scale or the ID Migraine questionnaire.

In some embodiments, treating according to the methods of the disclosure results in an improvement in cluster headaches compared to pre-treatment of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein. In some embodiments, the assessment is the Visual Analog Scale, the Numeric Rating Scale, the Short Form Health Survey, the Profile of Mood States, the Pittsburgh Sleep Quality Index, the Major Depression Inventory, the Perceived Stress Scale, the 5-Level EuroQoL-5D, the Headache Impact Test, or combinations thereof.

In some embodiments, after administration of the compound of Formula (I) administered as a solid dispersion, at least one symptom of headache disorder is alleviated for a period of at least about 5 minutes to at least about 1 year, for example, at least one symptom of anxiety is alleviated for a period of at least about 5 minutes, at least about 15 minutes, at least about 30 minutes, at least about 45 minutes, at least about 1 hour, at least about 2 hours, at least 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, at least about 2 months, at least about 3 months, at least about 4 months, at least about 5 months, at least about 6 months, at least about 7 months, at least about 8 months, at least about 9 months, at least about 10 months, at least about 11 months, at least about 12 months, or more. In some embodiments, at least one symptom of headache disorder is alleviated for a period of at least 1 month after administration of the compound of Formula (I) administered as a solid dispersion. In some embodiments, at least one symptom of headache disorder is alleviated for a period of at least 3 months after administration of the compound of Formula (I) administered as a solid dispersion. In some embodiments, at least one symptom of headache disorder is alleviated for a period of at least 1 year after administration of the compound of Formula (I) administered as a solid dispersion.

In some embodiments, the administration of the compound of Formula (I) administered as a solid dispersion prevents the occurrence of the headache disorder for at least about 1 week or more, for example, about 1 week, about 2 weeks, about 3 weeks, about 4 weeks, about 5 weeks, about 1 month, about 6 weeks, about 2 months, about 3 months, about 4 months, about 5 months, about 6 months, about 7 months, about 8 months, about 9 months, about 10 months, about 11 months, about 1 year, or more. In some embodiments, the administration of the compound of Formula (I) administered as a solid dispersion prevents the occurrence of the headache disorder for at least about 1 month. In some embodiments, the administration of the compound of Formula (I) administered as a solid dispersion prevents the occurrence of the headache disorder for at least about 3 months. In some embodiments, the administration of the compound of Formula (I) administered as a solid dispersion prevents the occurrence of the headache disorder for at least about 6 months. In some embodiments, the administration of the compound of Formula (I) administered as a solid dispersion prevents the occurrence of the headache disorder for at least about 12 months.

In some embodiments, after administration of the compound of Formula (I) administered as a solid dispersion, at least one symptom of headache disorder is alleviated within about 5 minutes to about 1 week, for example, about 5 minutes, about 15 minutes, about 30 minutes, about 45 minutes, about 1 hour, about 2 hours, at least 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 1 day, about 2 days, about 3 days, about 4 days, about 5 days, about 6 days, or about 7 days. In some embodiments, at least one symptom of headache disorder is alleviated or improved within 15 minutes of administration of the compound of Formula (I) administered as a solid dispersion. In some embodiments, at least one symptom of headache disorder is alleviated or improved within 30 minutes of administration of the compound of Formula (I) administered as a solid dispersion. In some embodiments, at least one symptom of headache disorder is alleviated or improved within 1 hour of administration of the compound of Formula (I) administered as a solid dispersion. In some embodiments, at least one symptom of headache disorder is alleviated or improved within 3 hours of administration of the compound of Formula (I) administered as a solid dispersion. In some embodiments, at least one symptom of headache disorder is alleviated or improved within 6 hours of administration of the compound of Formula (I) administered as a solid dispersion.

### Pre-Treatments and Combination Therapies

In some embodiments, the methods of treatment herein further comprise pretreating the subject with magnesium before administration of the compound of Formula (I) administered as a solid dispersion. Sometimes, magnesium is administered daily for a least 1 day, at least 2 days, at least 3 days, at least 4 days, at least 5 days, at least 6 days, at least 1 week, at least 2 weeks, at least 3 weeks, at least 4 weeks, at least 5 weeks, or at least 6 weeks before administration of the compound of Formula (I) administered as a solid dispersion. In some embodiments, about 10 mg to about 500 mg of magnesium are administered to the subject per day. In some embodiments, about 30 mg, about 75 mg, about 80 mg, about 130 mg, about 240 mg, about 310 mg, about 320 mg, about 360 mg, about 410 mg, about 400 mg, or about 420 mg are administered to the subject per day. In some embodiments the magnesium is administered to the subject on the same day as the compound of Formula (I) administered as a solid dispersion. In some embodiments, the magnesium is administered to the subject immediately before, concurrently with, or immediately after administration of the compound of Formula (I) administered as a solid dispersion. In some embodiments, magnesium supplements are administered to the subject until the subject's blood level for magnesium is about 1.5 to about 2.5 mEq/L. In some embodiments, the compound of Formula (I) is not administered to the subject if the subject's blood level of magnesium is less than about 1.5 to about 2.5 mEq/L.

In some embodiments, the methods of treatment herein further comprise pretreating the subject with niacin before administration of the compound of Formula (I) administered as a solid dispersion. Sometimes, niacin is administered daily for a least 1 day, at least 2 days, at least 3 days, at least 4 days, at least 5 days, at least 6 days, at least 1 week, at least 2 weeks, at least 3 weeks, at least 4 weeks, at least 5 weeks, or at least 6 weeks before administration of the compound of Formula (I) administered as a solid dispersion. In some embodiments, about 1 mg to about 5,000 mg of niacin are administered to the subject per day, for example about 1 mg to about 50 mg, about 10 mg to about 100 mg, about 100 mg to about 200 mg, about 1 mg to about 200 mg, about 100 mg to about 200 mg, about 10 mg to about 50 mg, about 10 to about 35 mg, about 100 mg to about 500 mg, or about 1,000 mg to about 3,000 mg. In some embodiments, about 10 mg, about 14 mg, about 15 mg, about 16 mg, about 20 mg, about 30 mg, about 35 mg, about 50 mg, about 60 mg, about 75 mg, about 100 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1500 mg, about 2000 mg, about 2500 mg, or about 3000 mg of niacin are administered to the subject per day (while avoiding any toxic exposure from excess niacin). In some embodiments, niacin is included as an ingredient/component, for example, to reduce risk of abuse and/or to improve efficacy. In some embodiments the niacin is administered to the subject on the same day as the compound of Formula (I) administered as a solid dispersion. In some embodiments, the niacin is administered to the subject immediately before, concurrently with, or immediately after administration of the compound of Formula (I) administered as a solid dispersion.

In some embodiments, the compound of Formula (I) in the form of a solid dispersion is administered to the subject in combination with one or more additional therapies. In some embodiments, the compound of Formula (I) is administered to the subject in combination with one or more anti-depressant or anti-anxiety drugs, such as SSRis, tricyclic antidepressants (TCAs), monoamine oxidase inhibitors (MAOis), or serotonin norepinephrine reuptake inhibitors (SNRis).

In some embodiments, the disclosure provides a method of reducing anxiety in a subject undergoing treatment herein, the method comprising administering to the subject: i) the compound of Formula (I) administered as a solid dispersion, and ii) one or more benzodiazepines. In some embodiments, the one or more benzodiazepines are administered to the subject at or around the same time as the compound of Formula (I) administered as a solid dispersion. In some embodiments, the one or more benzodiazepines are administered to the subject prior to administration of the compound of Formula (I) administered as a solid dispersion, such as about 10 minutes, about 15 minutes, about 20 minutes, about 30 minutes, about 45 minutes, about 60 minutes, about 75 minutes, about 90 minutes, about 105 minutes, about 120 minutes, about 150 minutes, or about 180 minutes before administration of the psilocybin or precursor or derivative thereof. In some embodiments, the one or more benzodiazepines are administered to the subject after the compound of Formula (I) administered as a solid dispersion, such as about 10 minutes, about 15 minutes, about 20 minutes, about 30 minutes, about 45 minutes, about 60 minutes, about 75 minutes, about 90 minutes, about 105 minutes, about 120 minutes, about 150 minutes, or about 180 minutes after said administration.

In some embodiments, the one or more benzodiazepines are administered at a dose that is lower than doses typically used to treat anxiety, such as about 10%, 20%, 25%, 30%, 40%, 50%, or 75% of a typical dose. In some embodiments, the one or more benzodiazepines are administered at a dose that is approximately equivalent to doses typically used to treat anxiety. In some embodiments, the one or more benzodiazepines are administered at a dose that is higher than doses typically used to treat anxiety, such as about 125%, 150%, 175%, 200%, 250%, or 300% of a typical dose. In some embodiments, the one or more benzodiazepine is administered orally to the subject.

In some embodiments, the benzodiazepine is selected from adinazolam, alprazolam, bentazepam, bretazenil, bromazepam, bromazolam, brotizolam, camazepam, chlordiazepoxide, cinazepam, cinolazepam, clobazam, clonazepam, clonazolam, clorazepate, clotiazepam, cloxazolam, delorazepam, deschloroetizolam, diazepam, diclazepam, estazolam, ethyl carfluzepate, ethyl loflazepate, etizolam, flualprazolam, flubromazepam, flubromazolam, fluclotizolam, flunitrazepam, flunitrazolam, flurazepam, flutazolam, flutoprazepam, halazepam, ketazolam, loprazolam, lorazepam, lormetazepam, meclonazepam, medazepam, metizolam, mexazolam, midazolam, nifoxipam, nimetazepam, nitemazepam, nitrazepam, nitrazolam, nordiazepam, norflurazepam, oxazepam, phenazepam, pinazepam, prazepam, premazepam, pyrazolam, quazepam, rilmazafone, temazepam, tetrazepam, or triazolam, and the like.

In some embodiments, a patient is administered the compound of Formula (I) administered as a solid dispersion as described herein along with one or more 5-HT_{2A} specific antagonists and/or inverse agonists. In some embodiments, the subject is administered the compound of Formula (I) administered as a solid dispersion and the one or more 5-HT_{2A} specific antagonists and/or inverse agonists at the same time. In other embodiments, the subject is administered one or more 5-HT_{2A} specific antagonists and/or inverse agonists prior to administration of the compound of Formula (I) such as, but not limited to about 10 minutes, about 15 minutes, about 20 minutes, about 30 minutes, about 45 minutes, about 60 minutes, about 75 minutes, about 90 minutes, about 105 minutes, about 120 minutes, about 150 minutes, or about 180 minutes before said administration. In some embodiments, the subject is administered one or more 5-HT_{2A} specific antagonists and/or inverse agonists after administration of the compound of Formula (I) administered as a solid dispersion, such as, but not limited to about 10 minutes, about 15 minutes, about 20 minutes, about 30 minutes, about 45 minutes, about 60 minutes, about 75 minutes, about 90 minutes, about 105 minutes, about 120 minutes, about 150 minutes, or about 180 minutes after said administration.

In some embodiments, the one or more 5-HT_{2A} specific antagonists and/or inverse agonists are administered at doses that are lower than doses typically used, e.g., about 10%, about 20%, about 25%, about 30%, about 40%, about 50%, or about 75% of a typical dose. In other embodiments, the one or more 5-HT_{2A} specific antagonists and/or inverse agonists are administered at doses that are equivalent to doses typically used. In yet other embodiments, the one or more 5-HT_{2A} specific antagonists and/or inverse agonists are administered at doses that are higher than doses typically used, e.g., about 125%, about 150%, about 175%, about 200%, about 250%, or about 300% of a typical dose.

Suitable 5-HT_{2A} antagonists include, but are not limited to, trazodone, mirtazapine, metergoline, ketanserin, ritanserin, nefazodone, clozapine, olanzapine, quetiapine, risperidone, asenapine, MDL-100907, cyproheptadine, pizotifen, LY-367,265, 2-alkyl-4-aryl-tetrahydro-pyrimidoazepine, 9-aminomethyl-9,10-dihydroanthracene (AMDA), haloperidol, chlorpromazine, hydroxyzine (atarax), 5-MeO-NBpBrT, niaprazine, altanserin, aripiprazole, etoperidone, setoperone, chlorprothixene, cinaserin, adatanserin, medifoxamine, rauwolscine, phenoxybenzamine, pruvanserin, deramciclane, nelotanserin, lubazodone, mepiprazole, xylamidine, R—(+)-alpha-(2,3-dimethoxyphenyl)-1-[2-(4-fluorophenethyl)]-4-piperidinemethanol (M100907), mianserin, AT 1015, DV 7028, eplivanserin, 4F 4PP, fanaserin, alpha-phenyl-1-(2-phenylethyl)-4-piperidinemethanol (MDL 11,939), melperone, mesulergine, paliperidone, 1-[2-(3,4-dihydro-1H-2-benzopyran-1-yl)ethyl]-4-(4-fluorophenyl) piperazine dihydrochloride (PNU 96415E), (2R,4R)-5-[2-[2-[2-(3-methoxyphenyl)ethyl]phenoxy]ethyl]-1-methyl-3-pyrrolidinol (R-96544), sarpogrelate, spiperone, ziprasidone, zotepine, and 7-[[4-[2-(4-fluorophenyl)ethyl]-1-piperazinyl]carbonyl]-1H-indole-3-carbonitrile (EMD 281014).

Suitable 5-HT_{2A} inverse agonists include but are not limited to, AC-90179, nelotanserin (APD-125), eplivanserin, pimavanserin (ACP-103), and volinaserin.

In some embodiments, the 5-HT_{2A} antagonist is selected from the group consisting of acepromazine, agomelatine, amitriptyline, arnoxapine, amperozide, APD791, aripiprazole, aripiprazole, lauroxil, blonanserin, brexpiprazole, butriptyline, captodiame, cariprazine, chlorpromazine, chlorprothixene, cimtapride, citalopram, clomiprarnine, clozapine, cyclobenzaprine, cyproheptadine, derarnciclane, desiprarnine, dosulepin, doxepin, epinastine, esmirtazapine, etoperidone, flibanserin, fluoxetine, flupentixol, fluspirilene, iloperidone, imiprarnine, lisuride, loxapine, lurasidone, mesoridazine, methotrimeprazine, metbysergide, mianserin, mirtazapine, nefazodone, nortriptyline, olanzapine, paliperidone, pimavanserin, pizotifen, promazine, propiomazine, PRX-08066, quetiapine, risperidone, sertindole, thioproperazine, thioridazine, trarnadol, trazodone, triflupromazine, trimiprarnine, YKP-1358, yohimbine, ziprasidone, zotepine, and zuclopentbixol.

In some embodiments, the disclosure provides a method of reducing the negative side effects associated with a traumatic psychedelic experience in a subject undergoing treatment, the method comprising administering to the subject: i) the compound of Formula (I) administered as a solid dispersion as described herein, and ii) one or more cannabinoids or cannabinoid derivatives.

In some embodiments, the cannabinoid is selected from THC (tetrahydrocannabinol), THCA (tetrahydrocannabinolic acid); CBD (cannabidiol); CBDA (cannabidiolic acid); CBN (cannabinol); CBG (cannabigerol); CBC (cannabichromene); CBL (cannabicyclol); CBV (cannabivarin); THCV (tetrahydrocannabivarin); CBDV (cannabidivarin); CBCV (cannabichromevarin); CBGV (cannabigerovarin); CBGM (cannabigerol monomethyl ether); CBE (cannabielsoin); or CBT (cannabicitran). In particular embodiments, the cannabinoid is CBD (cannabidiol).

In some embodiments, at least one symptom of a disease, disorder, or condition described herein is alleviated within 24 hours of administering the compound of Formula (I) administered as a solid dispersion as described herein. In some embodiments, at least one symptom of the disease, disorder, or condition is alleviated within 1 week of the administering. In some embodiments, at least one symptom of the disease, disorder, or condition is alleviated within 1 month of the administering. In some embodiments, at least one symptom of the disease, disorder, or condition is alleviated within 6 months of the administering. In some embodiments, at least one symptom of the disease, disorder, or condition is alleviated within 12 months of the administering.

In some embodiments, at least one symptom of the disease, disorder, or condition is alleviated for a period of at least 1 month after administering the compound of Formula (I) administered as a solid dispersion as described herein. In some embodiments, at least one symptom of the disease, disorder, or condition is alleviated for a period of at least 3 months after the administering. In some embodiments, at least one symptom of the disease, disorder, or condition is alleviated for a period of at least 6 months after the administering. In some embodiments, at least one symptom of the disease, disorder, or condition is alleviated for a period of at least 12 months after the administering.

In some embodiments, no other treatment is administered to the subject to treat the disease, disorder, or condition before administration of the compound of Formula (I) administered as a solid dispersion as described herein. In some embodiments, no other treatment is administered to the subject to treat the disease, disorder, or condition after the administering.

### Safety and Efficacy of Psilocybin

The present disclosure also relates to the safety and efficacy of the use of compound of Formula (I) administered as a solid dispersion as described herein. The following is a non-exhaustive list of tests that can be used to determine the effects of such treatment, and in particular the pharmaceutical compositions as disclosed herein administered as disclosed herein.

In some embodiments, the Spatial Working Memory (SWM) test is utilized to evaluate the safety and efficacy of treatment disclosed herein. SWM requires retention and manipulation of visuospatial information. Study subjects are required to find the blue tokens in the on-screen ‘boxes’. Boxes are searched by touching them to determine whether they contain a token. Once a token has been located it is ‘stacked’ in a column on the right of the screen. Study subjects then search for further tokens until they have all been located. The remaining tokens will thereafter only be found in boxes that have not so far yielded a token. Study subjects are explicitly told this is the case and it they revisit a box in which a token has been found they commit a ‘between error’, the usual primary metric for this test. Occasions on which the subject revisits a box in the same search are scored as a ‘within’ error. Many study subjects will adopt a search strategy via which they systematically search the array of boxes. This is also scored by the Cambridge Neuropsychological Test Automated Battery system and yields a ‘strategy’ score. SWM performance is impaired by damage to the prefrontal cortex, especially the dorsolateral prefrontal cortex. Similarly, in neuroimaging studies in healthy volunteers, SWM performance is associated with activations in the dorsolateral and mid-ventrolateral prefrontal cortex. This test takes approximately 4 min to complete.

In some embodiments, the efficacy of treatment herein is evaluated using the spatial working memory between errors (SWMBE) score. In some embodiments, after treating according to the methods of the disclosure, a subject's SWMBE score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the efficacy of treatment herein is evaluated using the spatial working memory strategy (SWMS) score. In some embodiments, after treating according to the methods of the disclosure, a subject's SWMS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the Rapid Visual Information Processing (RVP) test is utilized to evaluate the safety and efficacy of treatment herein. The RVP is a measure of sustained attention outputting measures of response accuracy, target sensitivity and reaction times. In this test, the study subject is required to monitor a stream of digits from 2 to 9 for specific sequences (e.g., 3-5-7) and to acknowledge detection of the sequence by touching the on-screen response button as quickly as possible after presentation of the third digit. Digits are presented pseudorandomly to create the possibility of ‘false alarm’ responses in which the first 2 digits of a sequence are not followed by a true target, e.g., when 3 is followed by a 5, but not then by a 7. In order to complete the task successfully study subjects must sustain attention to the white box in which the digits appear. Performance on this task is measured by the speed of response to the presentation of the final digit of a target, as well as the study subject's ability to detect specified sequences. This test takes approximately 7 min to complete. In some embodiments, performance on the Rapid Visual Information Processing test is reported using a RVP A Prime (RPVA) score. Higher scores RVPA scores indicated better performance. In some embodiments, after treating according to the methods of the disclosure, a subject's RVPA score increases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

In some embodiments, the Paired Associates Learning (PAL) test is utilized to evaluate safety and/or efficacy of treatment herein. The PAL task is a measure of visuo-spatial memory in which study subjects are required to remember locations at which visual stimuli are located. Boxes are displayed on the screen and are “opened” in a randomized order. One or more of them will contain a pattern. The patterns are then displayed in the middle of the screen, one at a time and the subject must select the box in which the pattern was originally located. If the subject makes an error, the boxes are opened in sequence again to remind the subject of the locations of the patterns. Increased difficulty levels can be used to test high-functioning, healthy individuals. The primary metric for this test is the number of errors made. This test takes approximately 8 min to complete. Successful performance of the PAL test is dependent on functional integrity of the temporal lobe, particularly the entorhinal cortex. In some embodiments, the Paired Associates Learning total errors adjusted (PALTEA) score is used to assess the efficacy of the disclosed treatment method. In some embodiments, after treating according to the methods of the disclosure, a subject's PALTEA score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the efficacy and/or safety of the treatment herein is evaluated using the cognitive flexibility panel test.

In some embodiments, the Emotion Recognition Task (ERT) test is utilized to evaluate the safety and/or efficacy of the treatment herein. The ERT measures the ability to identify 6 basic emotions in facial expressions along a continuum of expression magnitude. In some embodiments, the ERT is performed according to the following protocol: Subjects are shown computer morphed images derived from the facial features of real individuals each showing a specific emotion, on a screen, one at a time. Each face is displayed for 200 ms and then immediately covered up, and the subject must select which emotion the face displayed from the six options (happy, sad, anger, fear, surprise, disgust). The ERT percent correct (ERTPC) of correct responses (emotion selection) the subject made is assessed. A higher score indicates better performance. In some embodiments, after treating according to the methods of the disclosure, a subject's ERTPC increases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

In some embodiments, the Intra-Extra Dimensional Set Shift (IED) test is used to evaluate the safety and/or efficacy of the treatment herein. The IED consists of four 7-item subscales, each of which taps a separate aspect of the global concept “empathy.” In some embodiments, the Intra-Extra Dimensional Set Shift total errors (IEDYERT) score is used to assess the efficacy of the disclosed treatment method. In some embodiments, after treating according to the methods of the disclosure, a subject's IEDYERT score decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the One Touch Stockings (OTS) of Cambridge test is used to evaluate the safety and/or efficacy of treatment herein. The OTS is a test of executive function, based upon the Tower of Hanoi test. It assesses both the spatial planning and the working memory subdomains. This test takes approximately 10 min to perform. The OTS test reports a one touch stockings of Cambridge problems solved on first choice (OTSPSFC) score. A higher OTSPSFC score is associated with better executive function. In some embodiments, after treatment according to the methods of the disclosure, a subject's OTSPSFC score increases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, as compared to prior to treatment.

In some embodiments, verbal fluency is used to evaluate the safety and/or efficacy of treatment herein. In the verbal fluency test, the study subject is asked to name as many different category exemplars (e.g., ‘animals’) as they can in 1 min, subject to certain scoring rules, such as repetition. Successful performance on this test is reliant on the integrity of a number of cognitive abilities and especially those traditionally viewed as executive functions, such as planning and working memory. The primary metric for this test is the total number of acceptable words generated. In some embodiments, after treatment herein, a subject's verbal fluency category score improves by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, as compared to prior to treatment.

In some embodiments, the Digit Span Forward (DSF) test is used to evaluate the safety and/or efficacy of the treatment herein. DSF is used to measure number storage capacity. Subjects hear a sequence of digits and are tasked to recall the sequence correctly, with increasingly longer sequences being tested in each trial. The subject's span is the longest number of sequential digits that can accurately be remembered. Digit span tasks can be given forwards or backwards, meaning that once the sequence is presented, the subject is asked to either recall the sequence in normal or reverse order. For this study, subjects will be asked to recall the sequence in the order presented, i.e., Digit Span Forward. The primary metric for this test is the number of digit sequences successfully recalled. In some embodiments, after treatment herein, a subject's Digit Span Forward score improves by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, as compared to prior to treatment.

In some embodiments, the Five Dimension Altered States of Consciousness questionnaire (5D-ASC) is utilized to evaluate the safety and/or efficacy of treatment herein. The 5D-ASC measures the acute drug effects using 5 primary dimensions and 11 lower-order scales to assess alterations in mood, perception and experience of self in relation to environment and thought disorder. The dimensions include oceanic boundlessness, anxious ego dissolution, visionary restructuralization, auditory alterations and reduction of vigilance. In some embodiments, after treatment according to the methods of the disclosure, a subject experiences an increase on a dimension or a subscale compared to prior to treatment. The lower-order scales include “experience of unity,” “spiritual experience,” “blissful state,” “insightfulness,” “disembodiment,” “impaired control of cognition,” “anxiety,” “complex imagery,” “elementary imagery,” “audio-visual synesthesia,” and “changed meaning of percepts.” In some embodiments, the increase is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

In some embodiments, the Positive and Negative Affect Schedule (PANAS) is used to evaluate the safety and/or efficacy of treatment herein. The PANAS measures the acute emotional drug effects and comprises 2 mood scales that measure positive and negative affect. Positive affect refers to the propensity to experience positive emotions and interact with others positively. Negative affect involves experiencing the world in a more negative way. Subjects respond to 10 questions associated with negative affect and 10 questions associated with positive affect. The questions are scaled using a 5-point scale that ranges from “slightly or not at all (1)” to “extremely (5)”. A total higher score on the positive affect questions indicates more of a positive effect while a lower score on the negative affect questions indicates less of a negative effect. In some embodiments, after treating according to the methods of the disclosure, a subject experiences a decrease in negative affect score of the PANAS, between about 5% and about 100%, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment. In some embodiments, after treating according to the methods of the disclosure, a subject experiences an increase in positive affect score of the PANAS, between about 5% and about 100%, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

In some embodiments, the NEO-Five Factor Inventory (NEO-FFI) test is used to evaluate the safety and/or efficacy of treatment herein. The NEO-FFI evaluates 5 broad domains of personality-Neuroticism, Extroversion, Openness, Agreeableness and Conscientiousness.

In some embodiments, the Symptom Checklist-90 item (SCL-90) questionnaire is used to evaluate the safety and/or efficacy of treatment herein. The SCL-90 is a relatively brief self-report psychometric instrument designed to evaluate a broad range of psychological problems and symptoms of psychopathology. In some embodiments, the SCL-90 is used to assess somatization, obsessive-compulsive behaviors, interpersonal sensitivity, depression, anxiety, hostility, phobic anxiety, paranoid ideation, and psychoticism of a subject treated according to the methods of the disclosure. The 90 items in the questionnaire are scored on a 5-point Likert scale, indicating the rate of occurrence of the symptom during the time reference. In some embodiments, after treating according to the methods of the disclosure, a subject's SCL-90 score decreases by about 5% to about 100%, for example, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the Life Changes Inventory (LCI) questionnaire is utilized to evaluate the safety and/or efficacy of treatment herein. The LCI is designed as a questionnaire to investigate those variables present in the day-to-day experience of adults that might relate either to stability or decline of intellectual ability.

In some embodiments, Social Cognition Panel scales are utilized to evaluate the safety and/or efficacy of treatment. The social cognition panel scales comprise the pictorial empathy test (PET), reading the mind in the eyes test (RMET), social value orientation (SVO) test, the Toronto Empathy Questionnaire (TEQ), and the scale of social responsibility (SSR).

In some embodiments, the Pictorial Empathy Test (PET) is utilized to evaluate the effect of treatment herein on affective empathy.

In some embodiments, Reading the Mind in the Eyes Test (RMET) is utilized to evaluate the safety and/or efficacy of treatment herein. The RMET has 36 items, in which subjects are presented with a photograph of the eyes region of the face and must choose 1 of 4 adjectives or phrases to describe the mental state of the person pictured. A definition handout is provided at the beginning of the task and a practice item precedes the first trial.

In some embodiments, the Social Value Orientation (SVO) test is utilized to evaluate the safety and/or efficacy of treatment herein. The SVO Slider Measure has 6 primary items with 9 secondary (and optional) items. All of the items have the same general form. Each item is a resource allocation choice over a well-defined continuum of joint payoffs.

In some embodiments, after treating according to the methods of the disclosure, one or more of the subject's Social Cognition Panel Scales Score, i.e., PET, RMET, SVO, TEQ, and/or SSR score, improves by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

In some embodiments, the Toronto Empathy Questionnaire (TEQ) is utilized to evaluate the safety and/or efficacy of the treatment herein. The TEQ represents empathy as a primarily emotional process. The TEQ has exhibited good internal consistency and high test-retest reliability. The TEQ is a brief, reliable and valid instrument for the assessment of empathy. In some embodiments, after treating according to the methods of the disclosure, a subject's TEQ score increases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

In some embodiments, the Scale of Social Responsibility (SSR) is utilized to evaluate the safety and/or efficacy of treatment herein. The SSR measures perceptions regarding the importance of ethics and social responsibility.

In some embodiments, the Sheehan Suicidality Tracking Scale (SSTS) is utilized to evaluate the safety and/or efficacy of treatment herein. The SSTS is a 16-item scale that assesses the seriousness of suicidality phenomena on a Likert-type scale (0-4) ranging from “not at all” (0) to “extremely”. The SSTS assesses the frequency of key phenomena and the overall time spent in suicidality.

In some embodiments, the Mini International Neuropsychiatric Interview (MINI) (version 7.0.2) is utilized to evaluate the safety and efficacy of treatment herein. The MINI is a brief structured interview for the major Axis I psychiatric disorders in DSM-5 and International Classification of Diseases-10. In some embodiments, the MINI is used to diagnose a subject with a disorder.

In some embodiments, the McLean Screening Instrument for Borderline Personality Disorder (MSIBPD) is utilized for evaluating the safety and/or efficacy of treatment herein. The MSIBPD is a useful screening tool for identifying the presence of DMS-IV borderline personality disorder.

In some embodiments, the Tellegen Absorption Scale is utilized for evaluating the safety and/or efficacy of treatment herein. The Tellegen Absorption Scale is a 34-item multidimensional measure that assesses imaginative involvement and the tendency to become mentally absorbed in everyday activities.

In some embodiments, the safety and/or efficacy of treatment herein is evaluated by physical examination. A physical examination, includes, but is not limited to, an examination of the subject's general appearance, including an examination of the skin, neck, eyes, ears, nose, throat, heart, lungs, abdomen, lymph nodes, extremities and musculoskeletal system.

In some embodiments, body weight and height of a subject are assessed. In some embodiments, body mass index is used to assess the safety and/or efficacy of treatment herein.

In some embodiments, an electrocardiogram (ECG) is utilized to evaluate the safety and/or efficacy of treatment herein. In some embodiments, a Standard 12-lead ECG is obtained.

In some embodiments, vital signs of a subject are used to evaluate safety and/or efficacy of treatment herein. Vital signs include, but are not limited to, blood pressure (BP), respiratory rate, oral body temperature and pulse. In some embodiments, blood pressure is taken after a subject has been sitting down for at least three minutes.

In some embodiments, clinical laboratory tests are utilized to evaluate the safety and/or efficacy of treatment herein. In some embodiments, the clinical laboratory tests include blood samples and/or urine samples. In some embodiments, hemoglobin, hematocrit, red blood cell count, mean corpuscular hemoglobin, mean corpuscular volume, mean corpuscular hemoglobin concentration, white blood cell count (with differential) and platelet count are measured to evaluate safety and/or efficacy of treatment herein. In some embodiments, albumin, alkaline phosphatase, alanine aminotransferase (ALT), amylase, aspartate aminotransferase (AST), bicarbonate, bilirubin (direct, indirect and total), calcium, chloride, creatine kinase, creatinine, y-glutamyl transferase, glucose, lactate dehydrogenase, lipase, magnesium, phosphate, potassium, protein total, sodium, blood urea nitrogen and/or uric acid are measured to evaluate the safety and/or efficacy of the disclosed treatment method.

In some embodiments, urine is tested for pregnancy and/or illicit drugs.

In some embodiments, the safety and/or efficacy of treatment herein are evaluated by measuring adverse events. Adverse events are classified as mild, moderate, or severe. A mild adverse event does not interfere in a significant manner with the subject's normal level of functioning. A moderate adverse event produces some impairment of functioning, but is not hazardous to the subject's health. A serious adverse event produces significant impairment of functioning or incapacitation and is a definite hazard to the subject's health. Adverse events may include, for example, euphoric mood, dissociative disorder, hallucination, psychotic disorder, cognitive disorder, disturbances in attention, mood alterations, psychomotor skill impairments, inappropriate affects, overdoses, and intentional product misuse. In some embodiments, serious adverse events include death, life-threatening adverse events, inpatient hospitalization or prolongation of existing hospitalization, persistent or significant disability/incapacity, and congenital anomaly/birth defect in the offspring of a subject who received treatment. In some embodiments, serious adverse events include intensive treatment in an emergency room or at home for allergic bronchospasm, blood dyscrasias or convulsions that do not result in inpatient hospitalization, or the development of drug dependency or drug abuse.

Also disclosed herein is a method for decreasing time of therapeutic onset relative to a crystalline psilocybin-based drug, comprising administering a pharmaceutical composition comprising a solid dispersion as disclosed herein to a patient in need thereof. For example, solid dispersions of the present disclosure formulated with a therapeutically effective amount of a compound of Formula (I) in amorphous form dispersed in a polymer may provide a faster therapeutic onset compared to solid dosage forms formulated with a crystalline psilocybin-based drug administered in substantially the same way (e.g., each administered orally).

Also disclosed herein is a method of reducing psychedelic side effects relative to a crystalline psilocybin-based drug, comprising administering a pharmaceutical composition as disclosed herein to a patient in need thereof. For example, solid dispersions of the present disclosure formulated with a therapeutically effective amount of a compound of Formula (I) in amorphous form dispersed in a polymer may provide fewer psychedelic side effects compared to solid dosage forms formulated with a crystalline psilocybin-based drug administered in substantially the same way (e.g., each administered orally).

The terms “hallucinogenic” side effects and “psychedelic side effects” are used in the present disclosure interchangeably to refer to unwanted and/or unintended secondary effects caused by the administration of a medicament to an individual resulting in subjective experiences being qualitatively different from those of ordinary consciousness. These experiences can include derealization, depersonalization, hallucinations and/or sensory distortions in the visual, auditory, olfactory, tactile, proprioceptive and/or interoceptive spheres and/or any other perceptual modifications, and/or any other substantial subjective changes in cognition, memory, emotion and consciousness.

In some embodiments, the administration of pharmaceutical compositions as disclosed herein cause less hallucinogenic and/or psychedelic side effects and/or no hallucinogenic and/or psychedelic side effects relative to a psilocybin-based drug currently available (e.g., crystalline psilocybin-based drugs) or a psilocybin-based drug administered without alkaline phosphatase (ALP). In some embodiments, the administration of pharmaceutical compositions as disclosed herein alleviates, reduces, removes, and/or eliminates the hallucinogenic and/or psychedelic side effects caused by a psilocybin-based drug currently available (e.g., crystalline psilocybin-based drugs) or a psilocybin-based drug administered without ALP. In some embodiments, the administration of the pharmaceutical composition as disclosed herein alleviates, reduces, removes, and/or eliminates any neurologically toxic spikes relative to a psilocybin-based drug currently available (e.g., crystalline psilocybin-based drug) or a psilocybin-based drug administered without ALP.

Neurologically toxic spikes are spikes in the concentration of an active ingredient as described herein that can produce side-effects of sedation or psychotomimetic effects, e.g., hallucination, dizziness, and nausea; which can not only have immediate repercussions, but also effect treatment compliance. In particular, side effects may become more pronounced at blood concentration levels of about 250, 300, 400, 500 ng/L or more.

Also disclosed herein is a method of decreasing duration of therapeutic effect compared to a crystalline psilocybin-based drug, comprising administering the pharmaceutical composition as disclosed herein to a patient in need thereof.

Generally, a duration of therapeutic effect for a psilocybin-based drug currently available (e.g., a crystalline psilocybin-based drug or a psilocybin-based drug administered without ALP), is about 6-8 hours. In some embodiments, the duration of therapeutic effect of the pharmaceutical composition as disclosed herein is less than the duration of therapeutic effect for a crystalline psilocybin-based drug. In some embodiments, the duration of therapeutic effect of the pharmaceutical composition as disclosed herein is 300, 280, 240, 220, 200, 180, 160, 140, 120, 110, 100, 90, 80, 70, 60, 55, 50, 45, 40, 35, 30, 25, 20, 15, 10, 5 minutes or less. In some embodiments, the duration of therapeutic effect of the novel compositions discussed herein is less than the duration of therapeutic effect of current conventional psilocybin-based drugs or current conventional psilocybin-based drugs administered without ALP by 7, 6.5, 6, 5.5, 5, 4.5, 4, 3.5, 3, 2.5, 2, 1.5, or 1 hour or less, or 120, 110, 100, 90, 80, 70, 60, 55, 50, 45, 40, 35, 30, 25, 20, 15, 10, 5 minutes or less.

### EXAMPLES

### I. Compound Synthesis

### Synthesis of 3-(2-(bis(methyl-d₃)amino)ethyl-1,1,2,2-d₄)-1H-indol-4-yl dihydrogen phosphate (I-3)(psilocybin-d₁₀ or PY-d₁₀)

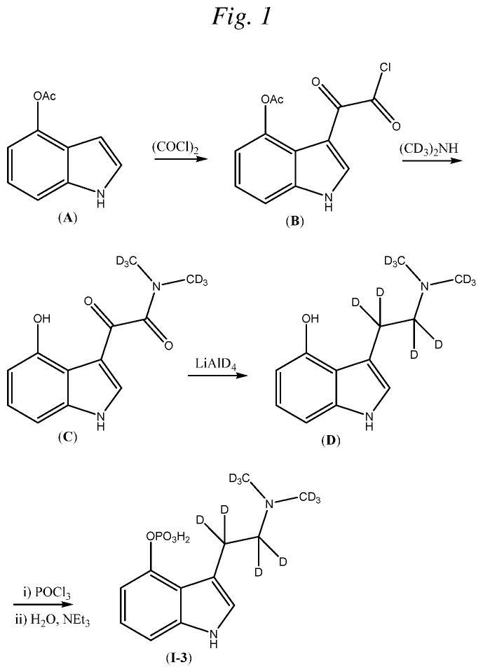

Compound 3-(2-(bis(methyl-d₃)amino)ethyl-1,1,2,2-d₄)-1H-indol-4-yl dihydrogen phosphate (I-3)(psilocybin-d₁₀ or PY-d₁₀) is synthesized according to FIG. 1. 4-acetoxyindole (A) is acylated using oxalyl chloride producing intermediate B as a yellow solid. Treatment of intermediate B with dimethyl-d₆-amine (Cambridge Isotopes Labs, Tewksbury, MA) results in amidation and de-acetylation forming intermediate C, which is then reduced by LiAlD₄ to form psilocin-d₁₀ (D).

Psilocin-d₁₀ (D) is converted into psilocybin-d₁₀ (I-3) following a direct phosphorylation procedure described by Kargbo et al. (Kargbo, Robert B et al. “Direct Phosphorylation of Psilocin Enables Optimized cGMP Kilogram-Scale Manufacture of Psilocybin.” ACS omega vol. 5, 27 16959-16966, 1 Jul. 2020). To a clean, dry reactor under a nitrogen atmosphere THF (28.0 L, 10 vol) and phosphorus oxychloride (3.15 kg, 20.6 mol, 1.5 equiv) are charged at 20 to 25° C. The vessel is cooled to −5 to −15° C. Separately, to a second clean, dry reactor under the nitrogen atmosphere is charged psilocin-d₁₀ (D)(2.80 kg, 13.7 mol, 1 equiv) and celite (2.80 kg, 1 wt) followed by THF (42.0 L, 15 vol). The resultant slurry is held at 18 to 25° C. for at least 2 h. The reactor contents are then cooled to 0 to −15° C. The intermediate D/celite/THF slurry is slowly charged to the POCl₃ solution via pump while maintaining the internal temperature at −15 to 0° C. The mixture is stirred for no more than 2 h at −15 to 0° C.

During this time, to a clean reactor is prepared a quench solution of THF/H₂O (70:30) (28.0 L, 10 vol) and Et₃N (8.32 kg, 82.2 mol, 6 equiv). The vessel containing the quench mixture is cooled to −20 to 0° C., and the crude reaction mixture is slowly added into the THF/H₂O/Et₃N solution, maintaining the internal temperature at −20 to 0° C. THF (2×5.60 L, 2×2 vol) is charged to the reaction mixture reactor, cooled to 0 to −5° C., and used as a rinse into the quench medium, maintaining the internal temperature of the quenched mixture at −20 to 0° C. Purified water (8.40 L, 3 vol) is charged to the reaction mixture reactor, cooled to 2 to 7° C., and used as a rinse into the quench medium, maintaining the internal temperature at −20 to 0° C. The mixture was stirred at −20 to 0° C. for at least 60 min. The mixture was filtered, and the cake was washed with water at 5 to 10° C. (2×5.60 L, 2×2 vol) to dissolve any product stuck to the celite cake. The biphasic filtrate is transferred back to the reactor. A rinse with water (1.40 L, 0.5 vol) can be used as part of the transfer. The temperature is adjusted to 18 to 25° C., and the lower aqueous phase is separated. The organic phase is removed. The lower aqueous phase contains product and the upper organic phase will typically contain residual psilocin-d₁₀ (D). The aqueous phase is transferred back to the reactor. A rinse with water (1.40 L, 0.5 vol) can be used as part of the transfer. Isopropyl alcohol (IPA, 28.0 L, 10 vol) is charged to the aqueous phase. The mixture is concentrated at <45° C. internal temp to ca. 5 vol of the remaining water. Further additions of IPA (14.0 L, 5 vol) or purified water (5.60 L, 2 vol) can be added to aid azeotropic distillation of water. Upon reaching the aqueous distillation, volume purified water (14.0 L, 5 vol) is charged at 18 to 25° C. and the solution is stirred for at least 24 h. Psilocybin-d₁₀ (I-3) will normally precipitate at this time. The reactor contents are filtered under the nitrogen atmosphere and the cake is washed with cold (2 to 6° C.) purified water (2×5.60 L, 2×2 vol) and pulled dry for at least 60 min under the nitrogen atmosphere. The solid is dried at 35 to 45° C. under vacuum for at least 24 h. The crude product is charged to a clean, dry reactor under the nitrogen atmosphere at 20 to 25° C. Methanol (10 vol, based on crude product discharge weight) is charged to the reactor at 20 to 25° C. and the contents are stirred for at least 12 h at 20 to 25° C. The mixture is filtered under nitrogen and the cake rinsed with methanol (2×1.5 vol, based on crude product discharge weight) at 20 to 25° C. The solid is pulled dry for at least 2 h under nitrogen then charged to a clean, dry reactor under the nitrogen atmosphere. Purified water (10 vol, based on crude product discharge weight) is charged to the reactor at 20 to 25° C., and the contents are heated to 45 to 55° C. for at least 24 h. The contents are cooled to 20 to 30° C. at a rate of 10 degrees per hour and held for at least 2 h. The mixture is filtered under the nitrogen atmosphere and washed in turn with 20 to 25° C. purified water (1×1 vol, 1×2 vol) (based on crude product discharge weight) and pulled dry under the nitrogen atmosphere for at least 2 h. The product is initially isolated in trihydrate form A by XRPD. The solid may be dried at 35 to 45° C. under vacuum for at least 24 h and subsequently at 50 to 60° C. (target 55° C.) under vacuum for at least 24 h, to convert the trihydrate form initially isolated to anhydrate form A by XRPD. A white solid is afforded as pure material by ultraperformance liquid chromatography (UPLC). The structure of the final product with deuterium enrichment over 90% will be confirmed by ¹H NMR and LC-MS.

### II. Solid Dispersions Prepared by Freeze-Drying (Lyophilization)

### Materials.

Gelatin (fish gelatin (super fine), available from Ajinomoto, USA) unless specified as bovine gelatin.

Mannitol (Sigma Aldrich).

KOLLIDON® 12PF (polyvinylpyrrolidone (PVP) with a weight average molecular weight of 2,500 g/mol; bulk density of 400-600 g/L; D(0.5) of 35 μm+5 μm, available from BASF).

METHOCEL™ E3 LV (low viscosity hydroxypropyl methyl cellulose (HPMC) with a 2910 substitution type: 28-30% methoxy substitution, 7-12% hydroxypropyl substitution; viscosity of 4.0-6.0 mPa s as 2% solution in water at 20° C., available from DuPont).

METHOCEL™ E6 premium LV (low viscosity hydroxypropyl methyl cellulose (HPMC) with a molecular weight of 70,000-80,000 g/mol, 2910 substitution type: 28-30% methoxy substitution, 7-12% hydroxypropyl substitution; viscosity of 4.8-7.2 mPa s as 2% solution in water at 20° C., available from DuPont).

KOLLIDON® VA 64 (a 60:40 copolymer of VP:VAc, 45,000-75,000 g/mol, available from BASF).

METHOCEL™ K100LV (hydroxypropyl methyl cellulose (HPMC) with a molecular weight of 164,000 g/mol, available from Colorcon, Inc.).

METHOCEL™ K4M (hydroxypropyl methyl cellulose (HPMC) with a molecular weight of 400,000 g/mol, available from Colorcon, Inc.).

METHOCEL™ K15M (hydroxypropyl methyl cellulose (HPMC) with a molecular weight of 575,000 g/mol, available from Colorcon, Inc.).

AQUASOLVE™ HPMCAS MF (HPMCAS polymer with a substitution pattern of 7-11% acetyl, 10-14% succinoyl, 21-25% methoxyl, 5-9% hydroxypropoxy; viscosity of 2.4-3.6 mPa s as 2% solution in water at 20° C.; less than 10 μm mean particle size, available from Ashland).

BENECEL™ K35M Pharm (hydroxypropyl methyl cellulose (HPMC) with a 2208 substitution type; 675,000 g/mol; available from Ashland).

BENECEL™ K100LV PH PRM (hydroxypropyl methyl cellulose (HPMC) with a 2208 substitution type; 164,000 g/mol; available from Ashland).

Psilocybin (3-(2-(dimethylamino)ethyl)-1H-indol-4-yl dihydrogen phosphate; PY; I-7) starting material used was in crystalline form as crystalline methanol solvate with a small quantity of crystalline Form B as described below, commercially available from Quality Chemical Labs.

### Analytical Methods.

### X-Ray Power Diffraction (XRPD):

The samples were prepared in silicon low background holders using light manual pressure to keep the sample surface flat and level with the reference surface of the sample holder. The single crystal Si low background holder has a circular recess (10 mm diameter and about 0.2 mm depth) that holds the sample.

The Rigaku Smart-Lab diffraction system used was configured for Bragg-Brentano reflection geometry using a line source X-ray beam. The Bragg-Brentano geometry was controlled by passive divergence and receiving slits with the sample itself acting as the focusing component for the optics. The figures were created using PlotMon V2.1.1.0. The XRPD parameters that were used are summarized in Table 2.

TABLE 2

| Parameter | Value | Parameter | Value |
|---|---|---|---|
| Geometry | Bragg- | Receiving Slit 1 | 18 |
| | Brentano | (mm) | |
| Tube Anode | Cu Kα | Receiving Slit 2 | open |
| | | (mm) | |
| Tube Type | Long Fine | Start Angle (°2θ) | 2 |
| | Focus | | |
| Tube Voltage | 40 | End Angle (°2θ) | 40 |
| (kV) | | | |
| Tube Current (mA) | 44 | Step Size (°2θ) | 0.02 |
| Detector | D/teX Ultra 250 | Scan Speed | 6 |
| | | (°2θ/min) | |
| Monochromatization | Kβ Filter | Spinning (rpm) | 11 |
| Incident Slit (°) | ⅓ | Sample Holder | Large well |
| | | | silicon low |
| | | | background |
| | | | holder |

### High-Resolution X-Ray Power Diffraction (XRPD):

High-resolution XRPD analysis was performed using Rigaku Smart-Lab diffraction system configured for Debye-Scherrer transmission geometry. The Debye-Scherrer convergent beam geometry utilizes a curved x-ray mirror to focus the incident beam through the sample and onto the detector plane. The axial divergence of the X-ray beam was controlled by 5.0° Soller slits in both the incident and diffracted beam paths. The high-resolution XRPD parameters that were used are summarized in Table 3.

TABLE 3

| Parameter | Value | Parameter | Value |
|---|---|---|---|
| Geometry | Transmission | Receiving Slit 1 | 18 |
| | | (mm) | |
| Tube Anode | Cu Kα | Receiving Slit 2 | 19.125 |
| | | (mm) | |
| Tube Type | Long Fine Focus | Start Angle (°2θ) | 5 |
| Tube Voltage | 40 | End Angle (°2θ) | 30 |
| (kV) | | | |
| Tube Current (mA) | 44 | Step Size (°2θ) | 0.02 |
| Detector | D/teX Ultra 250 | Scan Speed | 0.5 |
| | | (°2θ/min) | |
| Monochromatization | Kβ Filter | Spinning (rpm) | 12 |
| Incident Slit (mm) | 1.0 | Sample Holder | Etnom ® |
| | | | films |

Modulated Differential Scanning Calorimetry (mDSC):

The mDSC analyses were carried out using a TA Instruments Q2000 instrument. The instrument temperature calibration was performed using indium. For each analysis, approximately 1 to 3 mg of the sample were weighed into a Tzero aluminum pan that was covered with a lid, crimped, and loaded into the DSC instrument. An empty pan of the same configuration was loaded into the reference position. Each sample was heated from 25° C. to 220° C. or 250° C. at a rate of 2° C. per minute with a ±0.42° C. modulation every 40 seconds. The DSC cell was kept under a nitrogen purge of about 50 mL per minute during each analysis. Data collection was performed using Thermal Advantage 5.5.3 software. Data collection and analysis was performed using Trios v5.0.0.44608.

Each mDSC plot shows three heat flows: total heat flow (*), reversing heat flow (**) and non-reversing heat flow (***). In typical mDSC data, reversing heat flow shows glass transition, heat capacity change and melting events whereas the non-reserving heat flow shows enthalpic recovery, evaporation, crystallization, decomposition (including chemical reactions). Some melting events may also appear in the non-reversing heat flow curve.

Freeze-dried solid dispersions and experimental procedures. Freeze-dried solid dispersions containing psilocybin (PY; I-7) in various polymer matrices were prepared as outlined below.

### Example 1

Freeze-dried tablets (50 mg) were prepared with psilocybin (PY; I-7) (26.67 wt. %) according to composition described in Table 4. Gelatin was dissolved in water and incubated at 60° C. for 30 min until a clear solution was obtained. Crystalline psilocybin was added to the gelatin solution, vortexed vigorously, and was briefly incubated at 60° C. to ensure complete solubility of the drug. The aqueous formulation of gelatin/PY was flash frozen in liquid nitrogen (−196° C.), stored at −15° C. for 12 hours, and lyophilized at 0° C. for 12 hours under vacuum.

Reference Example 1a (placebo, matrix only) and Reference Example 1b (prepared by physical mixing (admixture of) PY+matrix) were also prepared and characterized for reference.

TABLE 4

| | | Reference | Reference |
|---|---|---|---|
| | Example 1 | Example 1a | Example 1b* |
| Matrix composition | wt. (g) | wt. (g) | wt. (g) |
| Gelatin | 0.0275 | 0.0275 | 0.0275 |
| Psilocybin | 0.01 | — | 0.01 |
| Water | 0.465 | 0.465 | 0.465 |
| Total Weight (g) | 0.5025 | 0.4925 | 0.5025 |
| Dry Weight (g) | 0.0375 | 0.0275 | 0.0375 |
| PY (g)/tablet | 0.01 | N/A | 0.01 |
| PY (wt. %) | 26.67 | N/A | 26.67 |
| in dry 50 mg tablet | | | |

*Physical mixture (admixture)

### Example 2

Freeze-dried tablets (50 mg) were prepared with psilocybin (PY; I-7) (26.67 wt. %) according to composition described in Table 5. PVP (KOLLIDON® 12PF) was dissolved in water and incubated at 60° C. for 30 to 60 min until a clear solution was obtained. Crystalline psilocybin was dissolved in water and was incubated at 60° C. and vortexed vigorously. Both solutions were mixed, and the resulting aqueous formulation of PVP/PY was flash frozen in liquid nitrogen (−196° C.), stored at −15° C. for 12 hours, and lyophilized at 0° C. for 12 hours under vacuum.

Reference Example 2a (placebo, matrix only) and Reference Example 2b (prepared by physical mixing (admixture of) PY+matrix) were also prepared and characterized for reference.

TABLE 5

| | | Reference | Reference |
|---|---|---|---|
| | Example 2 | Example 2a | Example 2b* |
| Matrix composition | wt. (g) | wt. (g) | wt. (g) |
| KOLLIDON ® 12PF | 0.0275 | 0.0275 | 0.0275 |
| Psilocybin | 0.01 | — | 0.01 |
| Water | 0.465 | 0.465 | 0.465 |
| Total Weight (g) | 0.5025 | 0.492 | 0.5025 |
| Dry Weight (g) | 0.0375 | 0.0275 | 0.0375 |
| PY (g)/tablet | 0.01 | N/A | 0.01 |
| PY (wt. %) | 26.67 | N/A | 26.67 |
| in dry 50 mg tablet | | | |

*Physical mixture (admixture)

### Example 3

Freeze-dried tablets (50 mg) were prepared with psilocybin (PY; I-7) (26.67 wt. %) according to composition described in Table 6. HPMC (METHOCEL™ E3 LV) was dissolved in water and incubated at 60° C. for 30 to 60 min until a clear solution was obtained. Crystalline psilocybin was dissolved in water and was incubated at 60° C. and vortexed vigorously. Both solutions were mixed, and the resulting aqueous formulation of HPMC/PY was flash frozen in liquid nitrogen (−196° C.), stored at −15° C. for 12 hours, and lyophilized at 0° C. for 12 hours under vacuum.

Reference Example 3a (placebo, matrix only) and Reference Example 3b (prepared by physical mixing (admixture of) PY+matrix) were also prepared and characterized for reference.

TABLE 6

| | | Reference | Reference |
|---|---|---|---|
| | Example 3 | Example 3a | Example 3b* |
| Matrix composition | wt. (g) | wt. (g) | wt. (g) |
| METHOCEL ™ E3 LV | 0.0275 | 0.0275 | 0.0275 |
| Psilocybin | 0.01 | — | 0.01 |
| Water | 0.465 | 0.465 | 0.465 |
| Total Weight (g) | 0.5025 | 0.492 | 0.5025 |
| Dry Weight (g) | 0.0375 | 0.0275 | 0.0375 |
| PY (g)/tablet | 0.01 | N/A | 0.01 |
| PY (wt. %) | 26.67 | N/A | 26.67 |
| in dry 50 mg tablet | | | |

*Physical mixture (admixture)

### Example 4

Freeze-dried tablets (50 mg) were prepared with psilocybin (PY; I-7) (15.4 wt. %) according to composition described in Table 7. HPMC (METHOCEL™ E3 LV) and PVP (KOLLIDON® 12PF) were dissolved in water and incubated at 60° C. for 30 to 60 min until a clear solution was obtained. Crystalline psilocybin was dissolved in water and was incubated at 60° C. and vortexed vigorously. Both solutions were mixed, and the resulting aqueous formulation of HPMC/PVP/PY was flash frozen in liquid nitrogen (−196° C.), stored at −15° C. for 12 hours, and lyophilized at 0° C. for 12 hours under vacuum.

Reference Example 4a (placebo, matrix only) and Reference Example 4b (prepared by physical mixing (admixture of) PY+matrix) were also prepared and characterized for reference.

TABLE 7

| | | Reference | Reference |
|---|---|---|---|
| | Example 4 | Example 4a | Example 4b* |
| Matrix composition | wt. (g) | wt. (g) | wt. (g) |
| KOLLIDON ® 12PF | 0.0275 | 0.0275 | 0.0275 |
| METHOCEL ™ E3 LV | 0.0275 | 0.0275 | 0.0275 |
| Psilocybin | 0.01 | — | 0.01 |
| Water | 0.435 | 0.435 | 0.435 |
| Total Weight (g) | 0.50 | 0.49 | 0.50 |
| Dry Weight (g) | 0.065 | 0.055 | 0.065 |
| PY (g)/tablet | 0.01 | N/A | 0.01 |
| PY (wt. %) | 15.4 | N/A | 15.4 |
| in dry 50 mg tablet | | | |

*Physical mixture (admixture)

### Example 5

Freeze-dried tablets (50 mg) were prepared with psilocybin (PY; I-7) (13.0 wt. %) according to composition described in Table 8. Gelatin and mannitol were dissolved in water and incubated at 60° C. for 30 to 60 min until a clear solution was obtained. Crystalline psilocybin was added to the gelatin/mannitol solution, which was vortexed vigorously, followed by pH modification using sodium hydroxide solution (7.5% w/w). The aqueous formulation of gelatin/mannitol/NaOH/PY was flash frozen in liquid nitrogen (−196° C.), stored at −15° C. for 12 hours, and lyophilized at 0° C. for 12 hours under vacuum.

Reference Example 5a (placebo, matrix/excipients only) and Reference Example 5b (prepared by physical mixing (admixture of) PY+matrix/excipients) were also prepared and characterized for reference.

TABLE 8

| | | Reference | Reference |
|---|---|---|---|
| Matrix | Example 5 | Example 5a | Example 5b* |
| composition | wt. (g) | wt. (g) | wt. (g) |
| Gelatin | 0.0275 | 0.0275 | 0.0275 |
| NaOH | 0.0175 | 0.0175 | 0.0175 |
| Mannitol | 0.022 | 0.022 | 0.022 |
| Psilocybin | 0.01 | — | 0.01 |
| Water | 0.423 | 0.433 | 0.423 |
| Total Weight (g) | 0.50 | 0.50 | 0.50 |
| Dry Weight (g) | 0.077 | 0.067 | 0.077 |
| PY (g)/tablet | 0.01 | N/A | 0.01 |
| PY (wt. %) in dry | 13.0 | N/A | 13.0 |
| 50 mg tablet | | | |

*Physical mixture (admixture)

### Example 6

Orally disintegrating tablets (ODT), also known as fast dissolving tablets (FDTs), were prepared according to the formulation of Example 5 in Table 8, but using the following procedure: A mixture of gelatin and mannitol was prepared in water and the solution was heated to 60° C. for 10 min. The solution was cooled to 12° C. followed by addition of crystalline psilocybin. The pH was modified using sodium hydroxide solution (7.5% w/w). The resulting aqueous formulation was dosed into blister pockets in an amount which provides 5 mg of psilocybin per tablet, and subjected to lyophilization by freezing at −90° C. for 4 minutes. The frozen product was placed in a freezer (−14° C.) for storage for ≥12 hours, and then dried in a freeze dryer under vacuum at a shelf temperature of 0° C. for 12 hours.

### Examples 7-8

Freeze-dried tablets (50 mg) were prepared with psilocybin (PY; I-7) (26.67 wt. %) according to composition described in Table 9. KOLLIDON® VA 64 (Example 7) or METHOCEL™ E6 premium LV (Example 8) were dissolved in water and incubated at 60° C. for 30 to 60 min until a clear solution was obtained. Crystalline psilocybin was dissolved in water and was incubated at 60° C. and vortexed vigorously. Both solutions were mixed, and the resulting aqueous formulation of polymer/PY was flash frozen in liquid nitrogen (−196° C.), stored at −15° C. for 12 hours, and lyophilized at 0° C. for 12 hours under vacuum.

TABLE 9

| | Example 7 | Example 8 |
|---|---|---|
| Matrix composition | wt. (g) | wt. (g) |
| KOLLIDON ® VA 64 | 0.0275 | — |
| METHOCEL ™ E6 premium LV | — | 0.0275 |
| Psilocybin | 0.01 | 0.01 |
| Water | 0.462 | 0.462 |
| Total Weight (g) | 0.4995 | 0.4995 |
| Dry Weight (g) | 0.0375 | 0.0375 |
| PY (g)/tablet | 0.01 | 0.01 |
| PY (wt. %) in dry 50 mg tablet | 26.67 | 26.67 |

### Examples 9-14

Freeze-dried tablets (50 mg) were prepared with psilocybin (PY; I-7) (26.67 wt. %) according to composition described in Table 10. METHOCEL™ K100LV (Example 9), METHOCEL™ K4M (Example 10), METHOCEL™ K15M (Example 11), AQUASOLVE™ HPMCAS MF (Example 12), BENECEL™ K35M Pharm (Example 13), or BENECEL™ K100LV PH PRM (Example 14) were dissolved in water and incubated at 60° C. for 30 to 60 min until a clear solution was obtained. Crystalline psilocybin was dissolved in water and was incubated at 60° C. and vortexed vigorously. Both solutions were mixed, and the resulting aqueous formulation of polymer/PY was flash frozen in liquid nitrogen (−196° C.), stored at −15° C. for 12 hours, and lyophilized at 0° C. for 12 hours under vacuum.

TABLE 10

| | Example 9 | Example 10 | Example 11 | Example 12 | Example 13 | Example 14 |
|---|---|---|---|---|---|---|
| Matrix composition | wt. (g) | wt. (g) | wt. (g) | wt. (g) | wt. (g) | wt. (g) |
| METHOCEL ™ K100LV | 0.0275 | — | — | — | — | — |
| METHOCEL ™ K4M | — | 0.0275 | — | — | — | — |
| METHOCEL ™ K15M | — | — | 0.0275 | — | — | — |
| AQUASOLVE ™ HPMCAS MF | — | — | — | 0.0275 | — | — |
| BENECEL ™ K35M | — | — | — | — | 0.0275 | — |
| BENECEL ™ K100LV PH PRM | — | — | — | — | — | 0.0275 |
| Psilocybin | 0.01 | 0.01 | 0.01 | 0.01 | 0.01 | 0.01 |
| Water | 0.465 | 0.465 | 0.465 | 0.465 | 0.465 | 0.465 |
| Total Weight (g) | 0.5025 | 0.5025 | 0.5025 | 0.5025 | 0.5025 | 0.5025 |
| Dry Weight (g) | 0.0375 | 0.0375 | 0.0375 | 0.0375 | 0.0375 | 0.0375 |
| PY (g)/tablet | 0.01 | 0.01 | 0.01 | 0.01 | 0.01 | 0.01 |
| PY (wt. %) | 26.67 | 26.67 | 26.67 | 26.67 | 26.67 | 26.67 |
| in dry 50 mg tablet | | | | | | |

### Example 15

Freeze dried wafers are prepared by dissolving crystalline psilocybin (20 mg) in 1 ml of a solution of 0.1 M phosphate buffer with 5 wt. % bovine gelatin and 4 wt. % mannitol, and adjusting the pH to 7.0 with sodium hydroxide solution (7.5% w/w). The aqueous formulation is transferred into a thin layer into wells, flash frozen in liquid nitrogen (−196° C.), stored at −15° C. for 12 hours, and lyophilized at 0° C. for 12 hours under vacuum. After lyophilization, the occurrence of the amorphous form of PY is confirmed by DSC, TGA and X-ray powder diffraction. The material, in the form of a wafer, will be stored at 4° C. and the stability pulls will be conducted at T=1, 3, 6, and 12 months confirming the presence of the amorphous form of PY.

The wafer displays good properties as an orally disintegrating wafer dosage form with a disintegration time of 25 s and very rapid dissolution kinetics, characterized by a dissolution time of 2 min.

### Characterization/Results.

Freeze-dried solid dispersions of Examples 1-5, and corresponding Reference Examples, were characterized using X-ray powder diffraction (XRPD). The results are summarized in Table 11.

TABLE 11

| Example No. | Descriptor | XRPD Results |
|---|---|---|
| Crystalline PY | commercially available | crystalline PY (methanol solvate)^{a} |
| starting material | from Quality Chemical | may contain small amount of Form B^{b} |
| | Labs | |
| Example 1 | freeze-dried gelatin/PY | amorphous PY |
| Reference | placebo | amorphous |
| Example 1a | | |
| Reference | physical mixture (admix) | crystalline PY (methanol solvate)^{a} present |
| Example 1b | | |
| Example 2 | freeze-dried PVP/PY | crystalline PY Form A^{c} present |
| Reference | placebo | amorphous |
| Example 2a | | |
| Reference | physical mixture (admix) | crystalline PY (methanol solvate)^{a} present |
| Example 2b | | |
| Example 3 | freeze-dried HPMC/PY | amorphous PY |
| Reference | placebo | amorphous |
| Example 3a | | |
| Reference | physical mixture (admix) | crystalline PY (methanol solvate)^{a} present |
| Example 3b | | |
| Example 4 | freeze-dried | amorphous PY |
| | HPMC/PVP/PY | |
| Reference | placebo | amorphous |
| Example 4a | | |
| Reference | physical mixture (admix) | crystalline PY (methanol solvate)^{a} present |
| Example 4b | | |
| Example 5 | freeze-dried | amorphous PY + |
| | gelatin/mannitol/NaOH/PY | crystalline mannitol (hemihydrate + delta) |
| Reference | placebo | crystalline mannitol (hemihydrate + delta) |
| Example 5a | | |
| Reference | physical mixture (admix) | crystalline PY (methanol solvate)^{a} + mannitol |
| Example 5b | | (hemihydrate + delta) |

^{a}CSD Reference Code PSILOC
^{b}CSD Reference Code TAVZID01
^{c}CSD Reference Code HATCAK & TAVZID

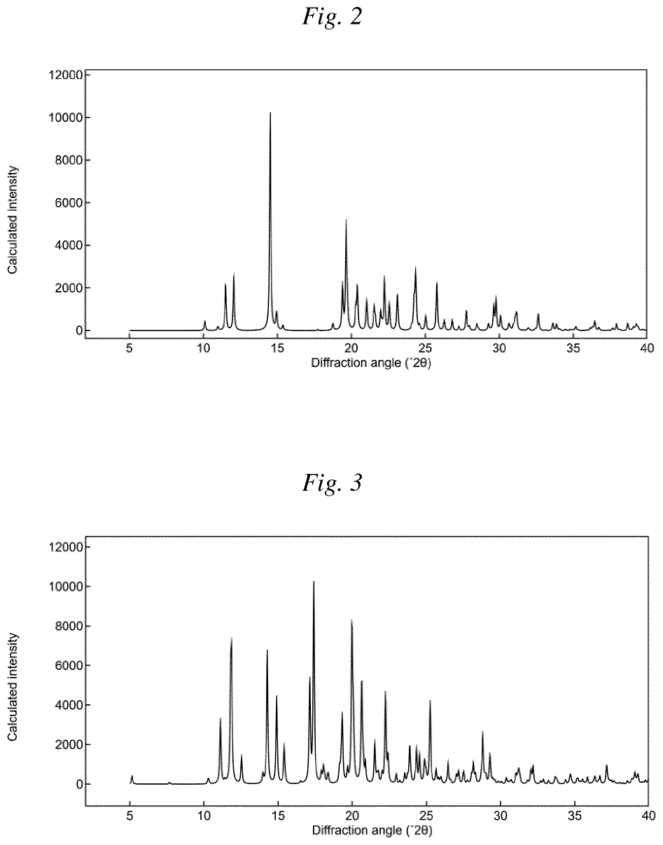

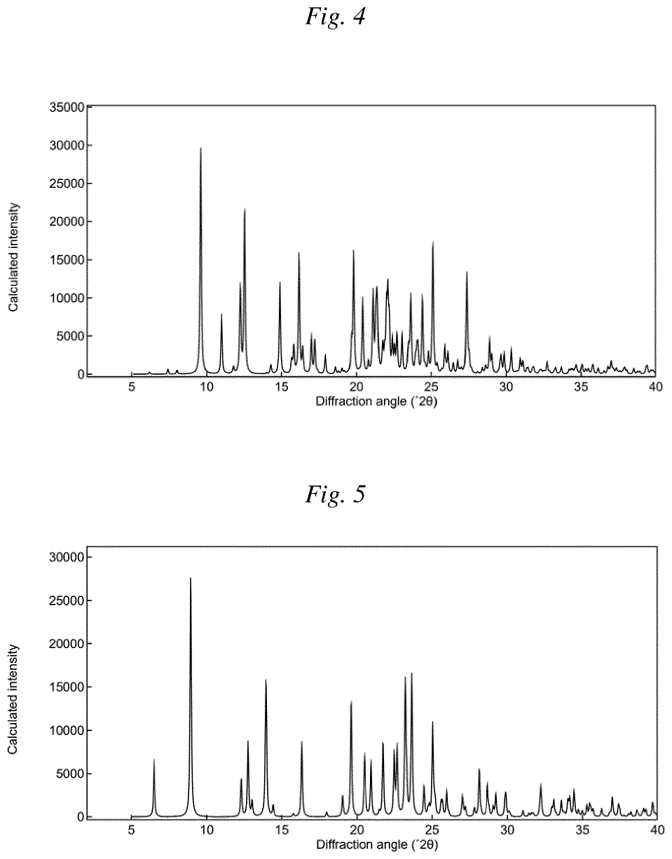

A total of five crystalline forms of psilocybin (PY; I-7) have been reported in the literature (with Cambridge structural database (CSD) Reference Codes identified in parentheses): Form A (HATCAK & TAVZID), Form B (TAVZID01), methanol solvate (PSILOC), trihydrate (OKOKAD), and ethanol solvate (KOWHOT), see Sherwood et al., “Psilocybin: crystal structure solutions enable phase analysis of prior art and recently patented examples”, Acta Cryst. (2022), C78 (1), 36-55. The XRPD calculated from the CSD for Form A (HATCAK & TAVZID), Form B (TAVZIDO1), methanol solvate (PSILOC), and trihydrate (OKOKAD) are presented in FIGS. 2-5, respectively.

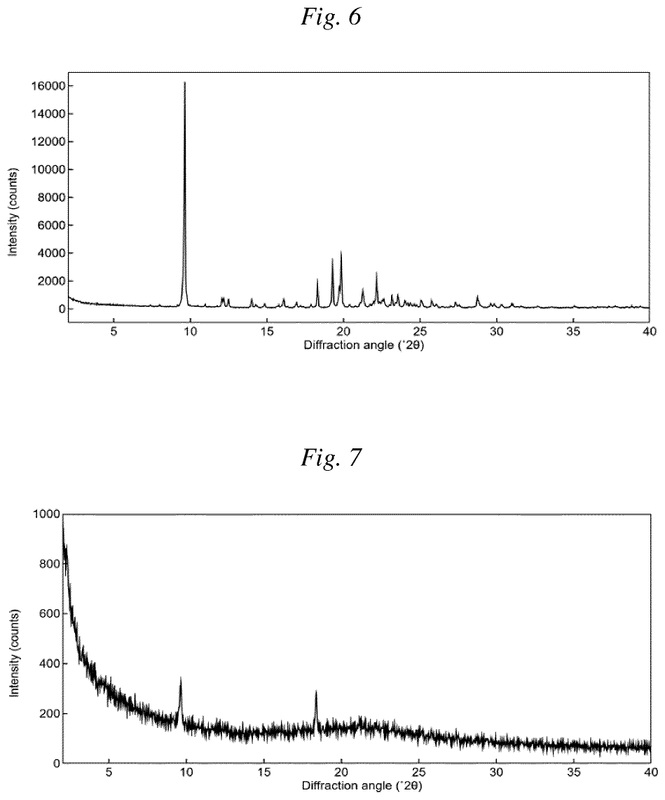

A comparison of XRPD results with the above reference psilocybin data from the Cambridge Structure Database (CSD) show that the crystalline psilocybin starting material used in these studies (commercially available from Quality Chemical Labs) is crystalline psilocybin methanol solvate with a small quantity of Form B (FIG. 6). Some preferred orientation is present and some of the intensities do not match the reference.

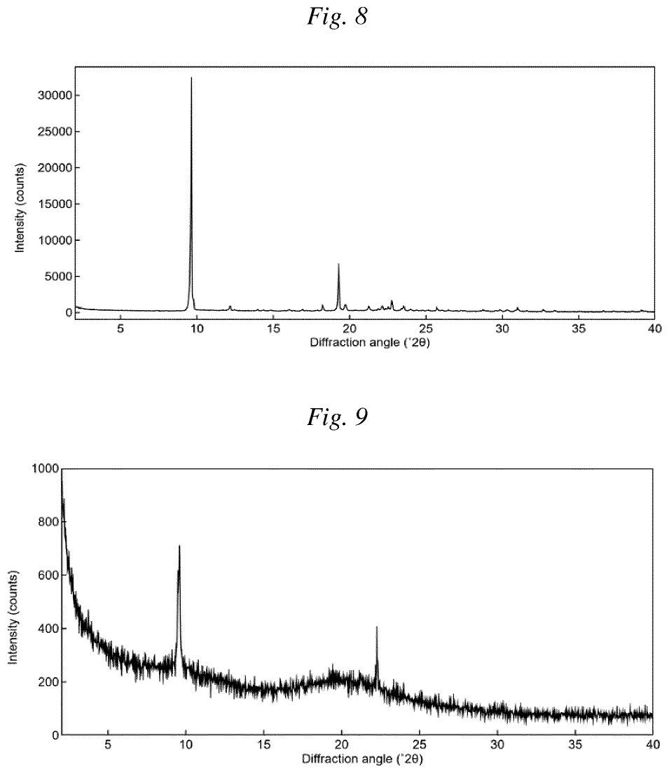

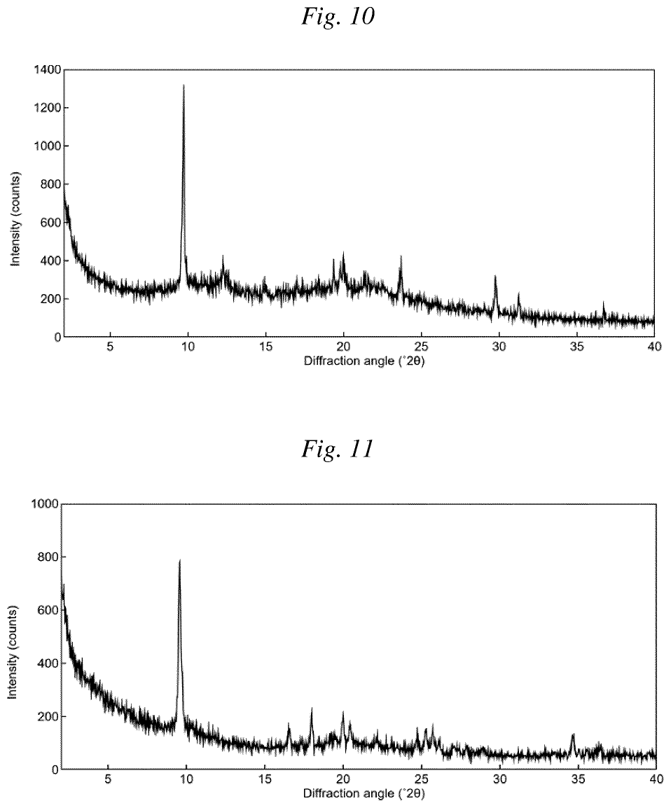

XRPD data showed that Reference Examples 1b, 2b, 3b, 4b, and 5b (physical mixtures (admixtures)) contain small amounts of crystalline psilocybin methanol solvate (FIGS. 7-11, respectively). Based on the peak broadening of crystalline psilocybin, psilocybin methanol solvate may be losing crystallinity upon mechanical mixing applied during the mixture preparations. XRPD data for Reference Example 5b also showed crystalline mannitol (mannitol hemihydrate and mannitol delta form) as shown in FIG. 11.

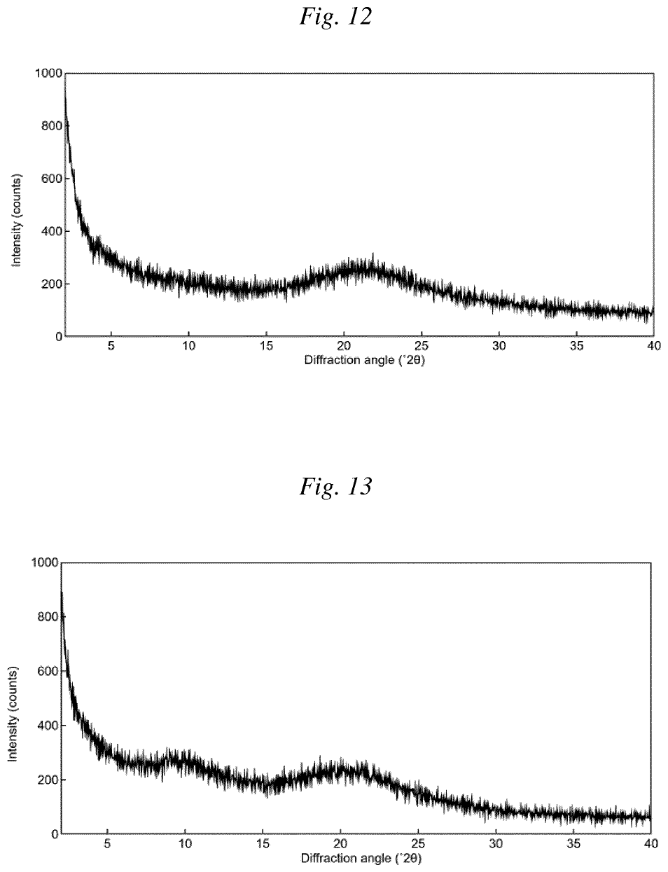

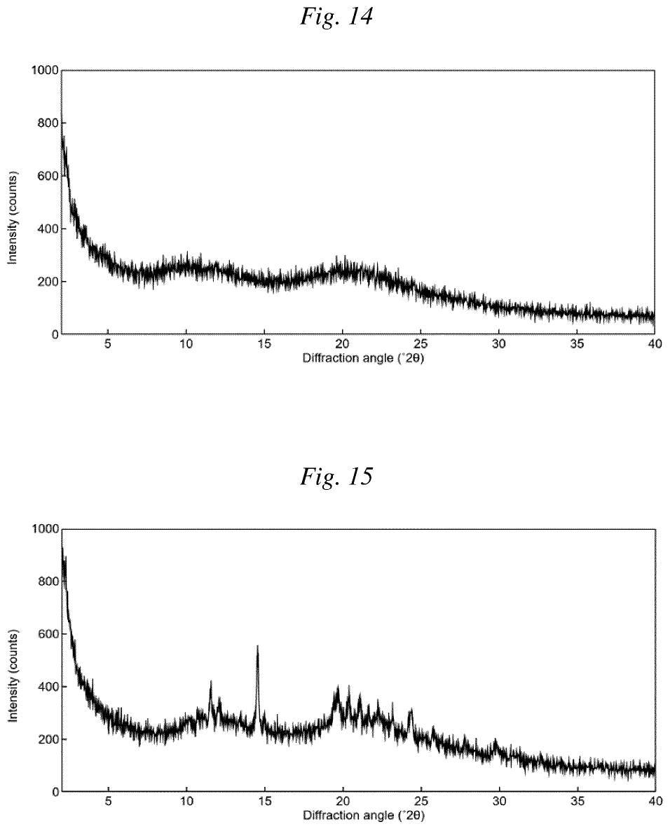

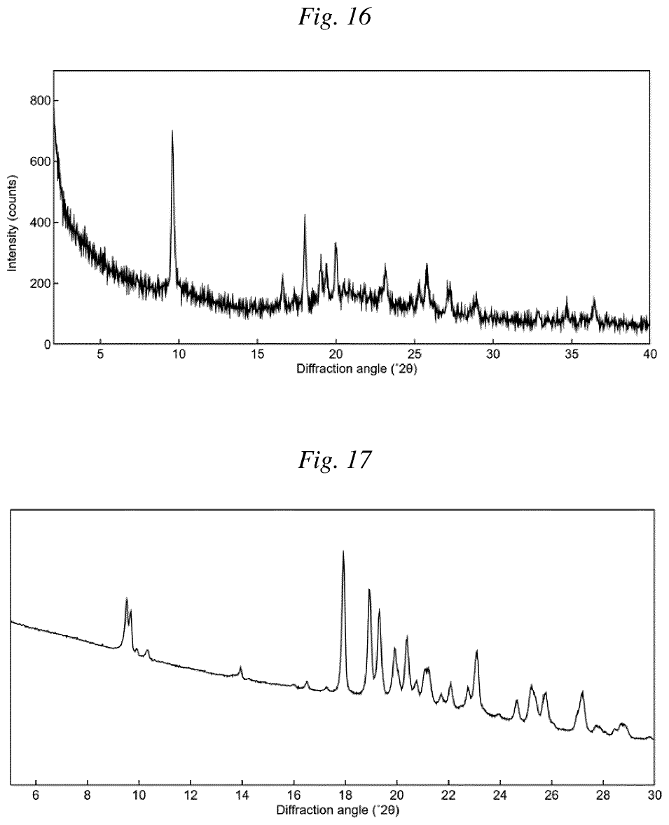

XRPD data for Examples 1, 3, and 4 indicated these solid dispersions were amorphous with no visible crystalline psilocybin peaks (FIGS. 12-14, respectively). To the contrary, XRPD of Example 2 showed peaks matching psilocybin Form A (FIG. 15), indicating that PVP alone was not sufficient for stabilizing amorphous psilocybin. XRPD pattern of Example 5 showed only crystalline mannitol (mannitol hemihydrate and mannitol delta form) as shown in FIG. 16.

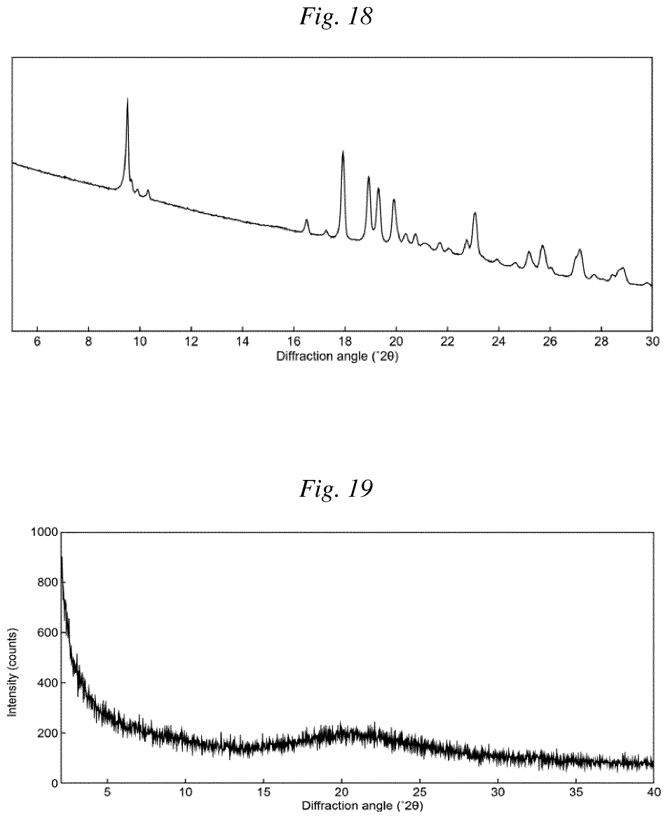

To improve the signal to noise and to attempt to detect even small levels of crystalline psilocybin present in freeze-dried formulations, high-resolution XRPD analysis was performed in transmission configuration on Reference Example 5b and Example 5. The XRPD data showed that the physical mixture (Reference Example 5b) contained crystalline psilocybin methanol solvate (FIG. 17), whereas the freeze-dried product (Example 5) did not show signals corresponding to crystalline psilocybin, only those of amorphous psilocybin (FIG. 18). XRPD patterns of both samples showed crystalline excipients consisting of two crystalline forms of mannitol (hemihydrate and delta forms). It is noted that the crystalline mannitol signals overlap with some of the intense signals of crystalline psilocybin, which somewhat complicated the analysis.

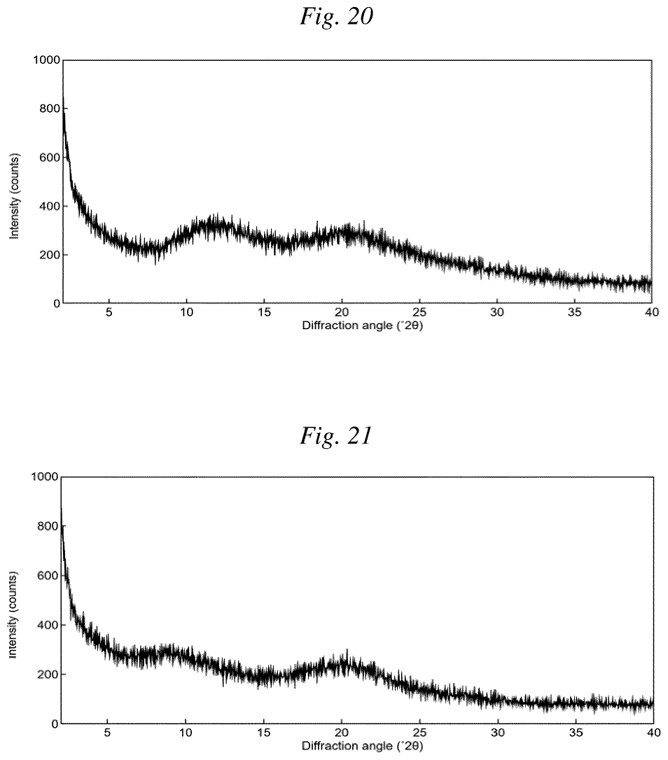

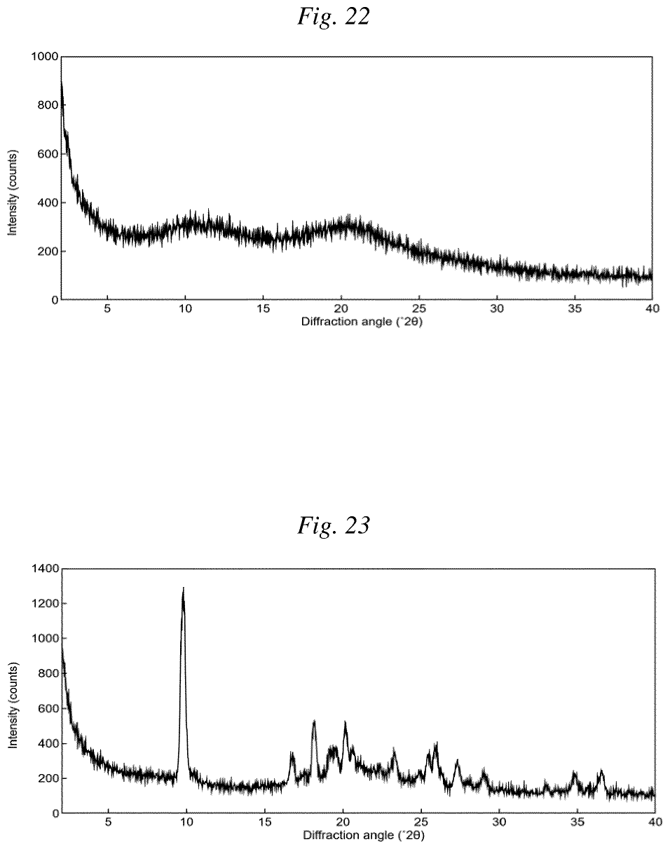

For comparison, XRPD was also performed on Reference Examples 1a-5a (placebo products), which are shown in FIGS. 19-23, respectively. All placebo products showed only amorphous material, except for Reference Example 5b, which showed crystalline mannitol, again in both hemihydrate and delta forms (FIG. 23).

In summary, XRPD analysis confirmed that psilocybin starting material used in these studies is a crystalline methanol solvate. XRPD analysis detected psilocybin crystalline Form A in Example 2, however no other crystalline psilocybin products were detected in Examples 1 and 3-5—these examples are characterized as amorphous solid dispersions.

Crystalline psilocybin starting material and freeze-dried solid dispersions of Examples 1 and 3-5 were also analyzed by mDSC, and the results are summarized in Table 12.

TABLE 12

| Example No. | Descriptor | mDSC Results |
|---|---|---|
| Crystalline PY | commercially available | Apparent melt onset (methanol solvate) |
| starting material | from Quality Chemical Labs | 129° C. |
| Example 1 | freeze-dried gelatin/PY | Tg onset 183° C., Tg midpoint 187° C., ΔCp |
| | | 0.44 J/(g · ° C.) |
| Example 3 | freeze-dried HPMC/PY | Tg onset 144° C., Tg midpoint 149° C., ΔCp |
| | | 0.26 J/(g · ° C.) |
| Example 4 | freeze-dried | Tg onset 119° C., Tg midpoint 127° C., ΔCp |
| | HPMC/PVP/PY | 0.16 J/(g · ° C.) |
| Example 5 | freeze-dried | Tg onset 126° C., Tg midpoint 129° C., ΔCp |
| | gelatin/mannitol/NaOH/PY | 0.73 J/(g · ° C.) |

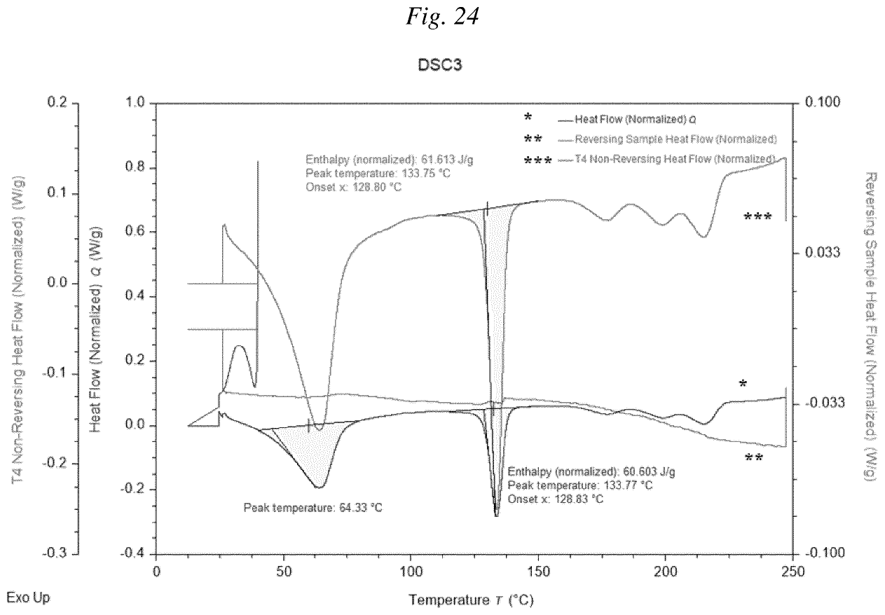

Crystalline psilocybin starting material showed a loss of the solvent near 65° C. and an apparent melting at 129° C. (onset temperature) (FIG. 24). Melting point of crystalline psilocybin Form A was reported at 212° C., with a small endothermic transition at 149° C. Tg of amorphous psilocybin has been reported at 163° C. (midpoint). See Greenan et al., Preparation and Characterization of Novel Crystalline solvates and Polymorphs of Psilocybin and Identification of Solid Forms Suitable for Clinical Development, 2020 pre-publication; DOI:10.13140/RG.2.2.32357.14560.

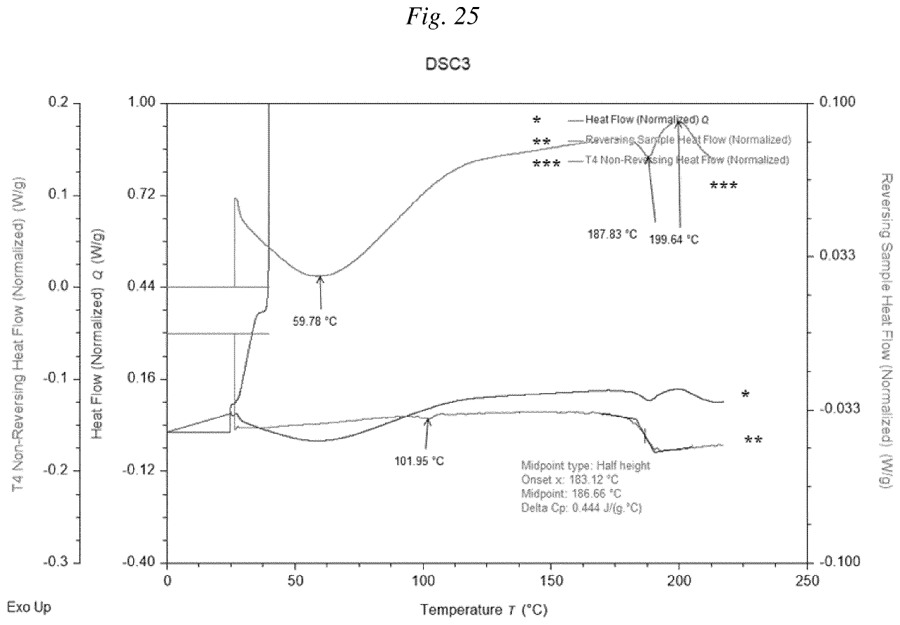

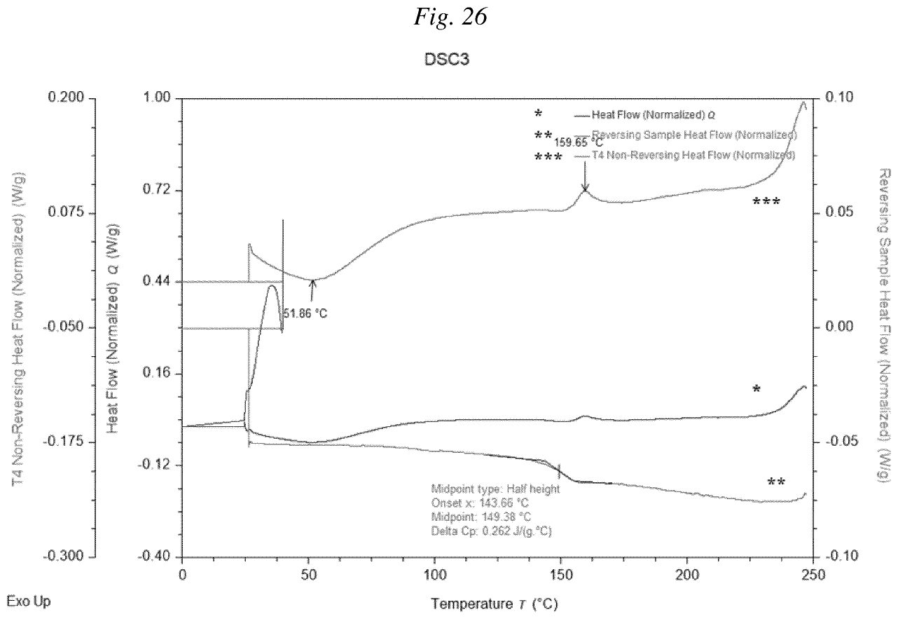

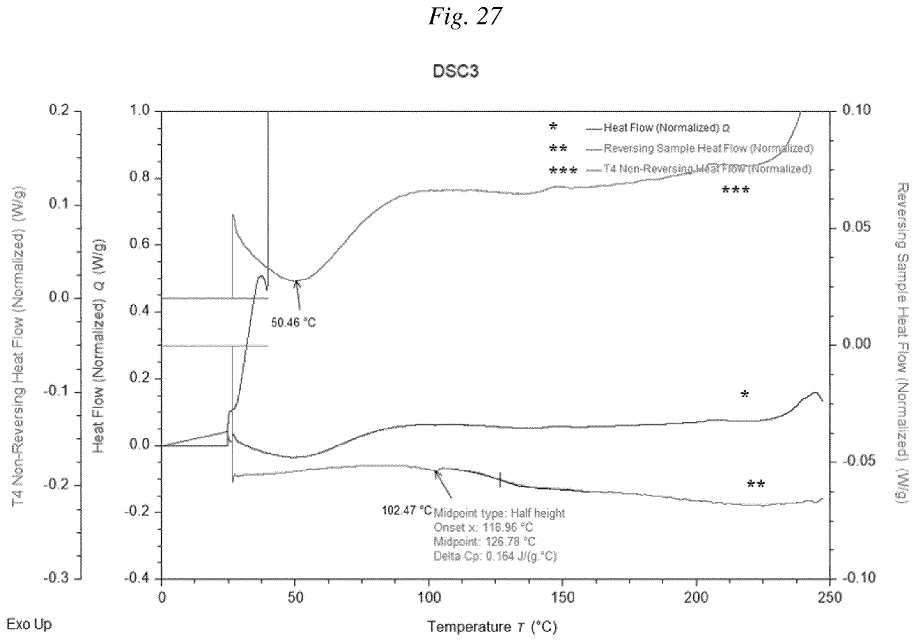

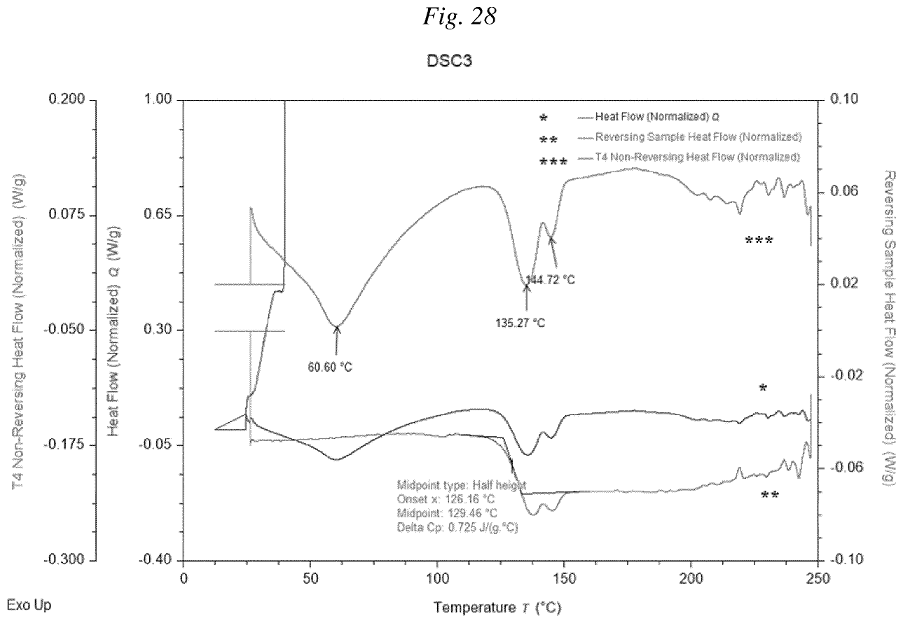

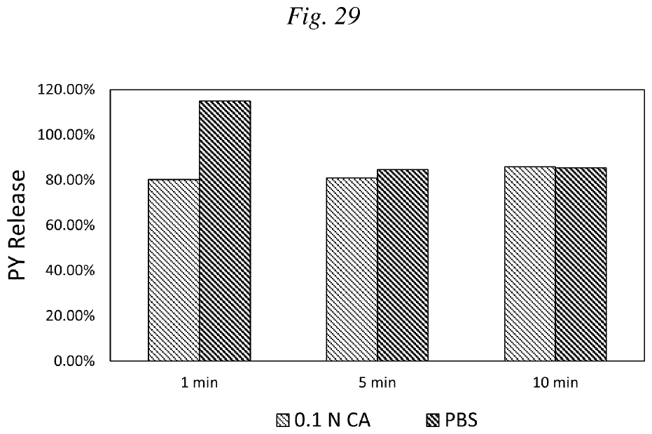

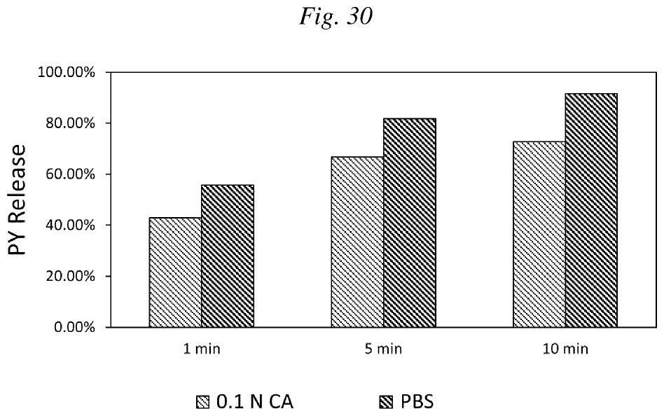

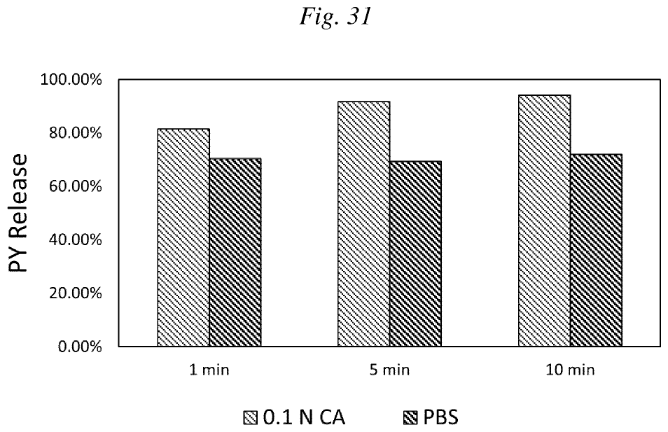

Examples 1 and 3-5 all showed broad endotherms between 50-60° C. These broad endotherms are most likely due to volatile loss. Further, these products also showed glass transition (Tg) events, ranging between 119 and 183° C. as shown on the reversing heat flow in the mDSC data (FIGS. 25-28, respectively), and also summarized in Table 12).

Example 1 exhibited the highest Tg at 183° C. (onset temperature) with ΔCp (heat capacity change) of 0.44 J/(g·° C.). Typical ΔCp for amorphous material is 0.5 J/(g·° C.). The high Tg and the value of ΔCp for Example 1 suggest the amorphous material would be physically stable. The nature of the small endotherm at 102° C. is unknown and a similar endotherm is also observed in Example 4. The non-reversing heat flow in Example 1 shows an endotherm at 188° C. which is an enthalpic relaxation corresponding to the glass transition event. The exothermic event at 200° C. in the non-reversing heat flow can be recrystallization or chemical reaction(s). A similar exothermic event immediately following the glass transition is also observed in Example 3. On the other hand, Example 4 exhibited the lowest Tg and lowest ΔCp.

The glass transition event of Example 5 overlaps with multiple events (probably due to excipients) and therefore the unusually high ΔCp value (0.73 J/(g·° C.)) may not be accurate.

In summary, mDSC analysis of four freeze-dried solid dispersions (Examples 1 and 3-5) showed that while all products exhibited glass transition temperatures, Example 1 exhibited the highest glass transition temperature.

### Dissolution Release Studies.

The percentage of dissolution/release of PY of Examples 1, 3-5, and 7-12 were measured using a standard curve in (i) 1× phosphate buffered saline (PBS)(pH 7.4) and (ii) 0.1 N citric acid (CA)(pH 1.2).

Diluent solution: 1M citric acid:H₂O:acetonitrile (ACN)=1:8:1. To prepare 1 liter, 100 mL of ACN was mixed with 800 mL of water and 100 mL of 1.0 M citric acid and mixed well.

Citric acid solution preparation: 1 M citric acid was prepared by dissolving 1.92 g citric acid in 10 mL of water. 0.1 N citric acid was prepared by dissolving 0.96 g citric acid in 50 ml water.

Reference standard solution for linearity curve preparation. Preparation of psilocybin (PY) reference standard solution was performed by diluting stock solution (1000 μg/ml) with diluent solution to 333.3, 200, 50, 10, 1 and 0.1 μg/ml in diluent solution. Table 13 shows the preparation of linearity curve of reference standard solution.

TABLE 13

| Target | Initial | Initial | |
|---|---|---|---|
| Concentration | concentration | concentration | Diluent |
| μg/mL | μg/ml | volume (μl) | (μl) |
| 200 | 1000 | 200 | 800 |
| 100 | 200 | 400 | 400 |
| 50 | 200 | 200 | 600 |
| 10 | 50 | 200 | 800 |
| 1 | 10 | 50 | 450 |
| 0.1 | 1 | 50 | 450 |

Freeze-dried samples (10 mg psilocybin) were charged into appropriately sized beakers or flasks, and then poured into 0.1 N citric acid or PBS in scintillation vials equipped with a stir bar, and the contents were stirred at 250 rpm at room temperature. Time course sampling at 1, 5 and 10 min with each solution with 300 μL of diluent was performed, whereby samples were diluted to 50% with 300 μL of diluent (50% 0.1% N citric acid/ACN) in each timepoint, centrifuged to separate solid/liquid, and 200-300 μL of the upper layer was taken up in an HPLC vial for dissolution kinetics testing using the following chromatographic conditions (see Table 14). For freeze-dried samples with 10 mg active per sample in the dissolution test, the target concentration was 200 μg/ml when completely dissolved.

### Chromatographic Conditions:

TABLE 14

| | | |
|---|---|---|
| Column | Stationary Phase | ZorbaxSB 18 3.5 mm |
| | Material/Dimensions | Stainless steel, 4.6 × 150 mm |
| | Mobile Phase A | 0.1% TFA in Water |
| | Mobile Phase B | 0.1% TFA in ACN |

| Time (min) | % A | % B |
|---|---|---|
| 0 | 95 | 5 |
| 1 | 95 | 5 |
| 14 | 60 | 40 |
| 15 | 5 | 95 |
| 18 | 5 | 95 |
| 18.1 | 95 | 5 |
| 20 | 95 | 5 |

| Flow rate | 1.0 | mL/min |
|---|---|---|
| Column Temp | 30° | C. |
| Injection Vol | 10 | μL |
| Needle wash | Diluent | |
| Detection wavelength | 267 | nm |
| Run Time | 20 | min |

Results. As can be seen in FIG. 29, Example 1 exhibited a similar release rate of PY in both acidic (0.1 N citric acid) and neutral (PBS) pH. A burst release of up to 80% was attained in 1 minute and remained consistent through 10 minutes, characteristic of an immediate release dosage form.

For Example 3, the release of PY was time dependent in both acidic (0.1 N citric acid) and neutral (PBS) conditions. PY release was higher/faster in PBS (100% at 10 min) than in citric acid buffer (70% at 10 min)(FIG. 30). Example 3 can be characterized as a fast release dosage form.

Contrary to Example 3, PY release in Example 4 was higher in acidic pH (95% at 10 min in 0.1 N citric acid) than it was in neutral pH (70% at 10 min in PBS)(FIG. 31). The release kinetics were characteristic of an immediate release dosage form.

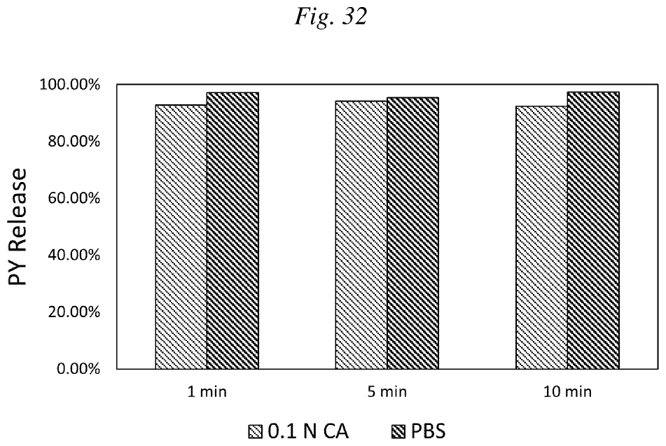

Example 5 exhibited no preference for pH for PY release and demonstrated maximum drug release (95-100%) within 1 minute for both acidic (0.1 N citric acid) and neutral (PBS) conditions (FIG. 32), characteristic of an immediate release dosage form.

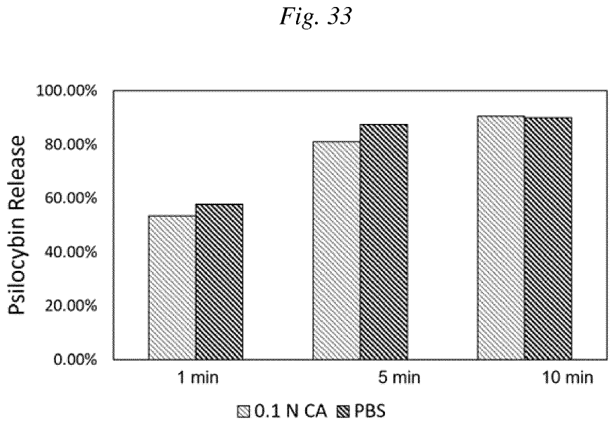

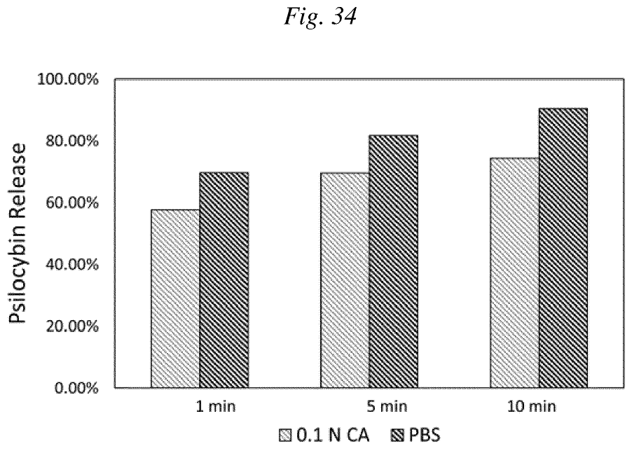

Examples 7 and 8 both exhibited similar release kinetics, despite their different matrix compositions, as can be seen in FIGS. 33 and 34, respectively. PY release for both was slightly higher/faster in PBS than in citric acid buffer. Both polymer matrices have a similar molecular weight range and provided similar release kinetics—a fast release profile with about 90% release within 10 minutes.

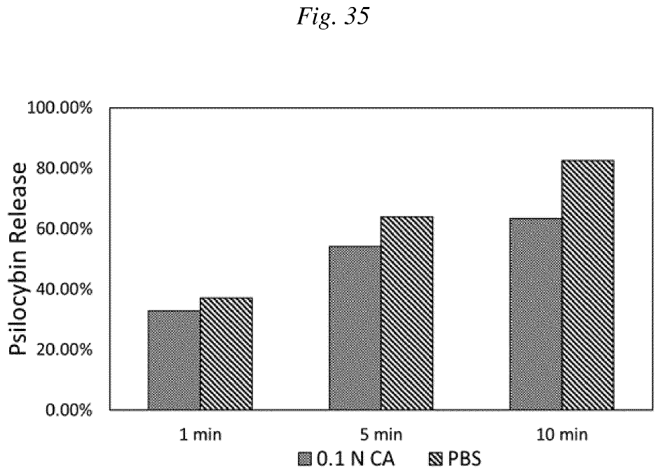

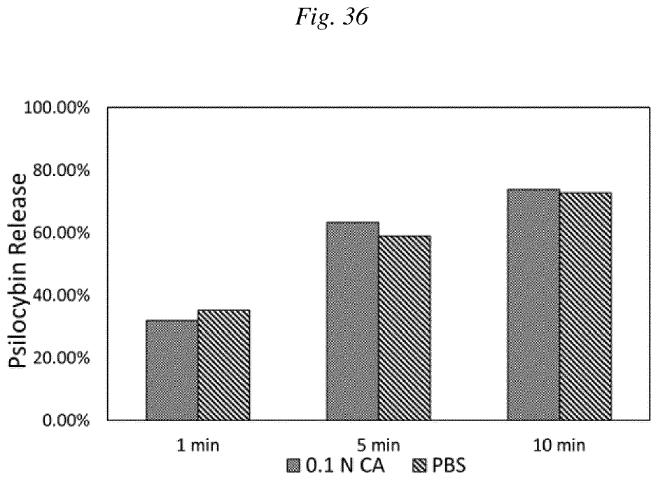

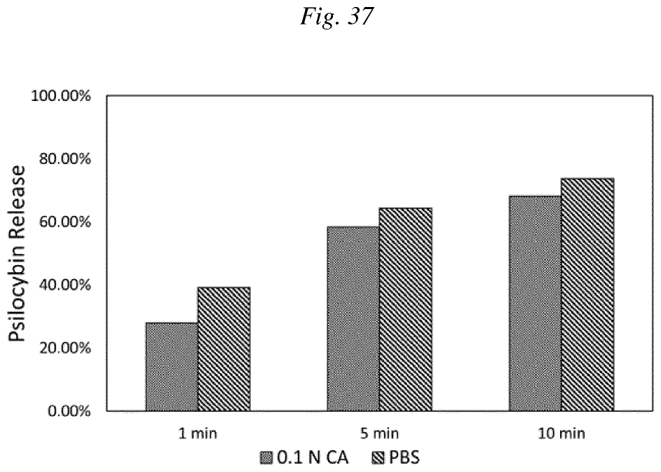

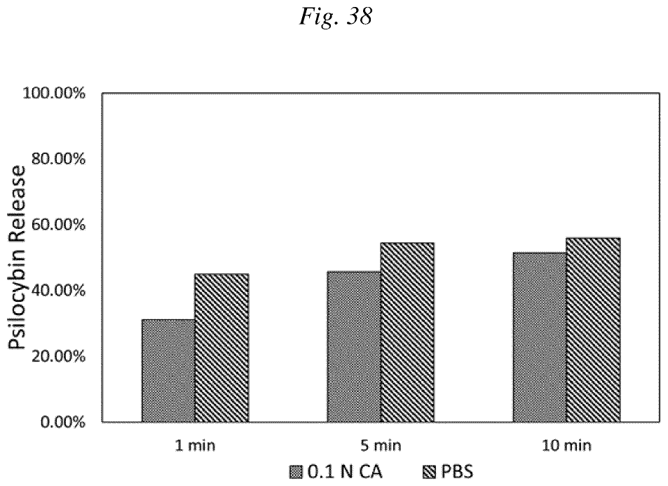

Examples 9-12 all exhibited extended-release kinetics, as can be seen in FIGS. 35-38, respectively. PY release in Example 9 was slightly higher/faster in PBS than in citric acid buffer, reaching 80%+ at 10 minutes. PY release was slowed in Examples 10 and 11 (made using HPMC with a molecular weight range of 400-600 kDa), providing less than about 80% release by the 10-minute timepoint. Example 12 prepared using HPMCAS used as enteric coating in extended-release applications exhibited an even slower release profile of about 50% at 5 minutes, which remained nearly the same up to the 10-minute timepoint.

Stability Studies. Freeze-dried solid dispersions of Examples 1 and 3-5 (in the form of lyophilized cakes) were stored under the following conditions: (i) 40° C., 75% relative humidity (RH); (ii) 40° C., 15% RH; or (iii) room temperature (r.t., about 20-22° C.). After storage for 27 days for Examples 1, 3, and 4 or 24 days for Example 5, the samples were analyzed by high-resolution XRPD to determine whether crystalline psilocybin was present.

The stability results are summarized in Table 15.

TABLE 15

| Example | | High-resolution XRPD Results | |
|---|---|---|---|
| No. | Storage conditions^{a} | Crystalline PY | Excipient(s) |
| 1 | (i) 40° C., 75% RH | Trihydrate^{b} | Not detected |
| | (ii) 40° C., 15% RH | Form A^{c} | Not detected |
| | (iii) r.t. | Form A^{c} (trace) | Not detected |
| 3 | (i) 40° C., 75% RH | Trihydrate^{b} | Not detected |
| | (ii) 40° C., 15% RH | Trihydrate^{b} | Not detected |
| | (iii) r.t. | Trihydrate^{b} | Not detected |
| 4 | (i) 40° C., 75% RH | Trihydrate^{b} | Not detected |
| | (ii) 40° C., 15% RH | Form A^{c} | Not detected |
| | (iii) r.t. | Form A^{c} (trace) | Not detected |
| 5 | (i) 40° C., 75% RH | Not detected | crystalline mannitol |
| | | | (delta + alpha) |
| | (ii) 40° C., 15% RH | Trihydrate | crystalline mannitol |
| | | | (delta + beta + alpha) |
| | (iii) r.t. | Not detected | crystalline mannitol |
| | | | (hemihydrate + delta) |

^{a}Samples were stored for 27 days under the listed conditions, except for those of Example 5, which were stored for 24 days
^{b}CSD Reference Code OKOKAD
^{c}CSD Reference Code HATCAK & TAVZID

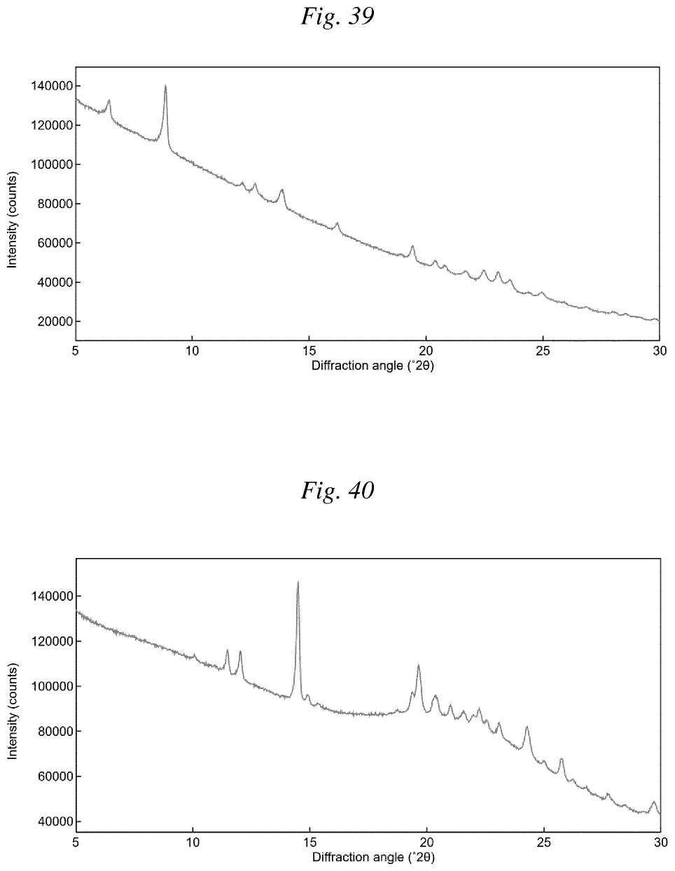

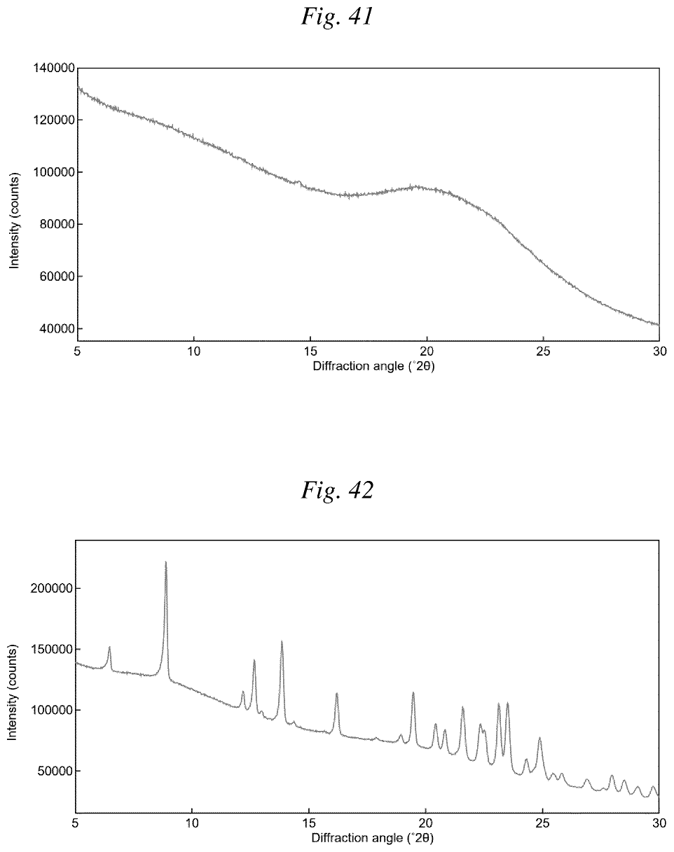

The presence of some crystalline PY was detected in Example 1 after 27 days of storage under stress conditions of (i) 40° C., 75% RH and (ii) 40° C., 15% RH (FIGS. 39 and 40, respectively). The room temperature sample on the other hand displayed only a trace of a peak at 14.5 °2θ that was consistent with the strongest peak in the reference pattern of psilocybin Form A, but was otherwise consistent with amorphous material (FIG. 41).

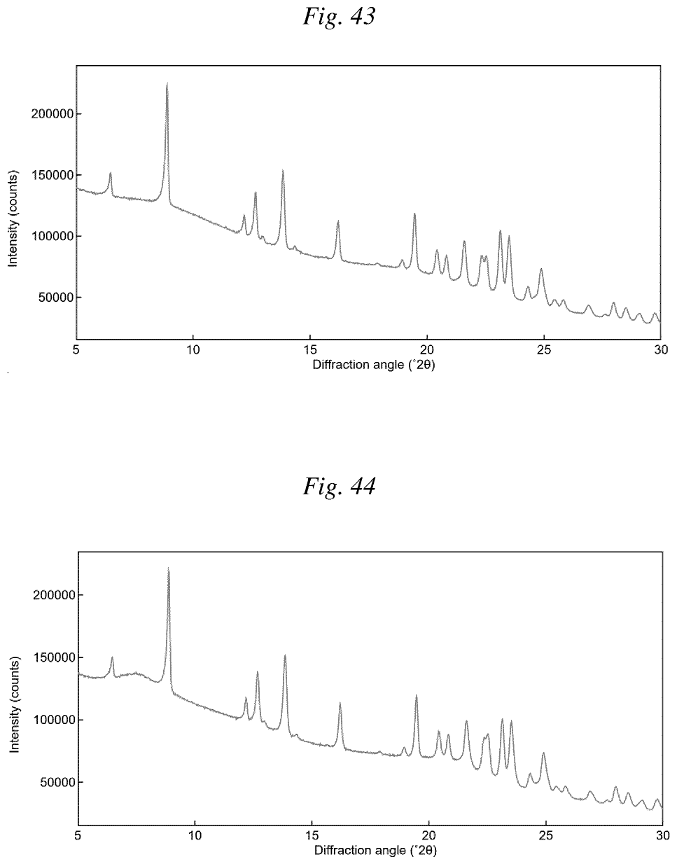

Crystalline PY (trihydrate) was detected in Example 3 after 27 days of storage under the storage conditions of (i) 40° C., 75% RH; (ii) 40° C., 15% RH; and (iii) room temperature (FIGS. 42-44, respectively), indicating that Example 3 was less stable to recrystallization under the tested stress conditions in comparison to other samples analyzed.

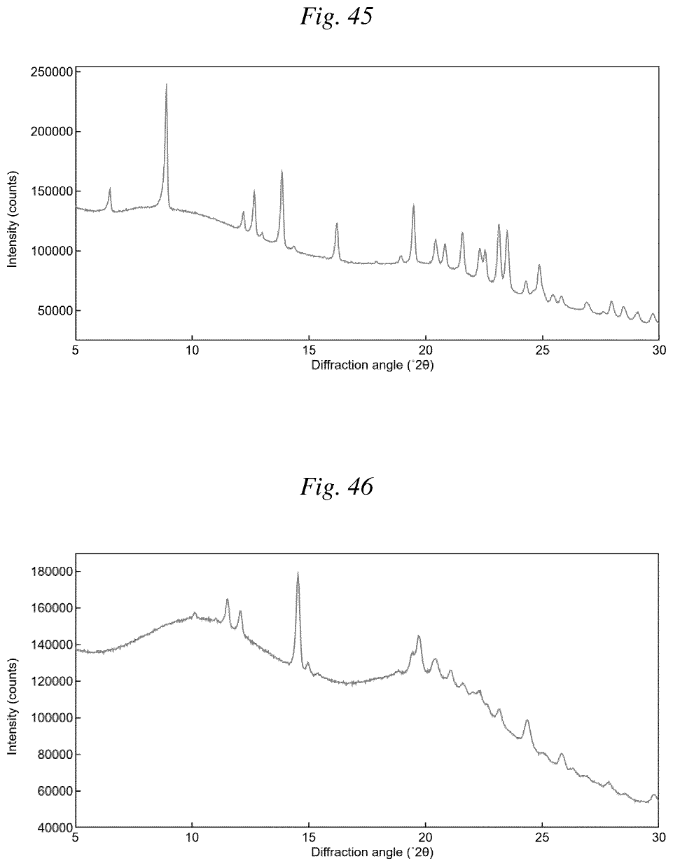

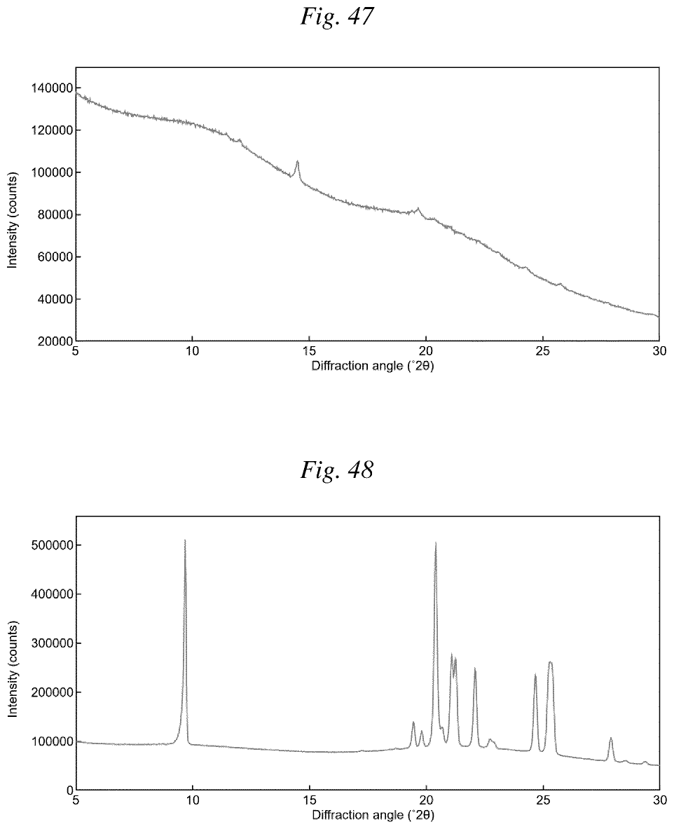

The presence of some crystalline PY was detected in Example 4 after 27 days of storage under stress conditions of (i) 40° C., 75% RH and (ii) 40° C., 15% RH (FIGS. 45 and 46, respectively). However, only trace crystalline PY was detected in the room temperature sample, indicating suitable stability under such storage conditions (FIG. 47).

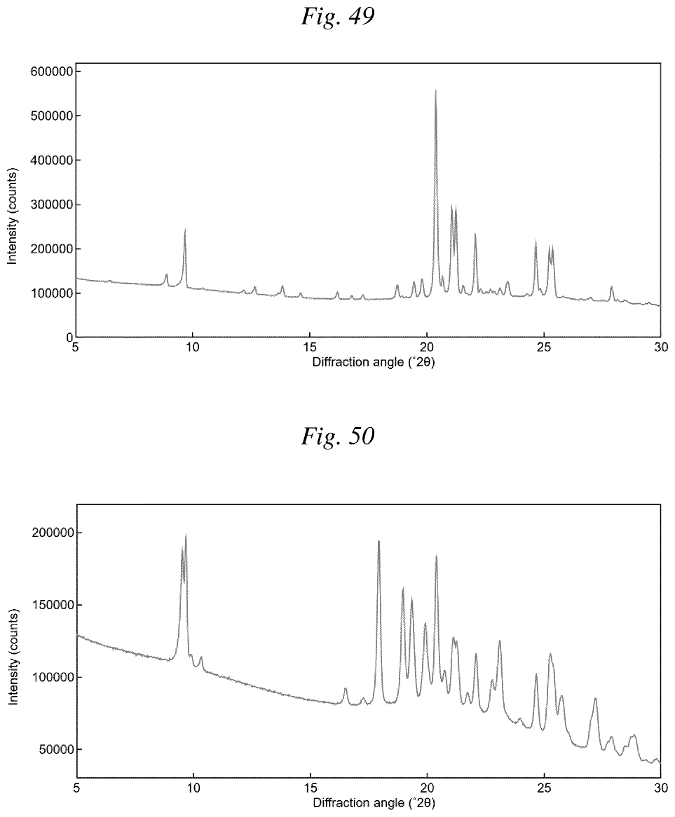

With respect to Example 5, crystalline PY (trihydrate) was detected only in the sample subjected to (ii) 40° C., 15% RH for 24 days (FIG. 49). The samples subjected to (i) 40° C., 75% RH and (iii) room temperature for 24 days showed no signs of crystalline PY, only crystalline mannitol excipient was detected (FIGS. 48 and 50, respectively).

Overall, these results indicate that room temperature conditions are more favorable storage conditions to prevent psilocybin recrystallization than higher temperature/high relative humidity conditions, which is not surprising given that freeze-dried pharmaceutical dosage forms are known to be sensitive to water.

### III. Solid Dispersions Prepared by Spray Drying

### Materials.

EUDRAGIT® E PO (EPO; a cationic low viscosity terpolymer based on N,N-dimethylaminoethyl methacrylate-methylmethacrylate-butylmethacrylate; 2:1:1; weight average molecular weight of about 47,000 g/mol; immediate release; soluble below and permeable above pH 5.0; available from Evonik).

KOLLIDON® 30 (also called PVP K-30, amorphous, water-soluble polyvinylpyrrolidone with a weight average molecular weight of 44,000-54,000 g/mol; available from BASF.

KOLLIDON® VA 64 (a 60:40 copolymer of VP:VAc, 45,000-75,000 g/mol, available from BASF).

PHARMACOAT® 606 (HPMC with a 2910 substitution type: 28-30% methoxy substitution, 7-12% hydroxypropyl substitution; viscosity of 6.0 mPa s as 2% solution in water at 20° C.), available from Shin-Etsu Chemical Co. Ltd.

AQOAT® AS-MG (HPMCAS with 9% acetyl, 11% succinoyl; 1,000 μm mean particle size; dissolution pH≥6.0), available from Shin-Etsu Chemical Co. Ltd.

EUDRAGIT® L 100-55 (an anionic 1:1 copolymer of methacrylic acid-ethyl acrylate; delayed release; dissolution above pH 5.5; available from Evonik).

Psilocybin (3-(2-(dimethylamino)ethyl)-1H-indol-4-yl dihydrogen phosphate; PY; I-7) starting material used was in crystalline form as polymorph Form A, commercially available from Biosynth Carbosynth.

### Analytical Methods.

X-ray power diffraction (XRPD): XRPD patterns were collected on a Bruker AXS D2 diffractometer using Cu Kα radiation (30 kV, 10 mA), 0-0 geometry, using a LynxEye detector from 5-42 °2θ.

The software used for data collection was DIFFRAC.SUITE and the data were analyzed and presented using Diffrac Plus EVA v 16.0.0.0.

The details of the data collection are:
Angular range: 5 to 42 °2θ
Step size: 0.024° 2θ
Collection time: 0.1 seconds per step.

Samples were run under ambient conditions and prepared as flat plate specimens using powder without grinding. Approximately 1-2 mg of the sample was lightly pressed on a silicon wafer to obtain a flat surface.

Modulated differential scanning calorimetry (mDSC): TOPEM® is a temperature modulated DSC method which differs from conventional DSC in allowing the total heat flow to be separated into reversing and non-reversing heat flow components. Such techniques help distinguish between processes or transitions that overlap or lie very close to one another. The glass transition temperature (Tg) was determined from the reversible heatflow component.

TOPEM® DSC data were collected on a Mettler DSC 3+ equipped with a 34 position auto-sampler. The instrument was calibrated for energy and temperature using certified indium. Typically, 3-5 mg of each sample, in a pin-holed aluminium pan, was heated at 2 K·min⁻¹ from 30° C. to 220° C. with a pulse height of 0.64 K and a pulse width 15-30 sec. A nitrogen purge at 50 mL·min⁻¹ was maintained over the sample. STARe v15.00 was used for instrument control and data processing.

Predicted Tg: A single glass transition (Tg) by DSC is often an indicator of miscibility. Tg predictions were made using the Fox equation (1) which assumes densities are equal.

$$1 / {T}_{gmix} = {w}_{1} / {T}_{g 1} + {w}_{2} / {T}_{g 2} ( w = weight ) \qquad ( 1 )$$

Determining the Tg is valuable for stability indication as above the Tg the material is prone to crystallization due to molecular motion. Amorphous solid dispersions (ASDs) can take up water, which acts as a plasticiser, and which will reduce the Tg. If the Tg is less than 90° C. there is a significant chance for water uptake, which could cause the Tg to drop to below 25° C. where recrystallization is likely to occur.

Thermogravimetric Analysis (TGA): TGA data were collected on a Mettler TGA 2 equipped with a 34 position auto-sampler. The instrument was temperature calibrated using certified isatherm and nickel. Typically, 5-30 mg of each sample was loaded into a pin-holed aluminium pan and heated at 10° C.·min⁻¹ from 30° C. to 400° C. A nitrogen purge at 50 mL min⁻¹ was maintained over the sample. STARe v15.00 was used for instrument control and data processing.

Miscibility assessment. The miscibility of psilocybin with a range of polymers was first assessed to determine which polymers were suitable for the preparation of amorphous solid dispersions through spray drying preparation methods. Physical mixtures (admixtures) of psilocybin with 6 different polymers (EUDRAGIT® E PO, KOLLIDON® 30, KOLLIDON® VA 64, PHARMACOAT® 606, AQOAT® AS-MG, and EUDRAGIT® L 100-55) were prepared at five different drug loadings (nominally 10, 25, 50, 75 and 90% w/w). Miscibility was assessed based on a change in melting point of the physical mixture when heated using DSC when compared to the psilocybin alone (melting point onset: 217° C.). For EUDRAGIT® L 100-55, miscibility was assessed by glass transition (Tg) temperature.

The outcomes from the different physical mixtures on melting point are summarized in Table 16.

TABLE 16

| | 10 wt. % | 25 wt. % | 50 wt. % | 75 wt. % | 90 wt. % |
|---|---|---|---|---|---|
| Polymer | psilocybin | psilocybin | psilocybin | psilocybin | psilocybin |
| | Melting temperature onset of physical mixture (° C.)* | | | | |
| EUDRAGIT ® E PO | 205 | N/A | 201 | 200 | 200 |
| KOLLIDON ® 30 | 198 | N/A | 198 | 200 | 200 |
| KOLLIDON ® VA 64 | 205 | 192 | 200 | 198 | 199 |
| PHARMACOAT ® 606 | 201 | 200 | 199 | 200 | 198 |
| AQOAT ® AS-MG | 202 | 201 | 202 | 201 | 200 |
| | Predicted Tg (° C.) of physical mixture | | | | |
| EUDRAGIT ® L 100- | 113 | 123 | 131 | 144 | 153 |
| 55 | | | | | |

*For reference, psilocybin alone has a melting point onset of 217° C.

All of the tested physical mixtures showed a decrease in melting point, compared to psilocybin alone, and no significant difference in the drop in melting points across the different psilocybin:polymer loading was observed. Thus, it is likely that psilocybin has at least some miscibility with each of the polymers. Miscibility experiments on EUDRAGIT® L 100-55 were not possible due to thermal degradation of polymer. Based on predicted Tg and historical use, it was considered for progression.

Based on the above and the miscibility results, KOLLIDON® VA 64, PHARMACOAT® 606, and AQOAT® AS-MG, and EUDRAGIT® L 100-55 were progressed to the next stage and prepared as solid dispersions via spray drying, using a 25 wt. % psilocybin loading.

### Spray Dried Solid Dispersions and Experimental Procedures.

### Examples 16-19

The polymers used to prepare solid dispersions were as follows: Example 16 (KOLLIDON® VA 64), Example 17 (PHARMACOAT® 606), Example 18 (AQOAT® AS-MG), and Example 19 (EUDRAGIT® L 100-55). Samples were prepared with a psilocybin/polymer ratio of 25/75 (% wt./wt.) on a 400 mg scale, using a 2 wt. % solid loading in solvent for the spray drying procedure. The solvent used was a 25/75 mixture by volume of dichloromethane (DCM)/methanol. Spray dried psilocybin (no polymer) was also prepared as a control.

Spray drying was performed using spray dyer-spray chiller/congealer PROCEPT 4M8-Trix, available from Procept, using parameters outlined in Table 17. After spray drying, the samples were dried under vacuum overnight at 6 mBar at 40° C.

TABLE 17

| Parameter | Set point | |
|---|---|---|
| Equipment | PROCEPT 4M8-Trix | |
| Solvent | DCM/MeOH (1:3) | |
| Solid content | 2 wt. % (PY:polymer 1:3 w/w) | |
| Cyclone | Large | |
| Nozzle size | 0.4 | mm |
| Inlet gas flow | 0.4 | m³/min |
| Inlet temperature | 120° | C. |
| Cyclone gas flow | 400 | L/min |
| Nozzle gas flow | 3 | L/min |
| Pump speed | 200 | rpm |

| | |
|---|---|
| Post processing condition | Dried under vacuum overnight |
| | (6 mBar; 40° C.) |

### Characterization/Results.

Spray dried solid dispersions of Examples 16-19 were characterized initially (t=0) using X-ray powder diffraction (XRPD) and modulated differential scanning calorimetry (mDSC), and the results are summarized in Table 18.

TABLE 18

| Example | | Characterization (t = 0) | | | |
|---|---|---|---|---|---|
| No. | Formulation | Yield (%) | XRPD | Tg (° C.) | Tg predicted |
| (control) | Psilocybin (PY) | N/A | Amorphous | 161 | N/A |
| 16 | PY/KOLLIDON ® VA 64 | 47.5 | Amorphous | 117 | 119 |
| 17 | PY/PHARMACOAT ® 606 | 57.7 | Amorphous | 153 | 161 |
| 18 | PY/AQOAT ® AS-MG | 60.0 | Amorphous | 127 | 130 |
| 19 | PY/EUDRAGIT ® L 100-55 | 57.5 | Amorphous | 131 | 125 |

The solid dispersions of Examples 16-19 provided amorphous material by XRPD. All four polymers tested produced solid dispersions with a single Tg event, indicating each was made as a single phase, therefore psilocybin and each polymer were miscible. The measured Tg values were close to predicted glass transition temperatures. Further, all Tg values measured were >90° C., and thus the amorphous solid dispersions are within typical stability limits.

Stability studies. To further probe the stability, the amorphous solid dispersions of Examples 16-19 were stored at 40° C. in closed vials and at set timepoints at 6 hours (t=6 hr), 24 hours (t=24 hr), 1 week (t=1 w), and/or 4 weeks (t=4 w) were analyzed by XRPD and/or mDSC to evaluate stability against crystallization, as well as by TGA for water uptake.

The stability results for Example 16 are summarized in Table 19.

TABLE 19

| | Timepoint | | | | |
|---|---|---|---|---|---|
| Example 16 | t = 0 | t = 6 hr | t = 24 hr | t = 1 w | t = 4 w |
| XRPD | Amorphous | Amorphous | Amorphous | Amorphous | Amorphous |
| Tg (° C.) | 117 | NT | NT | 115 | 111 |
| TGA | No mass loss | NT | No mass loss | ca. 2.2% | ca. 2.2% |
| (% mass loss) | observed | | observed | 32-87° C. | 30-95° C. |

NT = not tested

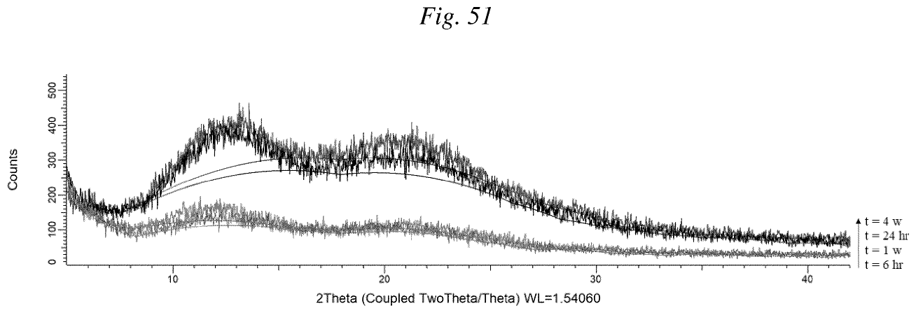

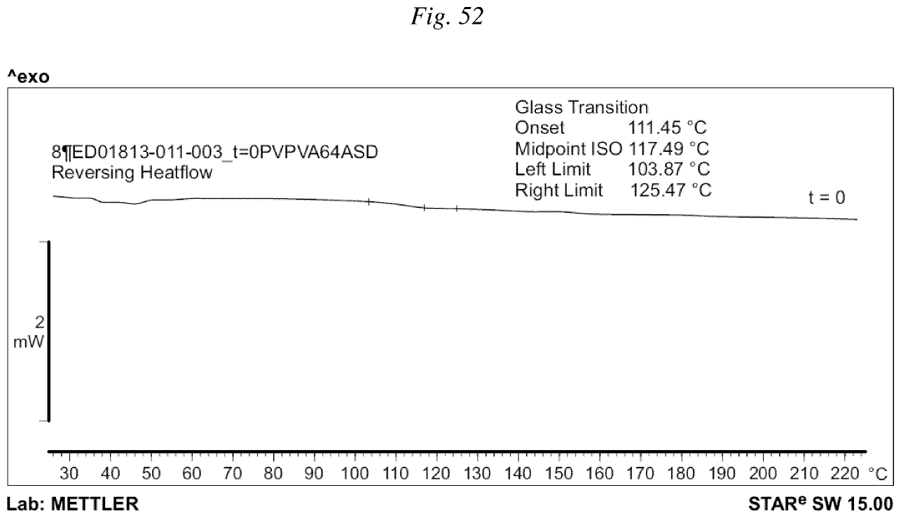

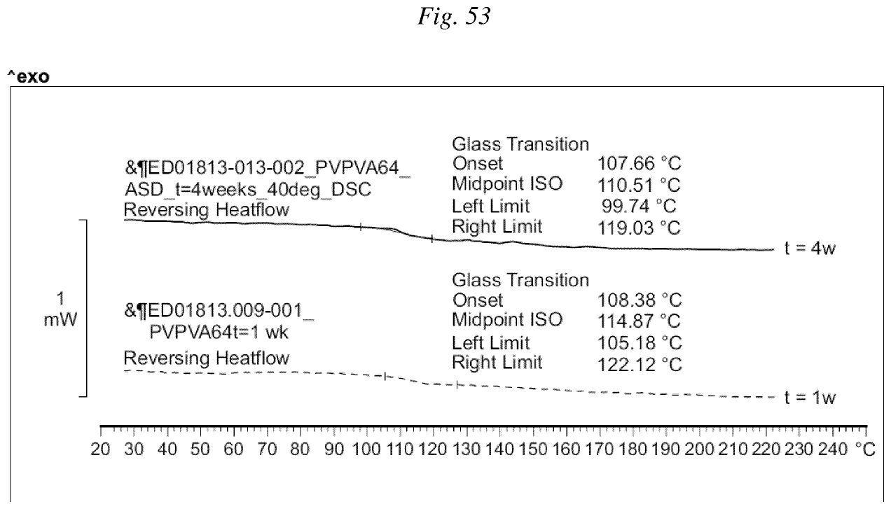

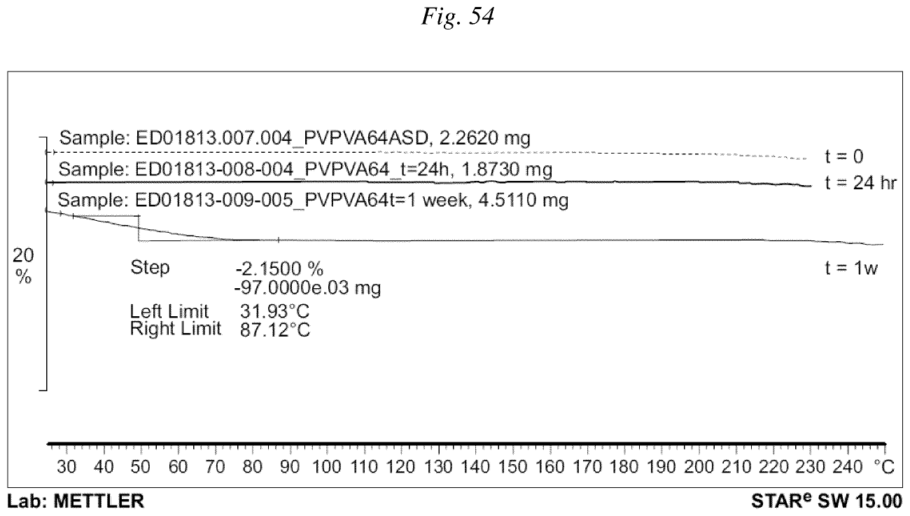

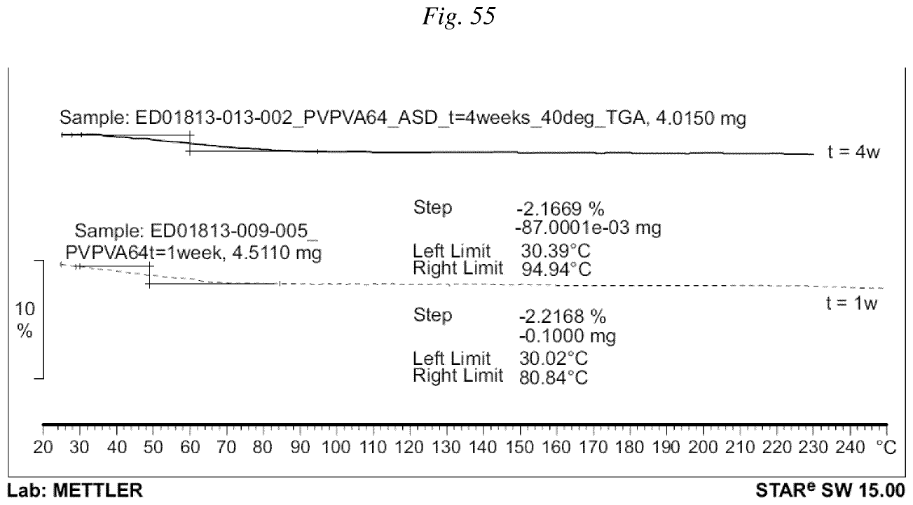

After 4 weeks of storage at 40° C. in a closed vial, the solid dispersion of Example 16 remained amorphous by XRPD with no evidence of psilocybin recrystallization (FIG. 51). This material had a single Tg event at 117° C. at t=0 (FIG. 52), and still had a single Tg event at 1 week and 4 weeks of storage at 40° C. (FIG. 53). The single Tg on storage indicates no changes in miscibility occurred. There was some evidence of water uptake after 1 week of storage at 40° C. (FIG. 54), however this was consistent at the 4 week timepoint (FIG. 55), suggesting water uptake was not increasing over time. Overall, Example 16 is considered stable under the tested storage conditions.

The stability results for Example 17 are summarized in Table 20.

TABLE 20

| | Timepoint | | | | |
|---|---|---|---|---|---|
| Example 17 | t = 0 | t = 6 hr | t = 24 hr | t = 1 w | t = 4 w |
| XRPD | Amorphous | Amorphous | Amorphous | Amorphous | Amorphous |
| Tg (° C.) | 153 | NT | NT | 153 | 153 |
| TGA | No mass loss | NT | No mass loss | No mass loss | ca. 1.4% |
| (% mass | observed | | observed | observed | 34-92° C. |
| loss) | | | | | |

NT = not tested

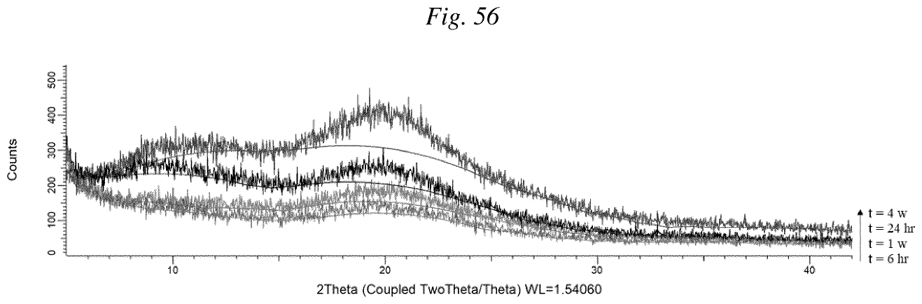

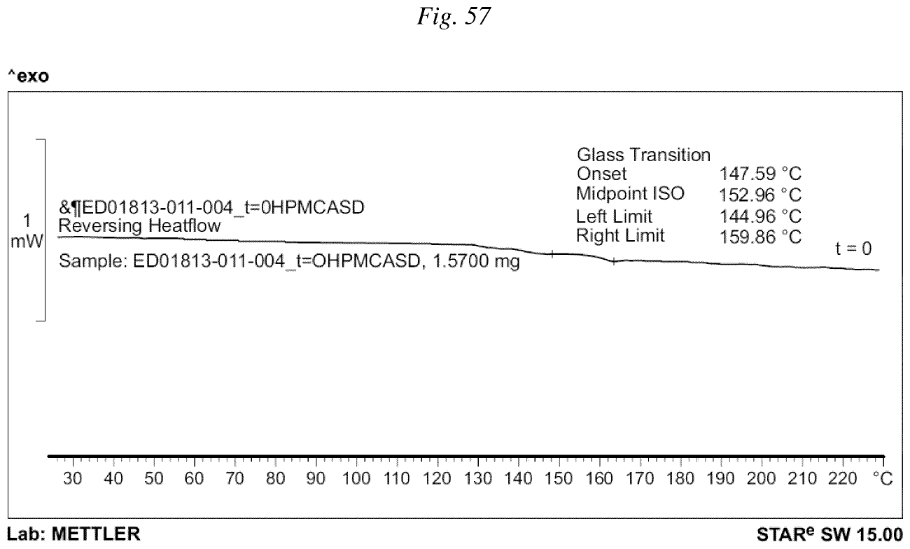

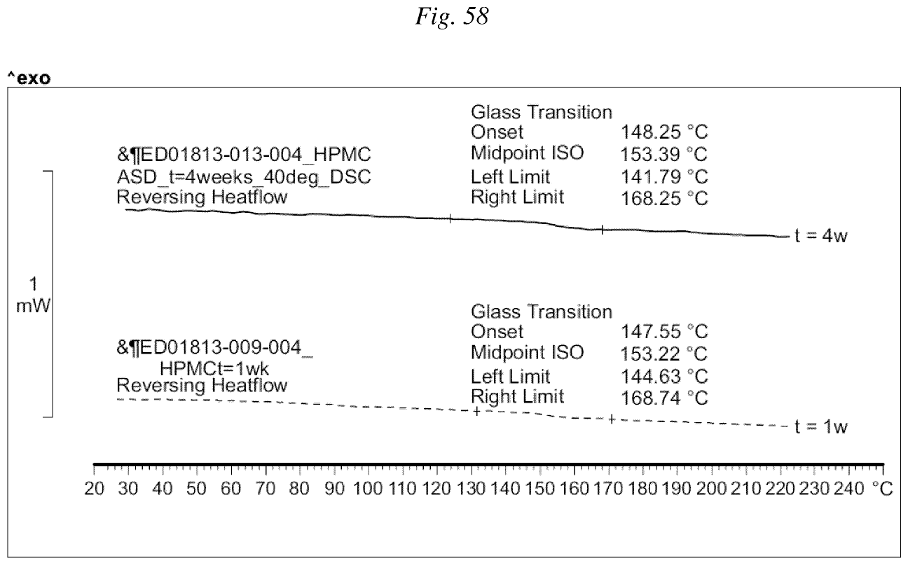

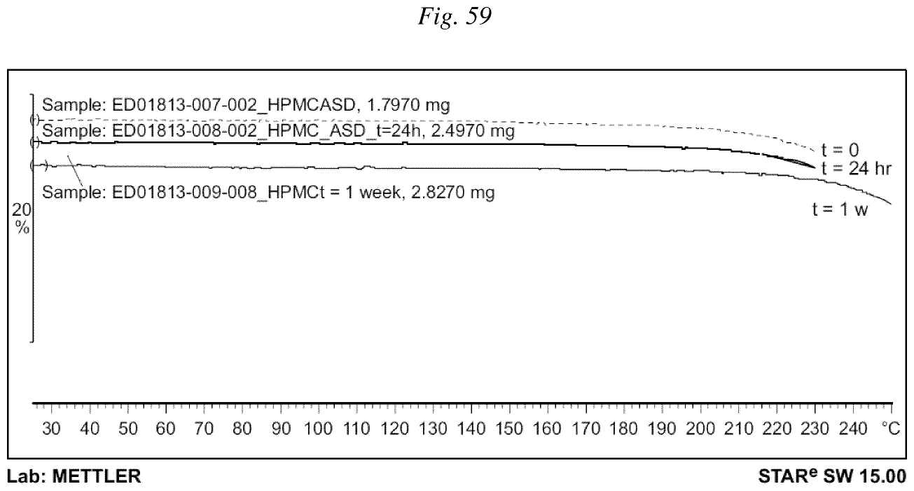

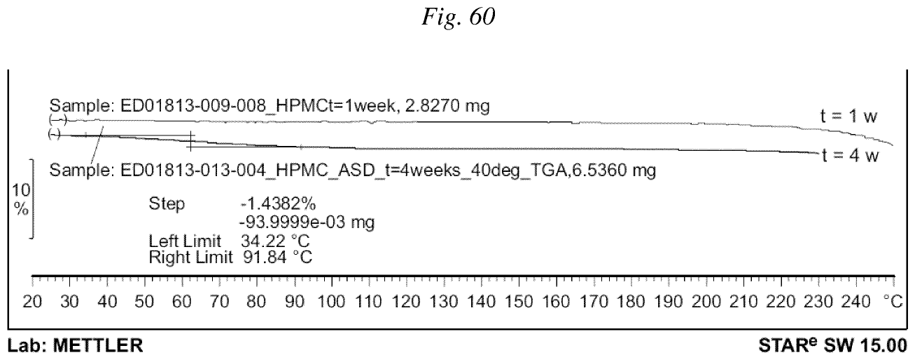

After 4 weeks of storage at 40° C. in a closed vial, the solid dispersion of Example 17 remained amorphous by XRPD with no evidence of psilocybin recrystallization (FIG. 56). This material had a single Tg event at 153° C. at t=0 (FIG. 57), and still had a single Tg event at 1 week and 4 weeks of storage at 40° C. (FIG. 58). The single Tg on storage indicates no changes in miscibility occurred. There was no evidence of water uptake after 1 week of storage at 40° C. (FIG. 59), however there was a slight mass loss of about 1.4% at the 4 week timepoint (FIG. 60), suggesting there may be some water uptake occurring over time. Overall, Example 17 is considered stable under the tested storage conditions.

The stability results for Example 18 are summarized in Table 21.

TABLE 21

| | Timepoint | | | | |
|---|---|---|---|---|---|
| Example 18 | t = 0 | t = 6 hr | t = 24 hr | t = 1 w | t = 4 w |
| XRPD | Amorphous | Amorphous | Amorphous | Amorphous | Amorphous |
| Tg (° C.) | 127 | NT | NT | 125 | 128, 152 |
| TGA | No mass loss | NT | No mass loss | No mass loss | ca. 0.8% |
| (% mass | observed | | observed | observed | 32-97° C. |
| loss) | | | | | |

NT = not tested

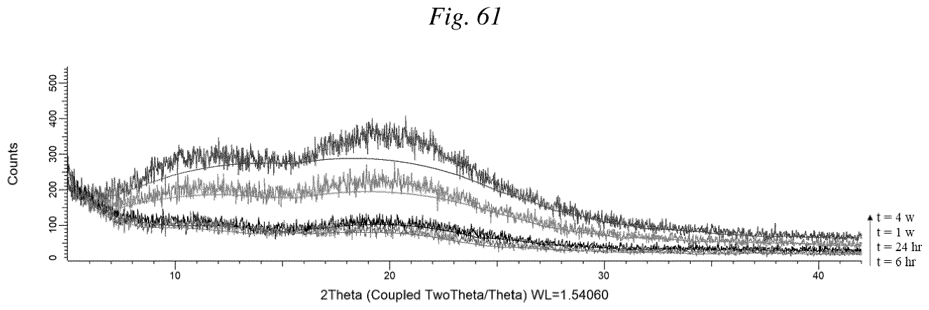

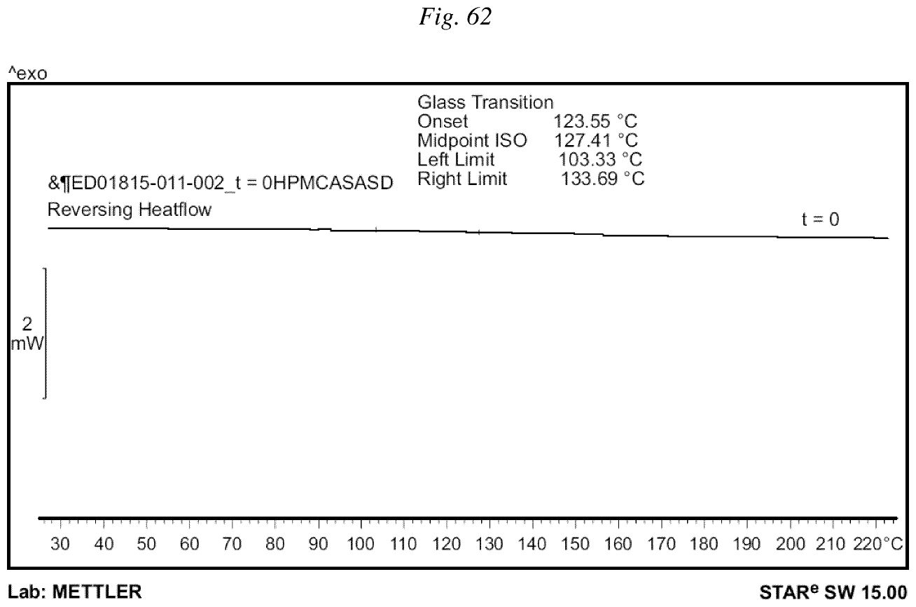

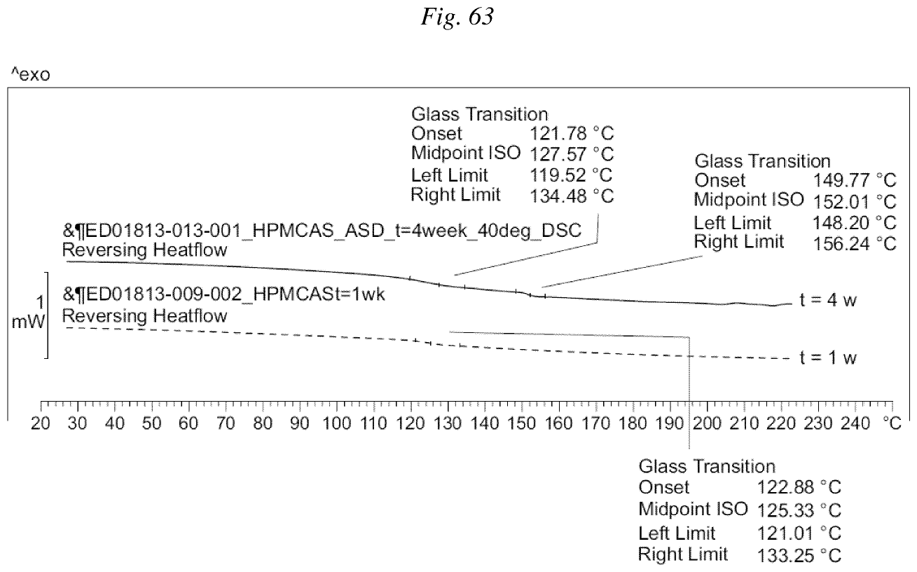

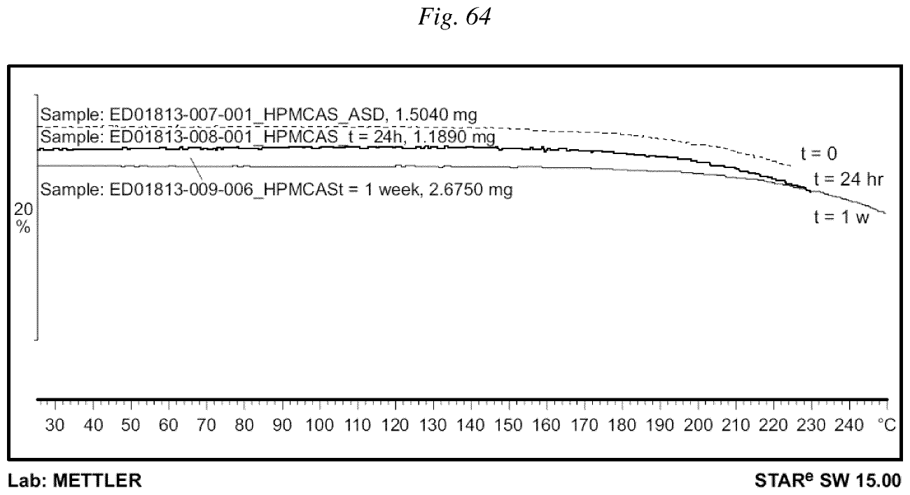

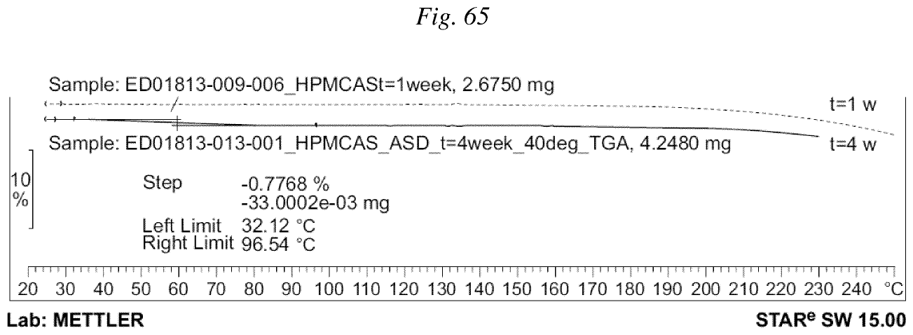

After 4 weeks of storage at 40° C. in a closed vial, the solid dispersion of Example 18 remained amorphous by XRPD with no evidence of psilocybin recrystallization (FIG. 61). This material had a single Tg event at 127° C. at t=0 (FIG. 62), but showed an additional Tg event at 152° C. at 4 weeks of storage at 40° C. (FIG. 63), indicating a potential second phase, which could potentially be free psilocybin. There was no evidence of water uptake after 1 week of storage at 40° C. (FIG. 64), however there was a slight mass loss of about 0.8% at the 4 week timepoint (FIG. 65), suggesting there may be some slight water uptake occurring over time. Overall, Example 18 is considered stable under the tested storage conditions, but perhaps less so than Examples 16 and 17 based on the appearance of a second phase overtime.

The stability results for Example 19 are summarized in Table 22.

TABLE 22

| | Timepoint | | | | |
|---|---|---|---|---|---|
| Example 19 | t = 0 | t = 6 hr | t = 24 hr | t = 1 w | t = 4 w |
| XRPD | Amorphous | Amorphous | Amorphous | Amorphous | Amorphous |
| Tg (° C.) | 131 | NT | 126 | 136, 147 | 131, 152 |
| TGA | No mass loss | NT | No mass loss | NT | ca. 0.7% |
| (% mass | observed | | observed | | 46-90° C. |
| loss) | | | | | |

NT = not tested

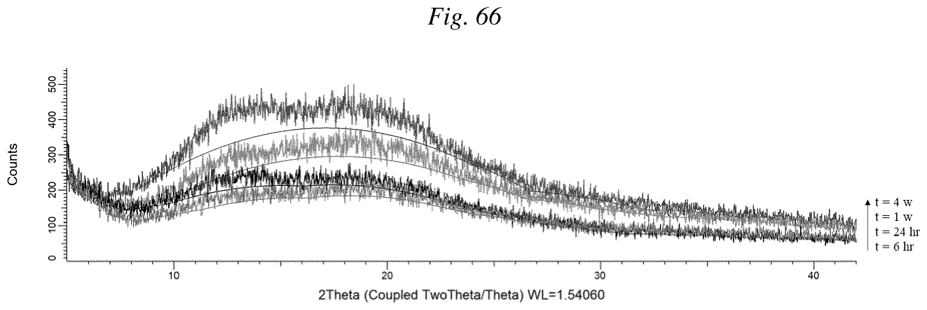

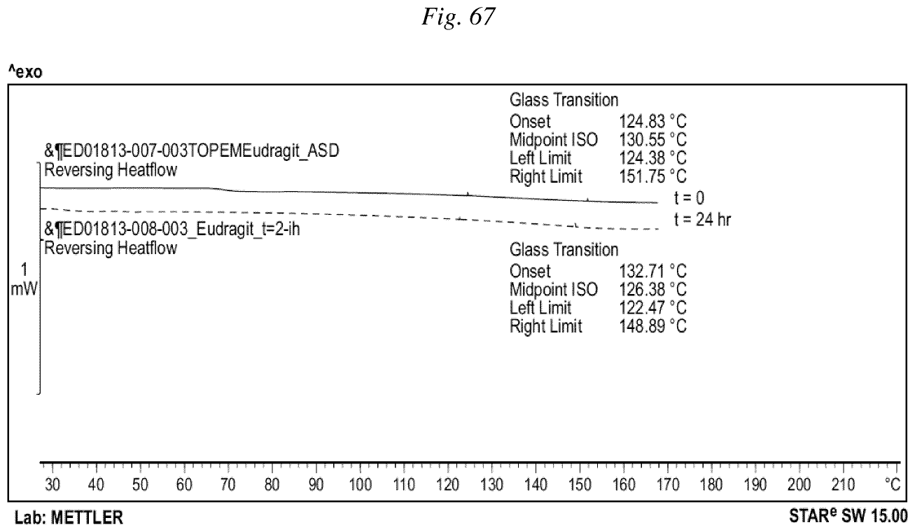

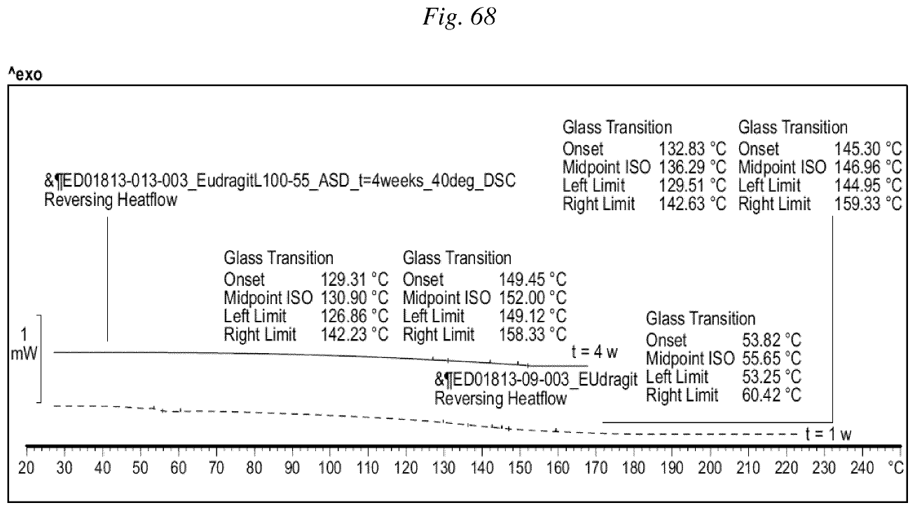

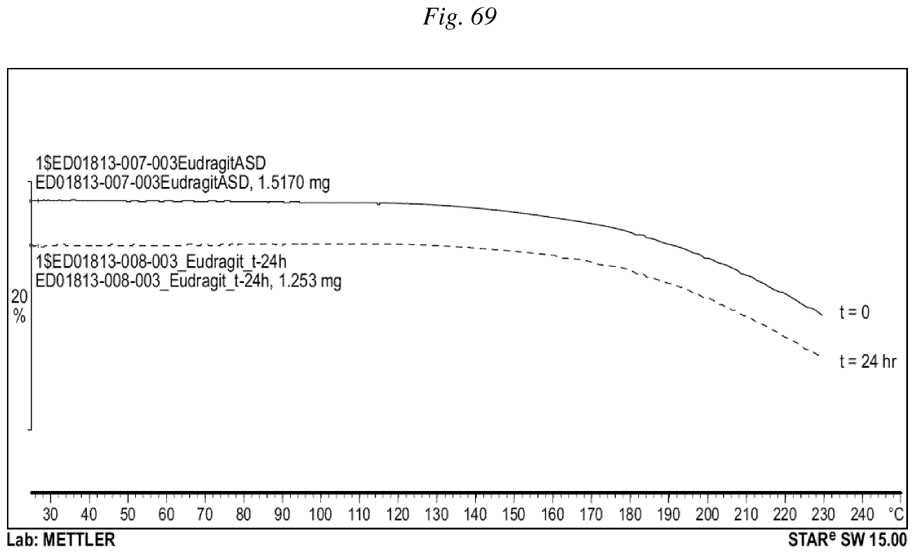

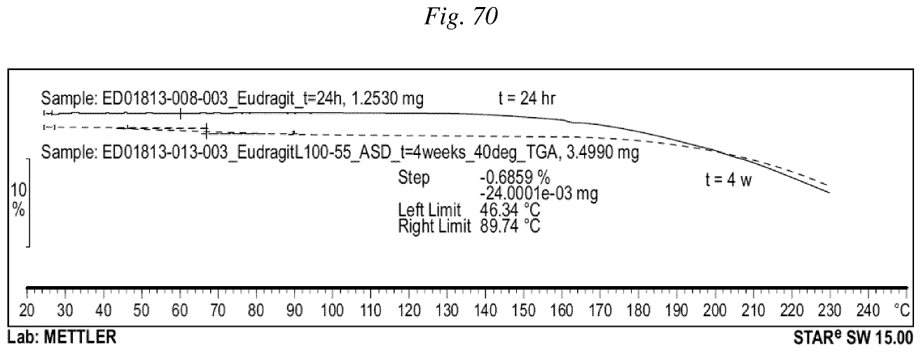

After 4 weeks of storage at 40° C. in a closed vial, the solid dispersion of Example 19 remained amorphous by XRPD with no evidence of psilocybin recrystallization (FIG. 66). This material had a single Tg event at 131° C. at t=0 (FIG. 67), but showed an additional Tg event at 147° C. at 1 week and at 152° C. after 4 weeks of storage at 40° C. (FIG. 68), indicating a potential second phase, which could potentially be free psilocybin. There was no evidence of water uptake after 24 hours of storage at 40° C. (FIG. 69), however there was a slight mass loss of about 0.7% at the 4 week timepoint (FIG. 70), suggesting there may be some slight water uptake occurring over time. Overall, Example 19 is considered stable under the tested storage conditions, but perhaps less so than Examples 16 and 17 based on the appearance of a second phase overtime.

A summary of the 4 week stability data is presented in Table 23. After 4 weeks of storage at 40° C. in a closed vial, all solid dispersions are still amorphous with no evidence of psilocybin recrystallisation. By comparison, amorphous psilocybin (without polymer) is known to crystallize under a variety of stress conditions (See Greenan et al., Preparation and Characterization of Novel Crystalline solvates and Polymorphs of Psilocybin and Identification of Solid Forms Suitable for Clinical Development, 2020 pre-publication; DOI:10.13140/RG.2.2.32357.14560). The amorphous solid dispersions of Examples 16 and 17 have a single Tg after 4 weeks of storage indicating they have remained as a single phase system. Examples 18 and 19 developed a second Tg, which could indicate changes in miscibility and a new phase developing. All of the tested dispersions showed evidence of a small amount of mass loss related to water uptake, but not to a significant degree.

TABLE 23

| Example | | Characterization (t = 4 weeks at 40° C.) | | |
|---|---|---|---|---|
| No. | Formulation | XRPD | Tg events | Mass loss observed |
| 16 | PY/KOLLIDON ® VA 64 | Amorphous | 1 | Yes (~2.2 wt. %) |
| 17 | PY/PHARMACOAT ® 606 | Amorphous | 1 | Yes (~1.4 wt. %) |
| 18 | PY/AQOAT ® AS-MG | Amorphous | 2 | Yes (~0.8 wt. %) |
| 19 | PY/EUDRAGIT ® L 100-55 | Amorphous | 2 | Yes (~0.7 wt. %) |

### IV. Solid Dispersion Prepared by Solution Casting

### Example 20

Psilocybin (3-(2-(dimethylamino)ethyl)-1H-indol-4-yl dihydrogen phosphate; PY; I-7)(200 mg) in crystalline form as crystalline methanol solvate with a small quantity of crystalline Form B as described above and commercially available from Quality Chemical Labs, is dissolved in 10 ml of a solution of 0.1 M phosphate buffer with 5% hydroxypropyl methyl cellulose (HPMC) and 10% polyvinylpyrrolidone (PVP), and the pH is adjusted to 7.0. The solution is then cast on a polymer liner and dried at ambient temperature for 8 h to form a film, which is subjected to additional drying at 50° C. for 2 h in a vacuum oven. The occurrence of the amorphous form of PY in the film will be confirmed by DSC, TGA and X-ray powder diffraction methods. The film is stored at 4° C. and the stability pulls conducted at T=1, 3, 6, and 12 months confirming the presence of the amorphous form of PY.

The film displays good properties as a prototype buccal dose form with disintegration time of 3 min and dissolution kinetics characterized by a dissolution time of 5 min.

### V. Animal Studies

### Pharmacokinetics of Psilocybin in the Male Beagle Dog Following Oral Administration by Powder in Capsule (PIC) or Orally Disintegrating Tablet (ODT)

The pharmacokinetic profile of psilocin from psilocybin after oral administration in oral disintegrating tablets (ODTs) or powder in capsule (PIC) dosage forms to male beagle dogs was compared.

Animals. Six, non-naive, male Beagle dogs aged ca 2-5 years and weighing ca 10-15 kg at dosing were used. These animals were supplied by a recognized supplier of laboratory animals and are currently held as part of a colony at the test facility. Following study completion, animals were returned to the colony for further use, following an appropriate washout period.

Housing. Animals were housed and maintained according to established procedures as detailed in the appropriate Standard Operating Procedures (SOPs). Animals were uniquely identified by tattoo or by microchip. During the pre-trial holding periods, the animals were group housed in caging appropriate to the species. The dogs were housed singly for up to 4 h per day and in this period, had access to their daily ration of diet. The dogs were exercised during the study. Animals were checked regularly throughout the duration of the study. Any clinical signs were closely monitored and recorded. Animals had access to 300-350 g/day of Special Diet Services (SDS) D3 (E) SQC diet throughout the study, except during designated procedures. Mains quality tap water was available ad libitum.

Test items. Orally disintegrating tablet (ODT) dosage forms of Example 6 were used containing 5 mg of amorphous psilocybin. Powder in capsule (PIC) dosage forms were prepared using 5 mg of crystalline psilocybin (polymorph Form A, commercially available from Biosynth Carbosynth) as powder inside a capsule.

### Dose Levels.

Psilocybin (amorphous) as ODT contains 5 mg of active; nominal 0.5 mg/kg active
Psilocybin (crystalline) as PIC contains 5 mg of active; nominal 0.5 mg/kg active

Experimental design. Animals received 5 mg of test item via ODT or by PIC. Each animal received a dose level of ca 0.5 mg/kg, but may vary according to the most recent bodyweight of each animal. Bodyweights were recorded for each animal prior to dosing. Oral administration was performed with either an ODT or PIC containing psilocybin. Capsules were placed at the back of the throat and the animals were encouraged to swallow. A flush of 5 mL of water was given if required. Orally disintegrating tablets were placed under the tongue (sublingual). Animal's mouth were held closed for 10 seconds to ensure the tablet was fully dissolved.

Sampling collection. PK samples (ca 1 mL) were collected from the jugular vein by venepuncture into tubes containing K₂EDTA anticoagulant at the following sampling times: Pre-dose, 5, 10, 15, 30, 60, 120, 240 min, 8 and 24 hrs post-dose. Immediately following collection, samples were inverted to ensure mixing with anti-coagulant and placed on wet ice. As soon as practically possible, plasma samples were generated by centrifugation (2500 g, 10 min, 4° C.). All plasma samples generated were transferred from K₂EDTA tube to aliquot A (per animal/timepoint). Then, 300 μL of plasma and 300 μL (1:1 (v/v)) of 200 mM ascorbic acid were decanted into Aliquot B and stored in a freezer set to maintain a temperature of −65° C., until analysis.

Bioanalysis. Plasma samples were analyzed using an established LC-MS/MS assay (BQL were set at zero prior to Cmax; BQL undefined after Cmax). Plasma samples from the psilocybin ODT and capsule groups were analyzed for psilocybin and psilocin.

Pharmacokinetic parameters. Noncompartmental pharmacokinetic parameters were determined from the plasma concentration-time profiles using commercially available software (Phoenix® WinNonlin®).

Results. The data relating to the individual PK parameters are presented in Table 24.

TABLE 24

| | Compound | | | T½ | Tmax | Cmax | AUClast | AUCINF_obs | Vz_F_obs | Cl_F_obs |
|---|---|---|---|---|---|---|---|---|---|---|
| Formulation | Dosed | Analyte | Stats | (hr) | (hr) | (ng/mL) | (hr*ng/mL) | (hr*ng/mL) | (mL/kg) | (mL/kg/hr) |
| ODT | Psilocybin | Psilocin | Mean | 2.06 | 0.25 | 28.9 | 71.8 | 77.0 | 16600 | 5570 |
| | | | SD | 0.475 | 0.25-0.5 | 5.75 | 13.3 | 13.1 | 4420 | 4420 |
| PIC | Psilocybin | Psilocin | Mean | 1.80 | 0.5 | 41 | 89.0 | 93.3 | 12000 | 4580 |
| | | | SD | 0.215 | 0.25-2 | 11.6 | 16.5 | 16.9 | 3400 | 872 |

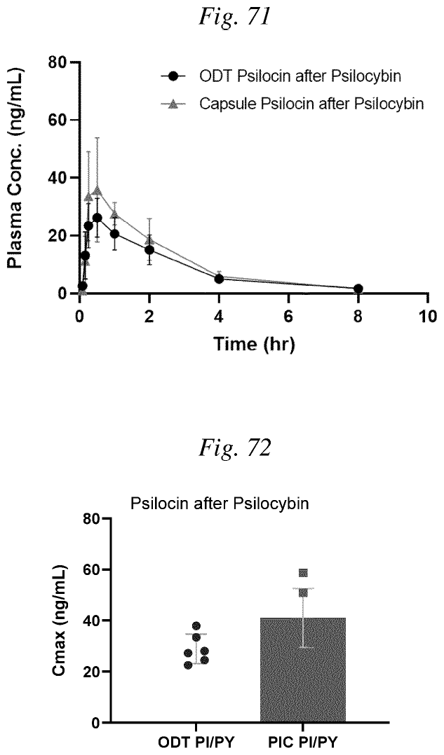

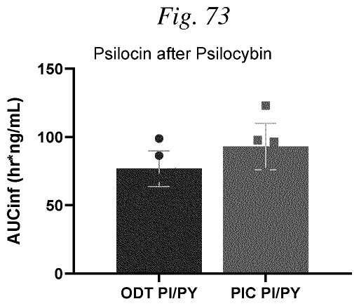

The results are also graphically represented in FIG. 71, which shows the plasma concentration-time profiles for psilocin after psilocybin dosing (ODT and PIC dosage forms), FIG. 72 showing the exposure comparison between psilocybin ODT and PIC dosage forms as assessed by Cmax, and FIG. 73 showing the exposure comparison between psilocybin ODT and PIC dosage forms as assessed by AUCinf.

As can be seen from these graphs, psilocin after psilocybin ODT formulation exposure is not significantly (p>0.5) different than PIC exposure. The ODTs produced a faster onset of action compared to PIC dosage forms as measured by time to maximum plasma concentrations—the time to maximum plasma concentration was twice as fast after ODT compared to PIC.

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

Accordingly, the preceding merely illustrates the principles of the methods and compositions. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles of the disclosure and the concepts contributed by the inventors to furthering the art, and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the following.

1. A pharmaceutical composition, comprising:
a solid dispersion comprising a therapeutically effective amount of a compound of Formula (I) in amorphous form dispersed in a polymer, wherein the compound of Formula (I) has a chemical purity of greater than 97% by high performance liquid chromatography (HPLC), 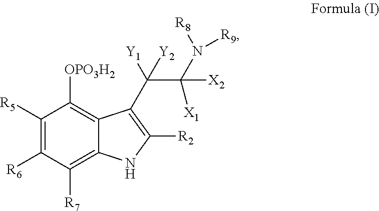
or a pharmaceutically acceptable salt, stereoisomer, a tautomer, or solvate thereof,
wherein:
R₂, R₅, R₆, and R₇ are independently selected from the group consisting of hydrogen and deuterium,
R₈ and R₉ are independently selected from the group consisting of —CH₃ and —CD₃, and
X₁, X₂, Y₁, and Y₂ are independently selected from the group consisting of hydrogen and deuterium.

2-9. (canceled)

10. The pharmaceutical composition of claim 1, wherein the compound of Formula (I) is at least one selected from the group consisting of: 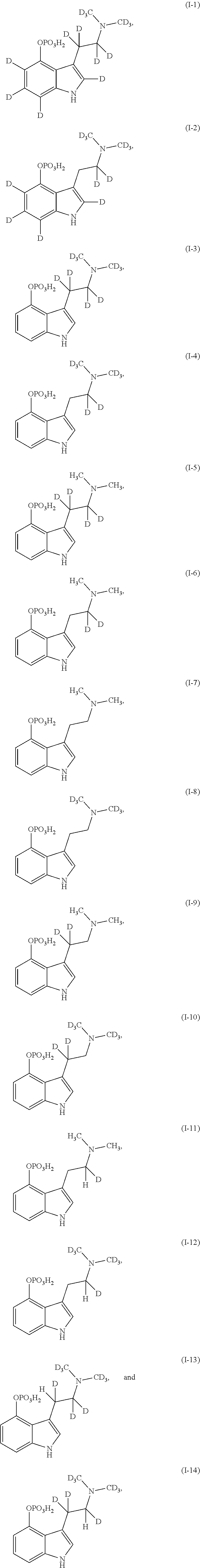
or a pharmaceutically acceptable salt, stereoisomer, a tautomer, or solvate thereof.

11. The pharmaceutical composition of claim 1, wherein the compound of Formula (I) is 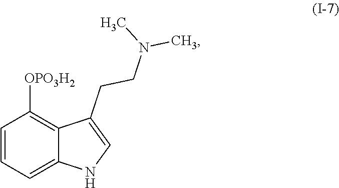
or a pharmaceutically acceptable salt, tautomer, or solvate thereof.

12-14. (canceled)

15. The pharmaceutical composition of claim 1, wherein the compound of Formula (I) is present in the solid dispersion in an amount of 0.1 wt. % to 90 wt. %, based on a total weight of the solid dispersion.

16. The pharmaceutical composition of claim 1, wherein a weight ratio of the compound of Formula (I) to the polymer in the solid dispersion is from 1:9 to 9:1.

17. (canceled)

18. The pharmaceutical composition of claim 1, wherein the polymer is at least one selected from the group consisting of gelatin, polyvinyl acetate, polyvinyl alcohol, polyvinylpyrrolidone, pullulan, hydroxypropyl methyl cellulose, hydroxypropyl methyl cellulose acetate succinate, and a methacrylate copolymer, or a blend or a copolymer thereof.

19. The pharmaceutical composition of claim 1, wherein the polymer comprises gelatin.

20-21. (canceled)

22. The pharmaceutical composition of claim 1, wherein the polymer comprises a copolymer of vinyl pyrrolidone and vinyl acetate (PVP-VAc).

23. The pharmaceutical composition of claim 1, wherein the polymer comprises a methacrylate copolymer.

24. The pharmaceutical composition of claim 1, wherein the polymer comprises a cellulose polymer.

25-26. (canceled)

27. The pharmaceutical composition of claim 24, wherein the cellulose polymer is hydroxypropyl methyl cellulose acetate succinate.

28. The pharmaceutical composition of claim 24, wherein the cellulose polymer is hydroxypropyl methyl cellulose.

29. The pharmaceutical composition of claim 1, wherein the polymer is a blend of hydroxypropyl methyl cellulose and polyvinylpyrrolidone.

30. The pharmaceutical composition of claim 1, further comprising a pharmaceutically acceptable excipient which is not dispersed with the solid dispersion.

31. (canceled)

32. The pharmaceutical composition of claim 1, wherein the solid dispersion has: (i) a glass transition (Tg) onset of from 110° C. to 200° C., as determined by modulated differential scanning calorimetry (mDSC); (ii) a heat capacity change (ΔCp), in J/(g·° C.), of from 0.1 to 0.75, as determined by modulated differential scanning calorimetry (mDSC); or both (i) and (ii).

33-38. (canceled)

39. The pharmaceutical composition of claim 1, which is adapted for oral administration.

40-41. (canceled)

42. A method of treating a subject with major depressive disorder (MDD), comprising:
administering to the subject the pharmaceutical composition of claim 1.

43-46. (canceled)

47. A method of treating a subject with treatment-resistant depression (TRD), comprising:
administering to the subject the pharmaceutical composition of claim 1.

48-51. (canceled)

52. A method of treating a subject with a substance use disorder, comprising:
administering to the subject the pharmaceutical composition of claim 1.

53-72. (canceled)

73. A method of treating a subject with one or more of an anxiety disorder, an eating disorder, and a headache disorder, comprising:
administering to the subject the pharmaceutical composition of claim 1.

74-82. (canceled)
